(19) United States
(12) Patent Application Publication
(10) Pub. No.: US 2004/0142981 A1
(43) Pub. Date: Jul. 22, 2004

(54) Antibacterial benzoic acid derivatives

(76) Inventors: Atli Thorarensen, O'Fallon, MO (US); J. Craig Ruble, Greenwood, IN (US); Donna Lee Romero, Chesterfield, MO (US)

Correspondence Address: PHARMACIA & UPJOHN, 301 HENRIETTA ST, 0228-32-LAW, KALAMAZOO, MI, 49007, US

(21) Appl. No.: 10/645,799

(22) Filed: Aug. 20, 2003

Related U.S. Application Data
(60) Provisional application No. 60/405,614, filed on Aug. 23, 2002.
(60) Provisional application No. 60/413,596, filed on Sep. 25, 2002.
(60) Provisional application No. 60/430,351, filed on Dec. 2, 2002.

(51) Int. Cl. C07D 213/46; C07D 213/57; A61K 31/455; A61K 31/401; C07D 207/46

(52) U.S. Cl. 514/355; 514/423; 514/357; 514/408; 546/330; 548/530; 546/314; 548/566

(57) ABSTRACT

The present invention relates to antibacterial agents that are useful for sterilization, sanitation, antisepsis, disinfection, and treatment of antibacterial infections in mammals.

### CROSS REFERENCE

This application claims the benefit of U.S. Serial Nos. 60/405,614, 60/413,596, and 60/430,351, filed on Aug. 23, 2002, Sep. 25, 2002, and Dec. 2, 2002, under 35 U.S.C. § 119(e)(i).

### FIELD OF THE INVENTION

The present invention relates to antibacterial agents that are useful for sterilization, sanitation, antisepsis, and disinfection.

### BACKGROUND

The inappropriate growth of a variety of bacteria has been a problem for many years. Bacteria have caused degradation of natural product materials, infection in humans and other animals, and spoilage of foods.

Sterilization denotes the use of either physical or chemical agents to eliminate all viable bacteria from a material, while disinfection generally refers to the use of germicidal chemical agents to destroy the potential infectivity of a material. Sanitizing refers to procedures used to simply lower the bacterial content of utensils used for food. Antisepsis refers to the topical application of chemicals to a body surface to kill or inhibit pathogenic microbes. Disinfectants are widely used for skin antisepsis in preparation for surgery.

Bacteria are the smallest organisms that contain all the machinery required for growth and self-replication. A bacterium includes a rigid cell wall surrounding the cytoplasmic membrane, which itself encloses a single naked chromosome without a nuclear membrane. The cytoplasmic membrane consists primarily of a bi-layer of lipid molecules.

The fundamental criterion of bactericidal action is loss of the ability of the organism to propagate indefinitely, when placed in a suitable environment. Bactericidal action suggests microbe damage of various types, including the triggering of irreversible damage to the cytoplasmic cell membrane or irreversible impairment of the DNA (or viral RNA replication. Accordingly, sterilization is not identical with destruction of microbes. Additionally, it is understood that damage to nucleic acids (DNA or RNA) is not always irreversible, as it is known that ultraviolet light-induced damage to viral nucleic acids can be repaired by enzymatic and genetic mechanisms.

### SUMMARY OF THE INVENTION

The invention relates to antibacterial agents that are useful for sterilization, sanitation, antisepsis, and disinfection.

In one aspect, the invention features methods of using antibacterial agents of formula I for sterilizing, sanitizing, antisepsis, or disinfecting. The method includes applying the antibacterial agent to a location in need of sterilization, sanitation, antisepsis, and disinfection. In general, the antibacterial agents have the formula

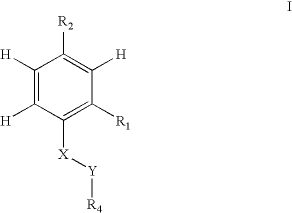

wherein

X═NH

Y═CO, CS, —C(═N—CN) or

X and Y together form an alkene, or C₃-C₅ cycloalkyl;

R₁ is -HET¹, —CO-HET¹, or —NH—S(O)₂-Q₁, the HET¹ being an optionally substituted HET¹;

Q₁ is selected from the group consisting of H, optionally substituted alkyl, or optionally substituted aryl;

R₂ is an electron withdrawing group; and

R₄ is an optionally substituted aryl provided that the aryl is not simultaneously substituted with a sulfonamide and a urea or thiourea, and further provided that the aryl is not solely substituted at the ortho-position relative to Y, or R₄ is an optionally substituted HET².

### DETAILED DESCRIPTION OF THE INVENTION

Mammal denotes human and animals.

The term “halo” refers to a halogen atom selected from Cl, Br, I, and F.

The term “alkyl” refers to both straight- and branched-chain moieties. Unless otherwise specifically stated alkyl moieties include between 1 and 9 carbon atoms.

The term “alkenyl” refers to both straight- and branched-chain moieties containing at least one —C═C—. Unless otherwise specifically stated alkenyl moieties include between 1 and 9 carbon atoms.

The term “alkynyl” refers to both straight- and branched-chain moieties containing at least one —C≡C—.

The term “alkoxy” refers to —O-alkyl groups.

The term “cycloalkyl” refers to a cyclic alkyl moiety. Unless otherwise specifically stated cycloalkyl moieties will include between 3 and 9 carbon atoms.

The term “cycloalkenyl” refers to a cyclic alkenyl moiety. Unless otherwise specifically stated cycloalkyl moieties will include between 3 and 9 carbon atoms and at least one —C═C— group within the cyclic ring.

The term “amino” refers to —NH₂.

The term “aryl” refers to phenyl and naphthyl.

The term “HET¹” refers to mono- or bi-cyclic ring systems containing at least one —N(Q₅₀)— and optionally 1-3 additional heteroatoms selected from O, S, and N. Each Q₅₀ is hydrogen or any prodrug substiutent which may be transformed into or cleaved by an enzymatic or chemical process in vivo such that—N(prodrug substituent)—is converted into —N(H)—. Each mono-cyclic ring may be aromatic, saturated, or partially unsaturated. A bi-cyclic ring system may include a mono-cyclic ring containing at least one heteroatom fused with an cycloalkyl or aryl group. A bi-cyclic ring system may also include a mono-cyclic ring containing at least one heteroatom fused with another het, mono-cyclic ring system.

The expression “prodrug” denotes a derivative of a known direct acting drug, which is transformed into the active drug by an enzymatic or chemical process. Prodrugs of the compounds of formula (I) are prepared by modifying functional groups present on the compound in such a way that the modifications are cleaved, either in routine manipulation or in vivo, to the parent compound. The modifications typically are achieved by synthesizing the parent compound with a prodrug substiutent. Prodrugs include, but are not limited to, compounds of structure (I) wherein hydroxy, amine or sulfhydryl groups are bonded to any group that, when administered to the animal, cleaves to form the free hydroxyl, amino or sulfhydryl group, respectively. Representative examples of prodrugs include, but are not limited to, acetate, formate, —CH2—O—C(O)-Q₁₅, and benzoate derivatives of alcohol and amine functional groups. See Notari, R. E., “Theory and Practice of Prodrug Kinetics,” Methods in Enzymology, 112:309-323 (1985); Bodor, N., “Novel Approaches in Prodrug Design,” Drugs of the Future, 6(3):165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985).

The term “HET²” refers to mono- or bi-cyclic ring systems containing at least one heteroatom selected from O, S, and N. Each mono-cyclic ring may be aromatic, saturated, or partially unsaturated. A bi-cyclic ring system may include a mono-cyclic ring containing at least one heteroatom fused with an cycloalkyl or aryl group. A bi-cyclic ring system may also include a mono-cyclic ring containing at least one heteroatom fused with another het, mono-cyclic ring system.

Examples of “HET²” include, but are not limited to, pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, and azabicyclo[2.2.1]heptyl.

The term “heteroaryl” refers to a mono- or bicylic het in which at least one cyclic ring is aromatic.

The term “substituted alkyl” refers to an alkyl moiety including 1-4 substituents selected from halo, het, cycloalkyl, cycloalkenyl, aryl, —OQ₁₀, —SQ₁₀, —S(O)₂Q₁₀,

—S(O)Q₁₀, —OS(O)₂Q₁₀, —C(═NQ₁₀)Q₁₀, —C(═N—O-Q₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(S)NQ₁₀ Q₁₀, —N(Q ₁₀)C(S)NQ₁₀Q₁₀, —C(O)NQ₁₀Q ₁₀, —C(S)NQ₁₀ Q ₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, and —SNQ₁₀Q₁₀. Each of the het, cycloalkyl, cycloalkenyl, and aryl being optionally substituted with 1-4 substituents independently selected from halo and Q₁₅.

The term “substituted aryl” refers to an aryl moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, —NQ₁₀C(O)Q₁₀, —N(Q₁₀)C(S)NQ₁₀Q₁₀, —N(Q₁₀)C(S)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀,

—NO₂, —SNQ₁₀Q₁₀, alkyl, substituted alkyl, alkenyl, alkynyl, het, halo, cycloalkyl, cycloalkenyl, and aryl. The het, cycloalkyl, cycloalkenyl, alkenyl, alkynyl, and aryl being optionally substituted with 1-3 substitutuents selected from halo and Q₁₅.

The term “substituted HET¹” or “substituted HET²” refers to a HET, or HET² moiety including 1-4 substituents selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(O)Q₁₀, —NQ₁₀C(S)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅

The term “substituted alkenyl” refers to a alkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted alkoxy” refers to an alkoxy moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted cycloalkenyl” refers to a cycloalkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₀Q₁₀, —C(S)NQ₁₀Q₁₀, —C(O)C(Q₁₆)₂OC(O)Q₁₀, —CN, ═O, ═S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted amino” refers to an amino moiety in which one or both of the amino hydrogens are replaced with a group selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(O)Q₁₀, —NQ₁₀C(S)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

Each Q₁₀ is independently selected from —H, alkyl, cycloalkyl, het, cycloalkenyl, and aryl. The het, cycloalkyl, cycloalkenyl, and aryl being optionally substituted with 1-3 substitutuents selected from halo and Q₁₃.

Each Q₁₁ is independently selected from —H, halo, alkyl, aryl, cycloalkyl, and het. The alkyl, aryl, cycloalkyl, and het being optionally substituted with 1-3 substituents independently selected from halo, —NO₂, —CN, ═S, ═O, and Q₁₄.

Each Q₁₃ is independently selected from Q₁₁, —OQ₁₁, —SQ₁₁, —S(O)₂Q₁₁, —S(O)Q₁₁, —OS(O)₂Q₁₁, —C(═NQ₁₁)Q₁₁, —S(O)₂—N═S(O)(Q ₁₁)₂, —S(O)₂—N═S(Q₁₁)₂, —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —C(S)NQ₁₁Q₁₁, —C(O)C(Q₁₆)₂OC(O)Q₁₀, —CN, ═O, ═S, —NQ₁₁C(O)Q₁₁, —NQ₁₁C(S)Q₁₁, —NQ₁₁C(O)NQ₁₁Q₁₁, —NQ₁₁C(S)NQ₁₁Q₁₁, —S(O)₂NQ₁₁Q₁₁, —NQ ₁₁S(O)₂Q₁₁, —NQ₁₁S(O)Q₁₁, —NQ₁₁SQ₁₁, —NO₂, and —SNQ₁₁Q₁₁.

Each Q₁₄ is —H or a substituent selected from alkyl, cycloalkyl, phenyl, or naphthyl, each optionally substituted with 1-4 substituents independently selected from

—F, —Cl, —Br, —I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —C(S)NQ₁₆Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, and —NQ₁₆S(O)₂Q₁₆. The alkyl, cycloalkyl, and cycloalkenyl being further optionally substituted with ═O or ═S.

Each Q₁₅ is alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ ₁₆Q₁₆, —C(S)NQ₁₆Q₁₆, —C(O)C(Q₁₆)₂OC(O)Q₁₆, —CN,

—NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S.

Each Q₁₆ is independently selected from —H, alkyl, and cycloalkyl. The alkyl and cycloalkyl optionally including 1-3 halos.

Each Q₁₇ is independently selected from —H, —OH, and alkyl optionally including 1-3 halos, —OH, and —OQ₁₆.

The term “electron withdrawing group” refers to the ability of a substituent to withdraw electrons relative to that of hydrogen if the hydrogen atom occupied the same position on the molecule. The term “electron withdrawing group” is well understood by one skilled in the art and is discussed in Advanced Organic Chemistry by J. March, John Wiley & Sons, New York, N.Y., (1985) and the discussion therein is incorporated herein by reference. Electron withdrawing groups include, but are not limited to, groups such as halo, nitro, carboxy, cyano, aryl optionally substituted, aromatic het (excluding pyridine) optionally substituted, —OC(Zₙ)₃, —C(Zₙ)₃, —C(Zₙ)₂—O—C(Zₘ)₃, —(CO)-Q₁₇, —SO₂—C(Zₙ)₃, —SO₂-aryl, —C(NQ₁₇)Q₁₇, —CH═C(Q₁₇)₂, —C≡C-Q₁₇, in which each Zₙ and Zₘ is independently H, halo, —CN, —NO₂—OH, or C₁₋₄alkyl optionally substituted with 1-3 halo, —OH, NO₂, and provided that at least one of Zₙ is halo, —CN, or NO₂, and further provided that Q₁₇ is not —OH when the the electron withdrawing group is —(CO)-Q₁₇.

It is to be understood that the present invention encompasses any racemic, optically-active, polymorphic, tautomeric, or stereoisomeric form, or mixture thereof, of a compound of the invention, which possesses the useful properties described herein.

In cases where compounds are sufficiently basic or acidic to form stable nontoxic acid or base salts, use of the compounds as pharmaceutically acceptable salts may be appropriate. Examples of pharmaceutically acceptable salts which are within the scope of the present invention include organic acid addition salts formed with acids which form a physiological acceptable anion and inorganic salts. Examples of pharmaceutically acceptable salts include, but are not limited to, the following acids acetic, aspartic, benzenesulfonic, benzoic, bicarbonic, bisulfuric, bitartaric, butyric, calcium edetate, camsylic, carbonic chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic toluenesulfonic, primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The antibacterial agents of this invention have useful activity against a variety of organisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA.

The antibacterial agents described herein are useful for sterilization, sanitation, antisepsis, and disinfection. The antibacterial agents can be applied to a location in need of sterilization, sanitation, antisepsis, or disinfection, by methods known to those skilled in the art. For instance, the antibacterial agents may be incorporated into a cleaning solution that is applied, such as by spraying or pouring, to an item in need of sterilization, sanitation, antisepsis, or disinfection. The antibacterial agents may be used alone or in combination, e.g., agents disclosed herein with one another or agent(s) disclosed herein with other antibacterial agents. The antibacterial agents may be applied in varying concentrations depending upon the bacterial susceptibility to antibacterial agent(s) being applied and the desired level of sterilization, sanitation, antisepsis, or disinfection.

In other embodiments, certain antibacterial agents described herein are useful for treating microbial infections in mammals, such as by administering an effective amount of the antibacterial agent compound to the mammal.

The antibacterial agent may be incorporated into a pharmaceutical composition.

The pharmaceutical compositions of this invention may be prepared by combining the compounds of this invention with a solid or liquid pharmaceutically acceptable carrier and, optionally, with pharmaceutically acceptable adjuvants and excipients employing standard and conventional techniques. Solid form compositions include powders, tablets, dispersible granules, capsules, cachets and suppositories. A solid carrier can be at least one substance which may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the compounds of this invention dissolved in water and water-propylene glycol systems, optionally containing suitable conventional coloring agents, flavoring agents, stabilizers and thickening agents.

Preferably, the pharmaceutical composition is provided employing conventional techniques in unit dosage form containing effective or appropriate amounts of the active component, that is, the compound according to this invention.

The quantity of active component, that is the compound according to this invention, in the pharmaceutical composition and unit dosage form thereof may be varied or adjusted widely depending upon the particular application, the potency of the particular compound and the desired concentration. Generally, the quantity of active component will range between 0.5% to 90% by weight of the composition.

In therapeutic use for treating, or combatting, bacterial infections in warm-blooded animals, the compounds or pharmaceutical compositions thereof will be administered orally, parenterally and/or topically at a dosage to obtain and maintain a concentration, that is, an amount, or blood-level of active component in the animal undergoing treatment which will be antibacterially effective. Generally, such antibacterially effective amount of dosage of active component will be in the range of about 0.1 to about 100, more preferably about 3.0 to about 50 mg/kg of body weight/day. It is to be understood that the dosages may vary depending upon the requirements of the patient, the severity of the bacterial infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., 2-4 four times per day.

The compounds according to this invention may be administered parenterally, i.e., by injection, for example, by intravenous injection or by other parenteral routes of administration. Pharmaceutical compositions for parenteral administration will generally contain a pharmaceutically acceptable amount of the compound or a soluble salt (acid addition salt or base salt) dissolved in a pharmaceutically acceptable liquid carrier such as, for example, water-for-injection and a buffer to provide a suitably buffered isotonic solution, for example, having a pH of about 3.5-6. Suitable buffering agents include, for example, trisodium orthophosphate, sodium bicarbonate, sodium citrate, N-methylglucamine, L(+)-lysine and L(+)-arginine to name but a few representative buffering agents. The compound of this invention generally will be dissolved in the carrier in an amount sufficient to provide a pharmaceutically acceptable injectable concentration in the range of about 1 mg/mL to about 400 mg/mL of solution. The resulting liquid pharmaceutical composition will be administered so as to obtain the above-mentioned antibacterially effective amount of dosage. The compounds according to this invention are advantageously administered orally in solid and liquid dosage forms.

As a topical treatment an effective amount of Formula I is admixed in a pharmaceutically acceptable gel or cream vehicle that can be applied to the patient's skin at the area of treatment. Preparation of such creams and gels is well known in the art and can include penetration enhancers.

The antibacterial agents of this invention have useful activity against a variety of organisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA.

In some embodiments, the antibacterial compounds are prodrugs of the compounds of formula I. The expression “prodrug” denotes a derivative of a known direct acting drug, which is transformed into the active drug by an enzymatic or chemical process. Prodrugs of the compounds of formula I are prepared by modifying functional groups present on the compound in such a way that the modifications are cleaved, either in routine manipulation or in vivo, to the parent compound. Prodrugs include, but are not limited to, compounds of structure (I) wherein hydroxy, amine or sulfhydryl groups are bonded to any group that, when administered to the animal, cleaves to form the free hydroxyl, amino or sulfhydryl group, respectively. Representative examples of prodrugs include, but are not limited to, acetate, formate and benzoate derivatives of alcohol and amine functional groups. See Notari, R. E., “Theory and Practice of Prodrug Kinetics,” Methods in Enzymology, 112:309-323 (1985); Bodor, N., “Novel Approaches in Prodrug Design,” Drugs of the Future, 6(3):165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985).

The antibacterial compounds of this invention may be synthesized by various methods known to those skilled in the art. Non-limiting examples of synthetic schemes for producing the antibacterial agents are described below.

### EXAMPLES

Without further elaboration, it is believed that one skilled in the art can, using the preceding description, practice the present invention to its fullest extent. The Allowing detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

Preparation of Methyl 5-bromo-2-({3-[(5-chloro-2,3-dihydro-1H-indol-1-yl)sulfonyl]benzoyl}amino)benzoate

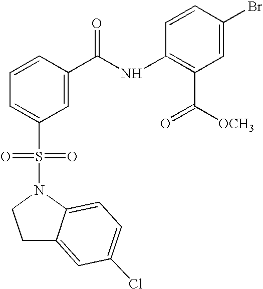

3-[(5-Chloro-2,3-dihydro-1H-indol-1-yl)sulfonyl]benzoic acid, 1.0 g, 2.96 mmol) was suspended in dry CH₂Cl₂ (20 mL) under N₂ and treated with DMF (5 μL) followed by oxalyl chloride (0.5 mL, 5.70 mmol). Gas evolved as the mixture rapidly became homogenous. After stirring for 40 minutes at RT, the solvent and excess oxalyl chloride were evaporated and the resultant residue was taken up in CH₂Cl₂ (20 mL). Methyl 2-amino-5-bromobenzoate (656 mg, 2.85 mmol, Avocado) was added as a solution in dry pyridine (5 mL) and the amber solution was stirred at RT until TLC showed the absence of the aniline. The reaction was diluted to 300 mL with CH₂Cl₂, and was washed 2× with 1.0M HCl (150 mL) and 1× with brine (200 mL). The organic layer was dried over MgSO₄, filtered and the solvent was evaporated. The resultant residue was purified on a Biotage Flash 40M (90 g) silica cartridge using CH₂Cl₂. The solvent was evaporated and the resultant product dried under vacuum at 120° C. to afford 1.22 g (78%) of white solid. ¹H NMR (400 MHz, CDCl₃) δ 12.07 (s, 1H), 8.79 (d, J=8.6 Hz, 1H), 8.50 (s, 1H), 8.19-8.23 (m, 2H), 7.95 (d, J=7.6 Hz, 1H), 7.72 (dd, J=9.2, 2.5 Hz, 1H), 7.59-7.66 (m, 2H), 7.16 (d, J=8.6 Hz, 1H), 7.05 (s, 1H), 4.05 (t, J=8.4 Hz, 2H), 4.00 (s, 3H), 2.94 (t, J=8.4 Hz, 2H).

Preparation of 5-Bromo-2-({3-[(5-chloro-2,3-dihydro-1H-indol-1-yl)sulfonyl]benzoyl}amino) benzoic acid

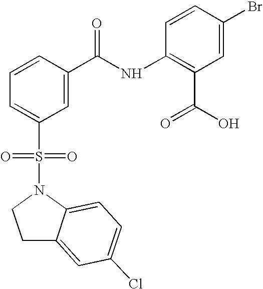

The corresponding methyl ester, 550 mg, 1.0 mmol) was dissolved in dioxane (10 mL), and was treated with 1.0M KOH (5.0 mL, 5.0 mmol). The resultant yellow solution was heated to 40° C., and after stirring for one hour, the reaction was complete by HPLC. The solution was poured into CH₂Cl₂ (150 mL), and then washed 2× with 1.0M HCl, and 1× with brine (150 mL each). The product precipitated but remained in the organic layer during the workup and re-dissolved upon equilibration with brine. The organics were dried over MgSO₄, filtered and the solvent evaporated. The crude product was re-crystallized from hot acetonitrile/THF and the resultant product was dried at 100° C. under vacuum to afford 243 mg (45%) of peach colored crystals. ¹H NMR (400 MHz, DMSO-d₆) δ 12.19 (s, 1H), 8.58 (d, J=9.2 Hz, 1H), 8.36 (s, J=1 Hz, 1H), 8.23 (d, J=8.1 Hz, 1H), 8.14 (d, J=2.5 Hz, 1H), 8.06 (d, J=8.1 Hz, 1H), 7.88 (dd, J=9.2, 2.5 Hz, 1H), 7.82 (t, J=7.9 Hz, 1H), 7.51 (d, J=8.6 Hz, 1H), 7.27 (d, J=8.6 Hz, 1H), 7.24 (s, 1H), 4.01 (t, J=8.4 Hz, 2H), 2.95 (t, J=8.4 Hz, 2H).

### Example 1

### N-[4-Bromo-2-(5-oxo-4,5-dihydro-1,2,4-oxadiazol-3-yl)phenyl]-3-[(5-chloro-2,3-dihydro-1H-indol-1-yl)sulfonyl]benzamide

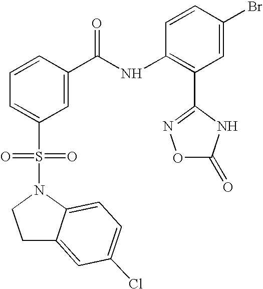

3-[(5-Chloro-2,3-dihydro-1H-indol-1-yl)sulfonyl]benzoic acid, 338 mg, 1.05 mmol) was suspended in dry CH₂Cl₂ (7 mL) under N₂ and treated with DMF (5 μL) followed by oxalyl chloride (0.166 mL, 2.10 mmol). Gas evolved as the mixture rapidly became homogenous. After stirring for one hour at RT, the solvent and excess oxalyl chloride were evaporated and the resultant residue was taken up in CH₂Cl₂ (7 mL). 3-(2-Amino-5-bromophenyl)-1,2,4-oxadiazol-5(4H)-one, 252 mg, 1.0 mmol) was added as a solution in dry pyridine (3 mL) and the amber solution was stirred at RT for 2 hours during which time some crystalline material appeared. The reaction was diluted to 100 mL with CH₂Cl₂, and washed 1× with 1.0M HCl (75 mL) which caused the product crystallized out of the organic layer. The organic layer was separated, chilled, and then evaporated to dryness. The resultant residue was re-crystallized from toluene/THF and the product dried under vacuum at 120° C. to afford 357 g (65%) of white solid. ¹H NMR (400 MHz, DMSO-d₆) δ 12.92 (s, 1H), 10.63 (s, 1H), 8.39 (s, 1H), 8.21 (d, J=7.6 Hz, 1H), 8.03 (d, J=7.6 Hz, 1H), 7.99 (d, J=8.6 Hz, 1H), 7.92 (s, 1H), 7.86 (d, J=8.6 Hz, 1H), 7.78 (t, J=7.9 Hz, 1H), 7.48 (d, J=9.2 Hz, 1H), 7.23-7.25 (m, 2H), 4.04 (t, J=8.4 Hz, 2H), 2.95 (t, J=8.4 Hz, 2H).

### Example 2

### 3-(Benzylthio)-N-[4-bromo-2-(5-oxo-4,5-dihydro-1,2,4-oxadiazol-3-yl)phenyl]benzamide

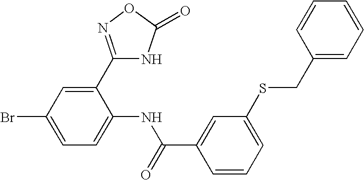

To 3-(benzylthio)benzoic acid, described by Behagnel, O.et al. in Chem. Ber. 1939, 6, 1257-1268, (387 mg, 1.58 mmol) in CH₂Cl₂ (25 mL) was added DMF (15 μL) and oxalyl chloride (165 μL, 1.89 mmol). The mixture was stirred for 2 hours, and the solvent and excess oxalyl chloride were removed by rotary evaporation. The residue was dissolved in CH₂Cl₂ (15 mL), and 3-(2-amino-5-bromophenyl)-1,2,4-oxadiazol-5(4H)-one (396 mg, 1.55 mmol) in pyridine (6 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ layer was then cooled in a freezer. The resulting precipitate was washed with pentane and recrystallized from 15 mL of hot ethanol. The crystals were washed with ethanol followed by heptane and then dried at 100° C. under vacuum yielding 328 mg of white solid. ¹H NMR (400 MHz, DMSO-d₆) δ 12.9 (br s, 1H), 10.47 (s, 1H), 8.07 (d, J=9.2 Hz, 1H), 7.92 (d, J=2.5 Hz, 1H), 7.9 (s, 1H), 7.85 (dd, J=8.6, 2.5 Hz, 1H), 7.71 (d, J=7.6 Hz, 1H), 7.57 (d, J=7.6 Hz, 1H), 7.48 (t, J=7.6 Hz, 1H), 7.39 (d, J=7.1 Hz, 2H), 7.31 (t, J=7.4 Hz, 2H), 7.24 (t, J=7.4 Hz, 2H), 4.34 (s, 1H).

### Example 3

### N-(4-Bromo-2-{[(methylsulfonyl)amino]carbonyl}phenyl)-3-{[(4-chlorophenyl)(methyl)amino]sulfonyl}benzamide

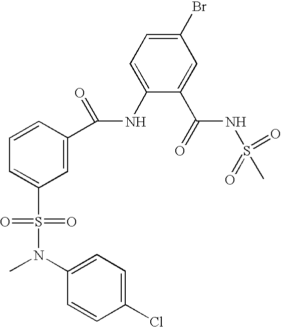

To a flask containing 5-bromo-2-[(3-{[(4-chlorophenyl)(methyl)amino]sulfonyl}benzoyl)amino]benzoic acid (843 mg, 1.61 mmol) and 1,1′-carbonyldiimidazole (505 mg, 3.11 mmol, Aldrich) was added THF (10 mL). This mixture was heated at reflux for 1.5 hours, after which methanesulfonamide (320 mg, 3.36 mmol, Aldrich) was added. The mixture was kept at reflux an additional 88 hours and then evaporated in the presence of silica gel, and the product was purified by chromatography using a Biotage Flash 40 M silica cartridge with a gradient from CH₂Cl₂ to 1% acetic acid/CH₂Cl₂ to 2% methanol/1% acetic acid/97% CH₂Cl₂ as eluent. Product was collected and dried at 100° C. under vacuum yielding 427 mg of white solid. ¹H NMR (of the benzamide) (400 MHz, DMSO-d₆) δ 11.5 (br s, 1H), 8.31 (d, J=7.8 Hz, 1H), 8.16 (t, J=1.6 Hz, 1H), 7.88-7.95 (m, 2H), 7.75-7.82 (m, 2H), 7.66 (d, J=8.6 Hz, 1H), 7.43 (d, J=8.8 Hz, 2H), 7.17 (d, J=8.8 Hz, 2H), 3.24 (s, 3H), 3.11 (s, 3H).

### Example 4

### N-[4-Bromo-2-({[(4-methylphenyl)sulfonyl]amino}carbonyl)phenyl]-3-{[(4-chlorophenyl)(methyl)amino]sulfonyl}benzamide

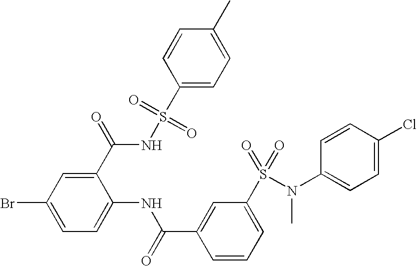

To a flask containing 5-bromo-2-[(3-{[(4-chlorophenyl) (methyl)amino]sulfonyl}benzoyl)amino]benzoic acid (878 mg, 1.68 mmol) and 1,1′-carbonyldiimidazole (676 mg, 4.17 mmol, Aldrich) and p-toluenesulfonamide (604 mg, 3.53 mmol, Aldrich) was added THF (20 mL). This mixture was heated at reflux overnight, after which pyridine (10 mL) and 4-dimethylaminopyridine (300 mg) were added. The mixture was kept at reflux for an additional 90 hours and then added to a separatory funnel with 100 mL of CH₂Cl₂. The CH₂Cl₂ was washed with 2×100 mL of 1 M aqueous HCl followed by 100 mL of water. The CH₂Cl₂ was evaporated in the presence of silica gel, and the product was purified by chromatography using a Biotage Flash 40 M silica cartridge with a gradient from CH₂Cl₂ to 1% acetic acid/CH₂Cl₂ to 2% methanol/1% acetic acid/97% CH₂Cl₂ as eluent. Product was collected and dried at 100° C. under vacuum yielding 934 mg of white solid. ¹H NMR (300 MHz, DMSO-d₆) δ 11.8 (br s, 1H), 8.14 (d, J=7.2 Hz, 1H), 8.09 (s, 1H), 7.89-7.96 (m, 2H), 7.71-7.83 (m, 4H), 7.66 (d, J=7.9 Hz, 1H), 7.43 (d, J=8.7 Hz, 2H), 7.29 (d, J=7.9 Hz, 2H), 7.17 (d, J=8.7 Hz, 2H), 3.18 (s, 3H), 2.31 (s, 3H).

Preparation of N-(4-Bromo-2-cyanophenyl)-4-{[(4-chlorophenyl)(methyl)amino]sulfonyl}benzamide

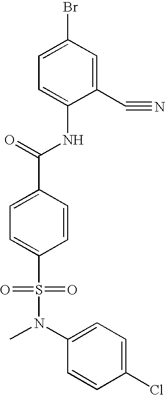

A solution of 4-{[(4-chlorophenyl)(methyl)amino]sulfonyl}benzoyl chloride (245 mg, 0.712 mmol) in CH₂Cl₂ (4 mL) was added to a solution of 2-amino-5-bromobenzonitrile (151 mg, 0.766 mmol) in pyridine (5 mL). After stirring overnight, the solvents were removed by rotary evaporation, and the residue was added to a separatory funnel with 100 mL of CH₂Cl₂. This solution was washed with 2×100 mL of 1 M aqueous HCl and 100 mL of brine. It was dried over MgSO₄ and evaporated. The crude product was adsorbed onto silica gel and purified by chromatography using a Biotage Flash 40 S silica cartridge with a gradient from 10%-25% EtOAc in heptane. Product was isolated as 278 mg of white solid.

### Example 5

### N-[4-Bromo-2-(1H-tetraazol-5-yl)phenyl]-4-{[(4-chlorophenyl)(methyl)amino]sulfonyl}benzamide

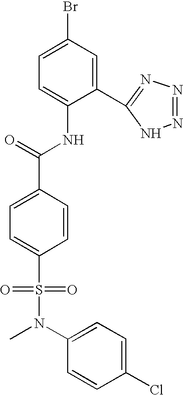

A mixture of the nitrile (324 mg, 0.642 mmol), sodium azide (77 mg, 1.18 mmol), and triethylamine hydrochloride (160 mg, 1.16) in toluene (4 mL) was placed under nitrogen and heated in a 100° C. oil bath overnight. An additional 30 mL of toluene was added, and the mixture was washed with 30 mL of water followed by 30 mL of 1 M NaOH. This resulted in a precipitate. CH₂Cl₂ (100 mL) was added, and the pH of the aqueous layer was adjusted to 1 with HCl. Most of the solid dissolved. The organics were dried over MgSO₄ and evaporated leaving the product as 97.6 mg of white solid. ¹H NMR (400 MHz, Acetone-d₆) δ 12.00 (s, 1H), 8.90 (d, J=9.1 Hz, 1H), 8.32-8.38 (m, 3H), 8.24-8.46 (m, 3H), 7.41 (d, J=8.8 Hz, 2H), 7.24 (d, J=8.7 Hz, 2H), 3.30 (s, 3H).

Preparation of 2-Amino-5-bromo-N′-hydroxybenzenecarboximidamide

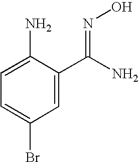

To 2-amino-5-bromobenzonitrile, (this material is commercially available from Acros Organics but may be produced by bromination of anthranilonitrile (Aldrich) with NBS), (4.55 g, 23.1 mmol) was added hydroxylamine hydrochloride (1.80 g, 25.9 mmol) in a minimal amount of water. Ethanol (20 mL) was added, followed by sodium methoxide (6 mL of 25 wt. % in methanol). The mixture was heated at reflux for 16 hours. After the reaction had cooled, 50 mL of 3 M NaOH was added, and the ethanol was removed by rotary evaporation. The mixture was added to a separatory funnel and washed with 2×30 mL of CH₂Cl₂. The aqueous layer was adjusted to pH=4 with HCl. The resulting precipitate was collected, washed with water, and recrystallized from toluene/ethanol. The resulting yellow needles were collected, washed with heptane, and dried under vacuum yielding 2.97 g of product. ¹H NMR (400 MHz, CD₃CN) δ 7.73 (s, 1H), 7.49 (d, J=2.3 Hz, 1H), 7.21 (dd, J=8.7, 2.4 Hz, 1H), 6.66 (d, J=8.7 Hz, 1H), 5.70 (br s, 2H), 5.17 (br s, 2H).

Preparation of 3-(2-Amino-5-bromophenyl)-1,2,4-oxadiazol-5 (4H)-one

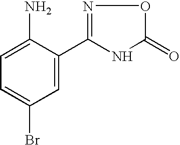

2-Amino-5-bromo-N′-hydroxybenzenecarboximidamide (1.38 g, 6.01 mmol) was dissolved in EtOH (30 mL) and treated with sodium methoxide solution (2.75 mL, 12.0 mmol, 25 wt % in methanol) and diethylcarbonate (3.13 mL, 25.9 mmol). A white precipitate appeared upon heating, and the mixture was refluxed overnight. When HPLC indicated no SM left, the alcohol was evaporated from the cloudy yellow solution; and the resultant solid was dissolved in water (150 mL). The product was precipitated with concentrated HCl (pH<3) and collected by vacuum filtration. After dissolving the crude product in methanol and evaporating the solvent, drying under vacuum at 100° C. afforded 1.48 g (96%) of fluffy tan solid. ¹H NMR (400 MHz, CD₃CN) δ ppm 7.50 (d, J=2.28 Hz, 1H), 7.37 (dd, J=8.71, 2.28 Hz, 1H), 6.80 (d, J=8.71 Hz, 1H).

### Example 6

### N-[4-Bromo-2-(5-oxo-4,5-dihydro-1,2,4-oxadiazol-3-yl)phenyl]-4-{[(4-chlorophenyl)(methyl)amino]sulfonyl}benzamide

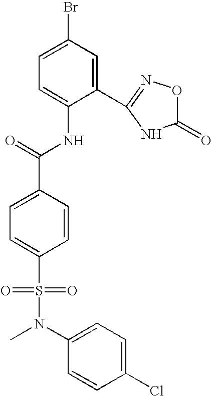

A solution of 4-{[(4-chlorophenyl)(methyl)amino]sulfonyl}benzoyl chloride (462 mg, 1.34 mmol) in CH₂Cl₂ (3 mL) was added to a solution of 3-(2-amino-5-bromophenyl)-1,2,4-oxadiazol-5 (4H)-one (300 mg, 1.17 mmol) in pyridine (3 mL). After stirring overnight, the solvents were removed by rotary evaporation, and the residue was added to a separatory funnel with 150 mL of CH₂Cl₂. This solution was washed with 100 mL of 1 M aqueous HCl after which a precipitate began to form in the organic layer. The precipitate was collected and dried at 100° C. under vacuum yielding 396 mg of off-white solid. ¹H NMR (400 MHz, DMSO-d₆) δ 10.65 (s, 1H), 8.08 (d, J=8.4 Hz, 2H), 7.90-7.95 (m, 2H), 7.85 (d, J=8.7 Hz, 1H), 7.71 (d, J=8.4 Hz, 2H), 7.43 (d, J=8.7 Hz, 2H), 7.17 (d, J=8.7 Hz, 2H), 3.18 (s, 3H).

Preparation of 4-(2,3-Dihydro-1H-indol-1-ylsulfonyl)benzoic acid

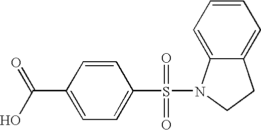

A chilled solution of indoline (3.22 g, 27.0 mmol, Aldrich) and triethylamine (3.0 mL, 22 mmol) in methanol (10 mL) was added by cannula to solid 4-(chlorosulfonyl)benzoic acid (4.04 g, 18.3 mmol, Aldrich) with stirring in an ice bath. The ice bath was removed after 30 minutes, and the mixture was stirred at room temperature for an additional 4.5 hours. The mixture was added to a separatory funnel with 90 mL of 1 M aqueous NaOH, and this solution was washed with 2×75 mL of CH₂Cl₂. The aqueous layer was then acidified with concentrated HCl. The resulting precipitate was collected, washed with water, and recrystallized from hot ethanol. The crystals were collected, washed with pentane, and dried at 120° C. under vacuum. Yield was 4.6 g of yellow-brown crystalline solid.

Preparation of 4-(2,3-Dihydro-1H-indol-1-ylsulfonyl)benzoyl chloride

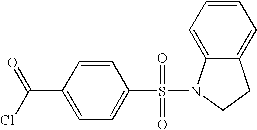

To a slurry of the carboxylic acid (3.26 g, 10.7 mmol) in CH₂Cl₂ (60 mL) was added DMF (3 drops) followed by oxalyl chloride (1.8 mL, 21 mmol). The resulting solution was stirred overnight and then concentrated by rotary evaporation yielding 3.46 g (100%) of a tan solid that was carried on without further purification.

### Example 7

### N-[4-Bromo-2-(5-oxo-4,5-dihydro-1,2,4-oxadiazol-3-yl)phenyl]-4-(2,3-dihydro-1H-indol-1-ylsulfonyl)benzamide

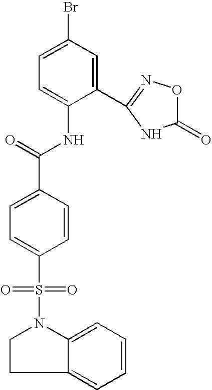

A solution of 4-(2,3-dihydro-1H-indol-1-ylsulfonyl)benzoyl chloride (282 mg, 0.876 mmol) in CH₂Cl₂ (4 mL) was added to a solution of 3-(2-amino-5-bromophenyl)-1,2,4-oxadiazol-5(4H)-one (194 mg, 0.758 mmol) in pyridine (4 mL). After stirring overnight, the mixture was added to a separatory funnel with 125 mL of CH₂Cl₂. This solution was washed with 2×100 mL of 1 M aqueous HCl and 100 mL of brine after which a precipitate began to form in the organic layer. The precipitate was collected, washed with CH₂Cl₂ and pentane, and dried at 100° C. under vacuum yielding 285 mg (69%) of white solid. ¹H NMR (400 MHz, DMSO-d₆) δ 10.58 (s, 1H), 8.05 (d, J=8.1 Hz, 2H), 8.01 (d, J=8.1 Hz, 2H), 7.89-7.92 (m, 2H), 7.84 (dd, J=8.7, 2.5 Hz, 1H), 7.50 (d, J=8.1 Hz, 1H), 7.21 (t, J=7.6 Hz, 1H), 7.17 (d, J=7.1 Hz, 1H), 7.00 (t, J=7.4 Hz, 1H), 3.98 (t, J=8.4 Hz, 2H), 2.94 (t, J=8.4 Hz, 2H).

Preparation of N-[4-Bromo-2-(hydrazinocarbonyl)phenyl]-4-{[(4-chlorophenyl)(methyl)amino]sulfonyl}benzamide

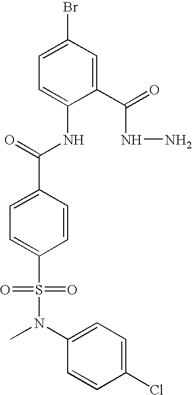

Hydrazine hydrate (1.0 mL) was added to a slurry of the methyl ester (1.12 g, 2.08 mmol) in ethanol (60 mL), and the mixture was heated in an 85° C. oil bath. The solids did not dissolve, so dioxane (10 mL) was added after 5 minutes. The solids still did not dissolve, so it was left to stir overnight. This resulted in a clear solution, but TLC indicated ester was still present, so additional hydrazine hydrate (1.0 mL) was added, and the mixture was again heated overnight. The ethanol was removed by rotary evaporation, and the residue was taken up in 200 mL of CH₂Cl₂. This was washed with 200 mL of water followed by 200 mL of brine, and the CH₂Cl₂ was evaporated in the presence of silica gel. The product was purified on a Biotage Flash 40 M silica cartridge with a gradient from 25%-50% ethyl acetate in heptane. The product was the more polar of the two new spots that were present. It was isolated as 596 mg of white solid.

### Example 8

### N-[4-Bromo-2-(5-thioxo-4,5-dihydro-1,3,4-oxadiazol-2-yl)phenyl]-4-{[(4-chlorophenyl)(methyl)amino]sulfonyl}benzamide

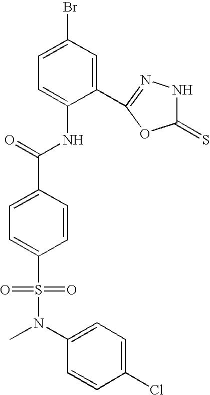

To a solution of the acyl hydrazide (203 mg, 0.378 mmol) in THF (20 mL) was added DBU (130 μL, 0.869 mmol) followed by thiophosgene (32 μL, 0.42 mmol) resulting in a white precipitate. The reaction mixture was stirred overnight and then evaporated in the presence of silica gel. Product was purified on a Biotage Flash 40 s silica cartridge with ethyl acetate as eluent. Product was isolated as 156 mg of light yellow solid. ¹H NMR (400 MHz, DMSO-d₆) δ 10.83 (s, 1H), 8.14 (d, J=8.1 Hz, 2H), 8.02 (d, J=8.7 Hz, 1H), 7.96 (d, J=2 Hz, 1H), 7.86 (dd, J=8.7, 2.0 Hz, 1H), 7.69 (d, J=8.1 Hz, 2H), 7.44 (d, J=8.7 Hz, 2H), 7.17 (d, J=8.7 Hz, 2H), 3.18 (s, 3H).

Preparation of 4-[(5-Chloro-2,3-dihydro-1H-indol-1-yl)sulfonyl]benzoic acid

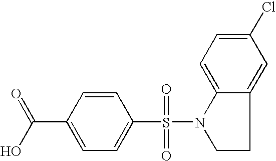

A chilled solution of 5-chloroindoline (2.37 g, 15.5 mmol) and triethylamine (2.2 mL, 16 mmol) in methanol was added by cannula to solid 4-(chlorosulfonyl)benzoic acid (2.72 g, 12.3 mmol, Aldrich) with stirring in an ice bath. The mixture was allowed to warm slowly to room temperature and stirred there overnight. The mixture was added to a separatory funnel with 60 mL of 1 M aqueous NaOH, and this solution was washed with 2×60 mL of CH₂Cl₂. The aqueous layer was then acidified with concentrated HCl. The resulting precipitate was collected, washed with water, and recrystallized from hot ethanol. The crystals were collected, washed with heptane, and dried at 100° C. under vacuum. Yield was 2.92 g of white solid.

Preparation of 4-[(5-Chloro-2,3-dihydro-1H-indol-1-yl)sulfonyl]benzoyl chloride

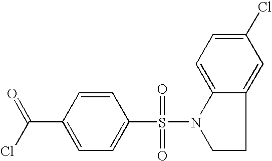

To a slurry of the carboxylic acid (2.13 g, 6.30 mmol) in CH₂Cl₂ (50 mL) was added DMF (2 drops) followed by oxalyl chloride (1.1 mL, 13 mmol). The resulting solution was stirred overnight and then concentrated by rotary evaporation yielding 2.26 g (101%) of yellow solid that was carried on without further purification.

Preparation of N-(4-Bromo-2-cyanophenyl)-4-[(5-chloro-2,3-dihydro-1H-indol-1-yl)sulfonyl]benzamide

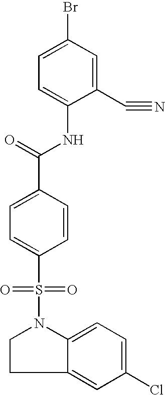

A solution of 4-[(5-chloro-2,3-dihydro-1H-indol-1-yl)sulfonyl]benzoyl chloride (767 mg, 2.15 mmol) in CH₂Cl₂ (8 mL) was added to a solution of 2-amino-5-bromobenzonitrile (354 mg, 1.80 mmol) in pyridine (6 mL). After stirring overnight, the mixture was added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from 30%-50% EtOAc in heptane. Product was isolated as 761 mg of white solid.

Preparation of 3-{[(4-Chlorophenyl)(methyl)amino]sulfonyl}benzoic acid

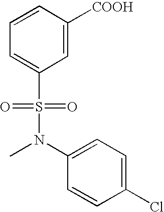

A chilled solution of 4-chloro-N-methylaniline (2.77 g, 19.6 mmol, Aldrich) and triethylamine (2.2 mL, 16 mmol) in methanol (10 mL) was added by cannula to solid 3-(chlorosulfonyl)benzoic acid (2.87 g, 13.0 mmol, Aldrich) with stirring in an ice bath. The ice bath was removed after 45 minutes, and the mixture was stirred at room temperature for 4.75 hours. The mixture was added to a separatory funnel with 60 mL of 1 M aqueous KOH, and this solution was washed with 2×60 mL of CH₂Cl₂. The aqueous layer was then acidified with concentrated HCl. The resulting precipitate was collected, washed with water, and recrystallized from hot toluene. The crystals were collected, washed with pentane, and dried at 100° C. under vacuum. Yield was 2.48 g of white solid.

Preparation of 3-{[(4-Chlorophenyl)(methyl)amino]sulfonyl}benzoyl chloride

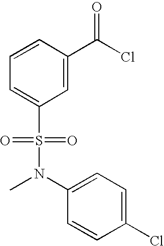

To a slurry of the carboxylic acid (1.21 g, 3.71 mmol) in CH₂Cl₂ (25 mL) was added DMF (3 drops) followed by oxalyl chloride (0.65 mL, 7.5 mmol). The resulting solution was stirred overnight and then concentrated by rotary evaporation yielding 1.31 g (103%) of yellow solid that was carried on without further purification.

### Example 9

### N-[4-Bromo-2-(5-oxo-4,5-dihydro-1,2,4-oxadiazol-3-yl)phenyl]-3-{[(4-chlorophenyl)(methyl)amino]sulfonyl}benzamide

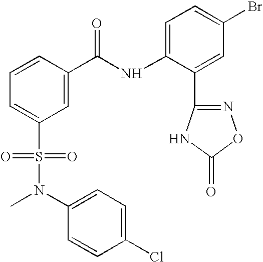

A solution of 3-{[(4-chlorophenyl)(methyl)amino]sulfonyl}benzoyl chloride (318 mg, 0.924 mmol) in CH₂Cl₂ (6 mL) was added to a solution of 3-(2-amino-5-bromophenyl)-1,2,4-oxadiazol-5(4H)-one (210 mg, 0.820 mmol) in pyridine (4 mL). After stirring overnight, the mixture was added to a separatory funnel with 100 mL of CH₂Cl₂. This solution was washed with 2×100 mL of 1 M aqueous HCl and 100 mL of brine, after which a precipitate began to form in the organic layer. The precipitate was collected, washed with pentane, and recrystallized from hot methanol/toluene. The crystals were collected, washed with pentane, and dried at 100° C. under vacuum yielding 164 mg of white solid. ¹H NMR (400 MHz, DMSO-d₆) δ 10.67 (s, 1H), 8.24 (d, J=7.6 Hz, 1H), 8.18 (s, 1H), 8.01 (d, J=8.7 Hz, 1H), 7.94 (d, J=2.0 Hz, 1H), 7.87 (dd, J=8.7, 2.0 Hz, 1H), 7.78 (t, J=7.9 Hz, 1H), 7.66 (d, J=8.1 Hz, 1H), 7.42 (d, J=8.7 Hz, 2H), 7.16 (d, J=9.2 Hz, 2H), 3.19 (s, 3H).

### Example 10

### 3-(Benzyloxy)-N-[4-bromo-2-(5-oxo-4,5-dihydro-1,2,4-oxadiazol-3-yl)phenyl]benzamide

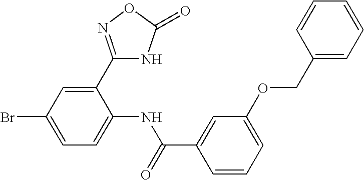

To 3-(benzyloxy)benzoic acid described by Bone et al. in Mol. Cryst. Liq. Cryst. 1988, 164, 117-134 (308 mg, 1.35 mmol) in CH₂Cl₂ (25 mL) was added DMF (20 μL) and oxalyl chloride (250 μL, 2.87 mmol). The mixture was stirred overnight, and the solvent and excess oxalyl chloride were removed by rotary evaporation. The residue was dissolved in CH₂Cl₂ (12 mL), and 3-(2-amino-5-bromophenyl)-1,2,4-oxadiazol-5(4H)-one (330 mg, 1.29 mmol) in pyridine (6 mL) was added. The mixture was stirred for 7 hours and then added to a separatory funnel with 70 mL of CH₂Cl₂. This solution was washed with 70 mL of 1 M aqueous HCl. The CH₂Cl₂ was quickly separated, and the product began to crystallize from it. After cooling in the freezer, the crystals were collected and recrystallized from hot ethanol/THF. The solids were washed with ethanol followed by heptane and then dried at 100° C. under vacuum. Yield was 221 mg of white solid. ¹H NMR (400 MHz, DMSO-d₆) δ 12.95 (br s, 1H), 10.47 (s, 1H), 8.11 (d, J=8.9 Hz, 1H), 7.92 (d, J=2.3 Hz, 1H), 7.85 (dd, J=8.9, 2.3 Hz, 1H), 7.58 (s, 1H), 7.47-7.54 (m, 4H), 7.41 (t, J=7.4 Hz, 2H), 7.35 (t, J=7.3 Hz, 1H), 7.28 (d, J=7.7 Hz, 1H), 5.20 (s, 2H).

Preparation of 2-Amino-5-bromobenzaidehyde

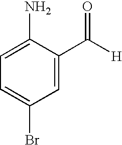

To a solution of 2-aminobenzaldehyde (1.96 g, 16.2 mmol, Sigma) in DMF was added NBS (2.87 g, 16.1 mmol, Aldrich). The solution was stirred for 40 minutes and then added to a separatory funnel with 200 mL of MTBE. This solution was washed with 5×200 mL of water and then dried over MgSO₄. Removal of the solvent left a yellow solid. This material was dissolved in CH₂Cl₂ and filtered through a plug of silica. The silica was rinsed with CH₂Cl₂ until all of the yellow color had eluted. Removal of the CH₂Cl₂ left the product as 3.04 g of yellow solid.

Preparation of N-(4-Bromo-2-formylphenyl)-3-[(5-chloro-2,3-dihydro-1H-indol-1-yl)sulfonyl]benzamide

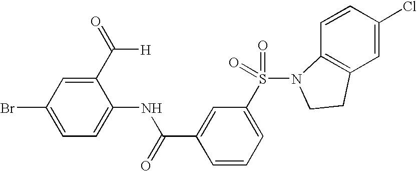

To 3-[(5-chloro-2,3-dihydro-1H-indol-1-yl)sulfonyl]benzoic acid (1.78 g, 5.27 mmol) in CH₂Cl₂ (50 mL) was added DMF (25 μL) and oxalyl chloride (800 μL, 9.17 mmol). The mixture was stirred for 3.5 hours, and the solvent and excess oxalyl chloride were removed by rotary evaporation. The residue was dissolved in CH₂Cl₂ (20 mL), and 2-amino-5-bromobenzaldehyde (0.993 g, 4.96 mmol) in pyridine (10 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from 75% CH₂Cl₂ in heptane to 100% CH₂Cl₂ as eluent. Yield was 2.19 g of white solid.

Preparation of N-(4-Bromo-2-formylphenyl)-3-(morpholin-4-ylsulfonyl)benzamide

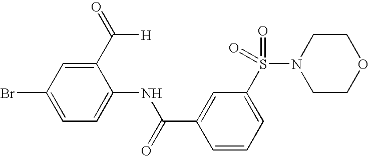

To 3-(morpholin-4-ylsulfonyl)benzoic acid (1.68 g, 6.19 mmol) in CH₂Cl₂ (50 mL) was added DMF (25 μL) and oxalyl chloride (800 μL, 9.17 mmol). The mixture was stirred for 2.5 hours, and the solvent and excess oxalyl chloride were removed by rotary evaporation. The residue was dissolved in CH₂Cl₂ (20 mL), and 2-amino-5-bromobenzaldehyde (1.10 g, 5.50 mmol) in pyridine (10 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from CH₂Cl₂ to 5% CH₂Cl₂ in heptane as eluent. Yield was 2.16 g of white solid.

### Example 11

### N-[4-Bromo-2-(1,1-dioxido-2H-1,2,4-benzothiadiazin-3-yl)phenyl]-3-(morpholin-4-ylsulfonyl)benzamide

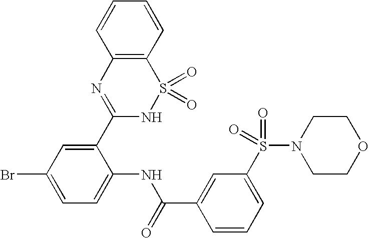

Solid sodium bisulfite (295 mg, 2.83 mmol, Mallinckrodt) was added to a solution of N-(4-bromo-2-formylphenyl)-3-(morpholin-4-ylsulfonyl)benzamide (777 mg, 1.72 mmol) and 2-aminobenzenesulfonamide (298 mg, 1.73 mmol, Aldrich) in dimethylacetamide (10 mL). This mixture was heated in a 150° C. oil bath for 2.5 hours. The solution was allowed to cool and then poured into 50 mL of water, resulting in a precipitate. The precipitate was washed with water followed by heptane and then dried at 100° C. under vacuum yielding 373 mg of white solid. ¹H NMR (400 MHz, DMSO-d₆) δ 12.46 (s, 1H), 10.88 (s, 1H), 8.22 (d, J=7.9 Hz, 1H), 8.17 (s, 1H), 7.98 (d, J=2.3 Hz, 1H), 7.92 (d, J=7.9 Hz, 1H), 7.88 (dd, J=8.7, 2.3 Hz, 1H), 7.75-7.81 (m, 2H), 7.65-7.71 (m, 2H), 7.44-7.48 (m, 2H), 3.60-3.65 (m, 4H), 2.85-2.90 (m, 4H).

### Example 12

### N-[4-Bromo-2-(4-oxo-3,4-dihydroquinazolin-2-yl)phenyl]-3-[(5-chloro-2,3-dihydro-1H-indol-1-yl)sulfonyl]benzamide

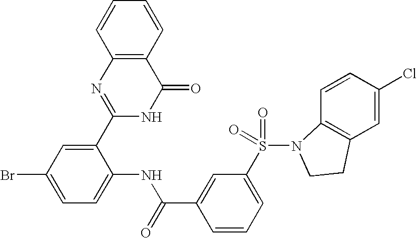

Dimethylacetamide (8 mL) was added to a flask containing sodium bisulfite (295 mg, 2.83 mmol, Mallinckrodt), N-(4-bromo-2-formylphenyl)-3-[(5-chloro-2,3-dihydro-1H-indol-1-yl)sulfonyl]benzamide (583 mg, 1.12 mmol) and 2-aminobenzamide (153 mg, 1.12 mmol, Aldrich). This mixture was heated in a 150° C. oil bath for 3 hours. The solution was allowed to cool and then poured into 50 mL of water, resulting in a precipitate. The precipitate was washed with water followed by methanol followed by heptane and then dried at 100° C. under vacuum yielding 566 mg of white solid. ¹H NMR (400 MHz, DMSO-d₆) δ 12.68 (s, 1H), 11.96 (s, 1H), 8.28 (s, 1H), 8.25 (d, J=8.9 Hz, 1H), 8.20 (d, J=7.9 Hz, 1H), 8.15 (d, J=7.1 Hz, 1H), 8.09 (d, J=1.9 Hz, 1H), 8.01 (d, J=8.5 Hz, 1H), 7.79-7.85 (m, 2H), 7.76 (t, J=7.9 Hz, 1H), 7.68 (d, J=7.9 Hz, 1H), 7.55 (t, J=7.2 Hz, 1H), 7.44 (d, J=9.1 Hz, 1H), 7.22-7.25 (m, 2H), 3.93 (t, J=8.5 Hz, 2H), 2.89 (t, J=8.4 Hz, 2H).

### Example 13

### N-[4-Bromo-2-(4-oxo-3,4-dihydroquinazolin-2-yl)phenyl]-3-(morpholin-4-ylsulfonyl)benzamide

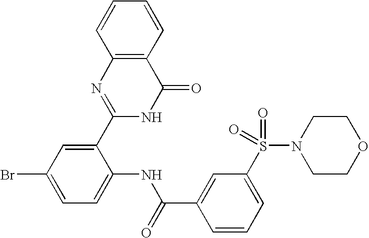

Dimethylacetamide (8 mL) was added to a flask containing sodium bisulfite (295 mg, 2.83 mmol, Mallinckrodt), N-(4-bromo-2-formylphenyl)-3-(morpholin-4-ylsulfonyl)benzamide (504 mg, 1.11 mmol) and 2-aminobenzamide (151 mg, 1.11 mmol, Aldrich). This mixture was heated in a 150° C. oil bath for 3.5 hours. The solution was allowed to cool, and 40 mL of water was added, resulting in a precipitate. The precipitate was washed with water followed by methanol followed by heptane and then dried at 100° C. under vacuum yielding 457 mg of white solid. ¹H NMR (400 MHz, DMSO-d₆) δ 12.69 (s, 1H), 11.94 (s, 1H), 8.24-8.28 (m, 2H), 8.18 (s, 1H), 8.15 (d, J=7.9 Hz, 1H), 8.09 (d, J=2.1 Hz, 1H), 7.96 (d, J=8.1 Hz, 1H), 7.81-7.87 (m, 3H), 7.73 (d, J=7.9 Hz, 1H), 7.55 (t, J=7.5 Hz, 1H), 3.58-3.63 (m, 4H), 2.84-2.88 (m, 4H).

### Example 14

### N-[4-Bromo-2-(1,1-dioxido-2H-1,2,4-benzothiadiazin-3-yl)phenyl]-3-[(5-chloro-2,3-dihydro-1H-indol-1-yl)sulfonyl]benzamide

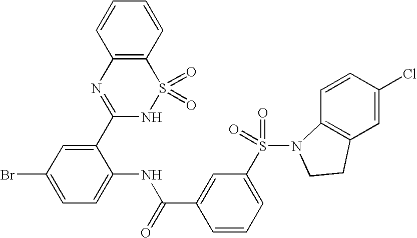

Solid sodium bisulfite (215 mg, 2.07 mmol, Mallinckrodt) was added to a solution of N-(4-bromo-2-formylphenyl)-3-[(5-chloro-2,3-dihydro-1H-indol-1-yl)sulfonyl]benzamide (705 mg, 1.36 mmol) and 2-aminobenzenesulfonamide (236 mg, 1.37 mmol, Aldrich) in dimethylacetamide (10 mL). This mixture was heated in a 150° C. oil bath for 2.5 hours. The solution was allowed to cool, and 40 mL of water were added, resulting in a precipitate. The precipitate was washed with water followed by heptane and then dried at 100° C. under vacuum yielding 629 mg of off-white solid that was impure by NMR. This material was recrystallized from a hot mixture of 10 mL of THF with 30 drops of DMSO added. The crystals were washed with THF followed by methanol followed by heptane and then dried at 100° C. under vacuum yielding 299 mg of white solid. ¹H NMR (400 MHz, DMSO-d₆) δ 12.46 (s, 1H), 10.86 (s, 1H), 8.29 (t, J=1.6 Hz, 1H), 8.17 (d, J=8.1 Hz, 1H), 7.98 (d, J=2.3 Hz, 1H), 7.93 (d, J=8.5 Hz, 1H), 7.88 (dd, J=8.7, 2.3 Hz, 1H), 7.79 (d, J=7.9 Hz, 1H), 7.65-7.69 (m, 3H), 7.43-7.49 (m, 3H), 7.22-7.25 (m, 2H), 3.97 (t, J=8.4 Hz, 2H), 2.91 (t, J=8.4 Hz, 2H).

### Example 15

### N-[4-Bromo-2-(5-oxo-4,5-dihydro-1,2,4-oxadiazol-3-yl)phenyl]-3-isopropoxybenzamide

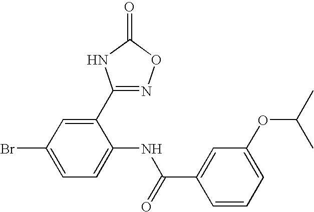

3-Isopropoxybenzoic acid, 144 mg, 0.80 mmol was dissolved in dry CH₂Cl₂ (10 mL) under N₂ and treated with DMF (15 μL) followed by oxalyl chloride (0.140 mL, 1.6 mmol). Gas evolved as the mixture was stirred for one hour at RT. The solvent and excess oxalyl chloride were evaporated and the resultant residue was taken up in CH₂Cl₂ (10 mL). 3-(2-Amino-5-bromophenyl)-1,2,4-oxadiazol-5(4H)-one (187 mg, 0.73 mmol) was added as a solution in dry pyridine (4 mL) and the yellow solution was stirred at RT overnight. The reaction was diluted to 50 mL with CH₂Cl₂, and the organic layer was washed 1× with 1.0M HCl causing the product to precipitate. The organic layer containing the precipitated product was evaporated, and the residue was re-crystallized from hot EtOH and the resultant product was dried at 100° C. under vacuum to afford 95 mg (31%) of pale yellow needles. ¹H NMR (400 MHz, DMSO-d₆) δ 12.95 (s, 1H), 10.43 (s, 1H), 8.13 (d, J=8.9 Hz, 1H), 7.92 (d, J=2.3 Hz, 1H), 7.84 (dd, J=8.8, 2.4 Hz, 1H), 7.43-7.49 (m, 3H), 7.15-7.19 (m, 1H), 4.71 (m, 1H), 1.32 (s, 3H), 1.30 (s, 3H).

### Example 16

### N-[4-Bromo-2-(5-oxo-4,5-dihydro-1,2,4-oxadiazol-3-yl)phenyl]-3-phenoxybenzamide, PHA-666967

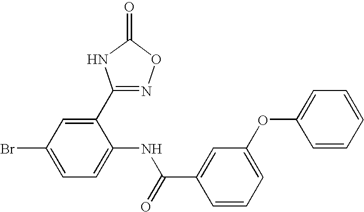

3-Phenoxybenzoic acid (, 236 mg, 1.1 mmol, Aldrich) was dissolved in dry CH₂Cl₂ (10 mL) under N₂ and treated with DMF (20 μL) followed by oxalyl chloride (0.192 mL, 2.2 mmol). Gas evolved as the mixture was stirred for one hour at RT. The solvent and excess oxalyl chloride were evaporated and the resultant residue was taken up in CH₂Cl₂ (10 mL). 3-(2-Amino-5-bromophenyl)-1,2,4-oxadiazol-5(4H)-one (256 mg, 1.0 mmol) was added as a solution in dry pyridine (4 mL) and the yellow solution was stirred at RT overnight. The reaction was diluted to 75 mL with CH₂Cl₂, and the organic layer was washed 2× with 1.0M HCl causing the product to precipitate. The organic layer containing the precipitated product was evaporated, and the residue was re-crystallized from hot EtOH/THF. The resultant product was dried at 100° C. under vacuum to afford 234 mg (47%) of pale yellow needles. ¹H NMR (400 MHz, DMSO-d₆) δ 12.92 (s, 1H), 10.46 (s, 1H), 8.04 (d, J=8.9 Hz, 1H), 7.91 (d, J=2.3 Hz, 1H), 7.84 (dd, J=8.7, 2.3 Hz, 1H), 7.70 (d, J=7.7 Hz, 1H), 7.59 (t, J=8.0 Hz, 1H), 7.55 (t, J=2.1 Hz, 1H), 7.41-7.47 (m, 2H), 7.27 (dd, J=7.7, 2.1 Hz, 1H), 7.20 (t, J=7.4 Hz, 1H), 7.07-7.12 (m, 2H).

Preparation of 5-Bromo-anthranilonitrile

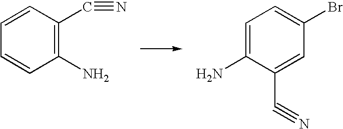

N-Bromosuccinimide (17.8 g, 0.10 mol) was added in portions at room temperature to a stirred solution of anthranilonitrile (Aldrich, 11.8 g, 0.10 mol) in DMF (100 mL). After a mild exothermic reaction occurred, the red solution was stirred under nitrogen overnight. The reaction mixture was diluted with water (˜100 mL) and CH₂Cl₂ (200 mL). The phases were separated. The organic phase was washed with H₂O (3×) and brine, dried (Na₂SO₄) and concentrated in vacuo to give 21 g of red residue which was dissolved in ether (25 mL), and hexane was added to knock out 13.6 (70% yield, 1^{st} crop) g of red solid product: mp 95-97° C. IR (diffuse reflectance) 3357, 2219, 1633, 1560, 1486, 1305, 1257, 1180, 1157, 889, 866, 830, 666, 656, 641 cm⁻¹. Anal. Calcd for C₇H₅BrN₂:C, 42.67; H, 2.56; N, 14.22; Br, 40.55. Found: C, 42.75; H, 2.54; N, 14.25; Br, 40.34.

Preparation of N-[4-bromo-2-cyanophenyl]-3-[morpholin-4yl-sulfonyl]benzamide

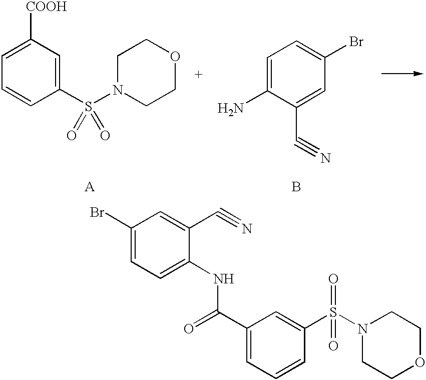

A mixture of A (0.29 g, 1.07 mmol), oxalyl chloride (4 mL), and CH₂Cl₂ (4 mL) was treated with DMF (ca. 2 μL). The mixture was stirred at room temperature for 1 hr and then heated to reflux for 1 h. The yellow solution was cooled and concentrated in vacuo to give crude acid chloride that was redissolved in CH₂Cl₂ (2 mL) and added slowly to a mixture of B (0.21 g, 1.07 mmol) in pyridine (5 mL). The mixture was stirred at room temperature for 2 h and then concentrated in vacuo to give a dark viscous liquid. The crude product was dissolved in CH₂Cl₂ and aqueous 1N HCl.

The phases were separated. The organic phase was washed with H₂O, dried (Na₂SO₄), and concentrated to give 0.46 g of a tan foamy residue that when triturated with ethanol gave 0.38 g of solid product: mp 185-187° C.MS (ESI−) for C₁₈H₁₆BrN₃O₄S m/z 449.8 (M-H)⁻. IR (diffuse reflectance) 3278, 1666, 1508, 1485, 1466, 1357, 1319, 1296, 1259, 1174, 1110, 943, 843, 747, 723 cm⁻¹. MS (EI) m/z (rel. intensity) 449 (M⁺, 0), 254 (99), 105 (34), 104 (41), 86 (61), 78 (45), 76 (58), 63 (67), 57 (46), 56 (73), 55 (34). Anal. Calcd for C₁₈H₁₆BrN₃O₄S: C, 48.01; H, 3.58; N, 9.33; Br, 17.74; S, 7.12. Found: C, 47.93; H, 3.59; N, 9.21; S, 7.09.

### Example 17

### N-[4-bromo-2-(1H-tetraazol-5-yl)phenyl]-3-(morpholin-4-ylsulfonyl)benzamide

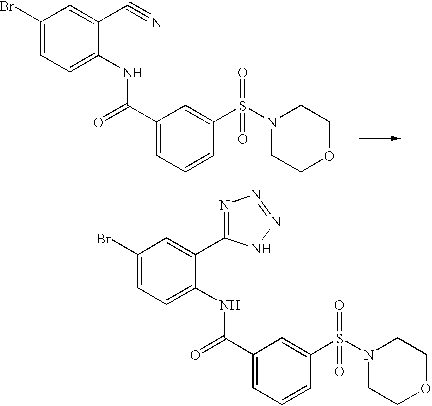

Trimethylaluminum in toluene (Aldrich, 2M, 1.20 mL, 2.4 mmol) was added to an oven-dried 10 mL round bottom flask under N₂. Azidotrimethylsilane (Aldrich, 0.32 mL, 2.4 mmol) was slowly added at 0° C. and stirred for 10 min. The colorless solution was treated in portions with solid N-[4-bromo-2-cynophenyl]-3-[morpholin-4yl-sulfonyl]benzamide (0.90 g, 2.0 mmol). The mixture was diluted with dry toluene (ca. 4 mL) to aid stirring and allowed to warm to room temperature before heating to 90° C. for 4 h. The mixture was cooled to room temperature and cautiously diluted with aqueous 1N HCl and CH₂Cl₂. The phases were separated. The aqueous phase was extracted 3× again with CH₂Cl₂—CHCl₃ mixtures. The combined organic phase was washed with brine, dried (Na₂SO₄), and concentrated to give crude solid which was digested in hot ethanol (20 mL), cooled, and filtered to give 0.47 g of white solid product; mp 228-230: MS (ESI−) for C₁₈H₁₇BrN₆O₄S m/z 492.8 (M-H)⁻. IR (diffuse reflectance) 1688, 1615, 1591, 1534, 1488, 1352, 1311, 1299, 1263, 1173, 1113, 1071, 945, 841, 730 cm⁻¹. Anal. Calcd for C₁₈H₁₇BrN₆O₄S: C, 43.82; H, 3.47; N, 17.03; Br, 16.20; S, 6.50. Found: C, 43.74; H, 3.45; N, 17.06; Br, 15.90.

Preparation of Methyl 5-cyano-2-{[3-(morpholin-4-ylsulfonyl) benzoyl]amino}benzoate

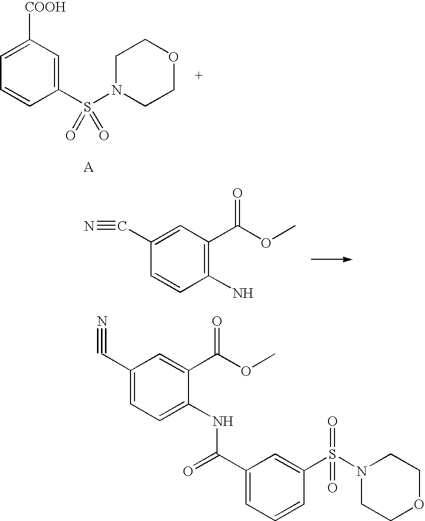

A mixture of A (2.0 g, 7.37 mmol), oxalyl chloride (10 mL), and CH₂Cl₂ (4 mL) was treated with DMF (ca. 2 μL). The mixture was stirred at room temperature for 1 hr and then heated to reflux for 5 h. The yellow solution was cooled and concentrated in vacuo to give crude acid chloride that was redissolved in CH₂Cl₂ (2 mL) and added slowly to a mixture of methyl 2-amino-5-cyanobenzoate (1.28 g, 7.37 mmol) in dry pyridine (10 mL). The red mixture was stirred at room temperature for 1 h and then heated to reflux for 3 h under N₂. The reaction mixture was cooled to room temperature, diluted with H₂O (2 mL), and concentrated in vacuo to a slurry mixture that was diluted with ethyl acetate. The mixture was filtered. The solids were washed with H₂O and dried in a vacuum oven to give 2.02 g (64% yield) of product: mp 184-185° C.: MS (ESI+) for C₂₀H₁₉N₃O₆S m/z 429.9 (M+H)⁺. IR (diffuse reflectance) 3457, 3362, 2224, 1642, 1551, 1485, 1401, 1306, 1261, 1178, 1156, 900, 858, 821, 634 cm⁻¹. MS (EI) m/z (rel. intensity) 244 (M⁺, 0), 245 (12), 244 (99), 127 (9), 118 (9), 117 (72), 90 (75), 84 (11), 64 (14), 63 (42), 62 (13). Anal. Calcd for C₇H₅₁N₂: C, 34.45; H, 2.07; N, 11.48. Found: C, 34.57; H, 2.08; N, 11.48.

Preparation of N-[4-cyano-2-(hydrazinocarbonyl)phenyl]-3-(morpholin-4-ylsulfonyl)benzamide

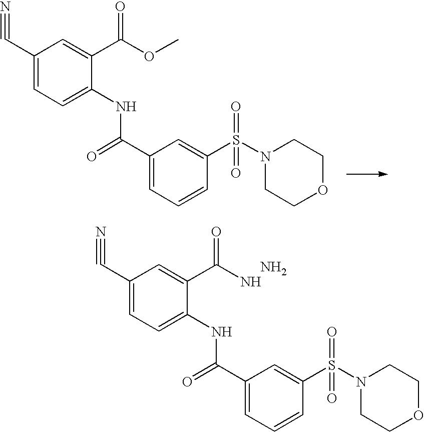

A mixture of methyl-5-cyano-2-{[3-(morpholin-4yl-sulfonyl)benzoyl]amino)benzoate (0.29 g, 0.68 mmol) and hydrazine monohydrate (0.048 mL, 0.96 mmol) in ethanol (3 mL) was heated to reflux under N₂ for 16 h. Additional hydrazine (0.40 mL) was added to the reaction mixture and heated to reflux for 2 h. The mixture was cooled to room temperature and filtered. The solids were washed with aqueous ethanol and dried in a vacuum oven (50° C.) to give 0.211 g. MS (ESI−) for C₁₉H₁₉N₅O₅S m/z 428.0 (M−H)⁻. ¹H NMR (400 MHz, DMSO-d₆) δ 2.95 (m, 4H), 3.65 (m, 4H), 4.78 (brd s, 2H), 7.92 (m, 1H), 8.03 (m, 2H), 8.26 (m, 3H), 8.78 (d, 1H), 10.3 (s, 1H), 13.0 (s, 1H).

### Example 18

### N-[4-cyano-2-(5-oxo-4,5-dihydro-1,3,4-oxadiazol-2-yl)phenyl]-3-(morpholin-4-ylsulfonyl)benzamide

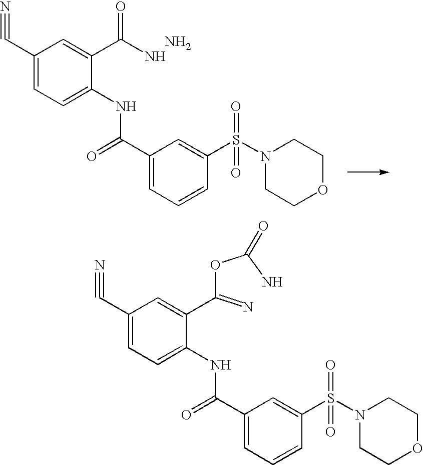

A mixture of N-[4-cyano-2-(hydrazine carbonyl)phenyl]-3-(morpholin-4yl-sulfonyl]benzamide (0.10 g, 0.24 mmol) and 1,1-carbonyldiimidazole (0.056 g, 0.345 mmol) in dry THF (7 mL) was heated slowly to 62° C. under N₂ for 1 h. The solution was cooled to room temperature and concentrated in vacuo give crude solids which were redissolved in CHCl₃-5% CH₃OH and washed with aqueous 1 N HCl and brine. The organic phase was dried (Na₂SO₄) and concentrated in vacuo to give a white solid which was triturated with CHCl₃— hexanes mixture and filtered to a give white solid (0.032 g); mp 283-285° C. IR (diffuse reflectance) 1802, 1787, 1700, 1601, 1530, 1358, 1325, 1302, 1283, 1264, 1170, 944, 929, 750, 715 cm⁻¹. OAMS supporting ions at: ESI-453.8. MS (EI) m/z (rel. intensity) 455 (M⁺, 2), 254 (62), 169 (37), 105 (63), 104 (83), 86 (34), 77 (33), 76 (83), 75 (28), 57 (36), 56 (99). HRMS (FAB) calcd for C₂₀H₁₇N₅O₆S+H₁ 456.0977, found 456.0986. Anal. Calcd for C₂₀H₁₇N₅O₆S: C, 52.74; H, 3.76; N, 15.38; S, 7.04. Found: C, 50.56; H, 3.65; N, 14.60.

Preparation of N-{2-[(Z)-amino(hydroxyimino)methyl]-4-bromophenyl}-3-(morpholin-4-ylsulfonyl)benzamide

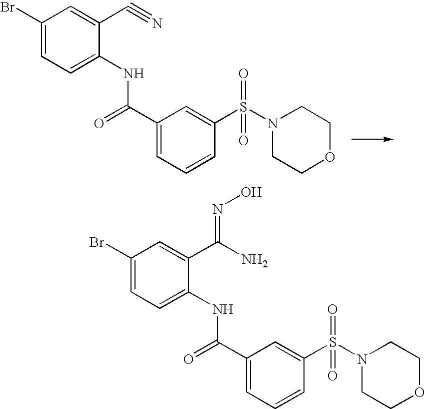

A mixture of N-[4-bromo-2-cyanophenyl]-3-morpholin-4yl-sulfonyl]benzamide (0.517 g, 1.15 mmol), hydroxylamine.HCl (0.089 g, 1.28 mmol) in H₂O (ca. 0.5 mL) and ethanol (10 mL) was treated with NaOMe in CH₃OH (25 wt %, 0.29 mL, 1.27 mmol). The mixture was heated to 90° C. overnight. The mixture was cooled to room temperature and diluted with H₂O (10 mL) and stirred for 30 minutes. The mixture was filtered and the yellow solid was washed with H₂O and dried under vacuum to give 0.49 g crude product that was digested in hot ethanol-1,2-dichloroethane. The cooled mixture was filtered to give 0.39 g of pale yellow product: mp 241-243° C. IR (diffuse reflectance) 3514, 3380, 1682, 1635, 1533, 1331, 1311, 1300, 1265, 1170, 1107, 1069, 946, 836, 721 cm⁻¹. OAMS supporting ions at: ESI− 482.7. MS (EI) m/z (rel. intensity) 482 (M⁺, 0), 335 (30), 333 (32), 254 (27), 86 (77), 84 (83), 65 (37), 64 (45), 63 (99), 56 (42), 51(51). Anal. Calcd for C₁₈H₁₉BrN₄O₅S: C, 44.73; H, 3.96; N, 11.59; Br, 16.53; S, 6.63. Found: C, 44.63; H, 4.02; N, 11.41.

### Example 19

### N-[4-bromo-2-(5-oxo-4,5-dihydro-1,2,4-oxadiazol-3-yl)phenyl]-3-(morpholin-4-ylsulfonyl)benzamide

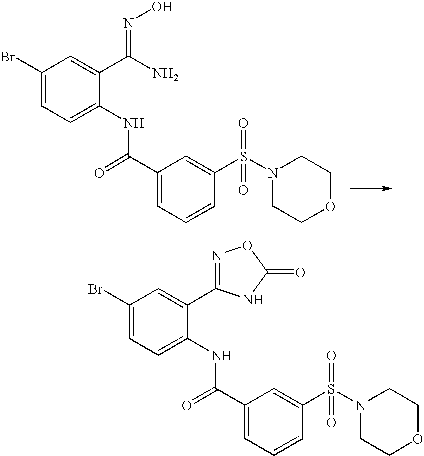

N {2-[(Z0-amino(hydroxyimino)methyl]-4-bromophenyl}-3-(morpholin-4yl sulfonyl)benzamide (0.312 g, 0.64 mmol) suspended in ethanol (5 mL) and DMF (2 mL) at room temperature was treated with NaOCH₃ in CH₃OH (Aldrich, 25 wt %, 0.29 mL, 1.28 mmol) and stirred for 10 minutes and followed by addition of dimethylcarbonate (0.5 mL, 4 mmol). The mixture was heated to reflux under N₂ 16 h. The red-yellow opaque solution was cooled to room temperature and concentrated in vacuo to a slurry mixture, diluted with H₂O (ca. 3 mL), and was treated cautiously with aqueous 1N HCl. The yellow mixture was stirred for 1 h, filtered, and dried in a vacuum oven at 50° C. to give 0.31 g of yellow solid from which a sample was recrystallized from ethanol to give a yellow solid product; mp 220-223° C. IR (diffuse reflectance) 1793, 1686, 1612, 1598, 1353, 1321, 1306, 1301, 1263, 1169, 1103, 945, 841, 725, 689 cm⁻¹. OAMS supporting ions at: ESI− 508.7. MS (EI) m/z (rel. intensity) 508 (M⁺, 0), 299 (18), 254 (15), 169 (11), 105 (24), 104 (19), 86 (70), 84 (23), 76 (20), 75 (12), 56 (99). MS (FAB) m/z (rel. intensity) 509 (MH⁺, 24), 511 (23), 509 (24), 467 (49), 466 (26), 465 (39), 254 (99), 104 (28), 86 (56), 84 (18), 56 (39). HRMS (FAB) calcd for C₁₉H₁₇BrN₄O₆S+H₁ 509.0131, found 509.0142. Anal. Calcd for C₁₉H₁₇BrN₄O₆S: C, 44.81; H, 3.36; N, 11.00; Br, 15.69; S, 6.29. Found: C, 43.02; H, 3.40; N, 10.68; Br, 15.47.

### Example 20

### N-[4-cyano-2-(1H-tetraazol-5-yl)phenyl]-3-(morpholin-4-ylsulfonyl)benzamide

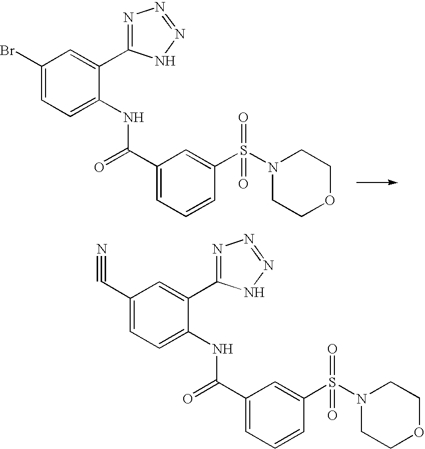

Moist DMF (syringe barrel was rinsed with water, 2 mL) that was purged with N₂ was added to a mixture of the compound from example 17 (0.155 g, 0.314 mmol), zinc cyanide (23 mg, 1.89 mmol), 1,1′-bis(diphenylphosphino)ferrocene (DPPF) (22 mg, 0.04 mmol), and tris(dibenzylideneacetone)dipalladium(0) (15.2 mg, 0.017 mmol). The reaction mixture was heated to 125° C. under N₂ for 18 h. The mixture was cooled to room temperature and diluted with aqueous 0.5 M HCl (ca. 5 mL). The tan mixture was stirred at room temperature for 1 h and filtered. The solid was washed with water, partially dried on the filter under vacuum, recrystallized from hot ethyl acetate-methanol mixture. After cooling in the refrigerator overnight, The mixture was filtered, washed with cold ethyl acetate to give 0.040 g of brown solid product; mp 230-232° C. IR (diffuse reflectance) 3183, 3174, 3081, 1701, 1598, 1526, 1352, 1317, 1303, 1263, 1171, 1103, 941, 760, 717 cm⁻¹. OAMS supporting ions at: ESI− 437.8. MS (CI) m/z (rel. intensity) 440 (MH⁺, 1), 457 (17), 263 (19), 105 (38), 103 (11), 89 (18), 88 (99), 87 (13), 86 (71), 56 (8), 52 (33). HRMS (FAB) calcd for C₁₉H₁₇N₇O₄S+H₁ 440.1141, found 440.1137.

### Example 21

### [5-(5-bromo-2-{[3-(morpholin-4-ylsulfonyl)benzoyl]amino}phenyl)-2H-tetraazol-2-yl]methyl pivalate

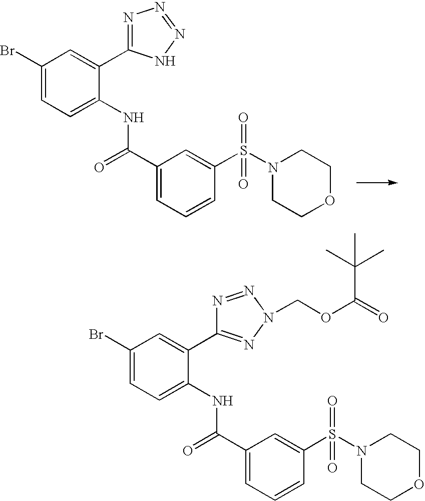

A mixture of the compound from example 17 (77.8 mg, 0.158 mmol), chloromethylpivalate (30 μL, 0.2 mmol), sodium iodide (30 mg, 0.2 mmol), and potassium acetate (20 mg, 0.2 mmol) in DMF (2 mL) was stirred at room temperature for 60 h. The mixture was diluted with H₂O and CH₂Cl₂. The phases were separated. The organic phase was washed with H₂O (2×), brine, dried (Na₂SO₄), and concentrated in vacuo to give 70 mg of a waxy pale yellow solid which was triturated with diethyl ether to give 35.5 mg of white solid; mp 210-212° C. MS (EI) m/z (rel. intensity) 606 (M⁺, 11), 254 (65), 169 (47), 128 (65), 105 (81), 104 (72), 86 (99), 85 (67), 76 (73), 57 (99), 56 (86). MS (FAB) m/z (rel. intensity) 607 (MH⁺, 47), 609 (49), 607 (47), 495 (22), 493 (34), 254 (44), 133 (26), 119 (23), 118 (41), 86 (22), 57 (99). HRMS (FAB) calcd for C₂₄H₂₇BRN₆O₆S+H₁ 607.0975, found 607.0966.

Preparation of Methyl 2-({4-[(5-chloro-2,3-dihydro-1H-indol-1-yl)sulfonyl]benzoyl}amino)-5-cyanobenzoate

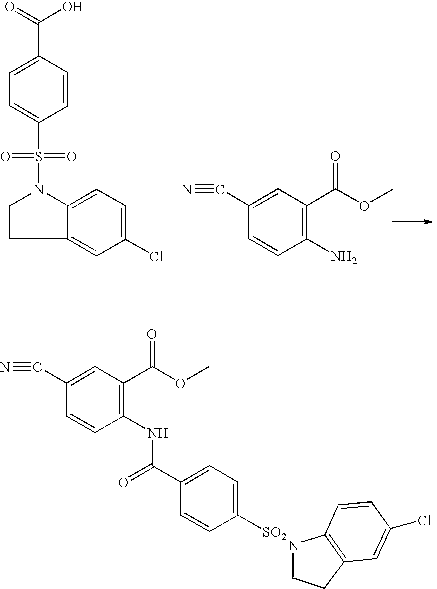

A mixture of 4-[(5-chloro-2,3-dihydro-1H-indol-1-yl)sulfonyl)benzoic acid. (0.50 g, 1.49 mmol) and oxalyl chloride (5 mL) in CH₂Cl₂ (3 mL) was treated with DMF (3 μL) and heated to 40° C. After 2 h, the mixture was cooed and concentrated in vacuo to give a yellow semi-solid residue that was dissolved in CH₂Cl₂ (2 mL) and pipetted into a mixture of methyl 2-amino-5-cyanobenzoate (0.25 g, 1.40 mmol) in pyridine (5 mL) at 5° C. The mixture was allowed to stir at room temperature overnight and then concentrated in vacuo. The residue was dissolved in CH₂Cl₂ and aqueous 1N HCl. The phases were separated. The organic phase was washed with aqueous 1 N HCl, and H₂O, dried (Na₂SO₄), and concentrated in vacuo to provide a tan solid that was digested in hot ethanol. The cooled mixture was filtered. The solid was dried in a vacuum oven at 60° C. to give 0.42 g (61%) of cream solid product. ¹H NMR (400 MHz, DMSO-d₆) δ 2.95 (t, 2H), 3.88 (s, 3H), 4.01 (t, 2H), 7.26 (m, 2H), 7.49 (d, 1H), 8.12 (m, 5H), 8.37 (s, 1H), 8.53 (d, 1H), 11.5 (s, 1H); MS (ESI−) for C₂₄H₁₈ClN₃O₅S m/z 493.9 (M−H)⁻.

Preparation of 4-[(5-chloro-2,3-dihydro-1H-indol-1-yl)sulfonyl]-N-[4-cyano-2-(hydrazinocarbonyl)phenyl]benzamide

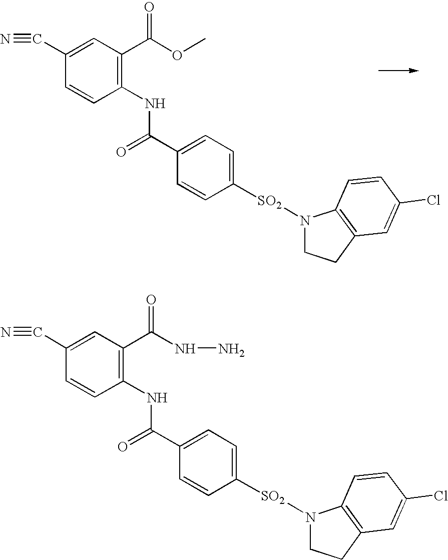

A mixture of methyl-2-({4-[(5-chloro-2,3-dihydro-1H-indol-1-yl)sulfonyl]benzoyl}amino)-5-cyano benzoate (0.335 g, 0.675 mmol) and hydrazine monohydrate (0.05 mL) in ethanol (5 mL) was heated to reflux for 16 h. The mixture was cooled and filtered. The solid was washed with H₂O and ethanol. ¹H NMR (400 MHz, DMSO-d₆) δ 2.95 (m, 2H), 4.0 (m, 2H), 4.75 (brd s, 1H), 7.25 (m, 3H), 7.49 (m, 1H), 8.1 (m, 5H), 8.24 (m, 1H), 8.73 (d, 1H), 10.3 (s, 1H), 12.9 (s, 1H);

### Example 22

### 4-[(5-chloro-2,3-dihydro-1H-indol-1-yl)sulfonyl]-N-[4-cyano-2-(5-oxo-4,5-dihydro-1,3,4-oxadiazol-2-yl)phenyl]benzamide

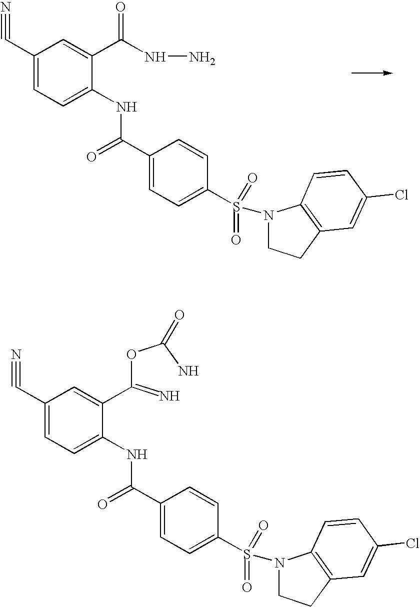

A mixture of 4[(5-chloro-2,3-dihydro-1H-indol-1-yl)sulfonyl]-N-[4-cyano-2-(hydrazine carbonyl)phenyl]benzamide (0.095 g, 0.19 mmol), 1,1-carbonyldiimidazole (0.056 g, 0.35 mmol) in THF (3 mL) was stirred at room temperature for 1 h and then heated to reflux under N₂ for 2 h. The mixture was cooled and diluted with ethyl acetate (1 mL) and hexane (2 mL). The mixture was filtered, washed with hexanes and dried in a vacuum oven overnight to give 0.040 g of white solid; mp 262-264° C. HRMS (FAB) calcd for C₂₄H₁₆CLN₅O₅S+H₁ 522.0638, found 522.0628. Anal. Calcd for C₂₄H₁₆ClN₅O₅S: C, 55.23; H, 3.09; N, 13.42; Cl, 6.79; S, 6.14. Found: C, 54.23; H, 3.16; N, 12.89.

### Example 23

### 4-[(5-chloro-2,3-dihydro-1H-indol-1-yl)sulfonyl]-N-[4-cyano-2-(5-thioxo-4,5-dihydro-1,3,4-oxadiazol-2-yl)phenyl]benzamide

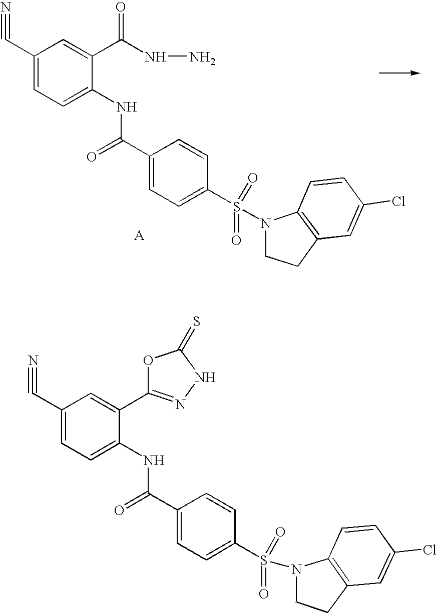

A mixture of A (0.217 g, 0.438 mmol), 1,1-thiocarbonyldiimidazole (0.12 g, 0.67 mmol) in THF (5 mL) was heated to reflux under N₂ for 2 h. The mixture was cooled and diluted with ethyl acetate and H₂O. The phases were separated. The organic phase was washed with a small portion of H₂O and concentrated in vacuo to give 0.22 g of solid which was digested in CHCl₃ (CH₃OH)/hexanes. The cooled mixture was filtered and dried to give 0.082 g of pale yellow product; mp 225-229° C. IR (diffuse reflectance) 3115, 3076, 1700, 1599, 1526, 1482, 1474, 1355, 1316, 1296, 1248, 1167, 1084, 741, 624 cm⁻¹. MS (EI) m/z (rel. intensity) 537 (M⁺, 5), 154 (94), 153 (93), 152 (99), 151 (84), 117 (93), 86 (88), 84 (85), 78 (95), 76 (86), 63 (88). HRMS (FAB) calcd for C₂₄H₁₆CLN₅O₄S₂+H₁ 538.0410, found 538.0393. Anal. Calcd for C₂₄H₁₆ClN₅O₄S₂: C, 53.58; H, 3.00; N, 13.02; Cl, 6.59; S, 11.92. Found: C, 53.18; H, 3.29; N, 12.50; Cl, 7.16.

Preparation of 2-amino-5-iodobenzonitrile

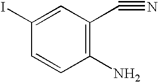

N-iodosuccinimide (11.35 g, 50.4 mmol) was added in several portions at room temperature to a solution of anthranilonitrile (Aldrich, 5.87 g, 49.7 mmol) in DMF (80 mL) and stirred under N₂ for 18 h. The reaction mixture was concentrated to 1/4 volume and diluted with H₂O (200 mL) and CH₂Cl₂. The phases were separated. The organic phase was washed with H₂O, dried (Na₂SO₄), and concentrated in vacuo to give 14 g of dark liquid that was dissolved in hot ethanol and diluted with ca. 100 mL of hot H₂O. The solution was cooled to room temperature overnight. The crystallized mixture was filtered and the pink mica-like sheets were washed with H₂O and dried in a vacuum oven at 40° C. to give 8.85 g (73%) of product; mp 76-78° C.:

MS (EI) m/z (rel. intensity) 244 (M⁺, 99), 245 (12), 127 (9), 118 (9), 117 (72), 90 (75), 84 (11), 64 (14), 63 (42), 62 (13).

Preparation of N-(2-cyano-4-iodophenyl)-7-(phenylsulfonyl)-1H-indole-2-carboxamide

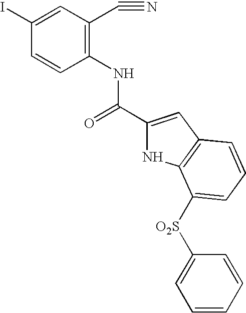

A mixture of 7-(phenylsulfonyl)-1H-indole-2-carboxylic acid (0.80 g, 2.67 mmol) and oxalyl chloride (10 mL) in CH₂Cl₂ (10 mL) was treated at room temperature with DMF (10 μL). The mixture was stirred at room temperature for 3 h. The solution was concentrated in vacuo to give a crude acid chloride that was suspended in CH₂Cl₂ (2 mL) and added slowly to a solution of 2-amino-5-iodobenzonitrile (0.65 g, 2.67 mmol) in dry pyridine (6 mL) at room temperature. The mixture was stirred overnight and then concentrated. The residue was partitioned with CH₂Cl₂ and aqueous 1N HCl. The phases were separated. The aqueous phase was extracted again with CH₂Cl_{2—CHCl}₃ mixture. The combined organic extracts were concentrated in vacuo to give a brown residue that was digested in boiling ethanol. The cooled mixture was filtered and the solid was washed with ethanol and dried to give 0.92 g (65%) of tan solid.

MS (EI) m/z (rel. intensity) 527 (M⁺, 59), 285 (52), 284 (99), 190 (31), 165 (25), 143 (27), 142 (50), 115 (26), 114 (24), 77 (27).

HRMS (FAB) calcd for C₂₂H₁₄IN₃O₃S+H₁ 527.9880, found 527.9877.

### Example 24

### N-[4-iodo-2-(1H-tetraazol-5-yl)phenyl]-7-(phenylsulfonyl)-1H-indole-2-carboxamide

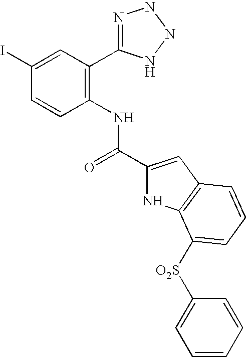

Neat azidotrimethylsilane (0.97 mL, ˜8.4 mmol) was added slowly to trimethylaluminum in toluene (2M, 0.97 mL, 1.94 mmol) at 0° C. under N₂. After 15 minutes, N-(2-cyano-4-iodophenyl)-7-(phenylsulfonyl)-1H-indole-2-carboxamide (0.78 g, 1.49 mmol) was added in several portions. The mixture was allowed to warm to room temperature, diluted with toluene (2 mL), and heated to 100° C. for 18 h. The cooled mixture was treated carefully with aqueous 4 N HCl and CH₂Cl₂. The mixture was filtered, and milk chocolate colored solid was washed with H₂O and CH₂Cl₂. The solid was then suspended in 1N aqueous NaOH and filtered. The solid was digested in hot ethanol, cooled, filtered, and dried to give 0.50 g a cream colored solid.

MS (EI) m/z (rel. intensity) 570 (M⁺, 0), 284 (32), 244 (35), 127 (37), 86 (74), 84 (99), 77 (52), 63 (65), 62 (50), 61 (27), 51 (92).

HRMS (FAB) calcd for C₂₂H₁₅IN₆O₃S+H₁ 571.0051, found 571.0045.

### Example 25

### N-[4-cyano-2-(1H-tetraazol-5-yl)phenyl]-7-(phenylsulfonyl)-1H-indole-2-carboxamide

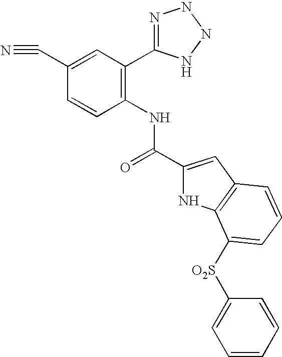

A mixture of N-[4-iodo-2-(1H-tetraazol-5-yl)phenyl]-7-(phenylsulfonyl)-1H-indole-2-carboxamide (0.42 g, 0.796 mmol), potassium cyanide (0.093 g, 1.42 mmol), tetrakis(triphenylphosphine)palladium(0) (Strem, yellow color, 0.041 g, 0.035 mmol), and CuI (14 mg, 0.07 mmol) was treated nitrogen-purged dry THF (3 mL) and stirred at room temperature for 20 min, then heated to reflux for 4 h before allowing to cool to room temperature overnight. The reaction mixture was diluted with aqueous HCl (˜1 mL), stirred for 30 min, and filtered. The aqueous washed solid was dissolved in ethyl acetate and aqueous 1N HCl. The phases were separated. The organic phase was washed with aqueous saturated sodium bicarbonate, filtered through a glass-wool plug, and concentrated in vacuo to give an off-white solid residue that was dissolved in hot ethyl acetate (CH₃OH) and hexane. After cooling to room temperature and then to 5° C., the mixture was filtered, and then solid was washed with hexane, dried in a vacuum oven at 40° C. to give 0.143 g of cream colored solid; mp 293-295° C.:

MS (EI) m/z (rel. intensity) 469 (M⁺, 9), 444 (18), 426 (28), 413 (34), 412 (16), 285 (24), 284 (99), 86 (66), 84 (74), 62 (21), 51 (26).

MS (FAB) m/z (rel. intensity) 470 (MH⁺, 9), 531 (27), 530 (76), 515 (27), 514 (82), 508 (42), 493 (25), 492 (99), 284 (36), 39 (83), 23 (52).

HRMS (FAB) calcd for C₂₃H₁₅N₇O₃S+H₁ 470.1035, found 470.1031.

### Example 26

### 2-({3-[(5-Chloro-2,3-dihydro-1H-indol-1-yl)sulfonyl]benzoyl}amino)-5-cyano-N-(1,3-thiazol-2-yl)benzamide

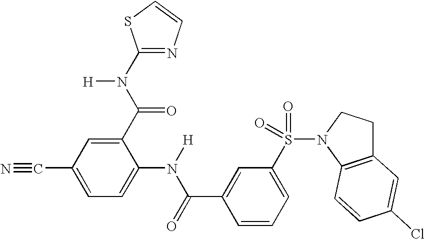

Tetrahydrofuran (12 mL) was added to a mixture of 2-({3-[(5-chloro-2,3-dihydro-1H-indol-1-yl)sulfonyl]benzoyl}amino)-5-cyanobenzoic acid (166 mg, 0.344 mmol), 1, 1′-carbonyldiimidazole (216 mg, 1.33 mmol, Aldrich), 2-aminothiazole (88 mg, 0.88 mmol, Aldrich), and 4-dimethylaminopyridine (56 mg, 0.46 mmol, Aldrich) in a screw capped vial. The solution was placed in a 90° C. shaker block for 17 hours. Additional portions of 2-aminothiazole (267 mg), DMAP (181 mg), and CDI (277 mg) were added, and the mixture was shaken at 100° C. for 20 hours. It was then added to a separatory funnel with 100 mL of CH₂Cl₂. This was washed with 100 mL of 1 M HCl and 100 mL of water. It was dried over MgSO₄ and evaporated leaving an orange solid that was purified by reverse phase preparative HPLC. The resulting product was recrystallized from hot ethanol/THF. The solids were washed with ethanol followed by heptane and then dried at 100° C. under vacuum yielding 47 mg of tan solid.

¹H NMR (400 MHz, DMSO-D6) δ ppm 2.95 (t, J=8.50 Hz, 2H) 4.05 (t, J=8.50 Hz, 2H) 7.22 (m, 2H) 7.32 (d, J=3.73 Hz, 1H) 7.55 (d, J=8.50 Hz, 1H) 7.61 (d, J=3.94 Hz, 1H) 7.82 (t, J=7.88 Hz, 1H) 8.07 (m, 2H) 8.25 (d, J=7.67 Hz, 1H) 8.40 (s, 1H) 8.56 (s, 2H).

### Example 27

### N-[4-Bromo-2-(1H-tetraazol-5-yl)phenyl]-4-{1(4-chlorophenyl)(methyl)amino]sulfonyl}-2-furamide

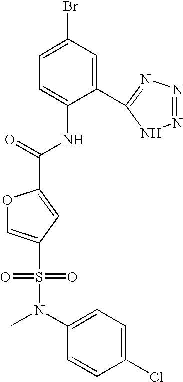

A mixture of the nitrile (199 mg, 0.402 mmol), sodium azide (34 mg, 0.52 mmol), and triethylamine hydrochloride (81 mg, 0.59 mmol) in toluene (10 mL) was placed under nitrogen and heated in a 100° C. oil bath overnight. The mixture was added to a separatory funnel with 100 mL of CH₂Cl₂, and the solution was washed with 2×100 mL of 1 M aqueous HCl and 100 mL of brine. The CH₂Cl₂ was evaporated, and the residue was recrystallized from hot methanol/THF. The crystals were washed with pentane and dried at 85° C. under vacuum yielding 64 mg of white solid.

¹H NMR (400 MHz, DMSO-d₆) δ 11.57 (s, 1H), 8.66 (s, 1H), 8.36 (d, J=8.7 Hz, 1H), 8.22 (d, J=2.5 Hz, 1H), 7.82 (dd, J=9.2, 2.0 Hz, 1H), 7.47 (d, J=8.7 Hz, 2H), 7.29-7.32 (m, 3H), 3.23 (s, 3H).

Preparation of N-(4-Bromo-2-cyanophenyl)-4-{[(4-chlorophenyl)(methyl)amino]sulfonyl}thiophene-2-carboxamide and N-(4-bromo-2-cyanophenyl)-5-{[(4-chlorophenyl)(methyl)amino]sulfonyl}thiophene-2-carboxamide

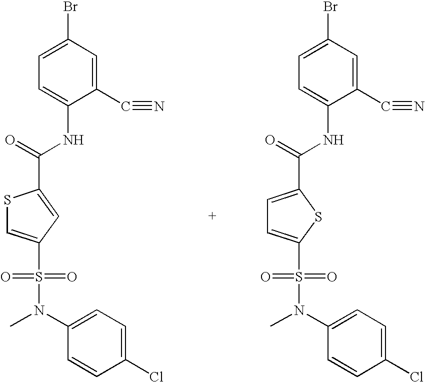

To a 2:1 mixture of 4-{[(4-chlorophenyl)(methyl)amino]sulfonyl}thiophene-2-carboxylic acid and 5-{[(4-chlorophenyl)(methyl)amino]sulfonyl}thiophene-2-carboxylic acid (1.90 g, 5.73 mmol) in CH₂Cl₂ (25 mL) was added DMF (15 μL) and oxalyl chloride (1.0 mL, 11 mmol). The mixture was stirred for 1 hour, and the solvent and excess oxalyl chloride were removed by rotary evaporation. The residue was dissolved in CH₂Cl₂ (20 mL), and 2-amino-5-bromobenzonitrile (0.97 g, 4.9 mmol) in pyridine (10 mL) was added. The mixture was stirred overnight and then added to a separatory funnel with 200 mL of CH₂Cl₂. This solution was washed with 2×100 mL of 1 M aqueous HCl and 100 mL of brine. The CH₂Cl₂ was evaporated in the presence of silica gel, and the products were purified by chromatography using a Biotage Flash 40 M silica cartridge with CH₂Cl₂ as eluent. The faster eluting spot yielded 480 mg of the 2,5-thiophene isomer while the slower eluting spot yielded 1.10 g of the 2,4-thiophene isomer.

### Example 28

### N-[4-Bromo-2-(1H-tetraazol-5-yl)phenyl]-4-{[(4-chlorophenyl)(methyl)amino]sulfonyl}thiophene-2-carboxamide

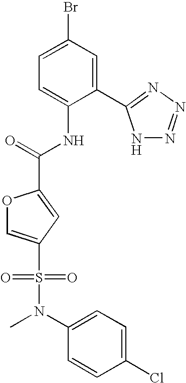

A mixture of the nitrile (614 mg, 1.20 mmol), sodium azide (101 mg, 1.55 mmol, EM Science), and triethylamine hydrochloride (270 mg, 1.96 mmol, Aldrich) in toluene (30 mL) was placed under nitrogen and heated in a 100° C. oil bath overnight. The mixture was added to a separatory funnel with 100 mL of CH₂Cl₂, and the solution was washed with 2×100 mL of 1 M aqueous HCl. A precipitate began to form in the CH₂Cl₂, so this solution was cooled in the freezer. The resulting crystals were washed with ethanol and dried at 100° C. under vacuum yielding 432 mg of white solid.

¹H NMR (400 MHz, DMSO-d₆) δ 11.31 (s, 1H), 8.35 (s, 1H), 8.18 (d, J=2.5 Hz, 1 H), 8.15 (d, J=9.2 Hz, 1H), 8.00 (s, 1H), 7.82 (dd, J=8.9, 2.3 Hz, 1H), 7.44 (d, J=8.7 Hz, 2H), 7.22 (d, J=8.7 Hz, 2H), 3.24 (s, 3H).

### Example 29

### N-[4-Bromo-2-(1H-tetraazol-5-yl)phenyl]-5-{[(4-chlorophenyl)(methyl)amino]sulfonyl}thiophene-2-carboxamide

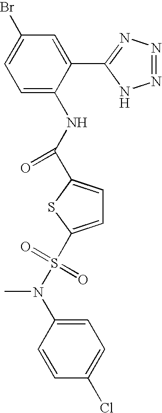

A mixture of the nitrile (399 mg, 0.781 mmol), sodium azide (67 mg, 1.0 mmol, EM Science), and triethylamine hydrochloride (245 mg, 1.78 mmol, Aldrich) in toluene (20 mL) was placed under nitrogen and heated in a 100° C. oil bath overnight. The mixture was added to a separatory funnel with 100 mL of CH₂Cl₂, and the solution was washed with 2×100 mL of 1 M aqueous HCl and 100 mL of brine. The product then spontaneously recrystallized from the CH₂Cl₂. The crystals were washed with ethanol and dried at 100° C. under vacuum yielding 230 mg of white solid.

¹H NMR (400 MHz, DMSO-d₆) δ 11.57 (s, 1H), 8.26 (d, J=8.7 Hz, 1H), 8.22 (d, J=2.5 Hz, 1H), 7.97 (d, J=4.1 Hz, 1H), 7.82 (dd, J=8.9, 2.3 Hz, 1H), 7.66 (d, J=4.1 Hz, 1H), 7.48 (d, J=8.7 Hz, 2H), 7.26 (d, J=9.2 Hz, 2H), 3.24 (s, 3H).

### Example 30

### N-[4-bromo-2-(5-oxo-4,5-dihydro-1,2,4-oxadiazol-3-yl)phenyl]-5-phenylisoxazole-3-carboxamide, PHA-689663

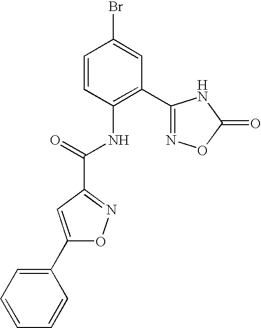

5-Phenylisoxazole-3-carboxylic acid (Shimizu, T.; Hayashi, Y.; Teramura, K. Bull. Chem. Soc. Jpn. 1985, 58, 2519; 245 mg, 1.29 mmol), DCM (40 ml) and oxyl chloride (4.0 ml) were place in a flask, followed by the addition of on drop of DMF (0.1 ml). The solution was stirred at room temperature for about 6 h. Then heptane (20 ml) was added and the solvent was removed. The residue was dissolved in DCM (10 ml). 3-(2-amino-5-bromophenyl)-1,2,4-oxadiazol-5(4H)-one (300 mg, 1.18 mmol) was added and followed by the addition of pyridine (0.3 ml). The resulting solution was stirred overnight, then diluted with MTBE (200 ml) and washed with 1N HCl, brine, dried (MgSO₄), filtered, and concentrated in vacuo. The residue was purified by flash chromatography (DCM/MeOH=1:0, 50:1) to afford 105 mg (20%) of the desired product as a white solid. ¹H NMR (300 MHz, DMSO-d₆) δ 11.03 (s, 1H), 8.27 (d, J=9.0 Hz, 1H), 8.00 (m, 2H), 7.97 (d, J=2.3 Hz, 1H), 7.89 (dd, J=2.3, 8.8 Hz, 1H), 7.57 (m, 4H).

Preparation of 6-(Isopropylthio)nicotinic acid

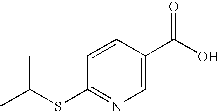

6,6′-Dithiodinicotinic acid (12.32 g, 40 mmol) was dissolved in DMF (100 ml). NaBH₄ (1.52 g, 40 mmol) was added. Gas evolution was observed then 5 min later, 2-iodo-propane (68 g, 400 mmol) was added. The resulting solution was stirred for 24 h, the DMF was removed in vacuo. The residue was re-dissolved in MeOH and DCM followed by the addition of 2M HCl (20 ml) ether solution, then silica gel was added and the mixture was concentrated in vacuo. The residue was purified by silica gel chromatography (MeOH/DCM: 0/1, 1/100) to afford 9.4 g (60%) of the desired product as a yellow solid. ¹H NMR (300 MHz, DMSO-d₆) δ 9.79 (s, 1H), 9.13 (d, J=1.8 Hz, 1H), 8.13 (dd, J=8.4, 2.2 Hz, 1H), 7.25 (d, J=8.5 Hz, 1H), 4.13 (hept, J=6.8 Hz, 1H), 1.46 (d, J=7.0 Hz, 6H).

### Example 31

### N-[4-bromo-2-(5-oxo-4,5-dihydro-1,2,4-oxadiazol-3-yl)phenyl]-6-(isopropylthio)nicotinamide, PHA-676847

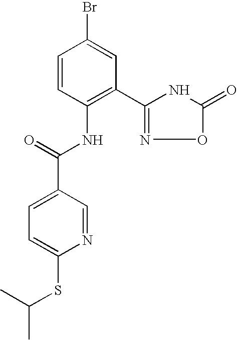

6-(Isopropylthio)nicotinic acid (212 mg, 1.08 mmol) was dissolved in thionyl chloride (40 ml). The solution was heated at refluxing temperature overnight. Toluene (30 ml) was added. Then the solution was concentrated in vacuo to dryness. The residue was re-dissolved in DCM (50 ml) followed by the addition of 3-(2-amino-5-bromophenyl)-1,2,4-oxadiazol-5(4H)-one (250 mg, 0.98 mmol) and pyridine (5 ml). The mixture was stirred overnight. The resulting solution was diluted with MTBE (200 ml) and washed with 1N HCl, brine and dried (MgSO4). The solution was concentrated in vacuo and the residue was purified by the flash chromatography (MeOH/DCM: 1/100, 1/50, 1/20) to afford 100 mg 24%) of the desired product as a yellow solid. ¹H NMR (300 MHz, DMSO-d₆) δ 10.51 (s, 1H), 8.95 (d, J=2.0 Hz, 1H), 8.08 (dd, J=2.4, 8.5 Hz, 1H), 8.00 (d, J=8.8 Hz, 1H), 7.90 (d, J=2.3 Hz, 1H), 7.85 (dd, J=2.4, 8.8 Hz, 1H), 7.46 (d, J=8.4 Hz, 1H), 4.04 (hept, J=6.8 Hz, 1H), 1.38 (d, J=6.74 Hz, 6H).

### Example 32

Additional compounds which may be produced by the methodology described above and known to those skilled in the art, includes, but is not limited to, the following:

N-[4-cyano-2-(1H-tetraazol-5-yl)phenyl]-1H-indole-2-carboxamide

N-[4-cyano-2-(1H-tetraazol-5-yl)phenyl]-5-methoxy-1H-indole-2-carboxamide

5-(benzyloxy)-N-[4-cyano-2-(1H-tetraazol-5-yl)phenyl]-1H-indole-2-carboxamide

N-[4-cyano-2-(1H-tetraazol-5-yl)phenyl]-1-methyl-1H-indole-2-carboxamide

6-(benzyloxy)-N-[4-cyano-2-(1H-tetraazol-5-yl)phenyl]-1H-indole-2-carboxamide

7-chloro-N-[4-cyano-2-(1H-tetraazol-5-yl)phenyl]-1H-indole-2-carboxamide

N-[4-cyano-2-(1H-tetraazol-5-yl)phenyl]-4-methoxy-1H-indole-2-carboxamide

6-chloro-N-[4-cyano-2-(1H-tetraazol-5-yl)phenyl]-1H-indole-2-carboxamide

1-benzyl-N-[4-cyano-2-(1H-tetraazol-5-yl)phenyl]-1H-indole-2-carboxamide

N-[4-cyano-2-(1H-tetraazol-5-yl)phenyl]-1-ethyl-1H-indole-2-carboxamide

N-[4-cyano-2-(1H-tetraazol-5-yl)phenyl]-7-(phenylsulfonyl)-1H-indole-2-carboxamide

1-allyl-N-[4-cyano-2-(1H-tetraazol-5-yl)phenyl]-1H-indole-2-carboxamide

N-[4-cyano-2-(1H-tetraazol-5-yl)phenyl]-1-(cyclohexylmethyl)-1H-indole-2-carboxamide

N-[4-cyano-2-(1H-tetraazol-5-yl)phenyl]-1-(2-methoxyethyl)-1H-indole-2-carboxamide

N-[4-cyano-2-(1H-tetraazol-5-yl)phenyl]-1-pentyl-1H-indole-2-carboxamide

1-butyl-N-[4-cyano-2-(1H-tetraazol-5-yl)phenyl]-1H-indole-2-carboxamide

N-[4-cyano-2-(1H-tetraazol-5-yl)phenyl]-1-propyl-1H-indole-2-carboxamide

N-[4-cyano-2-(1H-tetraazol-5-yl)phenyl]-1-isopropyl-1H-indole-2-carboxamide

N-[4-cyano-2-(1H-tetraazol-5-yl)phenyl]-1-isobutyl-1H-indole-2-carboxamide

N-[4-cyano-2-(1H-tetraazol-5-yl)phenyl]-1-(3-phenylpropyl)-1H-indole-2-carboxamide

N-[4-cyano-2-(1H-tetraazol-5-yl)phenyl]-1-methyl-7-(phenylsulfonyl)-1H-indole-2-carboxamide

N-[4-cyano-2-(1H-tetraazol-5-yl)phenyl]-7-[(phenylacetyl)amino]-1H-indole-2-carboxamide

7-(benzoylamino)-N-[4-cyano-2-(1H-tetraazol-5-yl)phenyl]-1H-indole-2-carboxamide

2-{[2-({[4-cyano-2-(1H-tetraazol-5-yl)phenyl]amino}carbonyl)-1H-indol-7-yl]amino}-2-oxoethyl acetate

N-[4-cyano-2-(1H-tetraazol-5-yl)phenyl]-7-[(cyclopentylcarbonyl)amino]-1H-indole-2-carboxamide

7-amino-N-[4-cyano-2-(1H-tetraazol-5-yl)phenyl]-1H-indole-2-carboxamide

N-[4-cyano-2-(1H-tetraazol-5-yl)phenyl]-1,2-benzisoxazole-3-carboxamide

7-{[(6-chloropyridin-3-yl)carbonyl]amino}-N-[4-cyano-2-(1H-tetraazol-5-yl)phenyl]-1H-indole-2-carboxamide

N-[4-cyano-2-(1H-tetraazol-5-yl)phenyl]-7-[(isoxazol-5-ylcarbonyl)amino]-1H-indole-2-carboxamide

N-[4-cyano-2-(1H-tetraazol-5-yl)phenyl]-7-[(2,4-difluorobenzoyl)amino]-1H-indole-2-carboxamide

N-[4-cyano-2-(1H-tetraazol-5-yl)phenyl]-7-[(fluoroacetyl)amino]-1H-indole-2-carboxamide

7-(acetylamino)-N-[4-cyano-2-(1H-tetraazol-5-yl)phenyl]-1H-indole-2-carboxamide

7-{[(4-chlorophenyl)acetyl]amino}-N-[4-cyano-2-(1H-tetraazol-5-yl)phenyl]-1H-indole-2-carboxamide

N-[4-cyano-2-(1H-tetraazol-5-yl)phenyl]-7-{[(4-methoxyphenyl)acetyl]amino}-1H-indole-2-carboxamide

N-[4-cyano-2-(1H-tetraazol-5-yl)phenyl]-7-[(cyclopentylacetyl)amino]-1H-indole-2-carboxamide

N-[4-cyano-2-(1H-tetraazol-5-yl)phenyl]-7-[(3-fluorobenzoyl)amino]-1H-indole-2-carboxamide

7-[(3-cyanobenzoyl)amino]-N-[4-cyano-2-(1H-tetraazol-5-yl)phenyl]-1H-indole-2-carboxamide

N-[4-cyano-2-(1H-tetraazol-5-yl)phenyl]-7-[(cyclohexylcarbonyl)amino]-1H-indole-2-carboxamide

N-[4-cyano-2-(1H-tetraazol-5-yl)phenyl]-7-(propionylamino)-1H-indole-2-carboxamide methyl 5-{[2-({[4-cyano-2-(1H-tetraazol-5-yl)phenyl]amino}carbonyl)-1H-indol-7-yl]amino}-5-oxopentanoate

7-(butyrylamino)-N-[4-cyano-2-(1H-tetraazol-5-yl)phenyl]-1H-indole-2-carboxamide

7-[(4-bromobenzoyl)amino]-N-[4-cyano-2-(1H-tetraazol-5-yl)phenyl]-1H-indole-2-carboxamide

N-[4-cyano-2-(1H-tetraazol-5-yl)phenyl]-7-[(3-phenylpropanoyl)amino]-1H-indole-2-carboxamide

N-[4-cyano-2-(1H-tetraazol-5-yl)phenyl]-7-[(phenoxyacetyl)amino]-1H-indole-2-carboxamide

N-[4-cyano-2-(1H-tetraazol-5-yl)phenyl]-7-[(3-cyclopentylpropanoyl)amino]-1H-indole-2-carboxamide methyl 3-{[2-({[4-cyano-2-(1H-tetraazol-5-yl)phenyl]amino}carbonyl)-1H-indol-7-yl]amino}-3-oxopropanoate

N-[4-cyano-2-(1H-tetraazol-5-yl)phenyl]-7-[(2-ethylhexanoyl)amino]-1H-indole-2-carboxamide

N-[4-cyano-2-(1H-tetraazol-5-yl)phenyl]-7-{[(3,4-dimethoxyphenyl)acetyl]amino}-1H-indole-2-carboxamide

N-[4-cyano-2-(1H-tetraazol-5-yl)phenyl]-7-[(3,5,5-trimethylhexanoyl)amino]-1H-indole-2-carboxamide

N-[4-cyano-2-(1H-tetraazol-5-yl)phenyl]-7-[(cyclopropylcarbonyl)amino]-1H-indole-2-carboxamide

N-[4-cyano-2-(1H-tetraazol-5-yl)phenyl]-7-[(methoxyacetyl)amino]-1H-indole-2-carboxamide

N-[4-cyano-2-(1H-tetraazol-5-yl)phenyl]-7-[(3-methylbutanoyl)amino]-1H-indole-2-carboxamide

N-[4-cyano-2-(1H-tetraazol-5-yl)phenyl]-7-(pentanoylamino)-1H-indole-2-carboxamide

N-[4-cyano-2-(1H-tetraazol-5-yl)phenyl]-7-{[(4,7,7-trimethyl-3-oxo-2-oxabicyclo[2.2.1]hept-1-yl)carbonyl]amino}-1H-indole-2-carboxamide

7-{[chloro(phenyl)acetyl]amino}-N-[4-cyano-2-(1H-tetraazol-5-yl)phenyl]-1H-indole-2-carboxamide

7-{[(benzyloxy)acetyl]amino}-N-[4-cyano-2-(1H-tetraazol-5-yl)phenyl]-1H-indole-2-carboxamide

ethyl 3-{[2-({[4-cyano-2-(1H-tetraazol-5-yl)phenyl]amino}carbonyl)-1H-indol-7-yl]amino}-3-oxopropanoate

7-L(1-adamantylcarbonyl)amino]-N-[4-cyano-2-(1H-tetraazol-5-yl)phenyl]-1H-indole-2-carboxamide

N-[4-cyano-2-(1H-tetraazol-5-yl)phenyl]-7-(hexanoylamino)-1H-indole-2-carboxamide

N-[4-cyano-2-(1H-tetraazol-5-yl)phenyl]-7-{[(2-phenylcyclopropyl)carbonyl]amino}-1H-indole-2-carboxamide

N-[4-cyano-2-(1H-tetraazol-5-yl)phenyl]-7-[(2-phenylbutanoyl)amino]-1H-indole-2-carboxamide

N-[4-cyano-2-(1H-tetraazol-5-yl)phenyl]-7-(heptanoylamino)-1H-indole-2-carboxamide

2-{[2-({[4-cyano-2-(1H-tetraazol-5-yl)phenyl]amino}carbonyl)-1H-indol-7-yl]amino}-2-oxo-1-phenylethyl acetate

N-[4-cyano-2-(1H-tetraazol-5-yl)phenyl]-7-[(thien-2-ylcarbonyl)amino]-1H-indole-2-carboxamide

N-[4-cyano-2-(1H-tetraazol-5-yl)phenyl]-7-[(2-methylbutanoyl)amino]-1H-indole-2-carboxamide

methyl 8-{[2-({[4-cyano-2-(1H-tetraazol-5-yl)phenyl]amino} carbonyl)-1H-indol-7-yl]amino}-8-oxooctanoate

N-[4-cyano-2-(1H-tetraazol-5-yl)phenyl]-7-[(2-ethylbutanoyl)amino]-1H-indole-2-carboxamide

N-[4-cyano-2-(1H-tetraazol-5-yl)phenyl]-7-(octanoylamino)-1H-indole-2-carboxamide

N-[4-cyano-2-(1H-tetraazol-5-yl)phenyl]-7-[(cyclobutylcarbonyl)amino]-1H-indole-2-carboxamide

N-[4-cyano-2-(1H-tetraazol-5-yl)phenyl]-7-(1,3-dioxo-1,3-dihydro-2H-isoindol-2-yl)-1H-indole-2-carboxamide

7-({[2-(benzylthio)-1,3-thiazol-4-yl]carbonyl}amino)-N-[4-cyano-2-(1H-tetraazol-5-yl)phenyl]-1H-indole-2-carboxamide

N-[4-cyano-2-(1H-tetraazol-5-yl)phenyl]-7-{[3-(morpholin-4-ylsulfonyl)benzoyl]amino}-1H-indole-2-carboxamide

N-[4-cyano-2-(1H-tetraazol-5-yl)phenyl]-7-[(1H-indol-2-ylcarbonyl)amino]-1H-indole-2-carboxamide

N-[2-({[-4-cyano-2-(1H-tetraazol-5-yl)phenyl]amino}carbonyl)-1H-indol-7-yl]-1-methyl-1H-indole-2-carboxamide

N-[4-cyano-2-(1H-tetraazol-5-yl)phenyl]-7-{[(5-phenylisoxazol-3-yl)carbonyl]amino}-1H-indole-2-carboxamide

N-[4-cyano-2-(1H-tetraazol-5-yl)phenyl]-7-[(5-phenylpentanoyl)amino]-1H-indole-2-carboxamide

N-[4-cyano-2-(1H-tetraazol-5-yl)phenyl]-7-[(4-phenylbutanoyl)amino]-1H-indole-2-carboxamide

N-[4-cyano-2-(1H-tetraazol-5-yl)phenyl]-7-{[4-(4-methoxyphenyl)butanoyl]amino}-1H-indole-2-carboxamide

7-{[(2-chlorophenyl)acetyl]amino}-N-[4-cyano-2-(1H-tetraazol-5-yl)phenyl]-1H-indole-2-carboxamide

N-[4-cyano-2-(1H-tetraazol-5-yl)phenyl]-7-{[(2,4-dichlorophenyl)acetyl]amino}-1H-indole-2-carboxamide

N-[4-cyano-2-(1H-tetraazol-5-yl)phenyl]-7-{[(3,4-dichlorophenyl)acetyl]amino}-1H-indole-2-carboxamide

7-{[(3-chlorophenyl)acetyl]amino}-N-[4-cyano-2-(1H-tetraazol-5-yl)phenyl]-1H-indole-2-carboxamide

N-[4-cyano-2-(1H-tetraazol-5-yl)phenyl]-7-({[3-(trifluoromethyl)phenyl]acetyl}amino)-1H-indole-2-carboxamide

N-[4-cyano-2-(1H-tetraazol-5-yl)phenyl]-7-{[(3-methylphenyl)acetyl]amino}-1H-indole-2-carboxamide

7-{[(4-tert-butylphenyl)acetyl]amino}-N-[4-cyano-2-(1H-tetraazol-5-yl)phenyl]-1H-indole-2-carboxamide

N-[4-cyano-2-(1H-tetraazol-5-yl)phenyl]-7-{[(3-methoxyphenyl)acetyl]amino}-1H-indole-2-carboxamide

N-[4-cyano-2-(1H-tetraazol-5-yl)phenyl]-7-{[(2-methoxyphenyl)acetyl]amino}-1H-indole-2-carboxamide

N-[4-cyano-2-(1H-tetraazol-5-yl)phenyl]-7-{[(2-methylphenyl)acetyl]amino}-1H-indole-2-carboxamide

N-[4-cyano-2-(1H-tetraazol-5-yl)phenyl]-7-({[4-(trifluoromethyl)phenyl]acetyl}amino)-1H-indole-2-carboxamide

N-[4-cyano-2-(1H-teraazol-5-yl)phenyl]7-{[(4-isopropylphenyl)acetyl]amino}-1H-indole-2-carboxamide

N-[4-cyano-2-(1H-tetraazol-5-yl)phenyl]-7-{[(4-methylphenyl)acetyl]amino}-1H-indole-2-carboxamide

N-[4-cyano-2-(1H-tetraazol-5-yl)phenyl]-7-{[(4-fluorophenyl)acetyl]amino}-1H-indole-2-carboxamide

N-[4-cyano-2-(1H-tetraazol-5-yl)phenyl]-7-({[2-(trifluoromethyl)phenyl]acetyl}amino)-1H-indole-2-carboxamide

N-[4-cyano-2-(1H-tetraazol-5-yl)phenyl]-7-{[(3-fluorophenyl)acetyl]amino}-1H-indole-2-carboxamide

N-[4-cyano-2-(1H-tetraazol-5-yl)phenyl]-7-{[(phenylthio)acetyl]amino}-1H-indole-2-carboxamide

N-[4-cyano-2-(1H-tetraazol-5-yl)phenyl]-7-[(2-naphthylacetyl)amino]-1H-indole-2-carboxamide

N-[4-cyano-2-(1H-tetraazol-5-yl)phenyl]-7-[(1-naphthylacetyl)amino]-1H-indole-2-carboxamide

N-[4-cyano-2-(1H-tetraazol-5-yl)phenyl]-7-{[(2-naphthyloxy)acetyl]amino}-1H-indole-2-carboxamide

N-[4-cyano-2-(1H-tetraazol-5-yl)phenyl]-7-[(2-propoxybenzoyl)amino]-1H-indole-2-carboxamide

N-[4-cyano-2-(1H-tetraazol-5-yl)phenyl]-7-[(tetrahydrofuran-3-ylcarbonyl)amino]-1H-indole-2-carboxamide

N-[4-cyano-2-(1H-tetraazol-5-yl)phenyl]-7-{[(1-methylcyclopropyl)carbonyl]amino}-1H-indole-2-carboxamide

N-[4-cyano-2-(1H-tetraazol-5-yl)phenyl]-7-{[(4-ethoxyphenyl)acetyl]amino}-1H-indole-2-carboxamide

7-[(1-benzothien-3-ylacetyl)amino]-N-[4-cyano-2-(1H-tetraazol-5-yl)phenyl]-1H-indole-2-carboxamide

7-[(1,1′-biphenyl-4-ylcarbonyl)amino]-N-[4-cyano-2-(1H-tetraazol-5-yl)phenyl]-1H-indole-2-carboxamide

7-[(4-butoxybenzoyl)amino]-N-[4-cyano-2-(1H-tetraazol-5-yl)phenyl]-1H-indole-2-carboxamide

N-[4-cyano-2-(1H-tetraazol-5-yl)phenyl]-7-{[2-(2-phenylethyl)benzoyl]amino}-1H-indole-2-carboxamide

7-[(1,1′-biphenyl-2-ylcarbonyl)amino]-N-[4-cyano-2-(1H-tetraazol-5-yl)phenyl]-1H-indole-2-carboxamide

N-[4-cyano-2-(1H-tetraazol-5-yl)phenyl]-7-{[4-(ethylthio)benzoyl]amino}-1H-indole-2-carboxamide

N-[4-cyano-2-(1H-tetraazol-5-yl)phenyl]-7-{[2-(methylsulfonyl)benzoyl]amino}-1H-indole-2-carboxamide

N-[4-cyano-2-(1H-tetraazol-5-yl)phenyl]-7-{[(2,6-dichlorophenyl)acetyl]amino}-1H-indole-2-carboxamide

7-[(1,1′-biphenyl-4-ylacetyl)amino]-N-[4-cyano-2-(1H-tetraazol-5-yl)phenyl]-1H-indole-2-carboxamide

7-[(1,3-benzodioxol-5-ylacetyl)amino]-N-[4-cyano-2-(1H-tetraazol-5-yl)phenyl]-1H-indole-2-carboxamide

N-[4-cyano-2-(1H-tetraazol-5-yl)phenyl]-7-[(3,3-dimethylbutanoyl)amino]-1H-indole-2-carboxamide

N-[4-cyano-2-(1H-tetraazol-5-yl)phenyl]-7-[(thien-2-ylacetyl)amino]-1H-indole-2-carboxamide

N-[4-cyano-2-(1H-tetraazol-5-yl)phenyl]-7-{[(3-methyl-5-phenylisoxazol-4-yl)carbonyl]amino}-1H-indole-2-carboxamide

N-[4-cyano-2-(1H-tetraazol-5-yl)phenyl]-7-({[2-(2-methoxyethoxy)ethoxy]acetyl}amino)-1H-indole-2-carboxamide

N-[4-cyano-2-(1H-tetraazol-5-yl)phenyl]-7-[(2-hydroxybenzoyl)amino]-1H-indole-2-carboxamide

N-[4-cyano-2-(1H-tetraazol-5-yl)phenyl]-7-({[4-(trifluoromethoxy)phenyl]sulfonyl} amino)-1H-indole-2-carboxamide

N-[4-cyano-2-(1H-tetraazol-5-yl)phenyl]-7-(prolylamino)-1H-indole-2-carboxamide

N-[4-cyano-2-(1H-tetraazol-5-yl)phenyl]-7-{[(3-methylisoxazol-5-yl)acetyl]amino}-1H-indole-2-carboxamide

N-[4-cyano-2-(1H-tetraazol-5-yl)phenyl]-6-methoxy-1H-indole-2-carboxamide

N-[4-cyano-2-(1H-tetraazol-5-yl)phenyl]-5-nitro-1,2-benzisoxazole-3-carboxamide

7-[(benzylsulfonyl)amino]-N-[4-cyano-2-(1H-tetraazol-5-yl)phenyl]-1H-indole-2-carboxamide

N-[4-cyano-2-(1H-tetraazol-5-yl)phenyl]-1-methyl-7-{[3-(morpholin-4-ylsulfonyl)benzoyl]amino}-1H-indole-2-carboxamide

N-[4-cyano-2-(1H-tetraazol-5-yl)phenyl]-7-{[(4-fluorophenyl)acetyl]amino}-1-methyl-1H-indole-2-carboxamide

N-[4-cyano-2-(1H-tetraazol-5-yl)phenyl]-7-[(fluoroacetyl)amino]-1-methyl-1H-indole-2-carboxamide

N-[4-cyano-2-(1H-tetraazol-5-yl)phenyl]-1-methyl-7-{[(1-methyl-1H-indol-2-yl)carbonyl]amino}-1H-indole-2-carboxamide

N-[4-cyano-2-(1H-tetraazol-5-yl)phenyl]-5-[(phenylacetyl)amino]-1,2-benzisoxazole-3-carboxamide

5-[(benzylsulfonyl)amino]-N-[4-cyano-2-(1H-tetraazol-5-yl)phenyl]-1,2-benzisoxazole-3-carboxamide

6-(benzyloxy)-N-[4-cyano-2-(1H-tetraazol-5-yl)phenyl]-1-methyl-1H-indole-2-carboxamide

N-[4-cyano-2-(1H-tetraazol-5-yl)phenyl]-6-methoxy-1-methyl-1H-indole-2-carboxamide

N-[4-cyano-2-(1H-tetraazol-5-yl)phenyl]-1-methyl-7-[(morpholin-4-ylcarbonyl)amino]-1H-indole-2-carboxamide

N-[4-cyano-2-(1H-tetraazol-5-yl)phenyl]-1-methyl-7-({[(tetrahydrofuran-2-ylmethyl)amino]carbonyl}amino)-1H-indole-2-carboxamide

N-[4-cyano-2-(1H-tetraazol-5-yl)phenyl]-7-hydroxy-1-methyl-1H-indole-2-carboxamide

7-{[(benzylamino)carbonyl]amino}-N-[4-cyano-2-(1H-tetraazol-5-yl)phenyl]-1-methyl-1H-indole-2-carboxamide

N-[4-cyano-2-(1H-tetraazol-5-yl)phenyl]-7-({[(2,3-dihydroxypropyl)amino]carbonyl}amino)-1-methyl-1H-indole-2-carboxamide

1-[({[2-({[4-cyano-2-(1H-tetraazol-5-yl)phenyl]amino}carbonyl)-1-methyl-1H-indol-7-yl]amino}carbonyl)(methyl)amino]-1-deoxyhexitol

N-[4-cyano-2-(1H-tetraazol-5-yl)phenyl]-7-(2,3-dihydro-1,4-benzodioxin-2-ylmethoxy)-1-methyl-1H-indole-2-carboxamide

7-(benzyloxy)-N-[4-cyano-2-(1H-tetraazol-5-yl)phenyl]-1H-indole-2-carboxamide

N-[4-cyano-2-(1H-tetraazol-5-yl)phenyl]-1-methyl-7-(3-phenoxypropoxy)-1H-indole-2-carboxamide

5-(acetylamino)-N-[4-cyano-2-(1H-tetraazol-5-yl)phenyl]-1,2-benzisoxazole-3-carboxamide

N-[4-cyano-2-(1H-tetraazol-5-yl)phenyl]-5-[(methylsulfonyl)amino]-1,2-benzisoxazole-3-carboxamide

5-(benzoylamino)-N-[4-cyano-2-(1H-tetraazol-5-yl)phenyl]-1,2-benzisoxazole-3-carboxamide

N-[4-cyano-2-(1H-tetraazol-5-yl)phenyl]-5-[(phenylsulfonyl)amino]-1,2-benzisoxazole-3-carboxamide

N-[4-cyano-2-(1H-tetraazol-5-yl)phenyl]-7-(cyclobutylmethoxy)-1-methyl-1H-indole-2-carboxamide

N-[4-cyano-2-(1H-tetraazol-5-yl)phenyl]-7-(2-furylmethoxy)-1-methyl-1H-indole-2-carboxamide

N-[4-cyano-2-(1H-tetraazol-5-yl)phenyl]-7-{[(4S)-2,2-dimethyl-1,3-dioxolan-4-yl]methoxy}-1-methyl-1H-indole-2-carboxamide

N-[4-cyano-2-(1H-tetraazol-5-yl)phenyl]-7-{[(2R)-2,3-dihydroxypropyl]oxy}-1methyl-1H-indole-2-carboxamide

N-[4-cyano-2-(1H-tetraazol-5-yl)phenyl]-7-(cyclobutyloxy)-1-methyl-1H-indole-2-carboxamide

N-[4-cyano-2-(1H-tetraazol-5-yl)phenyl]-7-(2-methoxy-1-methylethoxy)-1-methyl-1H-indole-2-carboxamide

N-[4-cyano-2-(1H-tetraazol-5-yl)phenyl]-7-isopropoxy-1-methyl-1H-indole-2-carboxamide

7-(benzyloxy)-N-[4-cyano-2-(1H-tetraazol-5-yl)phenyl]-1-methyl-1H-indole-2-carboxamide

6-chloro-N-[4-cyano-2-(1H-tetraazol-5-yl)phenyl]-1,2-benzisoxazole-3-carboxamide

6-sec-butoxy-N-[4-cyano-2-(1H-tetraazol-5-yl)phenyl]-1-methyl-1H-indole-2-carboxamide

6-butoxy-N-[4-cyano-2-(1H-tetraazol-5-yl)phenyl]-1-methyl-1H-indole-2-carboxamide

N-[4-cyano-2-(1H-tetraazol-5-yl)phenyl]-5-{[(4-fluorophenyl)sulfonyl]amino}-1,2-benzisoxazole-3-carboxamide

5-amino-N-[4-cyano-2-(1H-tetraazol-5-yl)phenyl]-1,2-benzisoxazole-3-carboxamide

5-bromo-N-[4-cyano-2-(1H-tetraazol-5-yl)phenyl]-1,2-benzisoxazole-3-carboxamide

6-[(benzylsulfonyl)amino]-N-[4-cyano-2-(1H-tetraazol-5-yl)phenyl]-1,2-benzisoxazole-3-carboxamide

N-[4-cyano-2-(1H-tetraazol-5-yl)phenyl]-6-[(phenylsulfonyl)amino]-1,2-benzisoxazole-3-carboxamide

N-[4-cyano-2-(1H-tetraazol-5-yl)phenyl]-7-(cyclohexylmethoxy)-1-methyl-1H-indole-2-carboxamide

N-[4-cyano-2-(1H-tetraazol-5-yl)phenyl]-7-(cyclopropylmethoxy)-1-methyl-1H-indole-2-carboxamide

N-[4-cyano-2-(1H-tetraazol-5-yl)phenyl]-1-methyl-7-(tetrahydro-2H-pyran-2-ylmethoxy)-1H-indole-2-carboxamide

N-[4-cyano-2-(1H-tetraazol-5-yl)phenyl]-1-methyl-7-(pentyloxy)-1H-indole-2-carboxamide

N-[4-cyano-2-(1H-tetraazol-5-yl)phenyl]-5-phenyl-1,2-benzisoxazole-3-carboxamide

N-[4-cyano-2-(1H-tetraazol-5-yl)phenyl]-5-[2-(trifluoromethyl)phenyl]-1,2-benzisoxazole-3-carboxamide

N-[4-cyano-2-(1H-tetraazol-5-yl)phenyl]-7-(2-methoxyethoxy)-1-methyl-1H-indole-2-carboxamide

N-[4-cyano-2-(1H-tetraazol-5-yl)phenyl]-7-(2-hydroxy-3-isopropoxypropoxy)-1-methyl-1H-indole-2-carboxamide

N-[4-cyano-2-(1H-tetraazol-5-yl)phenyl]-6-phenyl-1,2-benzisoxazole-3-carboxamide

N-[4-cyano-2-(1H-tetraazol-5-yl)phenyl]-6-[2-(trifluoromethyl)phenyl]-1,2-benzisoxazole-3-carboxamide

N-[4-cyano-2-(1H-tetraazol-5-yl)phenyl]-1-methyl-7-[2-(methylthio)ethoxy]-1H-indole-2-carboxamide

7-[(4-azido-3-iodobenzoyl)amino]-N-[4-cyano-2-(1H-tetraazol-5-yl)phenyl]-1-methyl-1H-indole-2-carboxamide

5-[(4-azido-3-iodobenzoyl)amino]-N-[4-cyano-2-(1H-tetraazol-5-yl)phenyl]-1,2-benzisoxazole-3-carboxamide

N-[4-cyano-2-(1H-tetraazol-5-yl)phenyl]-6-[(methylsulfonyl)amino]-1,2-benzisoxazole-3-carboxamide

N-[4-cyano-2-(1H-tetraazol-5-yl)phenyl]-6-{[(4-fluorophenyl)sulfonyl]amino}-1,2-benzisoxazole-3-carboxamide

6-amino-N-[4-cyano-2-(1H-tetraazol-5-yl)phenyl]-1,2-benzisoxazole-3-carboxamide

N-[4-cyano-2-(1H-tetraazol-5-yl)phenyl]-5-(2-fluorophenyl)-1,2-benzisoxazole-3-carboxamide

N-[4-cyano-2-(1H-tetraazol-5-yl)phenyl]-1-methyl-6-[2-(trifluoromethyl)phenyl]-1H-indole-2-carboxamide

N-[4-cyano-2-(1H-tetraazol-5-yl)phenyl]-1-methyl-6-(2,3,4-trimethoxyphenyl)-1H-indole-2-carboxamide

N-[4-cyano-2-(1H-tetraazol-5-yl)phenyl]-1-methyl-7-(pyridin-3-ylmethoxy)-1H-indole-2-carboxamide

N-[4-cyano-2-(1H-tetraazol-5-yl)phenyl]-6-(3,5-dimethylisoxazol-4-yl)-1-methyl-1H-indole-2-carboxamide

N-[4-cyano-2-(1H-tetraazol-5-yl)phenyl]-1-methyl-6-(2-methylphenyl)-1H-indole-2-carboxamide

7-amino-4,6-dibromo-N-[4-cyano-2-(1H-tetraazol-5-yl)phenyl]-1-methyl-1H-indole-2-carboxamide

4,6-dibromo-N-[4-cyano-2-(1H-tetraazol-5-yl)phenyl]-1-methyl-7-[(phenylacetyl)amino]-1H-indole-2-carboxamide

3-({[4-cyano-2-(1H-tetraazol-5-yl)phenyl]amino}carbonyl)-1,2-benzisoxazol-6-yl acetate

N-[4-cyano-2-(1H-tetraazol-5-yl)phenyl]-6-hydroxy-1,2-benzisoxazole-3-carboxamide

6-(benzyloxy)-N-[4-cyano-2-(1H-tetraazol-5-yl)phenyl]-1,2-benzisoxazole-3-carboxamide

N-[4-cyano-2-(1H-tetraazol-5-yl)phenyl]-6-methoxy-1,2-benzisoxazole-3-carboxamide

3-({[4-cyano-2-(1H-tetraazol-5-yl)phenyl]amino}carbonyl)-1,2-benzisoxazol-6-yl phenylmethanesulfonate

3-({[4-cyano-2-(1H-tetraazol-5-yl)phenyl]amino}carbonyl)-1,2-benzisoxazol-6-yl benzenesulfonate

7-amino-4-bromo-N-[4-cyano-2-(1H-tetraazol-5-yl)phenyl]-1-methyl-1H-indole-2-carboxamide

7-(benzyloxy)-3-bromo-N-[4-cyano-2-(1H-tetraazol-5-yl)phenyl]-1H-indole-2-carboxamide

2-{[2-({[4-cyano-2-(1H-tetraazol-5-yl)phenyl]amino}carbonyl)-1-methyl-1H-indol-6-yl]amino}-2-oxoethyl acetate

5-(anilinosulfonyl)-N-[4-cyano-2-(1H-tetraazol-5-yl)phenyl]-1,2-benzisoxazole-3-carboxamide

N-[4-cyano-2-(1H-tetraazol-5-yl)phenyl]-5-[(diethylamino)sulfonyl]-1,2-benzisoxazole-3-carboxamide

7-amino-6-bromo-N-[4-cyano-2-(1H-tetraazol-5-yl)phenyl]-1-methyl-1H-indole-2-carboxamide

N-[4-cyano-2-(1H-tetraazol-5-yl)phenyl]-1-methyl-7-[(phenylacetyl)amino]-4,6-bis[2-(trifluoromethyl)phenyl]-1H-indole-2-carboxamide

N-[4-cyano-2-(1H-tetraazol-5-yl)phenyl]-5-nitro-6-[4-nitro-2-(trifluoromethyl)phenyl]-1,2-benzisoxazole-3-carboxamide

7-(benzyloxy)-3-bromo-N-[4-cyano-2-(1H-tetraazol-5-yl)phenyl]-1-methyl-1H-indole-2-carboxamide

4-bromo-N-[4-cyano-2-(1H-tetraazol-5-yl)phenyl]-1-methyl-7-[(phenylacetyl)amino]-1H-indole-2-carboxamide

N-[4-cyano-2-(1H-tetraazol-5-yl)phenyl]-1-methyl-6-[(quinolin-8-ylsulfonyl)amino]-1H-indole-2-carboxamide

N-[4-cyano-2-(1H-tetraazol-5-yl)phenyl]-5-(morpholin-4-ylsulfonyl)-1,2-benzisoxazole-3-carboxamide

N-[4-cyano-2-(1H-tetraazol-5-yl)phenyl]-6-(cyclobutylmethoxy)-1-methyl-1H-indole-2-carboxamide

6-(butyrylamino)-N-[4-cyano-2-(1H-tetraazol-5-yl)phenyl]-1-methyl-1H-indole-2-carboxamide

N-[4-cyano-2-(1H-tetraazol-5-yl)phenyl]-6-[(methoxyacetyl)amino]-1-methyl-1H-indole-2-carboxamide

N-[4-cyano-2-(1H-tetraazol-5-yl)phenyl]-7-methyl-1H-indole-2-carboxamide

N-[4-cyano-2-(1H-tetraazol-5-yl)phenyl]-6-oxo-5,6-dihydro-4H-pyrrolo[3,2,1-ij]quinoline-2-carboxamide

5-(acetylamino)-6-[4-(acetylamino)-2-(trfluoromethyl)phenyl]-N-[4-cyano-2-(1H-tetraazol-5-yl)phenyl]-1,2-benzisoxazole-3-carboxamide

N-[4-cyano-2-(1H-tetraazol-5-yl)phenyl]-5-[(dimethylamino)sulfonyl]-1,2-benzisoxazole-3-carboxamide

N-[4-cyano-2-(1H-tetraazol-5-yl)phenyl]-5-[(methoxyacetyl)amino]-1,2-benzisoxazole-3-carboxamide

N-[4-cyano-2-(1H-tetraazol-5-yl)phenyl]-5-[(cyclobutylcarbonyl)amino]-1,2-benzisoxazole-3-carboxamide

N-[4-cyano-2-(1H-tetraazol-5-yl)phenyl]-6-(2-furoylamino)-1-methyl-1H-indole-2-carboxamide

N-[4-cyano-2-(1H-tetraazol-5-yl)phenyl]-1-methyl-6-({[methyl(phenyl)amino]carbonyl}amino)-1H-indole-2-carboxamide

6-(acetylamino)-N-[4-cyano-2-(1H-tetraazol-5-yl)phenyl]-1-methyl-1H-indole-2-carboxamide

N-[4-cyano-2-(1H-tetraazol-5-yl)phenyl]-1-methyl-6-{[4-(trifluoromethoxy)benzoyl]amino}-1H-indole-2-carboxamide

N-[4-cyano-2-(1H-tetraazol-5-yl)phenyl]-6-(2-fluorophenyl)-1-methyl-1H-indole-2-carboxamide

N-[4-cyano-2-(1H-tetraazol-5-yl)phenyl]-6-(cyclopropylmethoxy)-1-methyl-1H-indole-2-carboxamide

7-bromo-N-[4-cyano-2-(1H-tetraazol-5-yl)phenyl]-1-methyl-1H-indole-2-carboxamide

N-[4-cyano-2-(1H-tetraazol-5-yl)phenyl]-6-methoxy-5,6-dihydro-4H-pyrrolo[3,2,1-ij]quinoline-2-carboxamide

N-[4-cyano-2-(1H-tetraazol-5-yl)phenyl]-6-hydroxy-5,6-dihydro-4H-pyrrolo[3,2,1-ij]quinoline-2-carboxamide

N-[4-cyano-2-(1H-tetraazol-5-yl)phenyl]-6-phenoxy-5,6-dihydro-4H-pyrrolo[3,2,1-ij]quinoline-2-carboxamide

N-[4-cyano-2-(1H-tetraazol-5-yl)phenyl]-1-methyl-7-[2-(trifluoromethyl)phenyl]-1H-indole-2-carboxamide

N-[4-cyano-2-(1H-tetraazol-5-yl)phenyl]-1-methyl-7-phenyl-1H-indole-2-carboxamide

7-(4-tert-butylphenyl)-N-[4-cyano-2-(1H-tetraazol-5-yl)phenyl]-1-methyl-1H-indole-2-carboxamide

N-[4-cyano-2-(1H-tetraazol-5-yl)phenyl]-1-methyl-7-(5-methylthien-2-yl)-1H-indole-2-carboxamide

N-[4-cyano-2-(1H-tetraazol-5-yl)phenyl]-1-methyl-6-[(pyridin-3-ylcarbonyl)amino]-1H-indole-2-carboxamide

N-[4-cyano-2-(1H-tetraazol-5-yl)phenyl]-1-methyl-6-[(methylsulfonyl)amino]-1H-indole-2-carboxamide

N-[4-cyano-2-(1H-tetraazol-5-yl)phenyl]-6-({[(2,5-dimethoxyphenyl)amino]carbonyl 3 amino)-1-methyl-1H-indole-2-carboxamide

N-[4-cyano-2-(1H-tetraazol-5-yl)phenyl]-6-[(isoxazol-5-ylcarbonyl)amino]-1-methyl-1H-indole-2-carboxamide

N-[4-cyano-2-(1H-tetraazol-5-yl)phenyl]-1-methyl-6-{[(pentylamino)carbonyl]amino}-1H-indole-2-carboxamide

N-[4-cyano-2-(1H-tetraazol-5-yl)phenyl]-1-methyl-7-[4-(methylsulfonyl)phenyl]-1H-indole-2-carboxamide

N-[4-cyano-2-(1H-tetraazol-5-yl)phenyl]-7-(2-methoxyphenyl)-1-methyl-1H-indole-2-carboxamide

N-[4-cyano-2-(1H-tetraazol-5-yl)phenyl]-7-(2-fluorophenyl)-1-methyl-1H-indole-2-carboxamide

N-[4-cyano-2-(1H-tetraazol-5-yl)phenyl]-1-methyl-7-(2-methylphenyl)-1H-indole-2-carboxamide

N-[4-cyano-2-(1H-tetraazol-5-yl)phenyl]-6-{[(3,5-dimethylisoxazol-4-yl)sulfonyl]amino}-1-methyl-1H-indole-2-carboxamide

N-[4-cyano-2-(1H-tetraazol-5-yl)phenyl]-5-(dimethylamino)-1,2-benzisoxazole-3-carboxamide

N-[4-chloro-2-(1H-tetraazol-5-yl)phenyl]-1H-indole-2-carboxamide

N-[4-chloro-2-(1H-tetraazol-5-yl)phenyl]-5-methoxy-1H-indole-2-carboxamide

5-(benzyloxy)-N-[4-chloro-2-(1H-tetraazol-5-yl)phenyl]-1H-indole-2-carboxamide

N-[4-chloro-2-(1H-tetraazol-5-yl)phenyl]-1-methyl-1H-indole-2-carboxamide

6-(benzyloxy)-N-[4-chloro-2-(1H-tetraazol-5-yl)phenyl]-1H-indole-2-carboxamide

7-chloro-N-[4-chloro-2-(1H-tetraazol-5-yl)phenyl]-1H-indole-2-carboxamide

N-[4-chloro-2-(1H-tetraazol-5-yl)phenyl]-4-methoxy-1H-indole-2-carboxamide

6-chloro-N-[4-chloro-2-(1H-tetraazol-5-yl)phenyl]-1H-indole-2-carboxamide

1-benzyl-N-[4-chloro-2-(1H-tetraazol-5-yl)phenyl]-1H-indole-2-carboxamide

N-[4-chloro-2-(1H-tetraazol-5-yl)phenyl]-1-ethyl-1H-indole-2-carboxamide

N-[4-chloro-2-(1H-tetraazol-5-yl)phenyl]-7-(phenylsulfonyl)-1H-indole-2-carboxamide

1-allyl-N-[4-chloro-2-(1H-tetraazol-5-yl)phenyl]-1H-indole-2-carboxamide

N-[4-chloro-2-(1H-tetraazol-5-yl)phenyl]-1-(cyclohexylmethyl)-1H-indole-2-carboxamide

N-[4-chloro-2-(1H-tetraazol-5-yl)phenyl]-1-(2-methoxyethyl)-1H-indole-2-carboxamide

N-[4-chloro-2-(1H-tetraazol-5-yl)phenyl]-1-pentyl-1H-indole-2-carboxamide

1-butyl-N-[4-chloro-2-(1H-tetraazol-5-yl)phenyl]-1H-indole-2-carboxamide

N-[4-chloro-2-(1H-tetraazol-5-yl)phenyl]-1-propyl-1H-indole-2-carboxamide

N-[4-chloro-2-(1H-tetraazol-5-yl)phenyl]-1-isopropyl-1H-indole-2-carboxamide

N-[4-chloro-2-(1H-tetraazol-5-yl)phenyl]-1-isobutyl-1H-indole-2-carboxamide

N-[4-chloro-2-(1H-tetraazol-5-yl)phenyl]-1-(3-phenylpropyl)-1H-indole-2-carboxamide

N-[4-chloro-2-(1H-tetraazol-5-yl)phenyl]-1-methyl-7-(phenylsulfonyl)-1H-indole-2-carboxamide

N-[4-chloro-2-(1H-tetraazol-5-yl)phenyl]-7-[(phenylacetyl)amino]-1H-indole-2-carboxamide

7-(benzoylamino)-N-[4-chloro-2-(1H-tetraazol-5-yl)phenyl]-1H-indole-2-carboxamide

2-{[2-({[4-chloro-2-(1H-tetraazol-5-yl)phenyl]amino}carbonyl)-1H-indol-7-yl]amino}-2-oxoethyl acetate N-[4-chloro-2-(1H-tetraazol-5-yl)phenyl]-7-[(cyclopentylcarbonyl)amino]-1H-indole-2-carboxamide

7-amino-N-[4-chloro-2-(1H-tetraazol-5-yl)phenyl]-1H-indole-2-carboxamide

N-[4-chloro-2-(1H-tetraazol-5-yl)phenyl]-1,2-benzisoxazole-3-carboxamide

7-{[(6-chloropyridin-3-yl)carbonyl]amino}-N-[4-chloro-2-(1H-tetraazol-5-yl)phenyl]-1H-indole-2-carboxamide

N-[4-chloro-2-(1H-tetraazol-5-yl)phenyl]-7-[(isoxazol-5-ylcarbonyl)amino]-1H-indole-2-carboxamide

N-[4-chloro-2-(1H-tetraazol-5-yl)phenyl]-7-[(2,4-difluorobenzoyl)amino]-1H-indole-2-carboxamide

N-[4-chloro-2-(1H-tetraazol-5-yl)phenyl]-7-[(fluoroacetyl)amino]-1H-indole-2-carboxamide

7-(acetylamino)-N-[4-chloro-2-(1H-tetraazol-5-yl)phenyl]-1H-indole-2-carboxamide

7-{[(4-chlorophenyl)acetyl]amino}-N-[4-chloro-2-(1H-tetraazol-5-yl)phenyl]-1H-indole-2-carboxamide

N-[4-chloro-2-(1H-tetraazol-5-yl)phenyl]-7-{[(4-methoxyphenyl)acetyl]amino}-1H-indole-2-carboxamide

N-[4-chloro-2-(1H-tetraazol-5-yl)phenyl]-7-[(cyclopentylacetyl)amino]-1H-indole-2-carboxamide

N-[4-chloro-2-(1H-tetraazol-5-yl)phenyl]-7-[(3-fluorobenzoyl)amino]-1H-indole-2-carboxamide

N-[4-chloro-2-(1H-tetraazol-5-yl)phenyl]-7-[(3-cyanobenzoyl)amino]-1H-indole-2-carboxamide

N-[4-chloro-2-(1H-tetraazol-5-yl)phenyl]-7-[(cyclohexylcarbonyl)amino]-1H-indole-2-carboxamide

N-[4-chloro-2-(1H-tetraazol-5-yl)phenyl]-7-(propionylamino)-1H-indole-2-carboxamide

methyl 5-{[2-({[4-chloro-2-(1H-tetraazol-5-yl)phenyl]amino}carbonyl)-1H-indol-7-yl]amino}-5-oxopentanoate

7-(butyrylamino)-N-[4-chloro-2-(1H-tetraazol-5-yl)phenyl]-1H-indole-2-carboxamide

7-[(4-bromobenzoyl)amino]-N-[4-chloro-2-(1H-tetraazol-5-yl)phenyl]-1H-indole-2-carboxamide

N-[4-chloro-2-(1H-tetraazol-5-yl)phenyl]-7-[(3-phenylpropanoyl)amino]-1H-indole-2-carboxamide

N-[4-chloro-2-(1H-tetraazol-5-yl)phenyl]-7-[(phenoxyacetyl)amino]-1H-indole-2-carboxamide

N-[4-chloro-2-(1H-tetraazol-5-yl)phenyl]-7-[(3-cyclopentylpropanoyl)amino]-1H-indole-2-carboxamide

methyl 3-{[2-({[4-chloro-2-(1H-tetraazol-5-yl)phenyl]amino}carbonyl)-1H-indol-7-yl]amino}-3-oxopropanoate N-[4-chloro-2-(1H-tetraazol-5-yl)phenyl]-7-[(2-ethylhexanoyl)amino]-1H-indole-2-carboxamide

N-[4-chloro-2-(1H-tetraazol-5-yl)phenyl]-7-{[(3,4-dimethoxyphenyl)acetyl]amino}-1H-indole-2-carboxamide

N-[4-chloro-2-(1H-tetraazol-5-yl)phenyl]-7-[(3,5,5-trimethylhexanoyl)amino]-1H-indole-2-carboxamide

N-[4-chloro-2-(1H-tetraazol-5-yl)phenyl]-7-[(cyclopropylcarbonyl)amino]-1H-indole-2-carboxamide

N-[4-chloro-2-(1H-tetraazol-5-yl)phenyl]-7-[(methoxyacetyl)amino]-1H-indole-2-carboxamide

N-[4-chloro-2-(1H-tetraazol-5-yl)phenyl]-7-[(3-methylbutanoyl)amino]-1H-indole-2-carboxamide

N-[4-chloro-2-(1H-tetraazol-5-yl)phenyl]-7-(pentanoylamino)-1H-indole-2-carboxamide

N-[4-chloro-2-(1H-tetraazol-5-yl)phenyl]-7-{[(4,7,7-trimethyl-3-oxo-2-oxabicyclo[2.2.1]hept-1-yl)carbonyl]amino}-1H-indole-2-carboxamide

7-{[chloro(phenyl)acetyl]amino}-N-[4-chloro-2-(1H-tetraazol-5-yl)phenyl]-1H-indole-2-carboxamide

7-{[(benzyloxy)acetyl]amino}-N-[4-chloro-2-(1H-tetraazol-5-yl)phenyl]-1H-indole-2-carboxamide

ethyl 3-{[2-({[4-chloro-2-(1H-tetraazol-5-yl)phenyl]amino}carbonyl)-1H-indol-7-yl]amino}-3-oxopropanoate

7-[(1-adamantylcarbonyl)amino]-N-[4-chloro-2-(1H-tetraazol-5-yl)phenyl]-1H-indole-2-carboxamide

N-[4-chloro-2-(1H-tetraazol-5-yl)phenyl]-7-(hexanoylamino)-1H-indole-2-carboxamide

N-[4-chloro-2-(1H-tetraazol-5-yl)phenyl]-7-{[(2-phenylcyclopropyl)carbonyl]amino}-1H-indole-2-carboxamide

N-[4-chloro-2-(1H-tetraazol-5-yl)phenyl]-7-[(2-phenylbutanoyl)amino]-1H-indole-2-carboxamide

N-[4-chloro-2-(1H-tetraazol-5-yl)phenyl]-7-(heptanoylamino)-1H-indole-2-carboxamide

2-{[2-({[4-chloro-2-(1H-tetraazol-5-yl)phenyl]amino}carbonyl)-1H-indol-7-yl]amino}-2-oxo-1-phenylethyl acetate

N-[4-chloro-2-1H-tetrazo-5-yl)phenyl-7-[(thien-2-ylcarbonyl)amino]-1H-indole-2-carboxamide

N-[4-chloro-2-(1H-tetraazol-5-yl)phenyl]-7-[(2-methylbutanoyl)amino]-1H-indole-2-carboxamide

methyl 8-{[2-({[4-chloro-2-(1H-tetraazol-5-yl)phenyl]amino}carbonyl)-1H-indol-7-yl]amino}-8-oxooctanoate

N-[4-chloro-2-(1H-tetraazol-5-yl)phenyl]-7-[(2-ethylbutanoyl)amino]-1H-indole-2-carboxamide

N-[4-chloro-2-(1H-tetraazol-5-yl)phenyl]-7-(octanoylamino)-1H-indole-2-carboxamide

N-[4-chloro-2-(1H-tetraazol-5-yl)phenyl]-7-[(cyclobutylcarbonyl)amino]-1H-indole-2-carboxamide

N-[4-chloro-2-(1H-tetraazol-5-yl)phenyl]-7-(1,3-dioxo-1,3-dihydro-2H-isoindol-2-yl)-1H-indole-2-carboxamide

7-({[2-(benzylthio)-1,3-thiazol-4-yl]carbonyl}amino)-N-[4-chloro-2-(1H-tetraazol-5-yl)phenyl]-1H-indole-2-carboxamide

N-[4-chloro-2-(1H-tetraazol-5-yl)phenyl]-7-{[3-(morpholin-4-ylsulfonyl)benzoyl]amino}-1H-indole-2-carboxamide

N-[4-chloro-2-(1H-tetraazol-5-yl)phenyl]-7-[(1H-indol-2-ylcarbonyl)amino]-1H-indole-2-carboxamide

N-[2-({[4-chloro-2-(1H-tetraazol-5-yl)phenyl]amino}carbonyl)-1H-indol-7-yl]-1-methyl-1H-indole-2-carboxamide

N-[4-chloro-2-(1H-tetraazol-5-yl)phenyl]-7-{[(5-phenylisoxazol-3-yl)carbonyl]amino}-1H-indole-2-carboxamide

N-[4-chloro-2-(1H-tetraazol-5-yl)phenyl]-7-[(5-phenylpentanoyl)amino]-1H-indole-2-carboxamide

N-[4-chloro-2-(1H-tetraazol-5-yl)phenyl]-7-[(4-phenylbutanoyl)amino]-1H-indole-2-carboxamide

N-[4-chloro-2-(1H-tetraazol-5-yl)phenyl]-7-{[4-(4-methoxyphenyl)butanoyl]amino}-1H-indole-2-carboxamide

7-{[(2-chlorophenyl)acetyl]amino}-N-[4-chloro-2-(1H-tetraazol-5-yl)phenyl]-1H-indole-2-carboxamide

N-[4-chloro-2-(1H-tetraazol-5-yl)phenyl]-7-{[(2,4-dichlorophenyl)acetyl]amino}-1H-indole-2-carboxamide

N-[4-chloro-2-(1H-tetraazol-5-yl)phenyl]-7-{[(3,4-dichlorophenyl)acetyl]amino}-1H-indole-2-carboxamide

7-{[(3-chlorophenyl)acetyl]amino}-N-[4-chloro-2-(1H-tetraazol-5-yl)phenyl]-1H-indole-2-carboxamide

N-[4-chloro-2-(1H-tetraazol-5-yl)phenyl]-7-({[3-(trifluoromethyl)phenyl]acetyl}amino)-1H-indole-2-carboxamide

N-[4-chloro-2-(1H-tetraazol-5-yl)phenyl]-7-{[(3-methylphenyl)acetyl]amino}-1H-indole-2-carboxamide

7-{[(4-tert-butylphenyl)acetyl]amino}-N-[4-chloro-2-(1H-tetraazol-5-yl)phenyl]-1H-indole-2-carboxamide

N-[4-chloro-2-(1H-tetraazol-5-yl)phenyl]-7-{[(3-methoxyphenyl)acetyl]amino}-1H-indole-2-carboxamide

N-[4-chloro-2-(1H-tetraazol-5-yl)phenyl]-7-{[(2-methoxyphenyl)acetyl]amino}-1H-indole-2-carboxamide

N-[4-chloro-2-(1H-tetraazol-5-yl)phenyl]-7-{[(2-methylphenyl)acetyl]amino}-1H-indole-2-carboxamide

N-[4-chloro-2-(1H-tetraazol-5-yl)phenyl]-7-({[4-(trifluoromethyl)phenyl]acetyl}amino)-1H-indole-2-carboxamide

N-[4-chloro-2-(1H-tetraazol-5-yl)phenyl]-7-{[(4-isopropylphenyl)acetyl]amino}-1H-indole-2-carboxamide

N-[4-chloro-2-(1H-tetraazol-5-yl)phenyl]-7-{[(4-methylphenyl)acetyl]amino}-1H-indole-2-carboxamide

N-[4-chloro-2-(1H-tetraazol-5-yl)phenyl]-7-{[(4-fluorophenyl)acetyl]amino}-1H-indole-2-carboxamide

N-[4-chloro-2-(1H-tetraazol-5-yl)phenyl]-7-({[2-(trifluoromethyl)phenyl]acetyl}amino)-1H-indole-2-carboxamide

N-[4-chloro-2-(1H-tetraazol-5-yl)phenyl]-7-{[(3-fluorophenyl)acetyl]amino}-1H-indole-2-carboxamide

N-[4-chloro-2-(1H-tetraazol-5-yl)phenyl]-7-{[(phenylthio)acetyl]amino}-1H-indole-2-carboxamide

N-[4-chloro-2-(1H-tetraazol-5-yl)phenyl]-7-[(2-naphthylacetyl)amino]-1H-indole-2-carboxamide

N-[4-chloro-2-(1H-tetraazol-5-yl)phenyl]-7-[(1-naphthylacetyl)amino]-1H-indole-2-carboxamide

N-[4-chloro-2-(1H-tetraazol-5-yl)phenyl]-7-{[(2-naphthyloxy)acetyl]amino}-1H-indole-2-carboxamide

N-[4-chloro-2-(1H-tetraazol-5-yl)phenyl]-7-[(2-propoxybenzoyl)amino]-1H-indole-2-carboxamide

N-[4-chloro-2-(1H-tetraazol-5-yl)phenyl]-7-[(tetrahydrofuran-3-ylcarbonyl)amino]-1H-indole-2-carboxamide

N-[4-chloro-2-(1H-tetraazol-5-yl)phenyl]-7-{[(1-methylcyclopropyl)carbonyl]amino}-1H-indole-2-carboxamide

N-[4-chloro-2-(1H-tetraazol-5-yl)phenyl]-7-{[(4-ethoxyphenyl)acetyl]amino}-1H-indole-2-carboxamide

7-[(1-benzothien-3-ylacetyl)amino]-N-[4-chloro-2-(1H-tetraazol-5-yl)phenyl]-1H-indole-2-carboxamide

7-[(1,1′-biphenyl-4-ylcarbonyl)amino]-N-[4-chloro-2-(1H-tetraazol-5-yl)phenyl]-1H-indole-2-carboxamide

7-[(4-butoxybenzoyl)amino]-N-[4-chloro-2-(1H-tetraazol-5-yl)phenyl]-1H-indole-2-carboxamide

N-[4-chloro-2-(1H-tetraazol-5-yl)phenyl]-7-{[2-(2-phenylethyl)benzoyl]amino}-1H-indole-2-carboxamide

7-[(1,1′-biphenyl-2-ylcarbonyl)amino]-N-[4-chloro-2-(1H-tetraazol-5-yl)phenyl]-1H-indole-2-carboxamide

N-[4-chloro-2-(1H-tetraazol-5-yl)phenyl]-7-{[4-(ethylthio)benzoyl]amino}-1H-indole-2-carboxamide

N-[4-chloro-2-(1H-tetraazol-5-yl)phenyl]-7-{[2-(methylsulfonyl)benzoyl]amino}-1H-indole-2-carboxamide

N-[4-chloro-2-(1H-tetraazol-5-yl)phenyl]-7-{[(2,6-dichlorophenyl)acetyl]amino}-1H-indole-2-carboxamide

7-[(1,1′-biphenyl-4-ylacetyl)amino]-N-[4-chloro-2-(1H-tetraazol-5-yl)phenyl]-1H-indole-2-carboxamide

7-[(1,3-benzodioxol-5-ylacetyl)amino]-N-[4-chloro-2-(1H-tetraazol-5-yl)phenyl]-1H-indole-2-carboxamide

N-[4-chloro-2-(1H-tetraazol-5-yl)phenyl]-7-[(3,3-dimethylbutanoyl)amino]-1H-indole-2-carboxamide

N-[4-chloro-2-(1H-tetraazol-5-yl)phenyl]-7-[(thien-2-ylacetyl)amino]-1H-indole-2-carboxamide

N-[4-chloro-2-(1H-tetraazol-5-yl)phenyl]-7-{[(3-methyl-5-phenylisoxazol-4-yl)carbonyl]amino}-1H-indole-2-carboxamide

N-[4-chloro-2-(1H-tetraazol-5-yl)phenyl]-7-({[2-(2-methoxyethoxy)ethoxy]acetyl}amino)-1H-indole-2-carboxamide

N-[4-chloro-2-(1H-tetraazol-5-yl)phenyl]-7-[(2-hydroxybenzoyl)amino]-1H-indole-2-carboxamide

N-[4-chloro-2-(1H-tetraazol-5-yl)phenyl]-7-({[4-(trifluoromethoxy)phenyl]sulfonyl}amino)-1H-indole-2-carboxamide

N-[4-chloro-2-(1H-tetraazol-5-yl)phenyl]-7-(prolylamino)-1H-indole-2-carboxamide

N-[4-chloro-2-(1H-tetraazol-5-yl)phenyl]-7-{[(3-methylisoxazol-5-yl)acetyl]amino}-1H-indole-2-carboxamide

N-[4-chloro-2-(1H-tetraazol-5-yl)phenyl]-6-methoxy-1H-indole-2-carboxamide

N-[4-chloro-2-(1H-tetraazol-5-yl)phenyl]-5-nitro-1,2-benzisoxazole-3-carboxamide

7-[(benzylsulfonyl)amino]-N-[4-chloro-2-(1H-tetraazol-5-yl)phenyl]-1H-indole-2-carboxamide

N-[4-chloro-2-(1H-tetraazol-5-yl)phenyl]-1-methyl-7-{[3-(morpholin-4-ylsulfonyl)benzoyl]amino}-1H-indole-2-carboxamide

N-[4-chloro-2-(1H-tetraazol-5-yl)phenyl]-7-{[(4-fluorophenyl)acetyl]amino}-1-methyl-1H-indole-2-carboxamide

N-[4-chloro-2-(1H-tetraazol-5-yl)phenyl]-7-[(fluoroacetyl)amino]-1-methyl-1H-indole-2-carboxamide

N-[4-chloro-2-(1H-tetraazol-5-yl)phenyl]-1-methyl-7-{[(1-methyl-1H-indol-2-yl)carbonyl]amino}-1H-indole-2-carboxamide

N-[4-chloro-2-(1H-tetraazol-5-yl)phenyl]-5-[(phenylacetyl)amino]-1,2-benzisoxazole-3-carboxamide

5-[(benzylsulfonyl)amino]-N-[4-chloro-2-(1H-tetraazol-5-yl)phenyl]-1,2-benzisoxazole-3-carboxamide

6-(benzyloxy)-N-[4-chloro-2-(1H-tetraazol-5-yl)phenyl]-1-methyl-1H-indole-2-carboxamide

N-[4-chloro-2-(1H-tetraazol-5-yl)phenyl]-6-methoxy-1-methyl-1H-indole-2-carboxamide

N-[4-chloro-2-(1H-tetraazol-5-yl)phenyl]-1-methyl-7-[(morpholin-4-ylcarbonyl)amino]-1H-indole-2-carboxamide

N-[4-chloro-2-(1H-tetraazol-5-yl)phenyl]-1-methyl-7-({[(tetrahydrofuran-2-ylmethyl)amino]carbonyl}amino)-1H-indole-2-carboxamide

N-[4-chloro-2-(1H-tetraazol-5-yl)phenyl]-7-hydroxy-1-methyl-1H-indole-2-carboxamide

7-{[(benzylamino)carbonyl]amino}-N-[4-chloro-2-(1H-tetraazol-5-yl)phenyl]-1-methyl-1H-indole-2-carboxamide

N-[4-chloro-2-(1H-tetraazol-5-yl)phenyl]-7-({[(2,3-dihydroxypropyl)amino]carbonyl}amino)-1-methyl-1H-indole-2-carboxamide

1-[({[2-({[4-chloro-2-(1H-tetraazol-5-yl)phenyl]amino}carbonyl)-1-methyl-1H-indol-7-yl]amino}carbonyl)(methyl)amino]-1-deoxyhexitol

N-[4-chloro-2-(1H-tetraazol-5-yl)phenyl]-7-(2,3-dihydro-1,4-benzodioxin-2-ylmethoxy)-1-methyl-1H-indole-2-carboxamide

7-(benzyloxy)-N-[4-chloro-2-(1H-tetraazol-5-yl)phenyl]-1H-indole-2-carboxamide

N-[4-chloro-2-(1H-tetraazol-5-yl)phenyl]-1-methyl-7-(3-phenoxypropoxy)-1H-indole-2-carboxamide

5-(acetylamino)-N-[4-chloro-2-(1H-tetraazol-5-yl)phenyl]-1,2-benzisoxazole-3-carboxamide

N-[4-chloro-2-(1H-tetraazol-5-yl)phenyl]-5-[(methylsulfonyl)amino]-1,2-benzisoxazole-3-carboxamide

5-(benzoylamino)-N-[4-chloro-2-(1H-tetraazol-5-yl)phenyl]-1,2-benzisoxazole-3-carboxamide

N-[4-chloro-2-(1H-tetraazol-5-yl)phenyl]-5-[(phenylsulfonyl)amino]-1,2-benzisoxazole-3-carboxamide

N-[4-chloro-2-(1H-tetraazol-5-yl)phenyl]-7-(cyclobutylmethoxy)-1-methyl-1H-indole-2-carboxamide

N-[4-chloro-2-(1H-tetraazol-5-yl)phenyl]-7-(2-furylmethoxy)-1-methyl-1H-indole-2-carboxamide

N-[4-chloro-2-(1H-tetraazol-5-yl)phenyl]-7-{[(4S)-2,2-dimethyl-1,3-dioxolan-4-yl]methoxy}-1-methyl-1H-indole-2-carboxamide

N-[4-chloro-2-(1H-tetraazol-5-yl)phenyl]-7-{[(2R)-2,3-dihydroxypropyl]oxy}-1-methyl-1H-indole-2-carboxamide

N-[4-chloro-2-(1H-tetraazol-5-yl)phenyl]-7-(cyclobutyloxy)-1-methyl-1H-indole-2-carboxamide

N-[4-chloro-2-(1H-tetraazol-5-yl)phenyl]-7-(2-methoxy-1-methylethoxy)-1-methyl-1H-indole-2-carboxamide

N-[4-chloro-2-(1H-tetraazol-5-yl)phenyl]-7-isopropoxy-1-methyl-1H-indole-2-carboxamide

7-(benzyloxy)-N-[4-chloro-2-(1H-tetraazol-5-yl)phenyl]-1-methyl-1H-indole-2-carboxamide

6-chloro-N-[4-chloro-2-(1H-tetraazol-5-yl)phenyl]-1,2-benzisoxazole-3-carboxamide

6-sec-butoxy-N-[4-chloro-2-(1H-tetraazol-5-yl)phenyl]-1-methyl-1H-indole-2-carboxamide

6-butoxy-N-[4-chloro-2-(1H-tetraazol-5-yl)phenyl]-1-methyl-1H-indole-2-carboxamide

N-[4-chloro-2-(1H-tetraazol-5-yl)phenyl]-5-{[(4-fluorophenyl)sulfonyl]amino}-1,2-benzisoxazole-3-carboxamide

5-amino-N-[4-chloro-2-(1H-tetraazol-5-yl)phenyl]-1,2-benzisoxazole-3-carboxamide

5-bromo-N-[4-chloro-2-(1H-tetraazol-5-yl)phenyl]-1,2-benzisoxazole-3-carboxamide

6-[(benzylsulfonyl)amino]-N-[4-chloro-2-(1H-tetraazol-5-yl)phenyl]-1,2-benzisoxazole-3-carboxamide

N-[4-chloro-2-(1H-tetraazol-5-yl)phenyl]-6-[(phenylsulfonyl)amino]-1,2-benzisoxazole-3-carboxamide

N-[4-chloro-2-(1H-tetraazol-5-yl)phenyl]-7-(cyclohexylmethoxy)-1-methyl-1H-indole-2-carboxamide

N-[4-chloro-2-(1H-tetraazol-5-yl)phenyl]-7-(cyclopropylmethoxy)-1-methyl-1H-indole-2-carboxamide

N-[4-chloro-2-(1H-tetraazol-5-yl)phenyl]-1-methyl-7-(tetrahydro-2H-pyran-2-ylmethoxy)-1H-indole-2-carboxamide

N-[4-chloro-2-(1H-tetraazol-5-yl)phenyl]-1-methyl-7-(pentyloxy)-1H-indole-2-carboxamide

N-[4-chloro-2-(1H-tetraazol-5-yl)phenyl]-5-phenyl-1,2-benzisoxazole-3-carboxamide

N-[4-chloro-2-(1H-tetraazol-5-yl)phenyl]-5-[2-(trifluoromethyl)phenyl]-1,2-benzisoxazole-3-carboxamide

N-[4-chloro-2-(1H-tetraazol-5-yl)phenyl]-7-(2-methoxyethoxy)-1-methyl-1H-indole-2-carboxamide

N-[4-chloro-2-(1H-tetraazol-5-yl)phenyl]-7-(2-hydroxy-3-isopropoxypropoxy)-1-methyl-1H-indole-2-carboxamide

N-[4-chloro-2-(1H-tetraazol-5-yl)phenyl]-6-phenyl-1,2-benzisoxazole-3-carboxamide

N-[4-chloro-2-(1H-tetraazol-5-yl)phenyl]-6-[2-(trifluoromethyl)phenyl]-1,2-benzisoxazole-3-carboxamide

N-[4-chloro-2-(1H-tetraazol-5-yl)phenyl]-1-methyl-7-[2-(methylthio)ethoxy]-1H-indole-2-carboxamide

7-[(4-azido-3-iodobenzoyl)amino]-N-[4-chloro-2-(1H-tetraazol-5-yl)phenyl]-1-methyl-1H-indole-2-carboxamide

5-[(4-azido-3-iodobenzoyl)amino]-N-[4-chloro-2-(1H-tetraazol-5-yl)phenyl]-1,2-benzisoxazole-3-carboxamide

N-[4-chloro-2-(1H-tetraazol-5-yl)phenyl]-6-[(methylsulfonyl)amino]-1,2-benzisoxazole-3-carboxamide

N-[4-chloro-2-(1H-tetraazol-5-yl)phenyl]-6-{[(4-fluorophenyl)sulfonyl]amino}-1,2-benzisoxazole-3-carboxamide

6-amino-N-[4-chloro-2-(1H-tetraazol-5-yl)phenyl]-1,2-benzisoxazole-3-carboxamide

N-[4-chloro-2-(1H-tetraazol-5-yl)phenyl]-5-(2-fluorophenyl)-1,2-benzisoxazole-3-carboxamide

N-[4-chloro-2-(1H-tetraazol-5-yl)phenyl]-1-methyl-6-[2-(trifluoromethyl)phenyl]-1H-indole-2-carboxamide

N-[4-chloro-2-(1H-tetraazol-5-yl)phenyl]-1-methyl-6-(2,3,4-trimethoxyphenyl)-1H-indole-2-carboxamide

N-[4-chloro-2-(1H-tetraazol-5-yl)phenyl]-1-methyl-7-(pyridin-3-ylmethoxy)-1H-indole-2-carboxamide

N-[4-chloro-2-(1H-tetraazol-5-yl)phenyl]-6-(3,5-dimethylisoxazol-4-yl)-1-methyl-1H-indole-2-carboxamide

N-[4-chloro-2-(1H-tetraazol-5-yl)phenyl]-1-methyl-6-(2-methylphenyl)-1H-indole-2-carboxamide

7-amino-4,6-dibromo-N-[4-chloro-2-(1H-tetraazol-5-yl)phenyl]-1-methyl-1H-indole-2-carboxamide

4,6-dibromo-N-[4-chloro-2-(1H-tetraazol-5-yl)phenyl]-1-methyl-7-[(phenylacetyl)amino]-1H-indole-2-carboxamide

3-({[4-chloro-2-(1H-tetraazol-5-yl)phenyl]amino}carbonyl)-1,2-benzisoxazol-6-yl acetate

N-[4-chloro-2-(1H-tetraazol-5-yl)phenyl]-6-hydroxy-1,2-benzisoxazole-3-carboxamide

6-(benzyloxy)-N-[4-chloro-2-(1H-tetraazol-5-yl)phenyl]-1,2-benzisoxazole-3-carboxamide

N-[4-chloro-2-(1H-tetraazol-5-yl)phenyl]-6-methoxy-1,2-benzisoxazole-3-carboxamide

3-({[4-chloro-2-(1H-tetraazol-5-yl)phenyl]amino}carbonyl)-1,2-benzisoxazol-6-yl phenylmethanesulfonate 3-({[4-chloro-2-(1H-tetraazol-5-yl)phenyl]amino}carbonyl)-1,2-benzisoxazol-6-yl benzenesulfonate

7-amino-4-bromo-N-[4-chloro-2-(1H-tetraazol-5-yl)phenyl]-1-methyl-1H-indole-2-carboxamide

7-(benzyloxy)-3-bromo-N-[4-chloro-2-(1H-tetraazol-5-yl)phenyl]-1H-indole-2-carboxamide

2-{[2-({[4-chloro-2-(1H-tetraazol-5-yl)phenyl]amino}carbonyl)-1-methyl-1H-indol-6-yl]amino}-2-oxoethyl acetate

5-(anilinosulfonyl)-N-[4-chloro-2-(1H-tetraazol-5-yl)phenyl]-1,2-benzisoxazole-3-carboxamide

N-[4-chloro-2-(1H-tetraazol-5-yl)phenyl]-5-[(diethylamino)sulfonyl]-1,2-benzisoxazole-3-carboxamide

7-amino-6-bromo-N-[4-chloro-2-(1H-tetraazol-5-yl)phenyl]-1-methyl-1H-indole-2-carboxamide

N-[4-chloro-2-(1H-tetraazol-5-yl)phenyl]-1-methyl-7-[(phenylacetyl)amino]-4,6-bis[2-(trifluoromethyl)phenyl]-1H-indole-2-carboxamide

N-[4-chloro-2-(1H-tetraazol-45-yl)phenyl]-5-nitro-6-[4-nitro-2-(trifluoromethyl)phenyl]-1,2-benzisoxazole-3-carboxamide

7-(benzyloxy)-3-bromo-N-[4-chloro-2-(1H-tetraazol-5-yl)phenyl]-1-methyl-1H-indole-2-carboxamide

4-bromo-N-[4-chloro-2-(1H-tetraazol-5-yl)phenyl]-1-methyl-7-[(phenylacetyl)amino]-1H-indole-2-carboxamide

N-[4-chloro-2-(1H-tetraazol-5-yl)phenyl]-1-methyl-6-[(quinolin-8-ylsulfonyl)amino]-1H-indole-2-carboxamide

N-[4-chloro-2-(1H-tetraazol-5-yl)phenyl]-5-(morpholin-4-ylsulfonyl)-1,2-benzisoxazole-3-carboxamide

N-[4-chloro-2-(1H-tetraazol-5-yl)phenyl]-6-(cyclobutylmethoxy)-1-methyl-1H-indole-2-carboxamide

6-(butyrylamino)-N-[4-chloro-2-(1H-tetraazol-5-yl)phenyl]-1-methyl-1H-indole-2-carboxamide

N-[4-chloro-2-(1H-tetraazol-5-yl)phenyl]-6-[(methoxyacetyl)amino]-1-methyl-1H-indole-2-carboxamide

N-[4-chloro-2-(1H-tetraazol-5-yl)phenyl]-7-methyl-1H-indole-2-carboxamide

N-[4-chloro-2-(1H-tetraazol-5-yl)phenyl]-6-oxo-5,6-dihydro-4H-pyrrolo[3,2,1-ij]quinoline-2-carboxamide

5-(acetylamino)-6-[4-(acetylamino)-2-(trifluoromethyl)phenyl]-N-[4-chloro-2-(1H-tetraazol-5-yl)phenyl]-1,2-benzisoxazole-3-carboxamide

N-[4-chloro-2-(1H-tetraazol-5-yl)phenyl]-5-[(dimethylamino)sulfonyl]-1,2-benzisoxazole-3-carboxamide

N-[4-chloro-2-(1H-tetraazol-5-yl)phenyl]-5-[(methoxyacetyl)amino]-1,2-benzisoxazole-3-carboxamide

N-[4-chloro-2-(1H-tetraazol-5-yl)phenyl]-5-[(cyclobutylcarbonyl)amino]-1,2-benzisoxazole-3-carboxamide

N-[4-chloro-2-(1H-tetraazol-5-yl)phenyl]-6-(2-furoylamino)-1-methyl-1H-indole-2-carboxamide

N-[4-chloro-2-(1H-tetraazol-5-yl)phenyl]-1-methyl-6-({[methyl(phenyl)amino]carbonyl}amino)-1H-indole-2-carboxamide

6-(acetylamino)-N-[4-chloro-2-(1H-tetraazol-5-yl)phenyl]-1-methyl-1H-indole-2-carboxamide

N-[4-chloro-2-(1H-tetraazol-5-yl)phenyl]-1-methyl-6-{[4-(trifluoromethoxy)benzoyl]amino}-1H-indole-2-carboxamide

N-[4-chloro-2-(1H-tetraazol-5-yl)phenyl]-6-(2-fluorophenyl)-1-methyl-1H-indole-2-carboxamide

N-[4-chloro-2-(1H-tetraazol-5-yl)phenyl]-6-(cyclopropylmethoxy)-1-methyl-1H-indole-2-carboxamide

7-bromo-N-[4-chloro-2-(1H-tetraazol-5-yl)phenyl]-1-methyl-1H-indole-2-carboxamide

N-[4-chloro-2-(1H-tetraazol-5-yl)phenyl]-6-methoxy-5,6-dihydro-4H-pyrrolo[3,2,1-ij]quinoline-2-carboxamide

N-[4-chloro-2-(1H-tetraazol-5-yl)phenyl]-6-hydroxy-5,6-dihydro-4H-pyrrolo[3,2,1-ij]quinoline-2-carboxamide

N-[4-chloro-2-(1H-tetraazol-5-yl)phenyl]-6-phenoxy-5,6-dihydro-4H-pyrrolo[3,2,1-ij]quinoline-2-carboxamide

N-[4-chloro-2-(1H-tetraazol-5-yl)phenyl]-1-methyl-7-[2-(trifluoromethyl)phenyl]-1H-indole-2-carboxamide

N-[4-chloro-2-(1H-tetraazol-5-yl)phenyl]-1-methyl-7-phenyl-1H-indole-2-carboxamide

7-(4-tert-butylphenyl)-N-[4-chloro-2-(1H-tetraazol-5-yl)phenyl]-1-methyl-1H-indole-2-carboxamide

N-[4-chloro-2-(1H-tetraazol-5-yl)phenyl]-1-methyl-7-(5-methylthien-2-yl)-1H-indole-2-carboxamide

N-[4-chloro-2-(1H-tetraazol-5-yl)phenyl]-1-methyl-6-[(pyridin-3-ylcarbonyl)amino]-1H-indole-2-carboxamide

N-[4-chloro-2-(1H-tetraazol-5-yl)phenyl]-1-methyl-6-[(methylsulfonyl)amino]-1H-indole-2-carboxamide

N-[4-chloro-2-(1H-tetraazol-5-yl)phenyl]-6-({[(2,5-dimethoxyphenyl)amino]carbonyl}amino)-1-methyl-1H-indole-2-carboxamide

N-[4-chloro-2-(1H-tetraazol-5-yl)phenyl]-6-[(isoxazol-5-ylcarbonyl)amino]-1-methyl-1H-indole-2-carboxamide

N-[4-chloro-2-(1H-tetraazol-5-yl)phenyl]-1-methyl-6-{[(pentylamino)carbonyl]amino}-1H-indole-2-carboxamide

N-[4-chloro-2-(1H-tetraazol-5-yl)phenyl]-1-methyl-7-[4-(methylsulfonyl)phenyl]-1H-indole-2-carboxamide

N-[4-chloro-2-(1H-tetraazol-5-yl)phenyl]-7-(2-methoxyphenyl)-1-methyl-1H-indole-2-carboxamide

N-[4-chloro-2-(1H-tetraazol-5-yl)phenyl]-7-(2-fluorophenyl)-1-methyl-1H-indole-2-carboxamide

N-[4-chloro-2-(1H-tetraazol-5-yl)phenyl]-1-methyl-7-(2-methylphenyl)-1H-indole-2-carboxamide

N-[4-chloro-2-(1H-tetraazol-5-yl)phenyl]-6-{[(3,5-dimethylisoxazol-4-yl)sulfonyl]amino}-1-methyl-1H-indole-2-carboxamide

N-[4-chloro-2-(1H-tetraazol-5-yl)phenyl]-5-(dimethylamino)-1,2-benzisoxazole-3-carboxamide

N-[4-bromo-2-(1H-tetraazol-5-yl)phenyl]-1H-indole-2-carboxamide

N-[4-bromo-2-(1H-tetraazol-5-yl)phenyl]-5-methoxy-1H-indole-2-carboxamide

5-(benzyloxy)-N-[4-bromo-2-(1H-tetraazol-5-yl)phenyl]-1H-indole-2-carboxamide

N-[4-bromo-2-(1H-tetraazol-5-yl)phenyl]-1-methyl-1H-indole-2-carboxamide

6-(benzyloxy)-N-[4-bromo-2-(1H-tetraazol-5-yl)phenyl]-1H-indole-2-carboxamide

N-[4-bromo-2-(1H-tetraazol-5-yl)phenyl]-7-chloro-1H-indole-2-carboxamide

N-[4-bromo-2-(1H-tetraazol-5-yl)phenyl]-4-methoxy-1H-indole-2-carboxamide

N-[4-bromo-2-(1H-tetraazol-5-yl)phenyl]-6-chloro-1H-indole-2-carboxamide

1-benzyl-N-[4-bromo-2-(1H-tetraazol-5-yl)phenyl]-1H-indole-2-carboxamide

N-[4-bromo-2-(1H-tetraazol-5-yl)phenyl]-1-ethyl-1H-indole-2-carboxamide

N-[4-bromo-2-(1H-tetraazol-5-yl)phenyl]-7-(phenylsulfonyl)-1H-indole-2-carboxamide

1-allyl-N-[4-bromo-2-(1H-tetraazol-5-yl)phenyl]-1H-indole-2-carboxamide

N-[4-bromo-2-(1H-tetraazol-5-yl)phenyl]-1-(cyclohexylmethyl)-1H-indole-2-carboxamide

N-[4-bromo-2-(1H-tetraazol-5-yl)phenyl]-1-(2-methoxyethyl)-1H-indole-2-carboxamide

N-[4-bromo-2-(1H-tetraazol-5-yl)phenyl]-1-pentyl-1H-indole-2-carboxamide

N-[4-bromo-2-(1H-tetraazol-5-yl)phenyl]-1-butyl-1H-indole-2-carboxamide

N-[4-bromo-2-(1H-tetraazol-5-yl)phenyl]-1-propyl-1H-indole-2-carboxamide

N-[4-bromo-2-(1H-tetraazol-5-yl)phenyl]-1-isopropyl-1H-indole-2-carboxamide

N-[4-bromo-2-(1H-tetraazol-5-yl)phenyl]-1-isobutyl-1H-indole-2-carboxamide

N-[4-bromo-2-(1H-tetraazol-5-yl)phenyl]-1-(3-phenylpropyl)-1H-indole-2-carboxamide

N-[4-bromo-2-(1H-tetraazol-5-yl)phenyl]-1-methyl-7-(phenylsulfonyl)-1H-indole-2-carboxamide

N-[4-bromo-2-(1H-tetraazol-5-yl)phenyl]-7-[(phenylacetyl)amino]-1H-indole-2-carboxamide

7-(benzoylamino)-N-[4-bromo-2-(1H-tetraazol-5-yl)phenyl]-1H-indole-2-carboxamide

2-{[2-({[4-bromo-2-(1H-tetraazol-5-yl)phenyl]amino}carbonyl)-1H-indol-7-yl]amino}-2-oxoethyl acetate

N-[4-bromo-2-(1H-tetraazol-5-yl)phenyl]-7-[(cyclopentylcarbonyl)amino]-1H-indole-2-carboxamide

7-amino-N-[4-bromo-2-(1H-tetraazol-5-yl)phenyl]-1H-indole-2-carboxamide

N-[4-bromo-2-(1H-tetraazol-5-yl)phenyl]-1,2-benzisoxazole-3-carboxamide

N-[4-bromo-2-(1H-tetraazol-5-yl)phenyl]-7-{[(6-chloropyridin-3-yl)carbonyl]amino}-1H-indole-2-carboxamide

N-[4-bromo-2-(1H-tetraazol-5-yl)phenyl]-7-[(isoxazol-5-ylcarbonyl)amino]-1H-indole-2-carboxamide

N-[4-bromo-2-(1H-tetraazol-5-yl)phenyl]-7-[(2,4-difluorobenzoyl)amino]-1H-indole-2-carboxamide

N-[4-bromo-2-(1H-tetraazol-5-yl)phenyl]-7-[(fluoroacetyl)amino]-1H-indole-2-carboxamide

7-(acetylamino)-N-[4-bromo-2-(1H-tetraazol-5-yl)phenyl]-1H-indole-2-carboxamide

N-[4-bromo-2-(1H-tetraazol-5-yl)phenyl]-7-{[(4-chlorophenyl)acetyl]amino}-1H-indole-2-carboxamide

N-[4-bromo-2-(1H-tetraazol-5-yl)phenyl]-7-{[(4-methoxyphenyl)acetyl]amino}-1H-indole-2-carboxamide

N-[4-bromo-2-(1H-tetraazol-5-yl)phenyl]-7-[(cyclopentylacetyl)amino]-1H-indole-2-carboxamide

N-[4-bromo-2-(1H-tetraazol-5-yl)phenyl]-7-[(3-fluorobenzoyl)amino]-1H-indole-2-carboxamide

N-[4-bromo-2-(1H-tetraazol-5-yl)phenyl]-7-[(3-cyanobenzoyl)amino]-1H-indole-2-carboxamide

N-[4-bromo-2-(1H-tetraazol-5-yl)phenyl]-7-[(cyclohexylcarbonyl)amino]-1H-indole-2-carboxamide

N-[4-bromo-2-(1H-tetraazol-5-yl)phenyl]-7-(propionylamino)-1H-indole-2-carboxamide

methyl 5-{[2-({[4-bromo-2-(1H-tetraazol-5-yl)phenyl]amino}carbonyl)-1H-indol-7-yl]amino}-5-oxopentanoate

N-[4-bromo-2-(1H-tetraazol-5-yl)phenyl]-7-(butyrylamino)-1H-indole-2-carboxamide

7-[(4-bromobenzoyl)amino]-N-[4-bromo-2-(1H-tetraazol-5-yl)phenyl]-1H-indole-2-carboxamide

N-[4-bromo-2-(1H-tetraazol-5-yl)phenyl]-7-[(3-phenylpropanoyl)amino]-1H-indole-2-carboxamide

N-[4-bromo-2-(1H-tetraazol-5-yl)phenyl]-7-[(phenoxyacetyl)amino]-1H-indole-2-carboxamide

N-[4-bromo-2-(1H-tetraazol-5-yl)phenyl]-7-[(3-cyclopentylpropanoyl)amino]-1H-indole-2-carboxamide

methyl 3-{[2-({[4-bromo-2-(1H-tetraazol-5-yl)phenyl]amino}carbonyl)-1H-indol-7-yl]amino}-3-oxopropanoate

N-[4-bromo-2-(1H-tetraazol-5-yl)phenyl]-7-[(2-ethylhexanoyl)amino]-1H-indole-2-carboxamide

N-[4-bromo-2-(1H-tetraazol-5-yl)phenyl]-7-{[(3,4-dimethoxyphenyl)acetyl]amino}-1H-indole-2-carboxamide

N-[4-bromo-2-(1H-tetraazol-5-yl)phenyl]-7-[(3,5,5-trimethylhexanoyl)amino]-1H-indole-2-carboxamide

N-[4-bromo-2-(1H-tetraazol-5-yl)phenyl]-7-[(cyclopropylcarbonyl)amino]-1H-indole-2-carboxamide

N-[4-bromo-2-(1H-tetraazol-5-yl)phenyl]-7-[(methoxyacetyl)amino]-1H-indole-2-carboxamide

N-[4-bromo-2-(1H-tetraazol-5-yl)phenyl]-7-[(3-methylbutanoyl)amino]-1H-indole-2-carboxamide

N-[4-bromo-2-(1H-tetraazol-5-yl)phenyl]-7-(pentanoylamino)-1H-indole-2-carboxamide

N-[4-bromo-2-(1H-tetraazol-5-yl)phenyl]-7-{[(4,7,7-trimethyl-3-oxo-2-oxabicyclo[2.2.1]hept-1-yl)carbonyl]amino}-1H-indole-2-carboxamide

N-[4-bromo-2-(1H-tetraazol-5-yl)phenyl]-7-{[chloro(phenyl)acetyl]amino}-1H-indole-2-carboxamide

7-{[(benzyloxy)acetyl]amino}-N-[4-bromo-2-(1H-tetraazol-5-yl)phenyl]-1H-indole-2-carboxamide

ethyl 3-{[2-({[4-bromo-2-(1H-tetraazol-5-yl)phenyl]amino}carbonyl)-1H-indol-7-yl]amino}-3-oxopropanoate

7-[(1-adamantylcarbonyl)amino]-N-[4-bromo-2-(1H-tetraazol-5-yl)phenyl]-1H-indole-2-carboxamide

N-[4-bromo-2-(1H-tetraazol-5-yl)phenyl]-7-(hexanoylamino)-1H-indole-2-carboxamide

N-[4-bromo-2-(1H-tetraazol-5-yl)phenyl]-7-{[(2-phenylcyclopropyl)carbonyl]amino}-1H-indole-2-carboxamide

N-[4-bromo-2-(1H-tetraazol-5-yl)phenyl]-7-[(2-phenylbutanoyl)amino]-1H-indole-2-carboxamide

N-[4-bromo-2-(1H-tetraazol-5-yl)phenyl]-7-(heptanoylamino)-1H-indole-2-carboxamide

2-{[2-({[4-bromo-2-(1H-tetraazol-5-yl)phenyl]amino}carbonyl)-1H-indol-7-yl]amino}-2-oxo-1-phenylethyl acetate N-[4-bromo-2-(1H-tetraazol-5-yl)phenyl]-7-[(thien-2-ylcarbonyl)amino]-1H-indole-2-carboxamide

N-[4-bromo-2-(1H-tetraazol-5-yl)phenyl]-7-[(2-methylbutanoyl)amino]-1H-indole-2-carboxamide

methyl 8-{[2-({[4-bromo-2-(1H-tetraazol-5-yl)phenyl]amino}carbonyl)-1H-indol-7-yl]amino}-8-oxooctanoate

N-[4-bromo-2-(1H-tetraazol-5-yl)phenyl]-7-[(2-ethylbutanoyl)amino]-1H-indole-2-carboxamide

N-[4-bromo-2-(1H-tetraazol-5-yl)phenyl]-7-(octanoylamino)-1H-indole-2-carboxamide

N-[4-bromo-2-(1H-tetraazol-5-yl)phenyl]-7-[(cyclobutylcarbonyl)amino]-1H-indole-2-carboxamide

N-[4-bromo-2-(1H-tetraazol-5-yl)phenyl]-7-(1,3-dioxo-1,3-dihydro-2H-isoindol-2-yl)-1H-indole-2-carboxamide

7-({[2-(benzylthio)-1,3-thiazol-4-yl]carbonyl}amino)-N-[4-bromo-2-(1H-tetraazol-5-yl)phenyl]-1H-indole-2-carboxamide

N-[4-bromo-2-(1H-tetraazol-5-yl)phenyl]-7-{[3-(morpholin-4-ylsulfonyl)benzoyl]amino}-1H-indole-2-carboxamide

N-[4-bromo-2-(1H-tetraazol-5-yl)phenyl]-7-[(1H-indol-2-ylcarbonyl)amino]-1H-indole-2-carboxamide

N-[2-({[4-bromo-2-(1H-tetraazol-5-yl)phenyl]amino}carbonyl)-1H-indol-7-yl]-1-methyl-1H-indole-2-carboxamide

N-[4-bromo-2-(1H-tetraazol-5-yl)phenyl]-7-{[(5-phenylisoxazol-3-yl)carbonyl]amino}-1H-indole-2-carboxamide

N-[4-bromo-2-(1H-tetraazol-5-yl)phenyl]-7-[(5-phenylpentanoyl)amino]-1H-indole-2-carboxamide

N-[4-bromo-2-(1H-tetraazol-5-yl)phenyl]-7-[(4-phenylbutanoyl)amino]-1H-indole-2-carboxamide

N-[4-bromo-2-(1H-tetraazol-5-yl)phenyl]-7-{[4-(4-methoxyphenyl)butanoyl]amino}-1H-indole-2-carboxamide

N-[4-bromo-2-(1H-tetraazol-5-yl)phenyl]-7-{[(2-chlorophenyl)acetyl]amino}-1H-indole-2-carboxamide

N-[4-bromo-2-(1H-tetraazol-5-yl)phenyl]-7-{[(2,4-dichlorophenyl)acetyl]amino}-1H-indole-2-carboxamide

N-[4-bromo-2-(1H-tetraazol-5-yl)phenyl]-7-{[(3,4-dichlorophenyl)acetyl]amino}-1H-indole-2-carboxamide

N-[4-bromo-2-(1H-tetraazol-5-yl)phenyl]-7-{[(3-chlorophenyl)acetyl]amino}-1H-indole-2-carboxamide

N-[4-bromo-2-(1H-tetraazol-5-yl)phenyl]-7-({[3-(trifluoromethyl)phenyl]acetyl}amino)-1H-indole-2-carboxamide

N-[4-bromo-2-(1H-tetraazol-5-yl)phenyl]-7-{[(3-methylphenyl)acetyl]amino}-1H-indole-2-carboxamide

N-[4-bromo-2-(1H-tetraazol-5-yl)phenyl]-7-{[(4-tert-butylphenyl)acetyl]amino}-1H-indole-2-carboxamide

N-[4-bromo-2-(1H-tetraazol-5-yl)phenyl]-7-{[(3-methoxyphenyl)acetyl]amino}-1H-indole-2-carboxamide

N-[4-bromo-2-(1H-tetraazol-5-yl)phenyl]-7-{[(2-methoxyphenyl)acetyl]amino}-1H-indole-2-carboxamide

N-[4-bromo-2-(1H-tetraazol-5-yl)phenyl]-7-{[(2-methylphenyl)acetyl]amino}-1H-indole-2-carboxamide

N-[4-bromo-2-(1H-tetraazol-5-yl)phenyl]-7-({[4-(trifluoromethyl)phenyl]acetyl}amino)-1H-indole-2-carboxamide

N-[4-bromo-2-(1H-tetraazol-5-yl)phenyl]-7-{[(4-isopropylphenyl)acetyl]amino}-1H-indole-2-carboxamide

N-[4-bromo-2-(1H-tetraazol-5-yl)phenyl]-7-{[(4-methylphenyl)acetyl]amino}-1H-indole-2-carboxamide

N-[4-bromo-2-(1H-tetraazol-5-yl)phenyl]-7-{[(4-fluorophenyl)acetyl]amino}-1H-indole-2-carboxamide

N-[4-bromo-2-(1H-tetraazol-5-yl)phenyl]-7-({[2-(trifluoromethyl)phenyl]acetyl}amino)-1H-indole-2-carboxamide

N-[4-bromo-2-(1H-tetraazol-5-yl)phenyl]-7-{[(3-fluorophenyl)acetyl]amino}-1H-indole-2-carboxamide

N-[4-bromo-2-(1H-tetraazol-5-yl)phenyl]-7-{[(phenylthio)acetyl]amino}-1H-indole-2-carboxamide

N-[4-bromo-2-(1H-tetraazol-5-yl)phenyl]-7-[(2-naphthylacetyl)amino]-1H-indole-2-carboxamide

N-[4-bromo-2-(1H-tetraazol-5-yl)phenyl]-7-[(1-naphthylacetyl)amino]-1H-indole-2-carboxamide

N-[4-bromo-2-(1H-tetraazol-5-yl)phenyl]-7-{[(2-naphthyloxy)acetyl]amino}-1H-indole-2-carboxamide

N-[4-bromo-2-(1H-tetraazol-5-yl)phenyl]-7-[(2-propoxybenzoyl)amino]-1H-indole-2-carboxamide

N-[4-bromo-2-(1H-tetraazol-5-yl)phenyl]-7-[(tetrahydrofuran-3-ylcarbonyl)amino]-1H-indole-2-carboxamide

N-[4-bromo-2-(1H-tetraazol-5-yl)phenyl]-7-{[(1-methylcyclopropyl)carbonyl]amino}-1H-indole-2-carboxamide

N-[4-bromo-2-(1H-tetraazol-5-yl)phenyl]-7-{[(4-ethoxyphenyl)acetyl]amino}-1H-indole-2-carboxamide

7-[(1-benzothien-3-ylacetyl)amino]-N-[4-bromo-2-(1H-tetraazol-5-yl)phenyl]-1H-indole-2-carboxamide 7-[(1,1′-biphenyl-4-ylcarbonyl)amino]-N-[4-bromo-2-(1H-tetraazol-5-yl)phenyl]-1H-indole-2-carboxamide

N-[4-bromo-2-(1H-tetraazol-5-yl)phenyl]-7-[(4-butoxybenzoyl)amino]-1H-indole-2-carboxamide

N-[4-bromo-2-(1H-tetraazol-5-yl)phenyl]-7-{[2-(2-phenylethyl)benzoyl]amino}-1H-indole-2-carboxamide

7-[(1,1′-biphenyl-2-ylcarbonyl)amino]-N-[4-bromo-2-(1H-tetraazol-5-yl)phenyl]-1H-indole-2-carboxamide

N-[4-bromo-2-(1H-tetraazol-5-yl)phenyl]-7-{[4-(ethylthio)benzoyl]amino}-1H-indole-2-carboxamide

N-[4-bromo-2-(1H-tetraazol-5-yl)phenyl]-7-{[2-(methylsulfonyl)benzoyl]amino}-1H-indole-2-carboxamide

N-[4-bromo-2-(1H-tetraazol-5-yl)phenyl]-7-{[(2,6-dichlorophenyl)acetyl]amino}-1H-indole-2-carboxamide

7-[(1,1′-biphenyl-4-ylacetyl)amino]-N-[4-bromo-2-(1H-tetraazol-5-yl)phenyl]-1H-indole-2-carboxamide

7-[(1,3-benzodioxol-5-ylacetyl)amino]-N-[4-bromo-2-(1H-tetraazol-5-yl)phenyl]-1H-indole-2-carboxamide

N-[4-bromo-2-(1H-tetraazol-5-yl)phenyl]-7-[(3,3-dimethylbutanoyl)amino]-1H-indole-2-carboxamide

N-[4-bromo-2-(1H-tetraazol-5-yl)phenyl]-7-[(thien-2-ylacetyl)amino]-1H-indole-2-carboxamide

N-[4-bromo-2-(1H-tetraazol-5-yl)phenyl]-7-{[(3-methyl-5-phenylisoxazol-4-yl)carbonyl]amino}-1H-indole-2-carboxamide

N-[4-bromo-2-(1H-tetraazol-5-yl)phenyl]-7-({[2-(2-methoxyethoxy)ethoxy]acetyl}amino)-1H-indole-2-carboxamide

N-[4-bromo-2-(1H-tetraazol-5-yl)phenyl]-7-[(2-hydroxybenzoyl)amino]-1H-indole-2-carboxamide

N-[4-bromo-2-(1H-tetraazol-5-yl)phenyl]-7-({[4-(trifluoromethoxy)phenyl]sulfonyl}amino)-1H-indole-2-carboxamide

N-[4-bromo-2-(1H-tetraazol-5-yl)phenyl]-7-(prolylamino)-1H-indole-2-carboxamide

N-[4-bromo-2-(1H-tetraazol-5-yl)phenyl]-7-{[(3-methylisoxazol-5-yl)acetyl]amino}-1H-indole-2-carboxamide

N-[4-bromo-2-(1H-tetraazol-5-yl)phenyl]-6-methoxy-1H-indole-2-carboxamide

N-[4-bromo-2-(1H-tetraazol-5-yl)phenyl]-5-nitro-1,2-benzisoxazole-3-carboxamide

7-[(benzylsulfonyl)amino]-N-[4-bromo-2-(1H-tetraazol-5-yl)phenyl]-1H-indole-2-carboxamide

N-[4-bromo-2-(1H-tetraazol-5-yl)phenyl]-1-methyl-7-{[3-(morpholin-4-ylsulfonyl)benzoyl]amino}-1H-indole-2-carboxamide

N-[4-bromo-2-(1H-tetraazol-5-yl)phenyl]-7-{[(4-fluorophenyl)acetyl]amino}-1-methyl-1H-indole-2-carboxamide

N-[4-bromo-2-(1H-tetraazol-5-yl)phenyl]-7-[(fluoroacetyl)amino]-1-methyl-1H-indole-2-carboxamide

N-[4-bromo-2-(1H-tetraazol-5-yl)phenyl]-1-methyl-7-{[(1-methyl-1H-indol-2-yl)carbonyl]amino}-1H-indole-2-carboxamide

N-[4-bromo-2-(1H-tetraazol-5-yl)phenyl]-5-[(phenylacetyl)amino]-1,2-benzisoxazole-3-carboxamide

5-[(benzylsulfonyl)amino]-N-[4-bromo-2-(1H-tetraazol-5-yl)phenyl]-1,2-benzisoxazole-3-carboxamide

6-(benzyloxy)-N-[4-bromo-2-(1H-tetraazol-5-yl)phenyl]-1-methyl-1H-indole-2-carboxamide

N-[4-bromo-2-(1H-tetraazol-5-yl)phenyl]-6-methoxy-1-methyl-1H-indole-2-carboxamide

N-[4-bromo-2-(1H-tetraazol-5-yl)phenyl]-1-methyl-7-[(morpholin-4-ylcarbonyl)amino]-1H-indole-2-carboxamide

N-[4-bromo-2-(1H-tetraazol-5-yl)phenyl]-1-methyl-7-({[(tetrahydrofuran-2-ylmethyl)amino]carbonyl}amino)-1H-indole-2-carboxamide

N-[4-bromo-2-(1H-tetraazol-5-yl)phenyl]-7-hydroxy-1-methyl-1H-indole-2-carboxamide

7-{[(benzylamino)carbonyl]amino}-N-[4-bromo-2-(1H-tetraazol-5-yl)phenyl]-1-methyl-1H-indole-2-carboxamide

N-[4-bromo-2-(1H-tetraazol-5-yl)phenyl]-7-({[(2,3-dihydroxypropyl)amino]carbonyl}amino)-1-methyl-1H-indole-2-carboxamide

1-[({[2-({[4-bromo-2-(1H-tetraazol-5-yl)phenyl]amino}carbonyl)-1-methyl-1H-indol-7-yl]amino}carbonyl)(methyl)amino]-1-deoxyhexitol

N-[4-bromo-2-(1H-tetraazol-5-yl)phenyl]-7-(2,3-dihydro-1,4-benzodioxin-2-ylmethoxy)-1-methyl-1H-indole-2-carboxamide

7-(benzyloxy)-N-[4-bromo-2-(1H-tetraazol-5-yl)phenyl]-1H-indole-2-carboxamide

N-[4-bromo-2-(1H-tetraazol-5-yl)phenyl]-1-methyl-7-(3-phenoxypropoxy)-1H-indole-2-carboxamide

5-(acetylamino)-N-[4-bromo-2-(1H-tetraazol-5-yl)phenyl]-1,2-benzisoxazole-3-carboxamide

N-[4-bromo-2-(1H-tetraazol-5-yl)phenyl]-5-[(methylsulfonyl)amino]-1,2-benzisoxazole-3-carboxamide

5-(benzoylamino)-N-[4-bromo-2-(1H-tetraazol-5-yl)phenyl]-1,2-benzisoxazole-3-carboxamide

N-[4-bromo-2-(1H-tetraazol-5-yl)phenyl]-5-[(phenylsulfonyl)amino]-1,2-benzisoxazole-3-carboxamide

N-[4-bromo-2-(1H-tetraazol-5-yl)phenyl]-7-(cyclobutylmethoxy)-1-methyl-1H-indole-2-carboxamide

N-[4-bromo-2-(1H-tetraazol-5-yl)phenyl]-7-(2-furylmethoxy)-1-methyl-1H-indole-2-carboxamide

N-[4-bromo-2-(1H-tetraazol-5-yl)phenyl]-7-{[(4S)-2,2-dimethyl-1,3-dioxolan-4-yl]methoxy}-1-methyl-1H-indole-2-carboxamide

N-[4-bromo-2-(1H-tetraazol-5-yl)phenyl]-7-{[(2R)-2,3-dihydroxypropyl]oxy}-1-methyl-1H-indole-2-carboxamide

N-[4-bromo-2-(1H-tetraazol-5-yl)phenyl]-7-(cyclobutyloxy)-1-methyl-1H-indole-2-carboxamide

N-[4-bromo-2-(1H-tetraazol-5-yl)phenyl]-7-(2-methoxy-1-methylethoxy)-1-methyl-1H-indole-2-carboxamide

N-[4-bromo-2-(1H-tetraazol-5-yl)phenyl]-7-isopropoxy-1-methyl-1H-indole-2-carboxamide

7-(benzyloxy)-N-[4-bromo-2-(1H-tetraazol-5-yl)phenyl]-1-methyl-1H-indole-2-carboxamide

N-[4-bromo-2-(1H-tetraazol-5-yl)phenyl]-6-chloro-1,2-benzisoxazole-3-carboxamide

N-[4-bromo-2-(1H-tetraazol-5-yl)phenyl]-6-sec-butoxy-1-methyl-1H-indole-2-carboxamide

N-[4-bromo-2-(1H-tetraazol-5-yl)phenyl]-6-butoxy-1-methyl-1H-indole-2-carboxamide

N-[4-bromo-2-(1H-tetraazol-5-yl)phenyl]-5-{[(4-fluorophenyl)sulfonyl]amino}-1,2-benzisoxazole-3-carboxamide

5-amino-N-[4-bromo-2-(1H-tetraazol-5-yl)phenyl]-1,2-benzisoxazole-3-carboxamide

5-bromo-N-[4-bromo-2-(1H-tetraazol-5-yl)phenyl]-1,2-benzisoxazole-3-carboxamide

6-[(benzylsulfonyl)amino]-N-[4-bromo-2-(1H-tetraazol-5-yl)phenyl]-1,2-benzisoxazole-3-carboxamide

N-[4-bromo-2-(1H-tetraazol-5-yl)phenyl]-6-[(phenylsulfonyl)amino]-1,2-benzisoxazole-3-carboxamide

N-[4-bromo-2-(1H-tetraazol-5-yl)phenyl]-7-(cyclohexylmethoxy)-1-methyl-1H-indole-2-carboxamide

N-[4-bromo-2-(1H-tetraazol-5-yl)phenyl]-7-(cyclopropylmethoxy)-1-methyl-1H-indole-2-carboxamide

N-[4-bromo-2-(1H-tetraazol-5-yl)phenyl]-1-methyl-7-(tetrahydro-2H-pyran-2-ylmethoxy)-1H-indole-2-carboxamide

N-[4-bromo-2-(1H-tetraazol-5-yl)phenyl]-1-methyl-7-(pentyloxy)-1H-indole-2-carboxamide

N-[4-bromo-2-(1H-tetraazol-5-yl)phenyl]-5-phenyl-1,2-benzisoxazole-3-carboxamide

N-[4-bromo-2-(1H-tetraazol-5-yl)phenyl]-5-[2-(trifluoromethyl)phenyl]-1,2-benzisoxazole-3-carboxamide

N-[4-bromo-2-(1H-tetraazol-5-yl)phenyl]-7-(2-methoxyethoxy)-1-methyl-1H-indole-2-carboxamide

N-[4-bromo-2-(1H-tetraazol-5-yl)phenyl]-7-(2-hydroxy-3-isopropoxypropoxy)-1-methyl-1H-indole-2-carboxamide

N-[4-bromo-2-(1H-tetraazol-5-yl)phenyl]-6-phenyl-1,2-benzisoxazole-3-carboxamide

N-[4-bromo-2-(1H-tetraazol-5-yl)-phenyl]-6-[2-(trifluoromethyl)phenyl]-1,2-benzisoxazole-3-carboxamide

N-[4-bromo-2-(1H-tetraazol-5-yl)phenyl]-1-methyl-7-[2-(methylthio)ethoxy]-1H-indole-2-carboxamide

7-[(4-azido-3-iodobenzoyl)amino]-N-[4-bromo-2-(1H-tetraazol-5-yl)phenyl]-1-methyl-1H-indole-2-carboxamide

5-[(4-azido-3-iodobenzoyl)amino]-N-[4-bromo-2-(1H-tetraazol-5-yl)phenyl]-1,2-benzisoxazole-3-carboxamide

N-[4-bromo-2-(1H-tetraazol-5-yl)phenyl]-6-[(methylsulfonyl)amino]-1,2-benzisoxazole-3-carboxamide

N-[4-bromo-2-(1H-tetraazol-5-yl)phenyl]-6-{[(4-fluorophenyl)sulfonyl]amino}-1,2-benzisoxazole-3-carboxamide

6-amino-N-[4-bromo-2-(1H-tetraazol-5-yl)phenyl]-1,2-benzisoxazole-3-carboxamide

N-[4-bromo-2-(1H-tetraazol-5-yl)phenyl]-5-(2-fluorophenyl)-1,2-benzisoxazole-3-carboxamide

N-[4-bromo-2-(1H-tetraazol-5-yl)phenyl]-1-methyl-6-[2-(trifluoromethyl)phenyl]-1H-indole-2-carboxamide

N-[4-bromo-2-(1H-tetraazol-5-yl)phenyl]-1-methyl-6-(2,3,4-trimethoxyphenyl)-1H-indole-2-carboxamide

N-[4-bromo-2-(1H-tetraazol-5-yl)phenyl]-1-methyl-7-(pyridin-3-ylmethoxy)-1H-indole-2-carboxamide

N-[4-bromo-2-(1H-tetraazol-5-yl)phenyl]-6-(3,5-dimethylisoxazol-4-yl)-1-methyl-1H-indole-2-carboxamide

N-[4-bromo-2-(1H-tetraazol-5-yl)phenyl]-1-methyl-6-(2-methylphenyl)-1H-indole-2-carboxamide

7-amino-4,6-dibromo-N-[4-bromo-2-(1H-tetraazol-5-yl)phenyl]-1-methyl-1H-indole-2-carboxamide

4,6-dibromo-N-[4-bromo-2-(1H-tetraazol-5-yl)phenyl]-1-methyl-7-[(phenylacetyl)amino]-1H-indole-2-carboxamide

3-({[4-bromo-2-(1H-tetraazol-5-yl)phenyl]amino}carbonyl)-1,2-benzisoxazol-6-yl acetate

N-[4-bromo-2-(1H-tetraazol-5-yl)phenyl]-6-hydroxy-1,2-benzisoxazole-3-carboxamide

6-(benzyloxy)-N-[4-bromo-2-(1H-tetraazol-5-yl)phenyl]-1,2-benzisoxazole-3-carboxamide

N-[4-bromo-2-(1H-tetraazol-5-yl)phenyl]-6-methoxy-1,2-benzisoxazole-3-carboxamide

3-({[4-bromo-2-(1H-tetraazol-5-yl)phenyl]amino}carbonyl)-1,2-benzisoxazol-6-yl phenylmethanesulfonate

3-({[4-bromo-2-(1H-tetraazol-5-yl)phenyl]amino}carbonyl)-1,2-benzisoxazol-6-yl benzenesulfonate

7-amino-4-bromo-N-[4-bromo-2-(1H-tetraazol-5-yl)phenyl]-1-methyl-1H-indole-2-carboxamide

7-(benzyloxy)-3-bromo-N-[4-bromo-2-(1H-tetraazol-5-yl)phenyl]-1H-indole-2-carboxamide

2-{[2-({[4-bromo-2-(1H-tetraazol-5-yl)phenyl]amino}carbonyl)-1-methyl-1H-indol-6-yl]amino}-2-oxoethyl acetate

5-(anilinosulfonyl)-N-[4-bromo-2-(1H-tetraazol-5-yl)phenyl]-1,2-benzisoxazole-3-carboxamide

N-[4-bromo-2-(1H-tetraazol-5-yl)phenyl]-5-[(diethylamino)sulfonyl]-1,2-benzisoxazole-3-carboxamide

7-amino-6-bromo-N-[4-bromo-2-(1H-tetraazol-5-yl)phenyl]-1-methyl-1H-indole-2-carboxamide

N-[4-bromo-2-(1H-tetraazol-5-yl)phenyl]-1-methyl-7-[(phenylacetyl)amino]-4,6-bis[2-(trifluoromethyl)phenyl]-1H-indole-2-carboxamide

N-[4-bromo-2-(1H-tetraazol-5-yl)phenyl]-5-nitro-6-[4-nitro-2-(trifluoromethyl)phenyl]-1,2-benzisoxazole-3-carboxamide

7-(benzyloxy)-3-bromo-N-[4-bromo-2-(1H-tetraazol-5-yl)phenyl]-1-methyl-1H-indole-2-carboxamide

4-bromo-N-[4-bromo-2-(1H-tetraazol-5-yl)phenyl]-1-methyl-7-[(phenylacetyl)amino]-1H-indole-2-carboxamide

N-[4-bromo-2-(1H-tetraazol-5-yl)phenyl]-1-methyl-6-[(quinolin-8-ylsulfonyl)amino]-1H-indole-2-carboxamide

N-[4-bromo-2-(1H-tetraazol-5-yl)phenyl]-5-(morpholin-4-ylsulfonyl)-1,2-benzisoxazole-3-carboxamide

N-[4-bromo-2-(1H-tetraazol-5-yl)phenyl]-6-(cyclobutylmethoxy)-1-methyl-1H-indole-2-carboxamide

N-[4-bromo-2-(1H-tetraazol-5-yl)phenyl]-6-(butyrylamino)-1-methyl-1H-indole-2-carboxamide

N-[4-bromo-2-(1H-tetraazol-5-yl)phenyl]-6-[(methoxyacetyl)amino]-1-methyl-1H-indole-2-carboxamide

N-[4-bromo-2-(1H-tetraazol-5-yl)phenyl]-7-methyl-1H-indole-2-carboxamide

N-[4-bromo-2-(1H-tetraazol-5-yl)phenyl]-6-oxo-5,6-dihydro-4H-pyrrolo[3,2,1-ij]quinoline-2-carboxamide

5-(acetylamino)-6-[4-(acetylamino)-2-(trifluoromethyl)phenyl]-N-[4-bromo-2-(H-tetraazol-5-yl)phenyl]-1,2-benzisoxazole-3-carboxamide

N-[4-bromo-2-(1H-tetraazol-5-yl)phenyl]-5-[(dimethylamino)sulfonyl]-1,2-benzisoxazole-3-carboxamide

N-[4-bromo-2-(1H-tetraazol-5-yl)phenyl]-5-[(methoxyacetyl)amino]-1,2-benzisoxazole-3-carboxamide

N-[4-bromo-2-(1H-tetraazol-5-yl)phenyl]-5-[(cyclobutylcarbonyl)amino]-1,2-benzisoxazole-3-carboxamide

N-[4-bromo-2-(1H-tetraazol-5-yl)phenyl]-6-(2-furoylamino)-1-methyl-1H-indole-2-carboxamide

N-[4-bromo-2-(1H-tetraazol-5-yl)phenyl]-1-methyl-6-({[methyl(phenyl)amino]carbonyl}amino)-1H-indole-2-carboxamide

6-(acetylamino)-N-[4-bromo-2-(1H-tetraazol-5-yl)phenyl]-1-methyl-1H-indole-2-carboxamide

N-[4-bromo-2-(1H-tetraazol-5-yl)phenyl]-1-methyl-6-{[4-(trifluoromethoxy)benzoyl]amino}-1H-indole-2-carboxamide

N-[4-bromo-2-(1H-tetraazol-5-yl)phenyl]-6-(2-fluorophenyl)-1-methyl-1H-indole-2-carboxamide

N-[4-bromo-2-(1H-tetraazol-5-yl)phenyl]-6-(cyclopropylmethoxy)-1-methyl-1H-indole-2-carboxamide

7-bromo-N-[4-bromo-2-(1H-tetraazol-5-yl)phenyl]-1-methyl-1H-indole-2-carboxamide

N-[4-bromo-2-(1H-tetraazol-5-yl)phenyl]-6-methoxy-5,6-dihydro-4H-pyrrolo[3,2,1-ij]quinoline-2-carboxamide

N-[4-bromo-2-(1H-tetraazol-5-yl)phenyl]-6hydroxy-5,6-dihydro-4H-pyrrolo[3,2,1-ij]quinoline-2-carboxamide

N-[4-bromo-2-(1H-tetraazol-5-yl)phenyl]-6-phenoxy-5,6-dihydro-4H-pyrrolo[3,2,1-ij]quinoline-2-carboxamide

N-[4-bromo-2-(1H-tetraazol-5-yl)phenyl]-1-methyl-7-[2-(trifluoromethyl)phenyl]-1H-indole-2-carboxamide

N-[4-bromo-2-(1H-tetraazol-5-yl)phenyl]-1-methyl-7-phenyl-1H-indole-2-carboxamide

N-[4-bromo-2-(1H-tetraazol-5-yl)phenyl]-7-(4-tert-butylphenyl)-1-methyl-1H-indole-2-carboxamide

N-[4-bromo-2-(1H-tetraazol-5-yl)phenyl]-1-methyl-7-(5-methylthien-2-yl)-1H-indole-2-carboxamide

N-[4-bromo-2-(1H-tetraazol-5-yl)phenyl]-1-methyl-6-[(pyridin-3-ylcarbonyl)amino]-1H-indole-2-carboxamide

N-[4-bromo-2-(1H-tetraazol-5-yl)phenyl]-1-methyl-6-[(methylsulfonyl)amino]-1H-indole-2-carboxamide

N-[4-bromo-2-(1H-tetraazol-5-yl)phenyl]-6-({[(2,5-dimethoxyphenyl)amino]carbonyl}amino)-1-methyl-1H-indole-2-carboxamide

N-[4-bromo-2-(1H-tetraazol-5-yl)phenyl]-6-[(isoxazol-5-ylcarbonyl)amino]-1-methyl-1H-indole-2-carboxamide

N-[4-bromo-2-(1H-tetraazol-5-yl)phenyl]-1-methyl-6-{[(pentylamino)carbonyl]amino}-1H-indole-2-carboxamide

N-[4-bromo-2-(1H-tetraazol-5-yl)phenyl]-1-methyl-7-[4-(methylsulfonyl)phenyl]-1H-indole-2-carboxamide

N-[4-bromo-2-(1H-tetraazol-5-yl)phenyl]-7-(2-methoxyphenyl)-1-methyl-1H-indole-2-carboxamide

N-[4-bromo-2-(1H-tetraazol-5-yl)phenyl]-7-(2-fluorophenyl)-1-methyl-1H-indole-2-carboxamide

N-[4-bromo-2-(1H-tetraazol-5-yl)phenyl]-1-methyl-7-(2-methylphenyl)-1H-indole-2-carboxamide

N-[4-bromo-2-(1H-tetraazol-5-yl)phenyl]-6-{[(3,5-dimethylisoxazol-4-yl)sulfonyl]amino}-1-methyl-1H-indole-2-carboxamide

N-[4-bromo-2-(1H-tetraazol-5-yl)phenyl]-5-(dimethylamino)-1,2-benzisoxazole-3-carboxamide

N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-5-methoxy-1H-indole-2-carboxamide

5-(benzyloxy)-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-1-methyl-1H-indole-2-carboxamide

6-(benzyloxy)-N-[4-cyano-2-(5-oxo-4,5-dihydro-1,2,4-oxadiazol-3-yl)phenyl]-1H-indole-2-carboxamide

7-chloro-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-4-methoxy-1H-indole-2-carboxamide

6-chloro-N-[4-cyano-2-(5-oxo-4,5-dihydro-1,2,4-oxadiazol-3-yl)phenyl]-1H-indole-2-carboxamide

1-benzyl-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-1-ethyl-1H-indole-2-carboxamide

N-[4-cyano-2-(5-oxo-4,5-dihydro-1,2,4-oxadiazol-3-yl)phenyl]-7-(phenylsulfonyl)-1H-indole-2-carboxamide

1-allyl-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-1-(cyclohexylmethyl)-1H-indole-2-carboxamide

N-[4-cyano-2-(5-oxo-4,5-dihydro-1,2,4-oxadiazol-3-yl)phenyl]-1-(2-methoxyethyl)-1H-indole-2-carboxamide

N-[4-cyano-2-(5-oxo-4,5-dihydro-1,2,4-oxadiazol-3-yl)phenyl]-1-pentyl-1H-indole-2-carboxamide

1-butyl-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-1-propyl-1H-indole-2-carboxamide

N-[4-cyano-2-(5-oxo-4,5-dihydro-1,2,4-oxadiazol-3-yl)phenyl]-1-isopropyl-1H-indole-2-carboxamide

-N-[4-cyano-2-(5-oxo-4,5-dihydro-1,2,4-oxadiazol-3-yl)phenyl]-1-isobutyl-1H-indole-2-carboxamide

N-[4-cyano-2-(5-oxo-4,5-dihydro-1,2,4-oxadiazol-3-yl)phenyl]-1-(3-phenylpropyl)-1H-indole-2-carboxamide

N-[4-cyano-2-(5-oxo-4,5-dihydro-1,2,4-oxadiazol-3-yl)phenyl]-1-methyl-7-(phenylsulfonyl)-1H-indole-2-carboxamide

N-[4-cyano-2-(5-oxo-4,5-dihydro-1,2,4-oxadiazol-3-yl)phenyl]-7-[(phenylacetyl)amino]-1H-indole-2-carboxamide

7-(benzoylamino)-N-[4-cyano-2-(5-oxo-4,5-dihydro-1,2,4-oxadiazol-3-yl)phenyl]-1H-indole-2-carboxamide

2-{[2-({[4-cyano-2-(5-oxo-4,5-dihydro-1,2,4-oxadiazol-3-yl)phenyl]amino}carbonyl)-1H-indol-7-yl]amino}-2-oxoethyl acetate N-[4-cyano-2-(5-oxo-4,5-dihydro-1,2,4-oxadiazol-3-yl)phenyl]-7-[(cyclopentylcarbonyl)amino]-1H-indole-2-carboxamide

7-amino-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-1,2-benzisoxazole-3-carboxamide

7-{[(6-chloropyridin-3-yl)carbonyl]amino}-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-[(isoxazol-5-ylcarbonyl)amino]-1H-indole-2-carboxamide

N-[4-cyano-2-(5-oxo-4,5-dihydro-1,2,4-oxadiazol-3-yl)phenyl]-7-[(2,4-difluorobenzoyl)amino]-1H-indole-2-carboxamide

N-[4-cyano-2-(5-oxo-4,5-dihydro-1,2,4-oxadiazol-3-yl)phenyl]-7-[(fluoroacetyl)amino]-1H-indole-2-carboxamide

7-(acetylamino)-N-[4-cyano-2-(5-oxo-4,5-dihydro-1,2,4-oxadiazol-3-yl)phenyl]-1H-indole-2-carboxamide

7-{[(4-chlorophenyl)acetyl]amino}-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-{[(4-methoxyphenyl)acetyl]amino}-1H-indole-2-carboxamide

N-[4-cyano-2-(5-oxo-4,5-dihydro-1,2,4-oxadiazol-3-yl)phenyl]-7-[(cyclopentylacetyl)amino]-1H-indole-2-carboxamide

N-[4-cyano-2-(5-oxo-4,5-dihydro-1,2,4-oxadiazol-3-yl)phenyl]-7-[(3-fluorobenzoyl)amino]-1H-indole-2-carboxamide

7-[(3-cyanobenzoyl)amino]-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-[(cyclohexylcarbonyl)amino]-1H-indole-2-carboxamide

N-[4-cyano-2-(5-oxo-4,5-dihydro-1,2,4-oxadiazol-3-yl)phenyl]-7-(propionylamino)-1H-indole-2-carboxamide

methyl 5-{[2-({[4-cyano-2-(5-oxo-4,5-dihydro-1,2,4-oxadiazol-3-yl)phenyl]amino}carbonyl)-1H-indol-7-yl]amino}-5-oxopentanoate

7-(butyrylamino)-N-[4-cyano-2-(5-oxo-4,5-dihydro-1,2,4-oxadiazol-3-yl)phenyl]-1H-indole-2-carboxamide

7-[(4-bromobenzoyl)amino]-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-[(3-phenylpropanoyl)amino]-1H-indole-2-carboxamide

N-[4-cyano-2-(5-oxo-4,5-dihydro-1,2,4-oxadiazol-3-yl)phenyl]-7-[(phenoxyacetyl)amino]-1H-indole-2-carboxamide

N-[4-cyano-2-(5-oxo-4,5-dihydro-1,2,4-oxadiazol-3-yl)phenyl]-7-[(3-cyclopentylpropanoyl)amino]-1H-indole-2-carboxamide

methyl 3-{[2-({[4-cyano-2-(5-oxo-4,5-dihydro-1,2,4-oxadiazol-3-yl)phenyl]amino}carbonyl)-1H-indol-7-yl]amino}-3-oxopropanoate

N-[4-cyano-2-(5-oxo-4,5-dihydro-1,2,4-oxadiazol-3-yl)phenyl]-7-[(2-ethylhexanoyl)amino]-1H-indole-2-carboxamide

N-[4-cyano-2-(5-oxo-4,5-dihydro-1,2,4-oxadiazol-3-yl)phenyl]-7-{[(3,4-dimethoxyphenyl)acetyl]amino}-1H-indole-2-carboxamide

N-[4-cyano-2-(5-oxo-4,5-dihydro-1,2,4-oxadiazol-3-yl)phenyl]-7-[(3,5,5-trimethylhexanoyl)amino]-1H-indole-2-carboxamide

N-[4-cyano-2-(5-oxo-4,5-dihydro-1,2,4-oxadiazol-3-yl)phenyl]-7-[(cyclopropylcarbonyl)amino]-1H-indole-2-carboxamide

N-[4-cyano-2-(5-oxo-4,5-dihydro-1,2,4-oxadiazol-3-yl)phenyl]-7-[(methoxyacetyl)amino]-1H-indole-2-carboxamide

N-[4-cyano-2-(5-oxo-4,5-dihydro-1,2,4-oxadiazol-3-yl)phenyl]-7-[(3-methylbutanoyl)amino]-1H-indole-2-carboxamide

N-[4-cyano-2-(5-oxo-4,5-dihydro-1,2,4-oxadiazol-3-yl)phenyl]-7-(pentanoylamino)-1H-indole-2-carboxamide

N-[4-cyano-2-(5-oxo-4,5-dihydro-1,2,4-oxadiazol-3-yl)phenyl]-7-{[(4,7,7-trimethyl-3-oxo-2-oxabicyclo[2.2.1]hept-1-yl)carbonyl]amino}-1H-indole-2-carboxamide

7-{[chloro(phenyl)acetyl]amino}-N-[4-cyano-2-(5-oxo-4,5-dihydro-1,2,4-oxadiazol-3-yl)phenyl]-1H-indole-2-carboxamide

7-{[(benzyloxy)acetyl]amino}-N-[4-cyano-2-(5-oxo-4,5-dihydro-1,2,4-oxadiazol-3-yl)phenyl]-1H-indole-2-carboxamide

ethyl 3-{[2-({[4-cyano-2-(5-oxo-4,5-dihydro-1,2,4-oxadiazol-3-yl)phenyl]amino}carbonyl)-1H-indol-7-yl]amino}-3-oxopropanoate

7-[(1-adamantylcarbonyl)amino]-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-(hexanoylamino)-1H-indole-2-carboxamide

N-[4-cyano-2-(5-oxo-4,5-dihydro-1,2,4-oxadiazol-3-yl)phenyl]-7-{[(2-phenylcyclopropyl)carbonyl]amino}-1H-indole-2-carboxamide

N-[4-cyano-2-(5-oxo-4,5-dihydro-1,2,4-oxadiazol-3-yl)phenyl]-7-[(2-phenylbutanoyl)amino]-1H-indole-2-carboxamide

N-[4-cyano-2-(5-oxo-4,5-dihydro-1,2,4-oxadiazol-3-yl)phenyl]-7-(heptanoylamino)-1H-indole-2-carboxamide

2-{[2-({[4-cyano-2-(5-oxo-4,5-dihydro-1,2,4-oxadiazol-3-yl)phenyl]amino}carbonyl)-1H-indol-7-yl]amino}-2-oxo-1-phenylethyl acetate N-[4-cyano-2-(5-oxo-4,5-dihydro-1,2,4-oxadiazol-3-yl)phenyl]-7-[(thien-2-ylcarbonyl)amino]-1H-indole-2-carboxamide

N-[4-cyano-2-(5-oxo-4,5-dihydro-1,2,4-oxadiazol-3-yl)phenyl]-7-[(2-methylbutanoyl)amino]-1H-indole-2-carboxamide

methyl 8-{[2-({[4-cyano-2-(5-oxo-4,5-dihydro-1,2,4-oxadiazol-3-yl)phenyl]amino}carbonyl)-1H-indol-7-yl]amino}-8-oxooctanoate

N-[4-cyano-2-(5-oxo-4,5-dihydro-1,2,4-oxadiazol-3-yl)phenyl]-7-[(2-ethylbutanoyl)amino]-1H-indole-2-carboxamide

N-[4-cyano-2-(5-oxo-4,5-dihydro-1,2,4-oxadiazol-3-yl)phenyl]-7-(octanoylamino)-1H-indole-2-carboxamide

N-[4-cyano-2-(5-oxo-4,5-dihydro-1,2,4-oxadiazol-3-yl)phenyl]-7-[(cyclobutylcarbonyl)amino]-1H-indole-2-carboxamide

N-[4-cyano-2-(5-oxo-4,5-dihydro-1,2,4-oxadiazol-3-yl)phenyl]-7-(1,3-dioxo-1,3-dihydro-2H-isoindol-2-yl)-1H-indole-2-carboxamide

7-({[2-(benzylthio)-1,3-thiazol-4-yl]carbonyl}amino)-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-{[3-(morpholin-4-ylsulfonyl)benzoyl]amino)-1H-indole-2-carboxamide

N-[4-cyano-2-(5-oxo-4,5-dihydro-1,2,4-oxadiazol-3-yl)phenyl]-7-[(1H-indol-2-ylcarbonyl)amino]-1H-indole-2-carboxamide

N-[2-({[4-cyano-2-(5-oxo-4,5-dihydro-1,2,4-oxadiazol-3-yl)phenyl]amino}carbonyl)-1H-indol-7-yl]-1-methyl-1H-indole-2-carboxamide

N-[4-cyano-2-(5-oxo-4,5-dihydro-1,2,4-oxadiazol-3-yl)phenyl]-7-{[(5-phenylisoxazol-3-yl)carbonyl]amino}-1H-indole-2-carboxamide

N-[4-cyano-2-(5-oxo-4,5-dihydro-1,2,4-oxadiazol-3-yl)phenyl]-7-[(5-phenylpentanoyl)amino]-1H-indole-2-carboxamide

N-[4-cyano-2-(5-oxo-4,5-dihydro-1,2,4-oxadiazol-3-yl)phenyl]-7-[(4-phenylbutanoyl)amino]-1H-indole-2-carboxamide

N-[4-cyano-2-(5-oxo-4,5-dihydro-1,2,4-oxadiazol-3-yl)phenyl]-7-{[4-(4-methoxyphenyl)butanoyl]amino}-1H-indole-2-carboxamide

7-{[(2-chlorophenyl)acetyl]amino}-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-{[(2,4-dichlorophenyl)acetyl]amino}-1H-indole-2-carboxamide

N-[4-cyano-2-(5-oxo-4,5-dihydro-1,2,4-oxadiazol-3-yl)phenyl]-7-{[(3,4-dichlorophenyl)acetyl]amino}-1H-indole-2-carboxamide

7-{[(3-chlorophenyl)acetyl]amino}-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-({[3-(trifluoromethyl)phenyl]acetyl}amino)-1H-indole-2-carboxamide

N-[4-cyano-2-(5-oxo-4,5-dihydro-1,2,4-oxadiazol-3-yl)phenyl]-7-{[(3-methylphenyl)acetyl]amino}-1H-indole-2-carboxamide

7-{[(4-tert-butylphenyl)acetyl]amino}-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-{[(3-methoxyphenyl)acetyl]amino}-1H-indole-2-carboxamide

N-[4-cyano-2-(5-oxo-4,5-dihydro-1,2,4-oxadiazol-3-yl)phenyl]-7-{[(2-methoxyphenyl)acetyl]amino}-1H-indole-2-carboxamide

N-[4-cyano-2-(5-oxo-4,5-dihydro-1,2,4-oxadiazol-3-yl)phenyl]-7-{[(2-methylphenyl)acetyl]amino}-1H-indole-2-carboxamide

N-[4-cyano-2-(5-oxo-4,5-dihydro-1,2,4-oxadiazol-3-yl)phenyl]-7-({[4-(trifluoromethyl)phenyl]acetyl}amino)-1H-indole-2-carboxamide

N-[4-cyano-2-(5-oxo-4,5-dihydro-1,2,4-oxadiazol-3-yl)phenyl]-7-{[(4-isopropylphenyl)acetyl]amino}-1H-indole-2-carboxamide

N-[4-cyano-2-(5-oxo-4,5-dihydro-1,2,4-oxadiazol-3-yl)phenyl]-7-{[(4-methylphenyl)acetyl]amino}-1H-indole-2-carboxamide

N-[4-cyano-2-(5-oxo-4,5-dihydro-1,2,4-oxadiazol-3-yl)phenyl]-7-{[(4-fluorophenyl)acetyl]amino}-1H-indole-2-carboxamide

N-[4-cyano-2-(5-oxo-4,5-dihydro-1,2,4-oxadiazol-3-yl)phenyl]-7-({[2-(trifluoromethyl)phenyl]acetyl}amino)-1H-indole-2-carboxamide

N-[4-cyano-2-(5-oxo-4,5-dihydro-1,2,4-oxadiazol-3-yl)phenyl]-7-{[(3-fluorophenyl)acetyl]amino}-1H-indole-2-carboxamide

N-[4-cyano-2-(5-oxo-4,5-dihydro-1,2,4-oxadiazol-3-yl)phenyl]-7-{[(phenylthio)acetyl]amino}-1H-indole-2-carboxamide

N-[4-cyano-2-(5-oxo-4,5-dihydro-1,2,4-oxadiazol-3-yl)phenyl]-7-[(2-naphthylacetyl)amino]-1H-indole-2-carboxamide

N-[4-cyano-2-(5-oxo-4,5-dihydro-1,2,4-oxadiazol-3-yl)phenyl]-7-[(1-naphthylacetyl)amino]-1H-indole-2-carboxamide

N-[4-cyano-2-(5-oxo-4,5-dihydro-1,2,4-oxadiazol-3-yl)phenyl]-7-{[(2-naphthyloxy)acetyl]amino}-1H-indole-2-carboxamide

N-[4-cyano-2-(5-oxo-4,5-dihydro-1,2,4-oxadiazol-3-yl)phenyl]-7-[(2-propoxybenzoyl)amino]-1H-indole-2-carboxamide

N-[4-cyano-2-(5-oxo-4,5-dihydro-1,2,4-oxadiazol-3-yl)phenyl]-7-[(tetrahydrofuran-3-ylcarbonyl)amino]-1H-indole-2-carboxamide

N-[4-cyano-2-(5-oxo-4,5-dihydro-1,2,4-oxadiazol-3-yl)phenyl]-7-{[(1-methylcyclopropyl)carbonyl]amino}-1H-indole-2-carboxamide

N-[4-cyano-2-(5-oxo-4,5-dihydro-1,2,4-oxadiazol-3-yl)phenyl]-7-{[(4-ethoxyphenyl)acetyl]amino}-1H-indole-2-carboxamide

7-[(1-benzothien-3-ylacetyl)amino]-N-[4-cyano-2-(5-oxo-4,5-dihydro-1,2,4-oxadiazol-3-yl)phenyl]-1H-indole-2-carboxamide

7-[(1,1′-biphenyl-4-ylcarbonyl)amino]-N-[4-cyano-2-(5-oxo-4,5-dihydro-1,2,4-oxadiazol-3-yl)phenyl]-1H-indole-2-carboxamide

7-[(4-butoxybenzoyl)amino]-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-{[2-(2-phenylethyl)benzoyl]amino}-1H-indole-2-carboxamide

7-[(1,1′-biphenyl-2-ylcarbonyl)amino]-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-{[4-(ethylthio)benzoyl]amino}-1H-indole-2-carboxamide

N-[4-cyano-2-(5-oxo-4,5-dihydro-1,2,4-oxadiazol-3-yl)phenyl]-7-{[2-(methylsulfonyl)benzoyl]amino}-1H-indole-2-carboxamide

N-[4-cyano-2-(5-oxo-4,5-dihydro-1,2,4-oxadiazol-3-yl)phenyl]-7-{[(2,6-dichlorophenyl)acetyl]amino}-1H-indole-2-carboxamide

7-[(1,1′-biphenyl-4-ylacetyl)amino]-N-[4-cyano-2-(5-oxo-4,5-dihydro-1,2,4-oxadiazol-3-yl)phenyl]-1H-indole-2-carboxamide

7-[(1,3-benzodioxol-5-ylacetyl)amino]-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-7-[(3,3-dimethylbutanoyl)amino]-1H-indole-2-carboxamide

N-[4-cyano-2-(5-oxo-4,5-dihydro-1,2,4-oxadiazol-3-yl)phenyl]-7-[(thien-2-ylacetyl)amino]-1H-indole-2-carboxamide

N-[4-cyano-2-(5-oxo-4,5-dihydro-1,2,4-oxadiazol-3-yl)phenyl]-7-{[(3-methyl-5-phenylisoxazol-4-yl)carbonyl]amino}-1H-indole-2-carboxamide

N-[4-cyano-2-(5-oxo-4,5-dihydro-1,2,4-oxadiazol-3-yl)phenyl]-7-({[2-(2-methoxyethoxy)ethoxy]acetyl}amino)-1H-indole-2-carboxamide

N-[4-cyano-2-(5-oxo-4,5-dihydro-1,2,4-oxadiazol-3-yl)phenyl]-7-[(2-hydroxybenzoyl)amino]-1H-indole-2-carboxamide

N-[4-cyano-2-(5-oxo-4,5-dihydro-1,2,4-oxadiazol-3-yl)phenyl]-7-({[4-(trifluoromethoxy)phenyl]sulfonyl}amino)-1H-indole-2-carboxamide

N-[4-cyano-2-(5-oxo-4,5-dihydro-1,2,4-oxadiazol-3-yl)phenyl]-7-(prolylamino)-1H-indole-2-carboxamide

N-[4-cyano-2-(5-oxo-4,5-dihydro-1,2,4-oxadiazol-3-yl)phenyl]-7-{[(3-methylisoxazol-5-yl)acetyl]amino}-1H-indole-2-carboxamide

N-[4-cyano-2-(5-oxo-4,5-dihydro-1,2,4-oxadiazol-3-yl)phenyl]-6-methoxy-1H-indole-2-carboxamide

N-[4-cyano-2-(5-oxo-4,5-dihydro-1,2,4-oxadiazol-3-yl)phenyl]-5-nitro-1,2-benzisoxazole-3-carboxamide

7-[(benzylsulfonyl)amino]-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-1-methyl-7-{[3-(morpholin-4-ylsulfonyl)benzoyl]amino}-1H-indole-2-carboxamide

N-[4-cyano-2-(5-oxo-4,5-dihydro-1,2,4-oxadiazol-3-yl)phenyl]-7-{[(4-fluorophenyl)acetyl]amino}-1-methyl-1H-indole-2-carboxamide

N-[4-cyano-2-(5-oxo-4,5-dihydro-1,2,4-oxadiazol-3-yl)phenyl]-7-[(fluoroacetyl)amino]-1-methyl-1H-indole-2-carboxamide

N-[4-cyano-2-(5-oxo-4,5-dihydro-1,2,4-oxadiazol-3-yl)phenyl]-1-methyl-7-{[(1-methyl-1H-indol-2-yl)carbonyl]amino}-1H-indole-2-carboxamide

N-[4-cyano-2-(5-oxo-4,5-dihydro-1,2,4-oxadiazol-3-yl)phenyl]-5-[(phenylacetyl)amino]-1,2-benzisoxazole-3-carboxamide

5-[(benzylsulfonyl)amino]-N-[4-cyano-2-(5-oxo-4,5-dihydro-1,2,4-oxadiazol-3-yl)phenyl]-1,2-benzisoxazole-3-carboxamide

6-(benzyloxy)-N-[4-cyano-2-(5-oxo-4,5-dihydro-1,2,4-oxadiazol-3-yl)phenyl]-1-methyl-1H-indole-2-carboxamide N-[4-cyano-2-(5-oxo-4,5-dihydro-1,2,4-oxadiazol-3-yl)phenyl]-6-methoxy-1-methyl-1H-indole-2-carboxamide

N-[4-cyano-2-(5-oxo-4,5-dihydro-1,2,4-oxadiazol-3-yl)phenyl]-1-methyl-7-[(morpholin-4-ylcarbonyl)amino]-1H-indole-2-carboxamide

N-[4-cyano-2-(5-oxo-4,5-dihydro-1,2,4-oxadiazol-3-yl)phenyl]-1-methyl-7-({[(tetrahydrofuran-2-ylmethyl)amino]carbonyl}amino)-1H-indole-2-carboxamide

N-[4-cyano-2-(5-oxo-4,5-dihydro-1,2,4-oxadiazol-3-yl)phenyl]-7-hydroxy-1-methyl-1H-indole-2-carboxamide

7-{[(benzylamino)carbonyl]amino}-N-[4-cyano-2-(5-oxo-4,5-dihydro-1,2,4-oxadiazol-3-yl)phenyl]-1-methyl-1H-indole-2-carboxamide

N-[4-cyano-2-(5-oxo-4,5-dihydro-1,2,4-oxadiazol-3-yl)phenyl]-7-({[(2,3-dihydroxypropyl)amino]carbonyl}amino)-1-methyl-1H-indole-2-carboxamide

1-[({[2-({[4-cyano-2-(5-oxo-4,5-dihydro-1,2,4-oxadiazol-3-yl)phenyl]amino}carbonyl)-1-methyl-1H-indol-7-yl]amino}carbonyl)(methyl)amino]-1-deoxyhexitol

N-[4-cyano-2-(5-oxo-4,5-dihydro-1,2,4-oxadiazol-3-yl)phenyl]-7-(2,3-dihydro-1,4-benzodioxin-2-ylmethoxy)-1-methyl-1H-indole-2-carboxamide

7-(benzyloxy)-N-[4-cyano-2-(5-oxo-4,5-dihydro-1,2,4-oxadiazol-3-yl)phenyl]-1H-indole-2-carboxamide

N-[4-cyano-2-(5-oxo-4,5-dihydro-1,2,4-oxadiazol-3-yl)phenyl]-1-methyl-7-(3-phenoxypropoxy)-1H-indole-2-carboxamide

5-(acetylamino)-N-[4-cyano-2-(5-oxo-4,5-dihydro-1,2,4-oxadiazol-3-yl)phenyl]-1,2-benzisoxazole-3-carboxamide

N-[4-cyano-2-(5-oxo-4,5-dihydro-1,2,4-oxadiazol-3-yl)phenyl]-5-[(methylsulfonyl)amino]-1,2-benzisoxazole-3-carboxamide

5-(benzoylamino)-N-[4-cyano-2-(5-oxo-4,5-dihydro-1,2,4-oxadiazol-3-yl)phenyl]-1,2-benzisoxazole-3-carboxamide

N-[4-cyano-2-(5-oxo-4,5-dihydro-1,2,4-oxadiazol-3-yl)phenyl]-5-[(phenylsulfonyl)amino]-1,2-benzisoxazole-3-carboxamide

N-[4-cyano-2-(5-oxo-4,5-dihydro-1,2,4-oxadiazol-3-yl)phenyl]-7-(cyclobutylmethoxy)-1-methyl-1H-indole-2-carboxamide

N-[4-cyano-2-(5-oxo-4,5-dihydro-1,2,4-oxadiazol-3-yl)phenyl]-7-(2-furylmethoxy)-1-methyl-1H-indole-2-carboxamide

N-[4-cyano-2-(5-oxo-4,5-dihydro-1,2,4-oxadiazol-3-yl)phenyl]-7-{[(4S)-2,2-dimethyl-1,3-dioxolan-4-yl]methoxy}-1-methyl-1H-indole-2-carboxamide

N-[4-cyano-2-(5-oxo-4,5-dihydro-1,2,4-oxadiazol-3-yl)phenyl]-7-{[(2R)-2,3-dihydroxypropyl]oxy}-1-methyl-1H-indole-2-carboxamide

N-[4-cyano-2-(5-oxo-4,5-dihydro-1,2,4-oxadiazol-3-yl)phenyl]-7-(cyclobutyloxy)-1-methyl-1H-indole-2-carboxamide

N-[4-cyano-2-(5-oxo-4,5-dihydro-1,2,4-oxadiazol-3-yl)phenyl]-7-(2-methoxy-1-methylethoxy)-1-methyl-1H-indole-2-carboxamide

N-[4-cyano-2-(5-oxo-4,5-dihydro-1,2,4-oxadiazol-3-yl)phenyl]-7-isopropoxy-1-methyl-1H-indole-2-carboxamide

7-(benzyloxy)-N-[4-cyano-2-(5-oxo-4,5-dihydro-1,2,4-oxadiazol-3-yl)phenyl]-1-methyl-1H-indole-2-carboxamide

6-chloro-N-[4-cyano-2-(5-oxo-4,5-dihydro-1,2,4-oxadiazol-3-yl)phenyl]-1,2-benzisoxazole-3-carboxamide

6-sec-butoxy-N-[4-cyano-2-(5-oxo-4,5-dihydro-1,2,4-oxadiazol-3-yl)phenyl]-1-methyl-1H-indole-2-carboxamide

6-butoxy-N-[4-cyano-2-(5-oxo-4,5-dihydro-1,2,4-oxadiazol-3-yl)phenyl]-1-methyl-1H-indole-2-carboxamide

N-[4-cyano-2-(5-oxo-4,5-dihydro-1,2,4-oxadiazol-3-yl)phenyl]-5-{[(4-fluorophenyl)sulfonyl]amino}-1,2-benzisoxazole-3-carboxamide

5-amino-N-[4-cyano-2-(5-oxo-4,5-dihydro-1,2,4-oxadiazol-3-yl)phenyl]-1,2-benzisoxazole-3-carboxamide

5-bromo-N-[4-cyano-2-(5-oxo-4,5-dihydro-1,2,4-oxadiazol-3-yl)phenyl]-1,2-benzisoxazole-3-carboxamide

6-[(benzylsulfonyl)amino]-N-[4-cyano-2-(5-oxo-4,5-dihydro-1,2,4-oxadiazol-3-yl)phenyl]-1,2-benzisoxazole-3-carboxamide

N-[4-cyano-2-(5-oxo-4,5-dihydro-1,2,4-oxadiazol-3-yl)phenyl]-6-[(phenylsulfonyl)amino]-1,2-benzisoxazole-3-carboxamide

N-[4-cyano-2-(5-oxo-4,5-dihydro-1,2,4-oxadiazol-3-yl)phenyl]-7-(cyclohexylmethoxy)-1-methyl-1H-indole-2-carboxamide

N-[4-cyano-2-(5-oxo-4,5-dihydro-1,2,4-oxadiazol-3-yl)phenyl]-7-(cyclopropylmethoxy)-1-methyl-1H-indole-2-carboxamide

N-[4-cyano-2-(5-oxo-4,5-dihydro-1,2,4-oxadiazol-3-yl)phenyl]-1-methyl-7-(tetrahydro-2H-pyran-2-ylmethoxy)-1H-indole-2-carboxamide

N-[4-cyano-2-(5-oxo-4,5-dihydro-1,2,4-oxadiazol-3-yl)phenyl]-1-methyl-7-(pentyloxy)-1H-indole-2-carboxamide

N-[4-cyano-2-(5-oxo-4,5-dihydro-1,2,4-oxadiazol-3-yl)phenyl]-5-phenyl-1,2-benzisoxazole-3-carboxamide

N-[4-cyano-2-(5-oxo-4,5-dihydro-1,2,4-oxadiazol-3-yl)phenyl]-5-[2-(trifluoromethyl)phenyl]-1,2-benzisoxazole-3-carboxamide

N-[4-cyano-2-(5-oxo-4,5-dihydro-1,2,4-oxadiazol-3-yl)phenyl]-7-(2-methoxyethoxy)-1-methyl-1H-indole-2-carboxamide

N-[4-cyano-2-(5-oxo-4,5-dihydro-1,2,4-oxadiazol-3-yl)phenyl]-7-(2-hydroxy-3-isopropoxypropoxy)-1-methyl-1H-indole-2-carboxamide

N-[4-cyano-2-(5-oxo-4,5-dihydro-1,2,4-oxadiazol-3-yl)phenyl]-6-phenyl-1,2-benzisoxazole-3-carboxamide

N-[4-cyano-2-(5-oxo-4,5-dihydro-1,2,4-oxadiazol-3-yl)phenyl]-6-[2-(trifluoromethyl)phenyl]-1,2-benzisoxazole-3-carboxamide

N-[4-cyano-2-(5-oxo-4,5-dihydro-1,2,4-oxadiazol-3-yl)phenyl]-1-methyl-7-[2-(methylthio)ethoxy]-1H-indole-2-carboxamide

7-[(4-azido-3-iodobenzoyl)amino]-N-[4-cyano-2-(5-oxo-4,5-dihydro-1,2,4-oxadiazol-3-yl)phenyl]-1-methyl-1H-indole-2-carboxamide

5-[(4-azido-3-iodobenzoyl)amino]-N-[4-cyano-2-(5-oxo-4,5-dihydro-1,2,4-oxadiazol-3-yl)phenyl]-1,2-benzisoxazole-3-carboxamide

N-[4-cyano-2-(5-oxo-4,5-dihydro-1,2,4-oxadiazol-3-yl)phenyl]-6-[(methylsulfonyl)amino]-1,2-benzisoxazole-3-carboxamide

N-[4-cyano-2-(5-oxo-4,5-dihydro-1,2,4-oxadiazol-3-yl)phenyl]-6-{[(4-fluorophenyl)sulfonyl]amino}-1,2-benzisoxazole-3-carboxamide

6-amino-N-[4-cyano-2-(5-oxo-4,5-dihydro-1,2,4-oxadiazol-3-yl)phenyl]-1,2-benzisoxazole-3-carboxamide

N-[4-cyano-2-(5-oxo-4,5-dihydro-1,2,4-oxadiazol-3-yl)phenyl]-5-(2-fluorophenyl)-1,2-benzisoxazole-3-carboxamide

N-[4-cyano-2-(5-oxo-4,5-dihydro-1,2,4-oxadiazol-3-yl)phenyl]-1-methyl-6-[2-(trifluoromethyl)phenyl]-1H-indole-2-carboxamide

N-[4-cyano-2-(5-oxo-4,5-dihydro-1,2,4-oxadiazol-3-yl)phenyl]-1-methyl-6-(2,3,4-trimethoxyphenyl)-1H-indole-2-carboxamide

N-[4-cyano-2-(5-oxo-4,5-dihydro-1,2,4-oxadiazol-3-yl)phenyl]-1-methyl-7-(pyridin-3-ylmethoxy)-1H-indole-2-carboxamide

N-[4-cyano-2-(5-oxo-4,5-dihydro-1,2,4-oxadiazol-3-yl)phenyl]-6-(3,5-dimethylisoxazol-4-yl)-1-methyl-1H-indole-2-carboxamide

N-[4-cyano-2-(5-oxo-4,5-dihydro-1,2,4-oxadiazol-3-yl)phenyl]-1-methyl-6-(2-methylphenyl)-1H-indole-2-carboxamide

7-amino-4,6-dibromo-N-[4-cyano-2-(5-oxo-4,5-dihydro-1,2,4-oxadiazol-3-yl)phenyl]-1-methyl-1H-indole-2-carboxamide

4,6-dibromo-N-[4-cyano-2-(5-oxo-4,5-dihydro-1,2,4-oxadiazol-3-yl)phenyl]-1-methyl-7-[(phenylacetyl)amino]-1H-indole-2-carboxamide

3-({[4-cyano-2-(5-oxo-4,5-dihydro-1,2,4-oxadiazol-3-yl)phenyl]amino}carbonyl)-1,2-benzisoxazol-6-yl acetate

N-[4-cyano-2-(5-oxo-4,5-dihydro-1,2,4-oxadiazol-3-yl)phenyl]-6-hydroxy-1,2-benzisoxazole-3-carboxamide

6-(benzyloxy)-N-[4-cyano-2-(5-oxo-4,5-dihydro-1,2,4-oxadiazol-3-yl)phenyl]-1,2-benzisoxazole-3-carboxamide

N-[4-cyano-2-(5-oxo-4,5-dihydro-1,2,4-oxadiazol-3-yl)phenyl]-6-methoxy-1,2-benzisoxazole-3-carboxamide

3-({[4-cyano-2-(5-oxo-4,5-dihydro-1,2,4-oxadiazol-3-yl)phenyl]amino}carbonyl)-1,2-benzisoxazol-6-yl phenylmethanesulfonate

3-({[4-cyano-2-(5-oxo-4,5-dihydro-1,2,4-oxadiazol-3-yl)phenyl]amino}carbonyl)-1,2-benzisoxazol-6-yl benzenesulfonate

7-amino-4-bromo-N-[4-cyano-2-(5-oxo-4,5-dihydro-1,2,4-oxadiazol-3-yl)phenyl]-1-methyl-1H-indole-2-carboxamide

7-(benzyloxy)-3-bromo-N-[4-cyano-2-(5-oxo-4,5-dihydro-1,2,4-oxadiazol-3-yl)phenyl]-1H-indole-2-carboxamide

2-{[2-({[4-cyano-2-(5-oxo-4,5-dihydro-1,2,4-oxadiazol-3-yl)phenyl]amino}carbonyl)-1-methyl-1H-indol-6-yl]amino}-2-oxoethyl acetate

5-(anilinosulfonyl)-N-[4-cyano-2-(5-oxo-4,5-dihydro-1,2,4-oxadiazol-3-yl)phenyl]-1,2-benzisoxazole-3-carboxamide

N-[4-cyano-2-(5-oxo-4,5-dihydro-1,2,4-oxadiazol-3-yl)phenyl]-5-[(diethylamino)sulfonyl]-1,2-benzisoxazole-3-carboxamide

7-amino-6-bromo-N-[4-cyano-2-(5-oxo-4,5-dihydro-1,2,4-oxadiazol-3-yl)phenyl]-1-methyl-1H-indole-2-carboxamide

N-[4-cyano-2-(5-oxo-4,5-dihydro-1,2,4-oxadiazol-3-yl)phenyl]-1-methyl-7-[(phenylacetyl)amino]-4,6-bis[2-(trifluoromethyl)phenyl]-1H-indole-2-carboxamide

N-[4-cyano-2-(5-oxo-4,5-dihydro-1,2,4-oxadiazol-3-yl)phenyl]-5-nitro-6-[4-nitro-2-(trifluoromethyl)phenyl]-1,2-benzisoxazole-3-carboxamide

7-(benzyloxy)-3-bromo-N-[4-cyano-2-(5-oxo-4,5-dihydro-1,2,4-oxadiazol-3-yl)phenyl]-1-methyl-1H-indole-2-carboxamide

4-bromo-N-[4-cyano-2-(5-oxo-4,5-dihydro-1,2,4-oxadiazol-3-yl)phenyl]-1-methyl-7-[(phenylacetyl)amino]-1H-indole-2-carboxamide

N-[4-cyano-2-(5-oxo-4,5-dihydro-1,2,4-oxadiazol-3-yl)phenyl]-1-methyl-6-[(quinolin-8-ylsulfonyl)amino]-1H-indole-2-carboxamide

N-[4-cyano-2-(5-oxo-4,5-dihydro-1,2,4-oxadiazol-3-yl)phenyl]-5-(morpholin-4-ylsulfonyl)-1,2-benzisoxazole-3-carboxamide

N-[4-cyano-2-(5-oxo-4,5-dihydro-1,2,4-oxadiazol-3-yl)phenyl]-6-(cyclobutylmethoxy)-1-methyl-1H-indole-2-carboxamide

6-(butyrylamino)-N-[4-cyano-2-(5-oxo-4,5-dihydro-1,2,4-oxadiazol-3-yl)phenyl]-1-methyl-1H-indole-2-carboxamide

N-[4-cyano-2-(5-oxo-4,5-dihydro-1,2,4-oxadiazol-3-yl)phenyl]-6-[(methoxyacetyl)amino]-1-methyl-1H-indole-2-carboxamide

N-[4-cyano-2-(5-oxo-4,5-dihydro-1,2,4-oxadiazol-3-yl)phenyl]-7-methyl-1H-indole-2-carboxamide

N-[4-cyano-2-(5-oxo-4,5-dihydro-1,2,4-oxadiazol-3-yl)phenyl]-6-oxo-5,6-dihydro-4H-pyrrolo[3,2,1-ij]quinoline-2-carboxamide

5-(acetylamino)-6-[4-(acetylamino)-2-(trifluoromethyl)phenyl-N-[4-cyano-2-(5-oxo-4,5-dihydro-1,2,4-oxadiazol-3-yl)phenyl]-1,2-benzisoxazole-3-carboxamide

N-[4-cyano-2-(5-oxo-4,5-dihydro-1,2,4-oxadiazol-3-yl)phenyl]-5-[(dimethylamino)sulfonyl]-1,2-benzisoxazole-3-carboxamide

N-[4-cyano-2-(5-oxo-4,5-dihydro-1,2,4-oxadiazol-3-yl)phenyl]-5-[(methoxyacetyl)amino]-1,2-benzisoxazole-3-carboxamide

N-[4-cyano-2-(5-oxo-4,5-dihydro-1,2,4-oxadiazol-3-yl)phenyl]-5-[(cyclobutylcarbonyl)amino]-1,2-benzisoxazole-3-carboxamide

N-[4-cyano-2-(5-oxo-4,5-dihydro-1,2,4-oxadiazol-3-yl)phenyl]-6-(2-furoylamino)-1-methyl-1H-indole-2-carboxamide

N-[4-cyano-2-(5-oxo-4,5-dihydro-1,2,4-oxadiazol-3-yl)phenyl]-1-methyl-6-({[methyl(phenyl)amino]carbonyl}amino)-1H-indole-2-carboxamide

6-(acetylamino)-N-[4-cyano-2-(5-oxo-4,5-dihydro-1,2,4-oxadiazol-3-yl)phenyl]-1-methyl-1H-indole-2-carboxamide

N-[4-cyano-2-(5-oxo-4,5-dihydro-1,2,4-oxadiazol-3-yl)phenyl]-1-methyl-6-{[4-(trifluoromethoxy)benzoyl]amino}-1H-indole-2-carboxamide

N-[4-cyano-2-(5-oxo-4,5-dihydro-1,2,4-oxadiazol-3-yl)phenyl]-6-(2-fluorophenyl)-1-methyl-1H-indole-2-carboxamide

N-[4-cyano-2-(5-oxo-4,5-dihydro-1,2,4-oxadiazol-3-yl)phenyl]-6-(cyclopropylmethoxy)-1-methyl-1H-indole-2-carboxamide

7-bromo-N-[4-cyano-2-(5-oxo-4,5-dihydro-1,2,4-oxadiazol-3-yl)phenyl]-1-methyl-1H-indole-2-carboxamide

N-[4-cyano-2-(5-oxo-4,5-dihydro-1,2,4-oxadiazol-3-yl)phenyl]-6-methoxy-5,6-dihydro-4H-pyrrolo[3,2,1-ij]quinoline-2-carboxamide

N-[4-cyano-2-(5-oxo-4,5-dihydro-1,2,4-oxadiazol-3-yl)phenyl]-6-hydroxy-5,6-dihydro-4H-pyrrolo[3,2,1-ij]quinoline-2-carboxamide

N-[4-cyano-2-(5-oxo-4,5-dihydro-1,2,4-oxadiazol-3-yl)phenyl]-6-phenoxy-5,6-dihydro-4H-pyrrolo[3,2,1-ij]quinoline-2-carboxamide

N-[4-cyano-2-(5-oxo-4,5-dihydro-1,2,4-oxadiazol-3-yl)phenyl-1-methyl-7-[2-(trifluoromethyl)phenyl]-1H-indole-2-carboxamide

N-[4-cyano-2-(5-oxo-4,5-dihydro-1,2,4-oxadiazol-3-yl)phenyl]-1-methyl-7-phenyl-1H-indole-2-carboxamide

7-(4-tert-butylphenyl)-N-[4-cyano-2-(5-oxo-4,5-dihydro-1,2,4-oxadiazol-3-yl)phenyl]-1-methyl-1H-indole-2-carboxamide

N-[4-cyano-2-(5-oxo-4,5-dihydro-1,2,4-oxadiazol-3-yl)phenyl]-1-methyl-7-(5-methylthien-2-yl)-1H-indole-2-carboxamide

N-[4-cyano-2-(5-oxo-4,5-dihydro-1,2,4-oxadiazol-3-yl)phenyl]-1-methyl-6-[(pyridin-3-ylcarbonyl)amino]-1H-indole-2-carboxamide

N-[4-cyano-2-(5-oxo-4,5-dihydro-1,2,4-oxadiazol-3-yl)phenyl]-1-methyl-6-[(methylsulfonyl)amino]-1H-indole-2-carboxamide

N-[4-cyano-2-(5-oxo-4,5-dihydro-1,2,4-oxadiazol-3-yl)phenyl]-6-({[(2,5-dimethoxyphenyl)amino]carbonyl}amino)-1-methyl-1H-indole-2-carboxamide

N-[4-cyano-2-(5-oxo-4,5-dihydro-1,2,4-oxadiazol-3-yl)phenyl]-6-[(isoxazol-5-ylcarbonyl)amino]-1-methyl-1H-indole-2-carboxamide

N-[4-cyano-2-(5-oxo-4,5-dihydro-1,2,4-oxadiazol-3-yl)phenyl]-1-methyl-6-{[(pentylamino)carbonyl]amino}-1H-indole-2-carboxamide

N-[4-cyano-2-(5-oxo-4,5-dihydro-1,2,4-oxadiazol-3-yl)phenyl]-1-methyl-7-[4-(methylsulfonyl)phenyl]-1H-indole-2-carboxamide

N-[4-cyano-2-(5-oxo-4,5-dihydro-1,2,4-oxadiazol-3-yl)phenyl]-7-(2-methoxyphenyl)-1-methyl-1H-indole-2-carboxamide

N-[4-cyano-2-(5-oxo-4,5-dihydro-1,2,4-oxadiazol-3-yl)phenyl]-7-(2-fluorophenyl)-1-methyl-1H-indole-2-carboxamide

N-[4-cyano-2-(5-oxo-4,5-dihydro-1,2,4-oxadiazol-3-yl)phenyl]-1-methyl-7-(2-methylphenyl)-1H-indole-2-carboxamide

N-[4-cyano-2-(5-oxo-4,5-dihydro-1,2,4-oxadiazol-3-yl)phenyl]-6-{[(3,5-dimethylisoxazol-4-yl)sulfonyl]amino}-1-methyl-1H-indole-2-carboxamide

N-[4-cyano-2-(5-oxo-4,5-dihydro-1,2,4-oxadiazol-3-yl)phenyl]-5-(dimethylamino)-1,2-benzisoxazole-3-carboxamide

N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-5-methoxy-1H-indole-2-carboxamide

5-(benzyloxy)-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-1-methyl-1H-indole-2-carboxamide

6-(benzyloxy)-N-[4-chloro-2-(5-oxo-4,5-dihydro-1,2,4-oxadiazol-3-yl)phenyl]-1H-indole-2-carboxamide

7-chloro-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-4-methoxy-1H-indole-2-carboxamide

6-chloro-N-[4-chloro-2-(5-oxo-4,5-dihydro-1,2,4-oxadiazol-3-yl)phenyl]-1H-indole-2-carboxamide

1-benzyl-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-1-ethyl-1H-indole-2-carboxamide

N-[4-chloro-2-(5-oxo-4,5-dihydro-1,2,4-oxadiazol-3-yl)phenyl]-7-(phenylsulfonyl)-1H-indole-2-carboxamide

1-allyl-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-1-(cyclohexylmethyl)-1H-indole-2-carboxamide

N-[4-chloro-2-(5-oxo-4,5-dihydro-1,2,4-oxadiazol-3-yl)phenyl]-1-(2-methoxyethyl)-1H-indole-2-carboxamide

N-[4-chloro-2-(5-oxo-4,5-dihydro-1,2,4-oxadiazol-3-yl)phenyl]-1-pentyl-1H-indole-2-carboxamide

1-butyl-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-1-propyl-1H-indole-2-carboxamide

N-[4-chloro-2-(5-oxo-4,5-dihydro-1,2,4-oxadiazol-3-yl)phenyl]-1-isopropyl-1H-indole-2-carboxamide

N-[4-chloro-2-(5-oxo-4,5-dihydro-1,2,4-oxadiazol-3-yl)phenyl]-1-isobutyl-1H-indole-2-carboxamide

N-[4-chloro-2-(5-oxo-4,5-dihydro-1,2,4-oxadiazol-3-yl)phenyl]-1-(3-phenylpropyl)-1H-indole-2-carboxamide

N-[4-chloro-2-(5-oxo-4,5-dihydro-1,2,4-oxadiazol-3-yl)phenyl]-1-methyl-7-(phenylsulfonyl)-1H-indole-2-carboxamide

N-[4-chloro-2-(5-oxo-4,5-dihydro-1,2,4-oxadiazol-3-yl)phenyl]-7-[(phenylacetyl)amino]-1H-indole-2-carboxamide

7-(benzoylamino)-N-[4-chloro-2-(5-oxo-4,5-dihydro-1,2,4-oxadiazol-3-yl)phenyl]-1H-indole-2-carboxamide

2-{[2-({[4-chloro-2-(5-oxo-4,5-dihydro-1,2,4-oxadiazol-3-yl)phenyl]amino}carbonyl)-1H-indol-7-yl]amino}-2-oxoethyl acetate

N-[4-chloro-2-(5-oxo-4,5-dihydro-1,2,4-oxadiazol-3-yl)phenyl]-7-[(cyclopentylcarbonyl)amino]-1H-indole-2-carboxamide

7-amino-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-7-{[(6-chloropyridin-3-yl)carbonyl]amino}-1H-indole-2-carboxamide

N-[4-chloro-2-(5-oxo-4,5-dihydro-1,2,4-oxadiazol-3-yl)phenyl]-7-[(isoxazol-5-ylcarbonyl)amino]-1H-indole-2-carboxamide

N-[4-chloro-2-(5-oxo-4,5-dihydro-1,2,4-oxadiazol-3-yl)phenyl]-7-[(2,4-difluorobenzoyl)amino]-1H-indole-2-carboxamide

N-[4-chloro-2-(5-oxo-4,5-dihydro-1,2,4-oxadiazol-3-yl)phenyl]-7-[(fluoroacetyl)amino]-1H-indole-2-carboxamide

7-(acetylamino)-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-7-{[(4-chlorophenyl)acetyl]amino}-1H-indole-2-carboxamide

N-[4-chloro-2-(5-oxo-4,5-dihydro-1,2,4-oxadiazol-3-yl)phenyl]-7-{[(4-methoxyphenyl)acetyl]amino}-1H-indole-2-carboxamide

N-[4-chloro-2-(5-oxo-4,5-dihydro-1,2,4-oxadiazol-3-yl)phenyl]-7-[(cyclopentylacetyl)amino]-1H-indole-2-carboxamide

N-[4-chloro-2-(5-oxo-4,5-dihydro-1,2,4-oxadiazol-3-yl)phenyl]-7-[(3-fluorobenzoyl)amino]-1H-indole-2-carboxamide

N-[4-chloro-2-(5-oxo-4,5-dihydro-1,2,4-oxadiazol-3-yl)phenyl]-7-[(3-cyanobenzoyl)amino]-1H-indole-2-carboxamide

N-[4-chloro-2-(5-oxo-4,5-dihydro-1,2,4-oxadiazol-3-yl)phenyl]-7-[(cyclohexylcarbonyl)amino]-1H-indole-2-carboxamide

N-[4-chloro-2-(5-oxo-4,5-dihydro-1,2,4-oxadiazol-3-yl)phenyl]-7-(propionylamino)-1H-indole-2-carboxamide

methyl 5-{[2-({[4-chloro-2-(5-oxo-4,5-dihydro-1,2,4-oxadiazol-3-yl)phenyl]amino}carbonyl)-1H-indol-7-yl]amino}-5-oxopentanoate

7-(butyrylamino)-N-[4-chloro-2-(5-oxo-4,5-dihydro-1,2,4-oxadiazol-3-yl)phenyl]-1H-indole-2-carboxamide

7-[(4-bromobenzoyl)amino]-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-7-[(3-phenylpropanoyl)amino]-1H-indole-2-carboxamide

N-[4-chloro-2-(5-oxo-4,5-dihydro-1,2,4-oxadiazol-3-yl)phenyl]-7-[(phenoxyacetyl)amino]-1H-indole-2-carboxamide

N-[4-chloro-2-(5-oxo-4,5-dihydro-1,2,4-oxadiazol-3-yl)phenyl]-7-[(3-cyclopentylpropanoyl)amino]-1H-indole-2-carboxamide

methyl 3-{[2-({[4-chloro-2-(5-oxo-4,5-dihydro-1,2,4-oxadiazol-3-yl)phenyl]amino}carbonyl)-1H-indol-7-yl]amino}-3-oxopropanoate

N-[4-chloro-2-(5-oxo-4,5-dihydro-1,2,4-oxadiazol-3-yl)phenyl]-7-[(2-ethylhexanoyl)amino]-1H-indole-2-carboxamide

N-[4-chloro-2-(5-oxo-4,5-dihydro-1,2,4-oxadiazol-3-yl)phenyl]-7-{[(3,4-dimethoxyphenyl)acetyl]amino}-1H-indole-2-carboxamide

N-[4-chloro-2-(5-oxo-4,5-dihydro-1,2,4-oxadiazol-3-yl)phenyl]-7-[(3,5,5-trimethylhexanoyl)amino]-1H-indole-2-carboxamide

N-[4-chloro-2-(5-oxo-4,5-dihydro-1,2,4-oxadiazol-3-yl)phenyl]-7-[(cyclopropylcarbonyl)amino]-1H-indole-2-carboxamide

N-[4-chloro-2-(5-oxo-4,5-dihydro-1,2,4-oxadiazol-3-yl)phenyl]-7-[(methoxyacetyl)amino]-1H-indole-2-carboxamide

N-[4-chloro-2-(5-oxo-4,5-dihydro-1,2,4-oxadiazol-3-yl)phenyl]-7-[(3-methylbutanoyl)amino]-1H-indole-2-carboxamide

N-[4-chloro-2-(5-oxo-4,5-dihydro-1,2,4-oxadiazol-3-yl)phenyl]-7-(pentanoylamino)-1H-indole-2-carboxamide

N-[4-chloro-2-(5-oxo-4,5-dihydro-1,2,4-oxadiazol-3-yl)phenyl]-7-{[(4,7,7-trimethyl-3-oxo-2-oxabicyclo[2.2.1]hept-1-yl)carbonyl]amino}-1H-indole-2-carboxamide

N-[4-chloro-2-(5-oxo-4,5-dihydro-1,2,4-oxadiazol-3-yl)phenyl]-7-{[chloro(phenyl)acetyl]amino}-1H-indole-2-carboxamide

7-{[(benzyloxy)acetyl]amino}-N-[4-chloro-2-(5-oxo-4,5-dihydro-1,2,4-oxadiazol-3-yl)phenyl]-1H-indole-2-carboxamide

ethyl 3-{[2-({[4-chloro-2-(5-oxo-4,5-dihydro-1,2,4-oxadiazol-3-yl)phenyl]amino}carbonyl)-1H-indol-7-yl]amino}-3-oxopropanoate

7-[(1-adamantylcarbonyl)amino]-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-7-(hexanoylamino)-1H-indole-2-carboxamide

N-[4-chloro-2-(5-oxo-4,5-dihydro-1,2,4-oxadiazol-3-yl)phenyl]-7-{[(2-phenylcyclopropyl)carbonyl]amino}-1H-indole-2-carboxamide

N-[4-chloro-2-(5-oxo-4,5-dihydro-1,2,4-oxadiazol-3-yl)phenyl]-7-[(2-phenylbutanoyl)amino]-1H-indole-2-carboxamide

N-[4-chloro-2-(5-oxo-4,5-dihydro-1,2,4-oxadiazol-3-yl)phenyl]-7-(heptanoylamino)-1H-indole-2-carboxamide

2-{[2-({[4-chloro-2-(5-oxo-4,5-dihydro-1,2,4-oxadiazol-3-yl)phenyl]amino}carbonyl)-1H-indol-7-yl]amino}-2-oxo-1-phenylethyl acetate

N-[4-chloro-2-(5-oxo-4,5-dihydro-1,2,4-oxadiazol-3-yl)phenyl]-7-[(thien-2-ylcarbonyl)amino]-1H-indole-2-carboxamide

N-[4-chloro-2-(5-oxo-4,5-dihydro-1,2,4-oxadiazol-3-yl)phenyl]-7-[(2-methylbutanoyl)amino]-1H-indole-2-carboxamide

methyl 8-{[2-({[4-chloro-2-(5-oxo-4,5-dihydro-1,2,4-oxadiazol-3-yl)phenyl]amino}carbonyl)-1H-indol-7-yl]amino}-8-oxooctanoate

N-[4-chloro-2-(5-oxo-4,5-dihydro-1,2,4-oxadiazol-3-yl)phenyl]-7-[(2-ethylbutanoyl)amino]-1H-indole-2-carboxamide

N-[4-chloro-2-(5-oxo-4,5-dihydro-1,2,4-oxadiazol-3-yl)phenyl]v-7-(octanoylamino)-1H-indole-2-carboxamide

N-[4-chloro-2-(5-oxo-4,5-dihydro-1,2,4-oxadiazol-3-yl)phenyl]-7-[(cyclobutylcarbonyl)amino]-1H-indole-2-carboxamide

N-[4-chloro-2-(5-oxo-4,5-dihydro-1,2,4-oxadiazol-3-yl)phenyl]-7-(1,3-dioxo-1,3-dihydro-2H-isoindol-2-yl)-1H-indole-2-carboxamide

7-({[2-(benzylthio)-1,3-thiazol-4-yl]carbonyl}amino)-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-7-{[3-(morpholin-4-ylsulfonyl)benzoyl]amino}-1H-indole-2-carboxamide

N-[4-chloro-2-(5-oxo-4,5-dihydro-1,2,4-oxadiazol-3-yl)phenyl]-7-[(1H-indol-2-ylcarbonyl)amino]-1H-indole-2-carboxamide

N-[2-({[4-chloro-2-(5-oxo-4,5-dihydro-1,2,4-oxadiazol-3-yl)phenyl]amino}carbonyl)-1H-indol-7-yl]-1-methyl-1H-indole-2-carboxamide

N-[4-chloro-2-(5-oxo-4,5-dihydro-1,2,4-oxadiazol-3-yl)phenyl]-7-{[(5-phenylisoxazol-3-yl)carbonyl]amino}-1H-indole-2-carboxamide

N-[4-chloro-2-(5-oxo-4,5-dihydro-1,2,4-oxadiazol-3-yl)phenyl]-7-[(5-phenylpentanoyl)amino]-1H-indole-2-carboxamide

N-[4-chloro-2-(5-oxo-4,5-dihydro-1,2,4-oxadiazol-3-yl)phenyl]-7-[(4-phenylbutanoyl)amino]-1H-indole-2-carboxamide

N-[4-chloro-2-(5-oxo-4,5-dihydro-1,2,4-oxadiazol-3-yl)phenyl]-7-{[4-(4-methoxyphenyl)butanoyl]amino}-1H-indole-2-carboxamide

N-[4-chloro-2-(5-oxo-4,5-dihydro-1,2,4-oxadiazol-3-yl)phenyl]-7-{[(2-chlorophenyl)acetyl]amino}-1H-indole-2-carboxamide

N-[4-chloro-2-(5-oxo-4,5-dihydro-1,2,4-oxadiazol-3-yl)phenyl]-7-{[(2,4-dichlorophenyl)acetyl]amino}-1H-indole-2-carboxamide

N-[4-chloro-2-(5-oxo-4,5-dihydro-1,2,4-oxadiazol-3-yl)phenyl]-7-{[(3,4-dichlorophenyl)acetyl]amino}-1H-indole-2-carboxamide

N-[4-chloro-2-(5-oxo-4,5-dihydro-1,2,4-oxadiazol-3-yl)phenyl]-7-{[(3-chlorophenyl)acetyl]amino}-1H-indole-2-carboxamide

N-[4-chloro-2-(5-oxo-4,5-dihydro-1,2,4-oxadiazol-3-yl)phenyl]-7-({[3-(trifluoromethyl)phenyl]acetyl}amino)-1H-indole-2-carboxamide

N-[4-chloro-2-(5-oxo-4,5-dihydro-1,2,4-oxadiazol-3-yl)phenyl]-7-{[(3-methylphenyl)acetyl]amino}-1H-indole-2-carboxamide

7-{[(4-tert-butylphenyl)acetyl]amino}-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-7-{[(3-methoxyphenyl)acetyl]amino}-1H-indole-2-carboxamide

N-[4-chloro-2-(5-oxo-4,5-dihydro-1,2,4-oxadiazol-3-yl)phenyl]-7-{[(2-methoxyphenyl)acetyl]amino}-1H-indole-2-carboxamide

N-[4-chloro-2-(5-oxo-4,5-dihydro-1,2,4-oxadiazol-3-yl)phenyl]-7-{[(2-methylphenyl)acetyl]amino}-1H-indole-2-carboxamide

N-[4-chloro-2-(5-oxo-4,5-dihydro-1,2,4-oxadiazol-3-yl)phenyl]-7-({[4-(trifluoromethyl)phenyl]acetyl}amino)-1H-indole-2-carboxamide

N-[4-chloro-2-(5-oxo-4,5-dihydro-1,2,4-oxadiazol-3-yl)phenyl]-7-{[(4-isopropylphenyl)acetyl]amino}-1H-indole-2-carboxamide

N-[4-chloro-2-(5-oxo-4,5-dihydro-1,2,4-oxadiazol-3-yl)phenyl]-7-{[(4-methylphenyl)acetyl]amino}-1H-indole-2-carboxamide

N-[4-chloro-2-(5-oxo-4,5-dihydro-1,2,4-oxadiazol-3-yl)phenyl]-7-{[(4-fluorophenyl)acetyl]amino}-1H-indole-2-carboxamide

N-[4-chloro-2-(5-oxo-4,5-dihydro-1,2,4-oxadiazol-3-yl)phenyl]-7-({[2-(trifluoromethyl)phenyl]acetyl}amino)-1H-indole-2-carboxamide

N-[4-chloro-2-(5-oxo-4,5-dihydro-1,2,4-oxadiazol-3-yl)phenyl]-7-{[(3-fluorophenyl)acetyl]amino}-1H-indole-2-carboxamide

N-[4-chloro-2-(5-oxo-4,5-dihydro-1,2,4-oxadiazol-3-yl)phenyl]-7-{[(phenylthio)acetyl]amino}-1H-indole-2-carboxamide

N-[4-chloro-2-(5-oxo-4,5-dihydro-1,2,4-oxadiazol-3-yl)phenyl]-7-[(2-naphthylacetyl)amino]-1H-indole-2-carboxamide

N-[4-chloro-2-(5-oxo-4,5-dihydro-1,2,4-oxadiazol-3-yl)phenyl]-7-[(1-naphthylacetyl)amino]-1H-indole-2-carboxamide

N-[4-chloro-2-(5-oxo-4,5-dihydro-1,2,4-oxadiazol-3-yl)phenyl]-7-{[(2-naphthyloxy)acetyl]amino}-1H-indole-2-carboxamide

N-[4-chloro-2-(5-oxo-4,5-dihydro-1,2,4-oxadiazol-3-yl)phenyl]-7-[(2-propoxybenzoyl)amino]-1H-indole-2-carboxamide

N-[4-chloro-2-(5-oxo-4,5-dihydro-1,2,4-oxadiazol-3-yl)phenyl]-7-[(tetrahydrofuran-3-ylcarbonyl)amino]-1H-indole-2-carboxamide

N-[4-chloro-2-(5-oxo-4,5-dihydro-1,2,4-oxadiazol-3-yl)phenyl]-7-{[(1-methylcyclopropyl)carbonyl]amino}-1H-indole-2-carboxamide

N-[4-chloro-2-(5-oxo-4,5-dihydro-1,2,4-oxadiazol-3-yl)phenyl]-7-{[(4-ethoxyphenyl)acetyl]amino}-1H-indole-2-carboxamide

7-[(1-benzothien-3-ylacetyl)amino]-N-[4-chloro-2-(5-oxo-4,5-dihydro-1,2,4-oxadiazol-3-yl)phenyl]-1H-indole-2-carboxamide

7-[(1,1′-biphenyl-4-ylcarbonyl)amino]-N-[4-chloro-2-(5-oxo-4,5-dihydro-1,2,4-oxadiazol-3-yl)phenyl]-1H-indole-2-carboxamide

7-[(4-butoxybenzoyl)amino]-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-7-{[2-(2-phenylethyl)benzoyl]amino}-1H-indole-2-carboxamide

7-[(1,1′-biphenyl-2-ylcarbonyl)amino]-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-7-{[4-(ethylthio)benzoyl]amino}-1H-indole-2-carboxamide

N-[4-chloro-2-(5-oxo-4,5-dihydro-1,2,4-oxadiazol-3-yl)phenyl]-7-{[2-(methylsulfonyl)benzoyl]amino}-1H-indole-2-carboxamide

N-[4-chloro-2-(5-oxo-4,5-dihydro-1,2,4-oxadiazol-3-yl)phenyl]-7-{[(2,6-dichlorophenyl)acetyl]amino}-1H-indole-2-carboxamide

7-[(1,1′-biphenyl-4-ylacetyl)amino]-N-[4-chloro-2-(5-oxo-4,5-dihydro-1,2,4-oxadiazol-3-yl)phenyl]-1H-indole-2-carboxamide

7-[(1,3-benzodioxol-5-ylacetyl)amino]-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-7-[(3,3-dimethylbutanoyl)amino]-1H-indole-2-carboxamide

N-[4-chloro-2-(5-oxo-4,5-dihydro-1,2,4-oxadiazol-3-yl)phenyl]-7-[(thien-2-ylacetyl)amino]-1H-indole-2-carboxamide

N-[4-chloro-2-(5-oxo-4,5-dihydro-1,2,4-oxadiazol-3-yl)phenyl]-7-{[(3-methyl-5-phenylisoxazol-4-yl)carbonyl]amino}-1H-indole-2-carboxamide

N-[4-chloro-2-(5-oxo-4,5-dihydro-1,2,4-oxadiazol-3-yl)phenyl]-7-([2-(2-methoxyethoxy)ethoxy]acetyl}amino)-1H-indole-2-carboxamide

N-[4-chloro-2-(5-oxo-4,5-dihydro-1,2,4-oxadiazol-3-yl)phenyl]-7-[(2-hydroxybenzoyl)amino]-1H-indole-2-carboxamide

N-[4-chloro-2-(5-oxo-4,5-dihydro-1,2,4-oxadiazol-3-yl)phenyl]-7-({[4-(trifluoromethoxy)phenyl]sulfonyl}amino)-1H-indole-2-carboxamide

N-[4-chloro-2-(5-oxo-4,5-dihydro-1,2,4-oxadiazol-3-yl)phenyl]-7-(prolylamino)-1H-indole-2-carboxamide

N-[4-chloro-2-(5-oxo-4,5-dihydro-1,2,4-oxadiazol-3-yl)phenyl]-7-{[(3-methylisoxazol-5-yl)acetyl]amino}-1H-indole-2-carboxamide

N-[4-chloro-2-(5-oxo-4,5-dihydro-1,2,4-oxadiazol-3-yl)phenyl]-6-methoxy-1H-indole-2-carboxamide

N-[4-chloro-2-(5-oxo-4,5-dihydro-1,2,4-oxadiazol-3-yl)phenyl]-5-nitro-1,2-benzisoxazole-3-carboxamide

7-[(benzylsulfonyl)amino]-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-1-methyl-7-{[3-(morpholin-4-ylsulfonyl)benzoyl]amino}-1H-indole-2-carboxamide

N-[4-chloro-2-(5-oxo-4,5-dihydro-1,2,4-oxadiazol-3-yl)phenyl]-7-{[(4-fluorophenyl)acetyl]amino}-1-methyl-1H-indole-2-carboxamide

N-[4-chloro-2-(5-oxo-4,5-dihydro-1,2,4-oxadiazol-3-yl)phenyl]-7-[(fluoroacetyl)amino]-1-methyl-1H-indole-2-carboxamide

N-[4-chloro-2-(5-oxo-4,5-dihydro-1,2,4-oxadiazol-3-yl)phenyl]-1-methyl-7-{[(1-methyl-1H-indol-2-yl)carbonyl]amino}-1H-indole-2-carboxamide

N-[4-chloro-2-(5-oxo-4,5-dihydro-1,2,4-oxadiazol-3-yl)phenyl]-5-[(phenylacetyl)amino]-1,2-benzisoxazole-3-carboxamide

5-[(benzylsulfonyl)amino]-N-[4-chloro-2-(5-oxo-4,5-dihydro-1,2,4-oxadiazol-3-yl)phenyl]-1,2-benzisoxazole-3-carboxamide

6-(benzyloxy)-N-[4-chloro-2-(5-oxo-4,5-dihydro-1,2,4-oxadiazol-3-yl)phenyl]-1-methyl-1H-indole-2-carboxamide

N-[4-chloro-2-(5-oxo-4,5-dihydro-1,2,4-oxadiazol-3-yl)phenyl]-6-methoxy-1-methyl-1H-indole-2-carboxamide

N-[4-chloro-2-(5-oxo-4,5-dihydro-1,2,4-oxadiazol-3-yl)phenyl]-1-methyl-7-[(morpholin-4-ylcarbonyl)amino]-1H-indole-2-carboxamide

N-[4-chloro-2-(5-oxo-4,5-dihydro-1,2,4-oxadiazol-3-yl)phenyl]-1-methyl-7-({[(tetrahydrofuran-2-ylmethyl)amino]carbonyl}amino)-1H-indole-2-carboxamide

N-[4-chloro-2-(5-oxo-4,5-dihydro-1,2,4-oxadiazol-3-yl)phenyl]-7-hydroxy-1-methyl-1H-indole-2-carboxamide

7-{[(benzylamino)carbonyl]amino}-N-[4-chloro-2-(5-oxo-4,5-dihydro-1,2,4-oxadiazol-3-yl)phenyl]-1-methyl-1H-indole-2-carboxamide

N-[4-chloro-2-(5-oxo-4,5-dihydro-1,2,4-oxadiazol-3-yl)phenyl]-7-({[(2,3-dihydroxypropyl)amino]carbonyl}amino)-1-methyl-1H-indole-2-carboxamide

1-[({[2-({[4-chloro-2-(5-oxo-4,5-dihydro-1,2,4-oxadiazol-3-yl)phenyl]amino}carbonyl)-1-methyl-1H-indol-7-yl]amino}carbonyl)(methyl)amino]-1-deoxyhexitol

N-[4-chloro-2-(5-oxo-4,5-dihydro-1,2,4-oxadiazol-3-yl)phenyl]-7-(2,3-dihydro-1,4-benzodioxin-2-ylmethoxy)-1-methyl-1H-indole-2-carboxamide

7-(benzyloxy)-N-[4-chloro-2-(5-oxo-4,5-dihydro-1,2,4-oxadiazol-3-yl)phenyl]-1H-indole-2-carboxamide

N-[4-chloro-2-(5-oxo-4,5-dihydro-1,2,4-oxadiazol-3-yl)phenyl]-1-methyl-7-(3-phenoxypropoxy)-1H-indole-2-carboxamide

5-(acetylamino)-N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-5-[(methylsulfonyl)amino]-1,2-benzisoxazole-3-carboxamide

5-(benzoylamino)-N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-5-[(phenylsulfonyl)amino]-1,2-benzisoxazole-3-carboxamide

N-[4-chloro-2-(5-oxo-4,5-dihydro-1,2,4-oxadiazol-3-yl)phenyl]-7-(cyclobutylmethoxy)-1-methyl-1H-indole-2-carboxamide

N-[4-chloro-2-(5-oxo-4,5-dihydro-1,2,4-oxadiazol-3-yl)phenyl]-7-(2-furylmethoxy)-1-methyl-1H-indole-2-carboxamide

N-[4-chloro-2-(5-oxo-4,5-dihydro-1,2,4-oxadiazol-3-yl)phenyl]-7-{[(4S)-2,2-dimethyl-1,3-dioxolan-4-yl]methoxy}-1-methyl-1H-indole-2-carboxamide

N-[4-chloro-2-(5-oxo-4,5-dihydro-1,2,4-oxadiazol-3-yl)phenyl]-7-{[(2R)-2,3-dihydroxypropyl]oxy}-1-methyl-1H-indole-2-carboxamide

N-[4-chloro-2-(5-oxo-4,5-dihydro-1,2,4-oxadiazol-3-yl)phenyl]-7-(cyclobutyloxy)-1-methyl-1H-indole-2-carboxamide

N-[4-chloro-2-(5-oxo-4,5-dihydro-1,2,4-oxadiazol-3-yl)phenyl]-7-(2-methoxy-1-methylethoxy)-1-methyl-1H-indole-2-carboxamide

N-[4-chloro-2-(5-oxo-4,5-dihydro-1,2,4-oxadiazol-3-yl)phenyl]-7-isopropoxy-1-methyl-1H-indole-2-carboxamide

7-(benzyloxy)-N-[4-chloro-2-(5-oxo-4,5-dihydro-1,2,4-oxadiazol-3-yl)phenyl]-1-methyl-1H-indole-2-carboxamide

6-chloro-N-[4-chloro-2-(5-oxo-4,5-dihydro-1,2,4-oxadiazol-3-yl)phenyl]-1,2-benzisoxazole-3-carboxamide

6-sec-butoxy-N-[4-chloro-2-(5-oxo-4,5-dihydro-1,2,4-oxadiazol-3-yl)phenyl]-1-methyl-1H-indole-2-carboxamide

6-butoxy-N-[4-chloro-2-(5-oxo-4,5-dihydro-1,2,4-oxadiazol-3-yl)phenyl]-1-methyl-1H-indole-2-carboxamide

N-[4-chloro-2-(5-oxo-4,5-dihydro-1,2,4-oxadiazol-3-yl)phenyl]-5-{[(4-fluorophenyl)sulfonyl]amino}-1,2-benzisoxazole-3-carboxamide

5-amino-N-[4-chloro-2-(5-oxo-4,5-dihydro-1,2,4-oxadiazol-3-yl)phenyl]-1,2-benzisoxazole-3-carboxamide

5-bromo-N-[4-chloro-2-(5-oxo-4,5-dihydro-1,2,4-oxadiazol-3-yl)phenyl]-1,2-benzisoxazole-3-carboxamide

6-[(benzylsulfonyl)amino]-N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-6-[(phenylsulfonyl)amino]-1,2-benzisoxazole-3-carboxamide

N-[4-chloro-2-(5-oxo-4,5-dihydro-1,2,4-oxadiazol-3-yl)phenyl]-7-(cyclohexylmethoxy)-1-methyl-1H-indole-2-carboxamide

N-[4-chloro-2-(5-oxo-4,5-dihydro-1,2,4-oxadiazol-3-yl)phenyl]-7-(cyclopropylmethoxy)-1-methyl-1H-indole-2-carboxamide

N-[4-chloro-2-(5-oxo-4,5-dihydro-1,2,4-oxadiazol-3-yl)phenyl]-1-methyl-7-(tetrahydro-2H-pyran-2-ylmethoxy)-1H-indole-2-carboxamide

N-[4-chloro-2-(5-oxo-4,5-dihydro-1,2,4-oxadiazol-3-yl)phenyl]-1-methyl-7-(pentyloxy)-1H-indole-2-carboxamide

N-[4-chloro-2-(5-oxo-4,5-dihydro-1,2,4-oxadiazol-3-yl)phenyl]-5-phenyl-1,2-benzisoxazole-3-carboxamide

N-[4-chloro-2-(5-oxo-4,5-dihydro-1,2,4-oxadiazol-3-yl)phenyl]-5-[2-(trifluoromethyl)phenyl]-1,2-benzisoxazole-3-carboxamide

N-[4-chloro-2-(5-oxo-4,5-dihydro-1,2,4-oxadiazol-3-yl)phenyl]-7-(2-methoxyethoxy)-1-methyl-1H-indole-2-carboxamide

N-[4-chloro-2-(5-oxo-4,5-dihydro-1,2,4-oxadiazol-3-yl)phenyl]-7-(2-hydroxy-3-isopropoxypropoxy)-1-methyl-1H-indole-2-carboxamide

N-[4-chloro-2-(5-oxo-4,5-dihydro-1,2,4-oxadiazol-3-yl)phenyl]-6-phenyl-1,2-benzisoxazole-3-carboxamide

N-[4-chloro-2-(5-oxo-4,5-dihydro-1,2,4-oxadiazol-3-yl)phenyl]-6-[2-(trifluoromethyl)phenyl]-1,2-benzisoxazole-3-carboxamide

N-[4-chloro-2-(5-oxo-4,5-dihydro-1,2,4-oxadiazol-3-yl)phenyl]-1-methyl-7-[2-(methylthio)ethoxy]-1H-indole-2-carboxamide

7-[(4-azido-3-iodobenzoyl)amino]-N-[4-chloro-2-(5-oxo-4,5-dihydro-1,2,4-oxadiazol-3-yl)phenyl]-1-methyl-1H-indole-2-carboxamide

5-[(4-azido-3-iodobenzoyl)amino]-N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-6-[(methylsulfonyl)amino]-1,2-benzisoxazole-3-carboxamide

N-[4-chloro-2-(5-oxo-4,5-dihydro-1,2,4-oxadiazol-3-yl)phenyl]-6-{[(4-fluorophenyl)sulfonyl]amino}-1,2-benzisoxazole-3-carboxamide

6-amino-N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-5-(2-fluorophenyl)-1,2-benzisoxazole-3-carboxamide

N-[4-chloro-2-(5-oxo-4,5-dihydro-1,2,4-oxadiazol-3-yl)phenyl]-1-methyl-6-[2-(trifluoromethyl)phenyl]-1H-indole-2-carboxamide

N-[4-chloro-2-(5-oxo-4,5-dihydro-1,2,4-oxadiazol-3-yl)phenyl]-1-methyl-6-(2,3,4-trimethoxyphenyl)-1H-indole-2-carboxamide

N-[4-chloro-2-(5-oxo-4,5-dihydro-1,2,4-oxadiazol-3-yl)phenyl]-1-methyl-7-(pyridin-3-ylmethoxy)-1H-indole-2-carboxamide

N-[4-chloro-2-(5-oxo-4,5-dihydro-1,2,4-oxadiazol-3-yl)phenyl]-6-(3,5-dimethylisoxazol-4-yl)-1-methyl-1H-indole-2-carboxamide

N-[4-chloro-2-(5-oxo-4,5-dihydro-1,2,4-oxadiazol-3-yl)phenyl]-1-methyl-6-(2-methylphenyl)-1H-indole-2-carboxamide

7-amino-4,6-dibromo-N-[4-chloro-2-(5-oxo-4,5-dihydro-1,2,4-oxadiazol-3-yl)phenyl]-1-methyl-1H-indole-2-carboxamide

4,6-dibromo-N-[4-chloro-2-(5-oxo-4,5-dihydro-1,2,4-oxadiazol-3-yl)phenyl]-1-methyl-7-[(phenylacetyl)amino]-1H-indole-2-carboxamide

3-({[4-chloro-2-(5-oxo-4,5-dihydro-1,2,4-oxadiazol-3-yl)phenyl]amino}carbonyl)-1,2-benzisoxazol-6-yl acetate

N-[4-chloro-2-(5-oxo-4,5-dihydro-1,2,4-oxadiazol-3-yl)phenyl]-6-hydroxy-1,2-benzisoxazole-3-carboxamide

6-(benzyloxy)-N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-6-methoxy-1,2-benzisoxazole-3-carboxamide

3-({[4-chloro-2-(5-oxo-4,5-dihydro-1,2,4-oxadiazol-3-yl)phenyl]amino}carbonyl)-1,2-benzisoxazol-6-yl phenylmethanesulfonate

3-({[4-chloro-2-(5-oxo-4,5-dihydro-1,2,4-oxadiazol-3-yl)phenyl]amino}carbonyl)-1,2-benzisoxazol-6-yl benzenesulfonate

7-amino-4-bromo-N-[4-chloro-2-(5-oxo-4,5-dihydro-1,2,4-oxadiazol-3-yl)phenyl]-1-methyl-1H-indole-2-carboxamide

7-(benzyloxy)-3-bromo-N-[4-chloro-2-(5-oxo-4,5-dihydro-1,2,4-oxadiazol-3-yl)phenyl]-1H-indole-2-carboxamide

2-{[2-({[4-chloro-2-(5-oxo-4,5-dihydro-1,2,4-oxadiazol-3-yl)phenyl]amino}carbonyl)-1-methyl-1H-indol-6-yl]amino}-2-oxoethyl acetate

5-(anilinosulfonyl)-N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-5-[(diethylamino)sulfonyl]-1,2-benzisoxazole-3-carboxamide

7-amino-6-bromo-N-[4-chloro-2-(5-oxo-4,5-dihydro-1,2,4-oxadiazol-3-yl)phenyl]-1-methyl-1H-indole-2-carboxamide

N-[4-chloro-2-(5-oxo-4,5-dihydro-1,2,4-oxadiazol-3-yl)phenyl]-1-methyl-7-[(phenylacetyl)amino]-4,6-bis[2-(trifluoromethyl)phenyl]-1H-indole-2-carboxamide

N-[4-chloro-2-(5-oxo-4,5-dihydro-1,2,4-oxadiazol-3-yl)phenyl]-5-nitro-6-[4-nitro-2-(trifluoromethyl)phenyl]-1,2-benzisoxazole-3-carboxamide

7-(benzyloxy)-3-bromo-N-[4-chloro-2-(5-oxo-4,5-dihydro-1,2,4-oxadiazol-3-yl)phenyl]-1-methyl-1H-indole-2-carboxamide

4-bromo-N-[4-chloro-2-(5-oxo-4,5-dihydro-1,2,4-oxadiazol-3-yl)phenyl]-1-methyl-7-[(phenylacetyl)amino]-1H-indole-2-carboxamide

N-[4-chloro-2-(5-oxo-4,5-dihydro-1,2,4-oxadiazol-3-yl)phenyl]-1-methyl-6-[(quinolin-8-ylsulfonyl)amino]-1H-indole-2-carboxamide

N-[4-chloro-2-(5-oxo-4,5-dihydro-1,2,4-oxadiazol-3-yl)phenyl]-5-(morpholin-4-ylsulfonyl)-1,2-benzisoxazole-3-carboxamide

N-[4-chloro-2-(5-oxo-4,5-dihydro-1,2,4-oxadiazol-3-yl)phenyl]-6-(cyclobutylmethoxy)-1-methyl-1H-indole-2-carboxamide

6-(butyrylamino)-N-[4-chloro-2-(5-oxo-4,5-dihydro-1,2,4-oxadiazol-3-yl)phenyl]-1-methyl-1H-indole-2-carboxamide

N-[4-chloro-2-(5-oxo-4,5-dihydro-1,2,4-oxadiazol-3-yl)phenyl]-6-[(methoxyacetyl)amino]-1-methyl-1H-indole-2-carboxamide

N-[4-chloro-2-(5-oxo-4,5-dihydro-1,2,4-oxadiazol-3-yl)phenyl]-7-methyl-1H-indole-2-carboxamide

N-[4-chloro-2-(5-oxo-4,5-dihydro-1,2,4-oxadiazol-3-yl)phenyl]-6-oxo-5,6-dihydro-4H-pyrrolo[3,2,1-ij]quinoline-2-carboxamide

5-(acetylamino)-6-[4-(acetylamino)-2-(trifluoromethyl)phenyl]-N-[4-chloro-2-(5-oxo-4,5-dihydro-1,2,4-oxadiazol-3-yl)phenyl]-1,2-benzisoxazole-3-carboxamide

N-[4-chloro-2-(5-oxo-4,5-dihydro-1,2,4-oxadiazol-3-yl)phenyl]-5-[(dimethylamino)sulfonyl]-1,2-benzisoxazole-3-carboxamide

N-[4-chloro-2-(5-oxo-4,5-dihydro-1,2,4-oxadiazol-3-yl)phenyl]-5-[(methoxyacetyl)amino]-1,2-benzisoxazole-3-carboxamide

N-[4-chloro-2-(5-oxo-4,5-dihydro-1,2,4-oxadiazol-3-yl)phenyl]-5-[(cyclobutylcarbonyl)amino]-1,2-benzisoxazole-3-carboxamide

N-[4-chloro-2-(5-oxo-4,5-dihydro-1,2,4-oxadiazol-3-yl)phenyl]-6-(2-furoylamino)-1-methyl-1H-indole-2-carboxamide

N-[4-chloro-2-(5-oxo-4,5-dihydro-1,2,4-oxadiazol-3-yl)phenyl]-1-methyl-6-({[methyl(phenyl)amino]carbonyl}amino)-1H-indole-2-carboxamide

6-(acetylamino)-N-[4-chloro-2-(5-oxo-4,5-dihydro-1,2,4-oxadiazol-3-yl)phenyl]-1-methyl-1H-indole-2-carboxamide

N-[4-chloro-2-(5-oxo-4,5-dihydro-1,2,4-oxadiazol-3-yl)phenyl]-1-methyl-6-{[4-(trifluoromethoxy)benzoyl]amino}-1H-indole-2-carboxamide

N-[4-chloro-2-(5-oxo-4,5-dihydro-1,2,4-oxadiazol-3-yl)phenyl]-6-(2-fluorophenyl)-1-methyl-1H-indole-2-carboxamide

N-[4-chloro-2-(5-oxo-4,5-dihydro-1,2,4-oxadiazol-3-yl)phenyl]-6-(cyclopropylmethoxy)-1-methyl-1H-indole-2-carboxamide

7-bromo-N-[4-chloro-2-(5-oxo-4,5-dihydro-1,2,4-oxadiazol-3-yl)phenyl]-1-methyl-1H-indole-2-carboxamide

N-[4-chloro-2-(5-oxo-4,5-dihydro-1,2,4-oxadiazol-3-yl)phenyl]-6-methoxy-5,6-dihydro-4H-pyrrolo[3,2,1-ij]quinoline-2-carboxamide

N-[4-chloro-2-(5-oxo-4,5-dihydro-1,2,4-oxadiazol-3-yl)phenyl]-6-hydroxy-5,6-dihydro-4H-pyrrolo[3,2,1-ij]quinoline-2-carboxamide

N-[4-chloro-2-(5-oxo-4,5-dihydro-1,2,4-oxadiazol-3-yl)phenyl]-6-phenoxy-5,6-dihydro-4H-pyrrolo[3,2,1-ij]quinoline-2-carboxamide

N-[4-chloro-2-(5-oxo-4,5-dihydro-1,2,4-oxadiazol-3-yl)phenyl]-1-methyl-7-[2-(trifluoromethyl)phenyl]-1H-indole-2-carboxamide

N-[4-chloro-2-(5-oxo-4,5-dihydro-1,2,4-oxadiazol-3-yl)phenyl]-1-methyl-7-phenyl-1H-indole-2-carboxamide

7-(4-tert-butylphenyl)-N-[4-chloro-2-(5-oxo-4,5-dihydro-1,2,4-oxadiazol-3-yl)phenyl]-1-methyl-1H-indole-2-carboxamide

N-[4-chloro-2-(5-oxo-4,5-dihydro-1,2,4-oxadiazol-3-yl)phenyl]-1-methyl-7-(5-methylthien-2-yl)-1H-indole-2-carboxamide

N-[4-chloro-2-(5-oxo-4,5-dihydro-1,2,4-oxadiazol-3-yl)phenyl]-1-methyl-6-[(pyridin-3-ylcarbonyl)amino]-1H-indole-2-carboxamide

N-[4-chloro-2-(5-oxo-4,5-dihydro-1,2,4-oxadiazol-3-yl)phenyl]-1-methyl-6-[(methylsulfonyl)amino]-1H-indole-2-carboxamide

N-[4-chloro-2-(5-oxo-4,5-dihydro-1,2,4-oxadiazol-3-yl)phenyl]-6-(f [(2,5-dimethoxyphenyl)amino]carbonyl}amino)-1-methyl-1H-indole-2-carboxamide

N-[4-chloro-2-(5-oxo-4,5-dihydro-1,2,4-oxadiazol-3-yl)phenyl]-6-[(isoxazol-5-ylcarbonyl)amino]-1-methyl-1H-indole-2-carboxamide

N-[4-chloro-2-(5-oxo-4,5-dihydro-1,2,4-oxadiazol-3-yl)phenyl]-1-methyl-6-{[(pentylamino)carbonyl]amino}-1H-indole-2-carboxamide

N-[4-chloro-2-(5-oxo-4,5-dihydro-1,2,4-oxadiazol-3-yl)phenyl]-1-methyl-7-[4-(methylsulfonyl)phenyl]-1H-indole-2-carboxamide

N-[4-chloro-2-(5-oxo-4,5-dihydro-1,2,4-oxadiazol-3-yl)phenyl]-7-(2-methoxyphenyl)-1-methyl-1H-indole-2-carboxamide

N-[4-chloro-2-(5-oxo-4,5-dihydro-1,2,4-oxadiazol-3-yl)phenyl]-7-(2-fluorophenyl)-1-methyl-1H-indole-2-carboxamide

N-[4-chloro-2-(5-oxo-4,5-dihydro-1,2,4-oxadiazol-3-yl)phenyl]-1-methyl-7-(2-methylphenyl)-1H-indole-2-carboxamide

N-[4-chloro-2-(5-oxo-4,5-dihydro-1,2,4-oxadiazol-3-yl)phenyl]-6-{[(3,5-dimethylisoxazol-4-yl)sulfonyl]amino}-1-methyl-1H-indole-2-carboxamide

N-[4-chloro-2-(5-oxo-4,5-dihydro-1,2,4-oxadiazol-3-yl)phenyl]-5-(dimethylamino)-1,2-benzisoxazole-3-carboxamide

N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-5-methoxy-1H-indole-2-carboxamide

5-(benzyloxy)-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-1-methyl-1H-indole-2-carboxamide

6-(benzyloxy)-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-7-chloro-1H-indole-2-carboxamide

N-[4-bromo-2-(5-oxo-4,5-dihydro-1,2,4-oxadiazol-3-yl)phenyl]-4-methoxy-1H-indole-2-carboxamide

N-[4-bromo-2-(5-oxo-4,5-dihydro-1,2,4-oxadiazol-3-yl)phenyl]-6-chloro-1H-indole-2-carboxamide

1-benzyl-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-1-ethyl-1H-indole-2-carboxamide

N-[4-bromo-2-(5-oxo-4,5-dihydro-1,2,4-oxadiazol-3-yl)phenyl]-7-(phenylsulfonyl)-1H-indole-2-carboxamide

1-allyl-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-1-(cyclohexylmethyl)-1H-indole-2-carboxamide

N-[4-bromo-2-(5-oxo-4,5-dihydro-1,2,4-oxadiazol-3-yl)phenyl]-1-(2-methoxyethyl)-1H-indole-2-carboxamide

N-[4-bromo-2-(5-oxo-4,5-dihydro-1,2,4-oxadiazol-3-yl)phenyl]-1-pentyl-1H-indole-2-carboxamide

N-[4-bromo-2-(5-oxo-4,5-dihydro-1,2,4-oxadiazol-3-yl)phenyl]-1-butyl-1H-indole-2-carboxamide

N-[4-bromo-2-(5-oxo-4,5-dihydro-1,2,4-oxadiazol-3-yl)phenyl]-1-propyl-1H-indole-2-carboxamide

N-[4-bromo-2-(5-oxo-4,5-dihydro-1,2,4-oxadiazol-3-yl)phenyl]-1-isopropyl-1H-indole-2-carboxamide

N-[4-bromo-2-(5-oxo-4,5-dihydro-1,2,4-oxadiazol-3-yl)phenyl]-1-isobutyl-1H-indole-2-carboxamide

N-[4-bromo-2-(5-oxo-4,5-dihydro-1,2,4-oxadiazol-3-yl)phenyl]-1-(3-phenylpropyl)-1H-indole-2-carboxamide

N-[4-bromo-2-(5-oxo-4,5-dihydro-1,2,4-oxadiazol-3-yl)phenyl]-1-methyl-7-(phenylsulfonyl)-1H-indole-2-carboxamide

N-[4-bromo-2-(5-oxo-4,5-dihydro-1,2,4-oxadiazol-3-yl)phenyl]-7-[(phenylacetyl)amino]-1H-indole-2-carboxamide

7-(benzoylamino)-N-[4-bromo-2-(5-oxo-4,5-dihydro-1,2,4-oxadiazol-3-yl)phenyl]-1H-indole-2-carboxamide

2-{[2-({[4-bromo-2-(5-oxo-4,5-dihydro-1,2,4-oxadiazol-3-yl)phenyl]amino}carbonyl)-1H-indol-7-yl]amino}-2-oxoethyl acetate

N-[4-bromo-2-(5-oxo-4,5-dihydro-1,2,4-oxadiazol-3-yl)phenyl]-7-[(cyclopentylcarbonyl)amino]-1H-indole-2-carboxamide

7-amino-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-7-{[(6-chloropyridin-3-yl)carbonyl]amino}-1H-indole-2-carboxamide

N-[4-bromo-2-(5-oxo-4,5-dihydro-1,2,4-oxadiazol-3-yl)phenyl]-7-[(isoxazol-5-ylcarbonyl)amino]-1H-indole-2-carboxamide

N-[4-bromo-2-(5-oxo-4,5-dihydro-1,2,4-oxadiazol-3-yl)phenyl]-7-[(2,4-difluorobenzoyl)amino]-1H-indole-2-carboxamide

N-[4-bromo-2-(5-oxo-4,5-dihydro-1,2,4-oxadiazol-3-yl)phenyl]-7-[(fluoroacetyl)amino]-1H-indole-2-carboxamide

7-(acetylamino)-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-7-{[(4-chlorophenyl)acetyl]amino}-1H-indole-2-carboxamide

N-[4-bromo-2-(5-oxo-4,5-dihydro-1,2,4-oxadiazol-3-yl)phenyl]-7-{[(4-methoxyphenyl)acetyl]amino}-1H-indole-2-carboxamide

N-[4-bromo-2-(5-oxo-4,5-dihydro-1,2,4-oxadiazol-3-yl)phenyl]-7-[(cyclopentylacetyl)amino]-1H-indole-2-carboxamide

N-[4-bromo-2-(5-oxo-4,5-dihydro-1,2,4-oxadiazol-3-yl)phenyl]-7-[(3-fluorobenzoyl)amino]-1H-indole-2-carboxamide

N-[4-bromo-2-(5-oxo-4,5-dihydro-1,2,4-oxadiazol-3-yl)phenyl]-7-[(3-cyanobenzoyl)amino]-1H-indole-2-carboxamide

N-[4-bromo-2-(5-oxo-4,5-dihydro-1,2,4-oxadiazol-3-yl)phenyl]-7-[(cyclohexylcarbonyl)amino]-1H-indole-2-carboxamide

N-[4-bromo-2-(5-oxo-4,5-dihydro-1,2,4-oxadiazol-3-yl)phenyl]-7-(propionylamino)-1H-indole-2-carboxamide

methyl 5-{[2-({[4-bromo-2-(5-oxo-4,5-dihydro-1,2,4-oxadiazol-3-yl)phenyl]amino}carbonyl)-1H-indol-7-yl]amino}-5-oxopentanoate

N-[4-bromo-2-(5-oxo-4,5-dihydro-1,2,4-oxadiazol-3-yl)phenyl]-7-(butyrylamino)-1H-indole-2-carboxamide

7-[(4-bromobenzoyl)amino]-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-7-[(3-phenylpropanoyl)amino]-1H-indole-2-carboxamide

N-[4-bromo-2-(5-oxo-4,5-dihydro-1,2,4-oxadiazol-3-yl)phenyl]-7-[(phenoxyacetyl)amino]-1H-indole-2-carboxamide

N-[4-bromo-2-(5-oxo-4,5-dihydro-1,2,4-oxadiazol-3-yl)phenyl]-7-[(3-cyclopentylpropanoyl)amino]-1H-indole-2-carboxamide

methyl 3-{[2-({[4-bromo-2-(5-oxo-4,5-dihydro-1,2,4-oxadiazol-3-yl)phenyl]amino}carbonyl)-1H-indol-7-yl]amino}-3-oxopropanoate

N-[4-bromo-2-(5-oxo-4,5-dihydro-1,2,4-oxadiazol-3-yl)phenyl]-7-[(2-ethylhexanoyl)amino]-1H-indole-2-carboxamide

N-[4-bromo-2-(5-oxo-4,5-dihydro-1,2,4-oxadiazol-3-yl)phenyl]-7-{[(3,4-dimethoxyphenyl)acetyl]amino}-1H-indole-2-carboxamide

N-[4-bromo-2-(5-oxo-4,5-dihydro-1,2,4-oxadiazol-3-yl)phenyl]-7-[(3,5,5-trimethylhexanoyl)amino]-1H-indole-2-carboxamide

N-[4-bromo-2-(5-oxo-4,5-dihydro-1,2,4-oxadiazol-3-yl)phenyl]-7-[(cyclopropylcarbonyl)amino]-1H-indole-2-carboxamide

N-[4-bromo-2-(5-oxo-4,5-dihydro-1,2,4-oxadiazol-3-yl)phenyl]-7-[(methoxyacetyl)amino]-1H-indole-2-carboxamide

N-[4-bromo-2-(5-oxo-4,5-dihydro-1,2,4-oxadiazol-3-yl)phenyl]-7-[(3-methylbutanoyl)amino]-1H-indole-2-carboxamide

N-[4-bromo-2-(5-oxo-4,5-dihydro-1,2,4-oxadiazol-3-yl)phenyl]-7-(pentanoylamino)-1H-indole-2-carboxamide

N-[4-bromo-2-(5-oxo-4,5-dihydro-1,2,4-oxadiazol-3-yl)phenyl]-7-{[(4,7,7-trimethyl-3-oxo-2-oxabicyclo[2.2.1]hept-1-yl)carbonyl]amino}-1H-indole-2-carboxamide

N-[4-bromo-2-(5-oxo-4,5-dihydro-1,2,4-oxadiazol-3-yl)phenyl]-7-{[chloro(phenyl)acetyl]amino}-1H-indole-2-carboxamide

7-{[(benzyloxy)acetyl]amino}-N-[4-bromo-2-(5-oxo-4,5-dihydro-1,2,4-oxadiazol-3-yl)phenyl]-1H-indole-2-carboxamide

ethyl 3-{[2-({[4-bromo-2-(5-oxo-4,5-dihydro-1,2,4-oxadiazol-3-yl)phenyl]amino}carbonyl)-1H-indol-7-yl]amino}-3-oxopropanoate

7-[(1-adamantylcarbonyl)amino]-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-7-(hexanoylamino)-1H-indole-2-carboxamide

N-[4-bromo-2-(5-oxo-4,5-dihydro-1,2,4-oxadiazol-3-yl)phenyl]-7-{[(2-phenylcyclopropyl)carbonyl]amino}-1H-indole-2-carboxamide

N-[4-bromo-2-(5-oxo-4,5-dihydro-1,2,4-oxadiazol-3-yl)phenyl]-7-[(2-phenylbutanoyl)amino]-1H-indole-2-carboxamide

N-[4-bromo-2-(5-oxo-4,5-dihydro-1,2,4-oxadiazol-3-yl)phenyl]-7-(heptanoylamino)-1H-indole-2-carboxamide

2-{[2-({[4-bromo-2-(5-oxo-4,5-dihydro-1,2,4-oxadiazol-3-yl)phenyl]amino}carbonyl)-1H-indol-7-yl]amino}-2-oxo-1-phenylethyl acetate

N-[4-bromo-2-(5-oxo-4,5-dihydro-1,2,4-oxadiazol-3-yl)phenyl]-7-[(thien-2-ylcarbonyl)amino]-1H-indole-2-carboxamide

N-[4-bromo-2-(5-oxo-4,5-dihydro-1,2,4-oxadiazol-3-yl)phenyl]-7-[(2-methylbutanoyl)amino]-1H-indole-2-carboxamide

methyl 8-{[2-({[4-bromo-2-(5-oxo-4,5-dihydro-1,2,4-oxadiazol-3-yl)phenyl]amino}carbonyl)-1H-indol-7-yl]amino}-8-oxooctanoate

N-[4-bromo-2-(5-oxo-4,5-dihydro-1,2,4-oxadiazol-3-yl)phenyl]-7-[(2-ethylbutanoyl)amino]-1H-indole-2-carboxamide

N-[4-bromo-2-(5-oxo-4,5-dihydro-1,2,4-oxadiazol-3-yl)phenyl]-7-(octanoylamino)-1H-indole-2-carboxamide

N-[4-bromo-2-(5-oxo-4,5-dihydro-1,2,4-oxadiazol-3-yl)phenyl]-7-[(cyclobutylcarbonyl)amino]-1H-indole-2-carboxamide

N-[4-bromo-2-(5-oxo-4,5-dihydro-1,2,4-oxadiazol-3-yl)phenyl]-7-(1,3-dioxo-1,3-dihydro-2H-isoindol-2-yl)-1H-indole-2-carboxamide

7-({[2-(benzylthio)-1,3-thiazol-4-yl]carbonyl}amino)-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-7-[3-(morpholin-4-ylsulfonyl)benzoyl]amino}-1H-indole-2-carboxamide

N-[4-bromo-2-(5-oxo-4,5-dihydro-1,2,4-oxadiazol-3-yl)phenyl]-7-[(1H-indol-2-ylcarbonyl)amino]-1H-indole-2-carboxamide

N-[2-({[4-bromo-2-(5-oxo-4,5-dihydro-1,2,4-oxadiazol-3-yl)phenyl]amino}carbonyl)-1H-indol-7-yl]-1-methyl-1H-indole-2-carboxamide

N-[4-bromo-2-(5-oxo-4,5-dihydro-1,2,4-oxadiazol-3-yl)phenyl]-7-{[(5-phenylisoxazol-3-yl)carbonyl]amino}-1H-indole-2-carboxamide

N-[4-bromo-2-(5-oxo-4,5-dihydro-1,2,4-oxadiazol-3-yl)phenyl]-7-[(5-phenylpentanoyl)amino]-1H-indole-2-carboxamide

N-[4-bromo-2-(5-oxo-4,5-dihydro-1,2,4-oxadiazol-3-yl)phenyl]-7-[(4-phenylbutanoyl)amino]-1H-indole-2-carboxamide

N-[4-bromo-2-(5-oxo-4,5-dihydro-1,2,4-oxadiazol-3-yl)phenyl]-7-{[4-(4-methoxyphenyl)butanoyl]amino}-1H-indole-2-carboxamide

N-[4-bromo-2-(5-oxo-4,5-dihydro-1,2,4-oxadiazol-3-yl)phenyl]-7-{[(2-chlorophenyl)acetyl]amino}-1H-indole-2-carboxamide

N-[4-bromo-2-(5-oxo-4,5-dihydro-1,2,4-oxadiazol-3-yl)phenyl]-7-{[(2,4-dichlorophenyl)acetyl]amino}-1H-indole-2-carboxamide

N-[4-bromo-2-(5-oxo-4,5-dihydro-1,2,4-oxadiazol-3-yl)phenyl]-7-{[(3,4-dichlorophenyl)acetyl]amino}-1H-indole-2-carboxamide

N-[4-bromo-2-(5-oxo-4,5-dihydro-1,2,4-oxadiazol-3-yl)phenyl]-7-{[(3-chlorophenyl)acetyl]amino}-1H-indole-2-carboxamide

N-[4-bromo-2-(5-oxo-4,5-dihydro-1,2,4-oxadiazol-3-yl)phenyl]-7-({[3-(trifluoromethyl)phenyl]acetyl}amino)-1H-indole-2-carboxamide

N-[4-bromo-2-(5-oxo-4,5-dihydro-1,2,4-oxadiazol-3-yl)phenyl]-7-{[(3-methylphenyl)acetyl]amino}-1H-indole-2-carboxamide

N-[4-bromo-2-(5-oxo-4,5-dihydro-1,2,4-oxadiazol-3-yl)phenyl]-7-{[(4-tert-butylphenyl)acetyl]amino}-1H-indole-2-carboxamide

N-[4-bromo-2-(5-oxo-4,5-dihydro-1,2,4-oxadiazol-3-yl)phenyl]-7-{[(3-methoxyphenyl)acetyl]amino}-1H-indole-2-carboxamide

N-[4-bromo-2-(5-oxo-4,5-dihydro-1,2,4-oxadiazol-3-yl)phenyl]-7-{[(2-methoxyphenyl)acetyl]amino}-1H-indole-2-carboxamide

N-[4-bromo-2-(5-oxo-4,5-dihydro-1,2,4-oxadiazol-3-yl)phenyl]-7-{[(2-methylphenyl)acetyl]amino}-1H-indole-2-carboxamide

N-[4-bromo-2-(5-oxo-4,5-dihydro-1,2,4-oxadiazol-3-yl)phenyl]-7-({[4-(trifluoromethyl)phenyl]acetyl}amino)-1H-indole-2-carboxamide

N-[4-bromo-2-(5-oxo-4,5-dihydro-1,2,4-oxadiazol-3-yl)phenyl]-7-{[(4-isopropylphenyl)acetyl]amino}-1H-indole-2-carboxamide

N-[4-bromo-2-(5-oxo-4,5-dihydro-1,2,4-oxadiazol-3-yl)phenyl]-7-{[(4-methylphenyl)acetyl]amino}-1H-indole-2-carboxamide

N-[4-bromo-2-(5-oxo-4,5-dihydro-1,2,4-oxadiazol-3-yl)phenyl]-7-{[(4-fluorophenyl)acetyl]amino}-1H-indole-2-carboxamide

N-[4-bromo-2-(5-oxo-4,5-dihydro-1,2,4-oxadiazol-3-yl)phenyl]-7-({[2-(trifluoromethyl)phenyl]acetyl}amino)-1H-indole-2-carboxamide

N-[4-bromo-2-(5-oxo-4,5-dihydro-1,2,4-oxadiazol-3-yl)phenyl]-7-{[(3-fluorophenyl)acetyl]amino}-1H-indole-2-carboxamide

N-[4-bromo-2-(5-oxo-4,5-dihydro-1,2,4-oxadiazol-3-yl)phenyl]-7-{[(phenylthio)acetyl]amino}-1H-indole-2-carboxamide

N-[4-bromo-2-(5-oxo-4,5-dihydro-1,2,4-oxadiazol-3-yl)phenyl]-7-[(2-naphthylacetyl)amino]-1H-indole-2-carboxamide

N-[4-bromo-2-(5-oxo-4,5-dihydro-1,2,4-oxadiazol-3-yl)phenyl]-7-[(1-naphthylacetyl)amino]-1H-indole-2-carboxamide

N-[4-bromo-2-(5-oxo-4,5-dihydro-1,2,4-oxadiazol-3-yl)phenyl]-7-{[(2-naphthyloxy)acetyl]amino}-1H-indole-2-carboxamide

N-[4-bromo-2-(5-oxo-4,5-dihydro-1,2,4-oxadiazol-3-yl)phenyl]-7-[(2-propoxybenzoyl)amino]-1H-indole-2-carboxamide

N-[4-bromo-2-(5-oxo-4,5-dihydro-1,2,4-oxadiazol-3-yl)phenyl]-7-[(tetrahydrofuran-3-ylcarbonyl)amino]-1H-indole-2-carboxamide

N-[4-bromo-2-(5-oxo-4,5-dihydro-1,2,4-oxadiazol-3-yl)phenyl]-7-{[(1-methylcyclopropyl)carbonyl]amino}-1H-indole-2-carboxamide

N-[4-bromo-2-(5-oxo-4,5-dihydro-1,2,4-oxadiazol-3-yl)phenyl]-7-{[(4-ethoxyphenyl)acetyl]amino}-1H-indole-2-carboxamide

7-[(1-benzothien-3-ylacetyl)amino]-N-[4-bromo-2-(5-oxo-4,5-dihydro-1,2,4-oxadiazol-3-yl)phenyl]-1H-indole-2-carboxamide

7-[(1,1′-biphenyl-4-ylcarbonyl)amino]-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-7-[(4-butoxybenzoyl)amino]-1H-indole-2-carboxamide

N-[4-bromo-2-(5-oxo-4,5-dihydro-1,2,4-oxadiazol-3-yl)phenyl]-7-{[2-(2-phenylethyl)benzoyl]amino}-1H-indole-2-carboxamide

7-[(1,1′-biphenyl-2-ylcarbonyl)amino]-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-7-{[4-(ethylthio)benzoyl]amino}-1H-indole-2-carboxamide

N-[4-bromo-2-(5-oxo-4,5-dihydro-1,2,4-oxadiazol-3-yl)phenyl]-7-{[2-(methylsulfonyl)benzoyl]amino}-1H-indole-2-carboxamide

N-[4-bromo-2-(5-oxo-4,5-dihydro-1,2,4-oxadiazol-3-yl)phenyl]-7-{[(2,6-dichlorophenyl)acetyl]amino}-1H-indole-2-carboxamide

7-[(1,1′-biphenyl-4-ylacetyl)amino]-N-[4-bromo-2-(5-oxo-4,5-dihydro-1,2,4-oxadiazol-3-yl)phenyl]-1H-indole-2-carboxamide

7-[(1,3-benzodioxol-5-ylacetyl)amino]-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-7-[(3,3-dimethylbutanoyl)amino]-1H-indole-2-carboxamide

N-[4-bromo-2-(5-oxo-4,5-dihydro-1,2,4-oxadiazol-3-yl)phenyl]-7-[(thien-2-ylacetyl)amino]-1H-indole-2-carboxamide

N-[4-bromo-2-(5-oxo-4,5-dihydro-1,2,4-oxadiazol-3-yl)phenyl]-7-{[(3-methyl-5-phenylisoxazol-4-yl)carbonyl]amino}-1H-indole-2-carboxamide

N-[4-bromo-2-(5-oxo-4,5-dihydro-1,2,4-oxadiazol-3-yl)phenyl]-7-({[2-(2-methoxyethoxy)ethoxy]acetyl}amino)-1H-indole-2-carboxamide

N-[4-bromo-2-(5-oxo-4,5-dihydro-1,2,4-oxadiazol-3-yl)phenyl]-7-[(2-hydroxybenzoyl)amino]-1H-indole-2-carboxamide

N-[4-bromo-2-(5-oxo-4,5-dihydro-1,2,4-oxadiazol-3-yl)phenyl]-7-({[4-(trifluoromethoxy)phenyl]sulfonyl}amino)-1H-indole-2-carboxamide

N-[4-bromo-2-(5-oxo-4,5-dihydro-1,2,4-oxadiazol-3-yl)phenyl]-7-(prolylamino)-1H-indole-2-carboxamide

N-[4-bromo-2-(5-oxo-4,5-dihydro-1,2,4-oxadiazol-3-yl)phenyl-7-{[(3-methylisoxazol-5-yl)acetyl]amino}-1H-indole-2-carboxamide

N-[4-bromo-2-(5-oxo-4,5-dihydro-1,2,4-oxadiazol-3-yl)phenyl]-6-methoxy-1H-indole-2-carboxamide

N-[4-bromo-2-(5-oxo-4,5-dihydro-1,2,4-oxadiazol-3-yl)phenyl]-5-nitro-1,2-benzisoxazole-3-carboxamide

7-[(benzylsulfonyl)amino]-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-1-methyl-7-{[3-(morpholin-4-ylsulfonyl)benzoyl]amino}-1H-indole-2-carboxamide

N-[4-bromo-2-(5-oxo-4,5-dihydro-1,2,4-oxadiazol-3-yl)phenyl]-7-{[(4-fluorophenyl)acetyl]amino}-1-methyl-1H-indole-2-carboxamide

N-[4-bromo-2-(5-oxo-4,5-dihydro-1,2,4-oxadiazol-3-yl)phenyl]-7-[(fluoroacetyl)amino]-1-methyl-1H-indole-2-carboxamide

N-[4-bromo-2-(5-oxo-4,5-dihydro-1,2,4-oxadiazol-3-yl)phenyl]-1-methyl-7-{[(1-methyl-1H-indol-2-yl)carbonyl]amino}-1H-indole-2-carboxamide

N-[4-bromo-2-(5-oxo-4,5-dihydro-1,2,4-oxadiazol-3-yl)phenyl]-5-[(phenylacetyl)amino]-1,2-benzisoxazole-3-carboxamide

5-[(benzylsulfonyl)amino]-N-[4-bromo-2-(5-oxo-4,5-dihydro-1,2,4-oxadiazol-3-yl)phenyl]-1,2-benzisoxazole-3-carboxamide

6-(benzyloxy)-N-[4-bromo-2-(5-oxo-4,5-dihydro-1,2,4-oxadiazol-3-yl)phenyl]-1-methyl-1H-indole-2-carboxamide

N-[4-bromo-2-(5-oxo-4,5-dihydro-1,2,4-oxadiazol-3-yl)phenyl]-6-methoxy-1-methyl-1H-indole-2-carboxamide

N-[4-bromo-2-(5-oxo-4,5-dihydro-1,2,4-oxadiazol-3-yl)phenyl]-1-methyl-7-[(morpholin-4-ylcarbonyl)amino]-1H-indole-2-carboxamide

N-[4-bromo-2-(5-oxo-4,5-dihydro-1,2,4-oxadiazol-3-yl)phenyl]-1-methyl-7-({[(tetrahydrofuran-2-ylmethyl)amino]carbonyl}amino)-1H-indole-2-carboxamide

N-[4-bromo-2-(5-oxo-4,5-dihydro-1,2,4-oxadiazol-3-yl)phenyl]-7-hydroxy-1-methyl-1H-indole-2-carboxamide

7-{[(benzylamino)carbonyl]amino}-N-[4-bromo-2-(5-oxo-4,5-dihydro-1,2,4-oxadiazol-3-yl)phenyl]-1-methyl-1H-indole-2-carboxamide

N-[4-bromo-2-(5-oxo-4,5-dihydro-1,2,4-oxadiazol-3-yl)phenyl]-7-({[(2,3-dihydroxypropyl)amino]carbonyl}amino)-1-methyl-1H-indole-2-carboxamide

1-[({[2-({[4-bromo-2-(5-oxo-4,5-dihydro-1,2,4-oxadiazol-3-yl)phenyl]amino}carbonyl)-1-methyl-1H-indol-7-yl]amino}carbonyl)(methyl)amino]-1-deoxyhexitol

N-[4-bromo-2-(5-oxo-4,5-dihydro-1,2,4-oxadiazol-3-yl)phenyl]-7-(2,3-dihydro-1,4-benzodioxin-2-ylmethoxy)-1-methyl-1H-indole-2-carboxamide-

7-(benzyloxy)-N-[4-bromo-2-(5-oxo-4,5-dihydro-1,2,4-oxadiazol-3-yl)phenyl]-1H-indole-2-carboxamide

N-[4-bromo-2-(5-oxo-4,5-dihydro-1,2,4-oxadiazol-3-yl)phenyl]-1-methyl-7-(3-phenoxypropoxy)-1H-indole-2-carboxamide

5-(acetylamino)-N-[4-bromo-2)-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-5-[(methylsulfonyl)amino]-1,2-benzisoxazole-3-carboxamide

5-(benzoylamino)-N-[4-bromo-2-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-5-[(phenylsulfonyl)amino]-1,2-benzisoxazole-3-carboxamide

N-[4-bromo-2-(5-oxo-4,5-dihydro-1,2,4-oxadiazol-3-yl)phenyl]-7-(cyclobutylmethoxy)-1-methyl-1H-indole-2-carboxamide

N-[4-bromo-2-(5-oxo-4,5-dihydro-1,2,4-oxadiazol-3-yl)phenyl]-7-(2-furylmethoxy)-1-methyl-1H-indole-2-carboxamide

N-[4-bromo-2-(5-oxo-4,5-dihydro-1,2,4-oxadiazol-3-yl)phenyl]-7-{[(4S)-2,2-dimethyl-1,3-dioxolan-4-yl]methoxy}-1-methyl-1H-indole-2-carboxamide

N-[4-bromo-2-(5-oxo-4,5-dihydro-1,2,4-oxadiazol-3-yl)phenyl]-7-{[(2R)-2,3-dihydroxypropyl]oxy}-1-methyl-1H-indole-2-carboxamide

N-[4-bromo-2-(5-oxo-4,5-dihydro-1,2,4-oxadiazol-3-yl)phenyl]-7-(cyclobutyloxy)-1-methyl-1H-indole-2-carboxamide

N-[4-bromo-2-(5-oxo-4,5-dihydro-1,2,4-oxadiazol-3-yl)phenyl]-7-(2-methoxy-1-methylethoxy)-1-methyl-1H-indole-2-carboxamide

N-[4-bromo-2-(5-oxo-4,5-dihydro-1,2,4-oxadiazol-3-yl)phenyl]-7-isopropoxy-1-methyl-1H-indole-2-carboxamide

7-(benzyloxy)-N-[4-bromo-2-(5-oxo-4,5-dihydro-1,2,4-oxadiazol-3-yl)phenyl]-1-methyl-1H-indole-2-carboxamide

N-[4-bromo-2-(5-oxo-4,5-dihydro-1,2,4-oxadiazol-3-yl)phenyl]-6-chloro-1,2-benzisoxazole-3-carboxamide

N-[4-bromo-2-(5-oxo-4,5-dihydro-1,2,4-oxadiazol-3-yl)phenyl]-6-sec-butoxy-1-methyl-1H-indole-2-carboxamide

N-[4-bromo-2-(5-oxo-4,5-dihydro-1,2,4-oxadiazol-3-yl)phenyl]-6-butoxy-1-methyl-1H-indole-2-carboxamide

N-[4-bromo-2-(5-oxo-4,5-dihydro-1,2,4-oxadiazol-3-yl)phenyl]-5-{[(4-fluorophenyl)sulfonyl]amino}-1,2-benzisoxazole-3-carboxamide

5-amino-N-[4-bromo-2-(5-oxo-4,5-dihydro-1,2,4-oxadiazol-3-yl)phenyl]-1,2-benzisoxazole-3-carboxamide

5-bromo-N-[4-bromo-2-(5-oxo-4,5-dihydro-1,2,4-oxadiazol-3-yl)phenyl]-1,2-benzisoxazole-3-carboxamide

6-[(benzylsulfonyl)amino]-N-[4-bromo-2-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-6-[(phenylsulfonyl)amino]-1,2-benzisoxazole-3-carboxamide

N-[4-bromo-2-(5-oxo-4,5-dihydro-1,2,4-oxadiazol-3-yl)phenyl]-7-(cyclohexylmethoxy)-1-methyl-1H-indole-2-carboxamide

N-[4-bromo-2-(5-oxo-4,5-dihydro-1,2,4-oxadiazol-3-yl)phenyl]-7-(cyclopropylmethoxy)-1-methyl-1H-indole-2-carboxamide

N-[4-bromo-2-(5-oxo-4,5-dihydro-1,2,4-oxadiazol-3-yl)phenyl]-1-methyl-7-(tetrahydro-2H-pyran-2-ylmethoxy)-1H-indole-2-carboxamide

N-[4-bromo-2-(5-oxo-4,5-dihydro-1,2,4-oxadiazol-3-yl)phenyl]-1-methyl-7-(pentyloxy)-1H-indole-2-carboxamide

N-[4-bromo-2-(5-oxo-4,5-dihydro-1,2,4-oxadiazol-3-yl)phenyl]-5-phenyl-1,2-benzisoxazole-3-carboxamide

N-[4-bromo-2-(5-oxo-4,5-dihydro-1,2,4-oxadiazol-3-yl)phenyl]-5-[2-(trifluoromethyl)phenyl]-1,2-benzisoxazole-3-carboxamide

N-[4-bromo-2-(5-oxo-4,5-dihydro-1,2,4-oxadiazol-3-yl)phenyl]-7-(2-methoxyethoxy)-1-methyl-1H-indole-2-carboxamide

N-[4-bromo-2-(5-oxo-4,5-dihydro-1,2,4-oxadiazol-3-yl)phenyl]-7 (2-hydroxy-3-isopropoxypropoxy)-1-methyl-1H-indole-2-carboxamide

N-[4-bromo-2-(5-oxo-4,5-dihydro-1,2,4-oxadiazol-3-yl)phenyl]-6-phenyl-1,2-benzisoxazole-3-carboxamide

N-[4-bromo-2-(5-oxo-4,5-dihydro-1,2,4-oxadiazol-3-yl)phenyl]-6-[2-(trifluoromethyl)phenyl]-1,2-benzisoxazole-3-carboxamide

N-[4-bromo-2-(5-oxo-4,5-dihydro-1,2,4-oxadiazol-3-yl)phenyl]-1-methyl-7-[2-(methylthio)ethoxy]-1H-indole-2-carboxamide

7-[(4-azido-3-iodobenzoyl)amino]-N-[4-bromo-2-(5-oxo-4,5-dihydro-1,2,4-oxadiazol-3-yl)phenyl]-1-methyl-1H-indole-2-carboxamide

5-[(4-azido-3-iodobenzoyl)amino]-N-[4-bromo-2-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-6-[(methylsulfonyl)amino]-1,2-benzisoxazole-3-carboxamide

N-[4-bromo-2-(5-oxo-4,5-dihydro-1,2,4-oxadiazol-3-yl)phenyl]-6-{[(4-fluorophenyl)sulfonyl]amino}-1,2-benzisoxazole-3-carboxamide

6-amino-N-[4-bromo-2-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-5-(2-fluorophenyl)-1,2-benzisoxazole-3-carboxamide

N-[4-bromo-2-(5-oxo-4,5-dihydro-1,2,4-oxadiazol-3-yl)phenyl]-1-methyl-6-[2-(trifluoromethyl)phenyl]-1H-indole-2-carboxamide

N-[4-bromo-2-(5-oxo-4,5-dihydro-1,2,4-oxadiazol-3-yl)phenyl]-1-methyl-6-(2,3,4-trimethoxyphenyl)-1H-indole-2-carboxamide

N-[4-bromo-2-(5-oxo-4,5-dihydro-1,2,4-oxadiazol-3-yl)phenyl]-1-methyl-7-(pyridin-3-ylmethoxy)-1H-indole-2-carboxamide

N-[4-bromo-2-(5-oxo-4,5-dihydro-1,2,4-oxadiazol-3-yl)phenyl]-6-(3,5-dimethylisoxazol-4-yl)-1-methyl-1H-indole-2-carboxamide

N-[4-bromo-2-(5-oxo-4,5-dihydro-1,2,4-oxadiazol-3-yl)phenyl]-1-methyl-6-(2-methylphenyl)-1H-indole-2-carboxamide

7-amino-4,6-dibromo-N-[4-bromo-2-(5-oxo-4,5-dihydro-1,2,4-oxadiazol-3-yl)phenyl]-1-methyl-1H-indole-2-carboxamide

4,6-dibromo-N-[4-bromo-2-(5-oxo-4,5-dihydro-1,2,4-oxadiazol-3-yl)phenyl]-1-methyl-7-[(phenylacetyl)amino]-1H-indole-2-carboxamide

3-({[4-bromo-2-(5-oxo-4,5-dihydro-1,2,4-oxadiazol-3-yl)phenyl]amino}carbonyl)-1,2-benzisoxazol-6-yl acetate

N-[4-bromo-2-(5-oxo-4,5-dihydro-1,2,4-oxadiazol-3-yl)phenyl]-6-hydroxy-1,2-benzisoxazole-3-carboxamide

6-(benzyloxy)-N-[4-bromo-2-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-6-methoxy-1,2-benzisoxazole-3-carboxamide

3-({[4-bromo-2-(5-oxo-4,5-dihydro-1,2,4-oxadiazol-3-yl)phenyl]amino}carbonyl)-1,2-benzisoxazol-6-yl phenylmethanesulfonate

3-({[4-bromo-2-(5-oxo-4,5-dihydro-1,2,4-oxadiazol-3-yl)phenyl]amino}carbonyl)-1,2-benzisoxazol-6-yl benzenesulfonate

7-amino-4-bromo-N-[4-bromo-2-(5-oxo-4,5-dihydro-1,2,4-oxadiazol-3-yl)phenyl]-1-methyl-1H-indole-2-carboxamide

7-(benzyloxy)-3-bromo-N-[4-bromo-2-(5-oxo-4,5-dihydro-1,2,4-oxadiazol-3-yl)phenyl]-1H-indole-2-carboxamide

2-{[2-({[4-bromo-2-(5-oxo-4,5-dihydro-1,2,4-oxadiazol-3-yl)phenyl]amino}carbonyl)-1-methyl-1H-indol-6-yl]amino}-2-oxoethyl acetate

5-(anilinosulfonyl)-N-[4-bromo-2-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-5-[(diethylamino)sulfonyl]-1,2-benzisoxazole-3-carboxamide

7-amino-6-bromo-N-[4-bromo-2-(5-oxo-4,5-dihydro-1,2,4-oxadiazol-3-yl)phenyl]-1-methyl-1H-indole-2-carboxamide

N-[4-bromo-2-(5-oxo-4,5-dihydro-1,2,4-oxadiazol-3-yl)phenyl]-1-methyl-7-[(phenylacetyl)amino]-4,6-bis[2-(trifluoromethyl)phenyl]-1H-indole-2-carboxamide

N-[4-bromo-2-(5-oxo-4,5-dihydro-1,2,4-oxadiazol-3-yl)phenyl]-5-nitro-6-[4-nitro-2-(trifluoromethyl)phenyl]-1,2-benzisoxazole-3-carboxamide

7-(benzyloxy)-3-bromo-N-[4-bromo-2-(5-oxo-4,5-dihydro-1,2,4-oxadiazol-3-yl)phenyl]-1-methyl-1H-indole-2-carboxamide

4-bromo-N-[4-bromo-2-(5-oxo-4,5-dihydro-1,2,4-oxadiazol-3-yl)phenyl]-1-methyl-7-[(phenylacetyl)amino]-1H-indole-2-carboxamide

N-[4-bromo-2-(5-oxo-4,5-dihydro-1,2,4-oxadiazol-3-yl)phenyl]-1-methyl-6-[(quinolin-8-ylsulfonyl)amino]-1H-indole-2-carboxamide

N-[4-bromo-2-(5-oxo-4,5-dihydro-1,2,4-oxadiazol-3-yl)phenyl]-5-(morpholin-4-ylsulfonyl)-1,2-benzisoxazole-3-carboxamide

N-[4-bromo-2-(5-oxo-4,5-dihydro-1,2,4-oxadiazol-3-yl)phenyl]-6-(cyclobutylmethoxy)-1-methyl-1H-indole-2-carboxamide

N-[4-bromo-2-(5-oxo-4,5-dihydro-1,2,4-oxadiazol-3-yl)phenyl]-6-(butyrylamino)-1-methyl-1H-indole-2-carboxamide

N-[4-bromo-2-(5-oxo-4,5-dihydro-1,2,4-oxadiazol-3-yl)phenyl]-6-[(methoxyacetyl)amino]-1-methyl-1H-indole-2-carboxamide

N-[4-bromo-2-(5-oxo-4,5-dihydro-1,2,4-oxadiazol-3-yl)phenyl]-7-methyl-1H-indole-2-carboxamide

N-[4-bromo-2-(5-oxo-4,5-dihydro-1,2,4-oxadiazol-3-yl)phenyl]-6-oxo-5,6-dihydro-4H-pyrrolo[3,2,1-ij]quinoline-2-carboxamide

5-(acetylamino)-6-[4-(acetylamino)-2-(trifluoromethyl)phenyl]-N-[4-bromo-2-(5-oxo-4,5-dihydro-1,2,4-oxadiazol-3-yl)phenyl]-1,2-benzisoxazole-3-carboxamide

N-[4-bromo-2-(5-oxo-4,5-dihydro-1,2,4-oxadiazol-3-yl)phenyl]-5-[(dimethylamino)sulfonyl]-1,2-benzisoxazole-3-carboxamide

N-[4-bromo-2-(5-oxo-4,5-dihydro-1,2,4-oxadiazol-3-yl)phenyl]-5-[(methoxyacetyl)amino]-1,2-benzisoxazole-3-carboxamide

N-[4-bromo-2-(5-oxo-4,5-dihydro-1,2,4-oxadiazol-3-yl)phenyl]-5-[(cyclobutylcarbonyl)amino]-1,2-benzisoxazole-3-carboxamide

N-[4-bromo-2-(5-oxo-4,5-dihydro-1,2,4-oxadiazol-3-yl)phenyl]-6-(2-furoylamino)-1-methyl-1H-indole-2-carboxamide

N-[4-bromo-2-(5-oxo-4,5-dihydro-1,2,4-oxadiazol-3-yl)phenyl]-1-methyl-6-({[methyl(phenyl)amino]carbonyl}amino)-1H-indole-2-carboxamide

6-(acetylamino)-N-[4-bromo-2-(5-oxo-4,5-dihydro-1,2,4-oxadiazol-3-yl)phenyl]-1-methyl-1H-indole-2-carboxamide

N-[4-bromo-2-(5-oxo-4,5-dihydro-1,2,4-oxadiazol-3-yl)phenyl]-1-methyl-6-{[4-(trifluoromethoxy)benzoyl]amino}-1H-indole-2-carboxamide

N-[4-bromo-2-(5-oxo-4,5-dihydro-1,2,4-oxadiazol-3-yl)phenyl]-6-(2-fluorophenyl)-1-methyl-1H-indole-2-carboxamide

N-[4-bromo-2-(5-oxo-4,5-dihydro-1,2,4-oxadiazol-3-yl)phenyl]-6-(cyclopropylmethoxy)-1-methyl-1H-indole-2-carboxamide

7-bromo-N-[4-bromo-2-(5-oxo-4,5-dihydro-1,2,4-oxadiazol-3-yl)phenyl]-1-methyl-1H-indole-2-carboxamide

N-[4-bromo-2-(5-oxo-4,5-dihydro-1,2,4-oxadiazol-3-yl)phenyl]-6-methoxy-5,6-dihydro-4H-pyrrolo[3,2,1-ij]quinoline-2-carboxamide

N-[4-bromo-2-(5-oxo-4,5-dihydro-1,2,4-oxadiazol-3-yl)phenyl]-6-hydroxy-5,6-dihydro-4H-pyrrolo[3,2,1-ij]quinoline-2-carboxamide

N-[4-bromo-2-(5-oxo-4,5-dihydro-1,2,4-oxadiazol-3-yl)phenyl]-6-phenoxy-5,6-dihydro-4H-pyrrolo[3,2,1-ij]quinoline-2-carboxamide

N-[4-bromo-2-(5-oxo-4,5-dihydro-1,2,4-oxadiazol-3-yl)phenyl]-1-methyl-7-[2-(trifluoromethyl)phenyl]-1H-indole-2-carboxamide

N-[4-bromo-2-(5-oxo-4,5-dihydro-1,2,4-oxadiazol-3-yl)phenyl]-1-methyl-7-phenyl-1H-indole-2-carboxamide

N-[4-bromo-2-(5-oxo-4,5-dihydro-1,2,4-oxadiazol-3-yl)phenyl]-7-(4-tert-butylphenyl)-1-methyl-1H-indole-2-carboxamide

N-[4-bromo-2-(5-oxo-4,5-dihydro-1,2,4-oxadiazol-3-yl)phenyl]-1-methyl-7-(5-methylthien-2-yl)-1H-indole-2-carboxamide

N-[4-bromo-2-(5-oxo-4,5-dihydro-1,2,4-oxadiazol-3-yl)phenyl]-1-methyl-6-[(pyridin-3-ylcarbonyl)amino]-1H-indole-2-carboxamide

N-[4-bromo-2-(5-oxo-4,5-dihydro-1,2,4-oxadiazol-3-yl)phenyl]-1-methyl-6-[(methylsulfonyl)amino]-1H-indole-2-carboxamide

N-[4-bromo-2-(5-oxo-4,5-dihydro-1,2,4-oxadiazol-3-yl)phenyl]-6-({[(2,5-dimethoxyphenyl)amino]carbonyl}amino)-1-methyl-1H-indole-2-carboxamide

N-[4-bromo-2-(5-oxo-4,5-dihydro-1,2,4-oxadiazol-3-yl)phenyl]-6-[(isoxazol-5-ylcarbonyl)amino]-1-methyl-1H-indole-2-carboxamide

N-[4-bromo-2-(5-oxo-4,5-dihydro-1,2,4-oxadiazol-3-yl)phenyl]-1-methyl-6-{[(pentylamino)carbonyl]amino}-1H-indole-2-carboxamide

N-[4-bromo-2-(5-oxo-4,5-dihydro-1,2,4-oxadiazol-3-yl)phenyl]-1-methyl-7-[4-(methylsulfonyl)phenyl]-1H-indole-2-carboxamide

N-[4-bromo-2-(5-oxo-4,5-dihydro-1,2,4-oxadiazol-3-yl)phenyl]=7-(2-methoxyphenyl)-1-methyl-1H-indole-2-carboxamide

N-[4-bromo-2-(5-oxo-4,5-dihydro-1,2,4-oxadiazol-3-yl)phenyl]-7-(2-fluorophenyl)-1-methyl-1H-indole-2-carboxamide

N-[4-bromo-2-(5-oxo-4,5-dihydro-1,2,4-oxadiazol-3-yl)phenyl]-1-methyl-7-(2-methylphenyl)-1H-indole-2-carboxamide

N-[4-bromo-2-(5-oxo-4,5-dihydro-1,2,4-oxadiazol-3-yl)phenyl]-6-{[(3,5-dimethylisoxazol-4-yl)sulfonyl]amino}-1-methyl-1H-indole-2-carboxamide

N-[4-bromo-2-(5-oxo-4,5-dihydro-1,2,4-oxadiazol-3-yl)phenyl]-5-(dimethylamino)-1,2-benzisoxazole-3-carboxamide

### Example 33

### Antibacterial Activity

MIC Test Method

The in vitro MICs of test compounds were determined by a standard agar dilution method. A stock drug solution of each analog was prepared in the preferred solvent, usually DMSO:H₂O (1:3). Serial 2-fold dilutions of each sample are made using 1.0 ml aliquots of sterile distilled water. To each 1.0 ml aliquot of drug was added 9 ml of molten Mueller Hinton agar medium. The drug-supplemented agar was mixed, poured into 15×100 mm petri dishes, and allowed to solidify and dry prior to inoculation.

Vials of each of the test organisms are maintained frozen in the vapor phase of a liquid nitrogen freezer. Test cultures are grown overnight at 35° C. on the medium appropriate for the organism. Colonies are harvested with a sterile swab, and cell suspensions are prepared in Trypticase Soy broth (TSB) to equal the turbidity of a 0.5 McFarland standard. A 1:20 dilution of each suspension was made in TSB. The plates containing the drug supplemented agar are inoculated with a 0.001 ml drop of the cell suspension using a Steers replicator, yielding approximately 10⁴ to 10⁵ cells per spot. The plates are incubated overnight at 35° C.

Following incubation the Minimum Inhibitory Concentration (MIC μg/ml), the lowest concentration of drug that inhibits visible growth of the organism, was read and recorded.

TABLE 1

| Ex- | | | |
|---|---|---|---|
| am- | | | SAUR |
| ple | | 9218 | |
| # | Structure | Compound Name(s) | MIC |
| 1 | 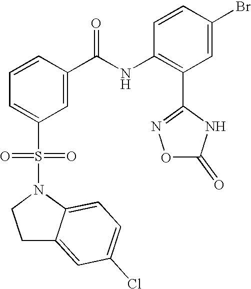 | N-[4-bromo-2-(5- oxo-4,5-dihydro- 1,2,4-oxadiazol-3- yl)phenyl]-3-[(5- chloro-2,3-dihydro- 1H-indol-1- yl)sulfonyl]benzamide | 4 |
| 2 | 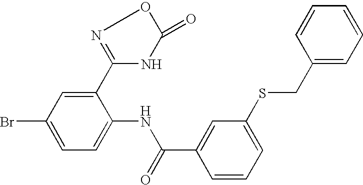 | 3-(benzylsulfanyl)- N-[4-bromo-2-(5- oxo-4,5-dihydro- 1,2,4-oxadiazol-3- yl)phenyl]benzamide | 8 |
| 3 | 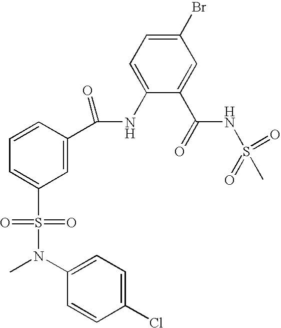 | N-(4-bromo-2- {[(methylsulfonyl) amino]carbonyl}phenyl)-3-{[4- chloro(methyl)anilino]sulfonyl}benzamide | 2 |
| 4 | 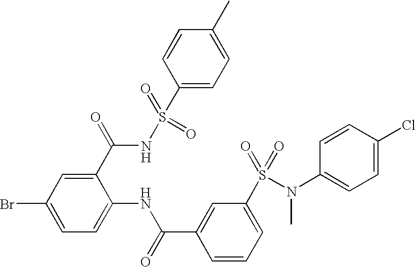 | N-[4-bromo-2- ({[(4- methylphenyl)sulfo- nyl]amino}carbonyl) phenyl[-3-{[(4- chlorophenyl)(meth yl)amino]sulfonyl}benzamide | 8 |
| 5 | 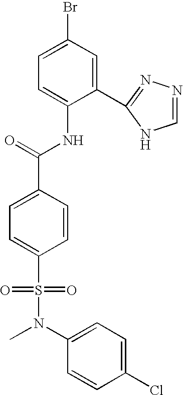 | N-[4-bromo-2-(1H- tetraazol-5- yl)phenyl]-4-{[(4- chlorophenyl)(meth yl)amino]sulfonyl}benzamide | 16 |
| 6 | 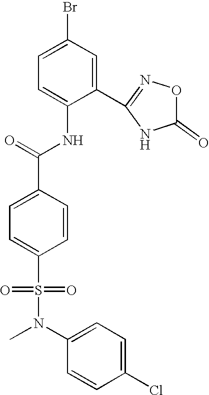 | N-{4-bromo-2-(5- oxo-4,5-dihydro- 1,2,4-oxadiazol-3- yl)phenyl]-4-{[(4- chlorophenyl)(meth yl)amino]sulfonyl}benzamide | 16 |
| 7 | 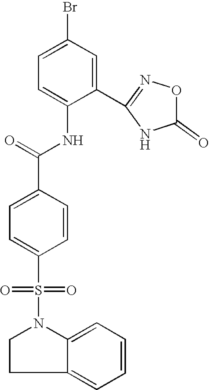 | N-[4-bromo-2-(5- oxo-4,5-dihydro- 1,2,4-oxadiazol-3- yl)phenyl]-4-(2,3- dihydro-1H-indol- 1-ylsulfonyl) benzamide | 16 |
| 8 | 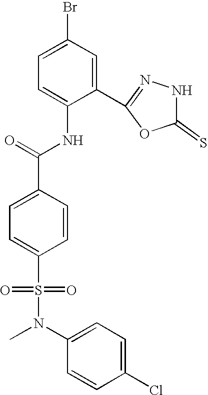 | N-[4-bromo-2-(5- thioxo-4,5-dihydro- 1,3,4-oxadiazol-2- yl)phenyl]-4-{[4- chloro(methyl)anilino]sulfonyl}benzamide | 4 |
| 9 | 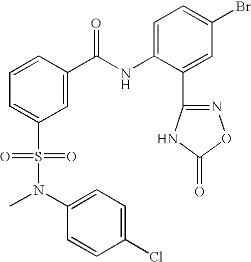 | N-[4-bromo-2-(5- oxo-4,5-dihydro- 1,2,4-oxadiazol-3- yl)phenyl]-3-{[4- chloro(methyl)anilino]sulfonyl}benzamide | 16 |
| 10 | 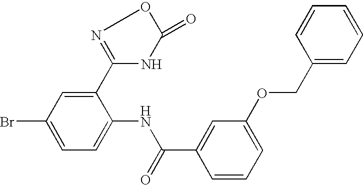 | 3-(benzyloxy)-N- [4-bromo-2-(5-oxo- 4,5-dihydro-1,2,4- oxadiazol-3- yl)phenyl]benzamide | 8 |
| 11 | 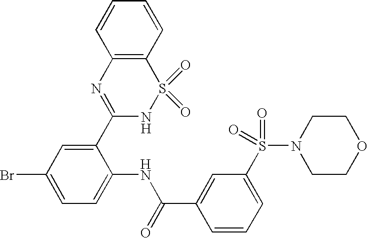 | N-[4-bromo-2-(1,1- dioxido-2H-1,2,4- benzothiadiazin-3- yl)phenyl]-3- (morpholin-4- ylsulfonyl)benzamide | 16 |
| 12 | 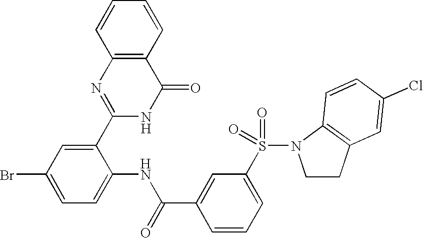 | N-[4-bromo-2-(4- oxo-3,4- dihydroquinazolin- 2-yl)phenyl]-3-[(5- chloro-2,3-dihydro- 1H-indol-1- yl)sulfonyl]benzamide | 4 |
| 13 | 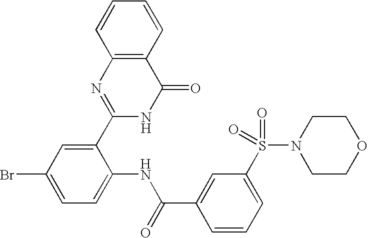 | N-[4-bromo-2-(4- oxo-3,4- dihydroquinazolin- 2-yl)phenyl]-3- (morpholin-4- ylsulfonyl)benzami de | 4 |
| 14 | 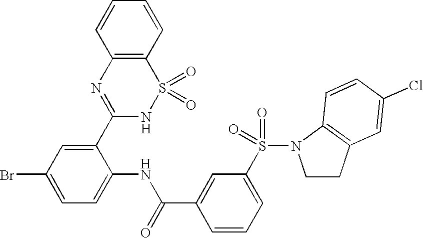 | N-{4-bromo-2-(1,1- dioxido-2H-1,2,4- benzothiadiazin-3- yl)phenyl]-3-[(5- chloro-2,3-dihydro- 1H-indol-1- yl)sulfonyl]benzamide | 64 |
| 15 | 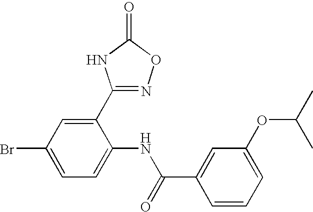 | N-[4-bromo-2-(5- oxo-4,5-dihydro- 1,2,4-oxadiazol-3- yl)phenyl]-3- isopropoxybenzamide | 32 |
| 16 | 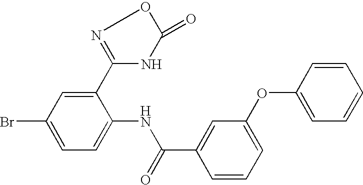 | N-[4-bromo-2-(5- oxo-4,5-dihydro- 1,2,4-oxadiazol-3- yl)phenyl]-3- phenoxybenzamide | 16 |
| 17 | 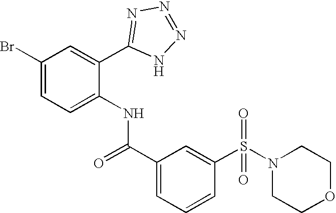 | N-[4-bromo-2-(1H- tetraazol-5- yl)phenyl]-3- (morpholin-4- ylsulfonyl)benzamide | 64 |
| 18 | 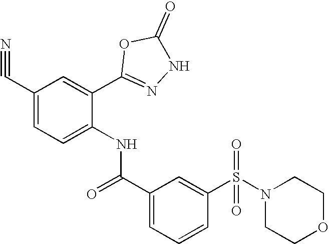 | N-[4-cyano-2-(5- oxo-4,5-dihydro- 1,3,4-oxadiazol-2- yl)phenyl]-3- (morpholin-4- ylsulfonyl)benzamide | >128 |
| 19 | 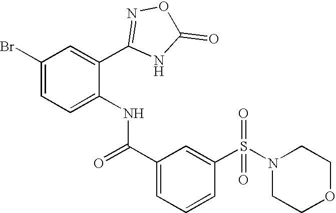 | N-[4-bromo-2-(5- oxo-4,5-dihydro- 1,2,4-oxadiazol-3- yl)phenyl]-3- (morpholin-4- ylsulfonyl)benzamide | 128 |
| 20 | 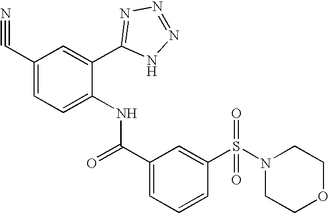 | N-[4-cyano-2-(1H- tetraazol-5- yl)phenyl]-3- (morpholin-4- ylsulfonyl)benzamide | 64 |
| 21 | 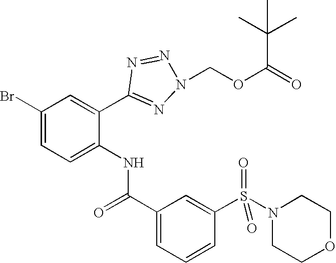 | [5-(5-bromo-2-{[3- (morpholin-4- ylsulfonyl)benzoyl]amino}phenyl)-2H- yl]methyl pivalate | >64 |
| 22 | 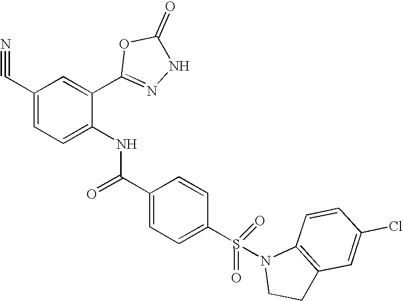 | 4-[(5-chloro-2,3- dihydro-1H-indol- 1-yl)sulfonyl]-N-[4- cyano-2-(5-oxo-4,5- dihydro-1,3,4- oxadiazol-2- yl)phenyl]benzamide | 32 |
| 23 | 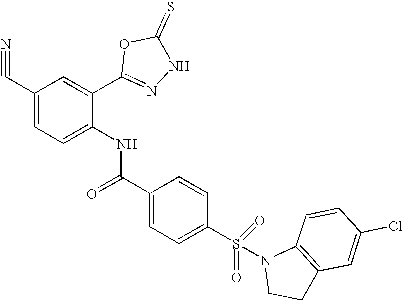 | 4-[(5-chloro-2,3- dihydro-1H-indol- 1-yl)sulfonyl]-N-[4- cyano-2-(5-thioxo-8 4,5-dihydro-1,3,4- oxadiazol-2- yl)phenyl]benzamide | 8 |
| 24 | 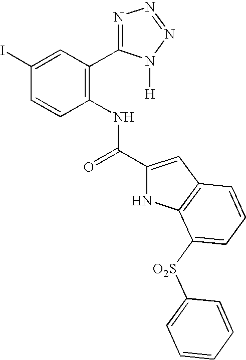 | N-[4-iodo-2-(1H- tetraazol-5- yl)phenyl]-7- (phenylsulfonyl)- 1H-indole-2- carboxamide | 0.5 |
| 25 | 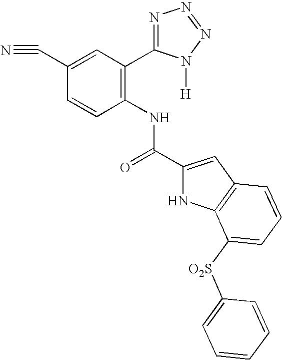 | N-[4-cyano-2-(1H- tetraazol-5- yl)phenyl]-7- (phenylsulfonyl)- 1H-indole-2- carboxamide | 0.125 |
| 26 | 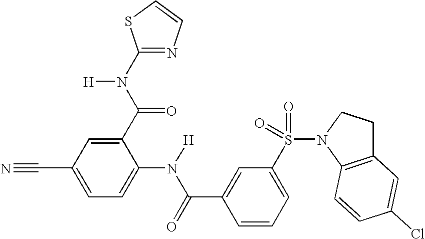 | 2-({3-[(5-chloro- 2,3-dihydro-1H- indol-1- yl)sulfonyl]benzoyl}amino)-5-cyano- N-(1,3-thiazol-2- yl)benzamide | 8 |
| 27 | 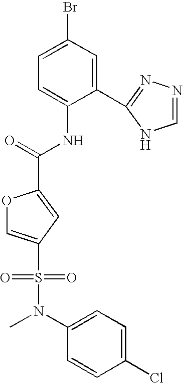 | N-[4-bromo-2-(1H- 1,2,3,4-tetraazol-5- yl)phenyl]-4-{[4- chloro(methyl)anili no]sulfonyl}-2- furamide | 16 |
| 28 | 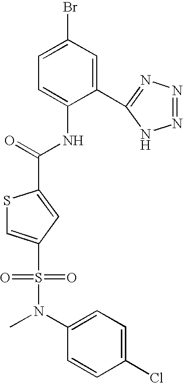 | N-[4-bromo-2-(1H- tetraazol-5- yl)phenyl]-4-{[4- chloro(methyl)anili no]sulfonyl}-2- thiophenecarboxamide | 16 |
| 29 | 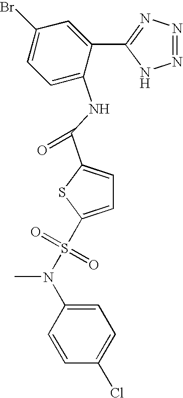 | N-[4-bromo-2-(1H- tetraazol-5- yl)phenyl]-5-{[4- chloro(methyl)anili no]sulfonyl}-2- thiophenecarboxamide | 8 |
| 30 | 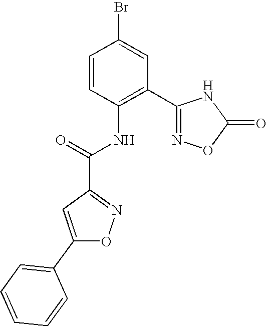 | N-[4-bromo-2-(5- oxo-4,5-dihydro- 1,2,4-oxadiazol-3- yl)phenyl]-5- phenylisoxazole-3- carboxamide | 1 |
| 31 | 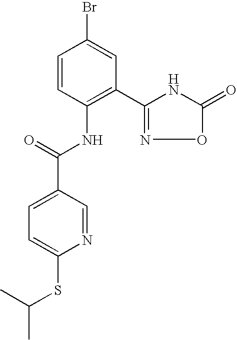 | N-[4-bromo-2-(5- oxo-4,5-dihydro- 1,2,4-oxadiazol-3- yl)phenyl]-6- (isopropylthio)nicot inamide | 8 |
| | 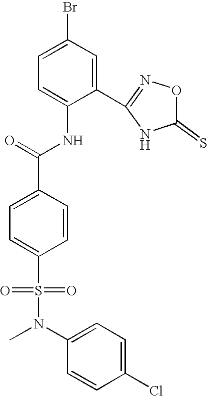 | N-[4-bromo-2-(5- thioxo-4,5-dihydro- 1,2,4-oxadiazol-3- yl)phenyl]-4-{[4- chloro(methyl)anilino]sulfonyl}benzamide | 16 |
| | 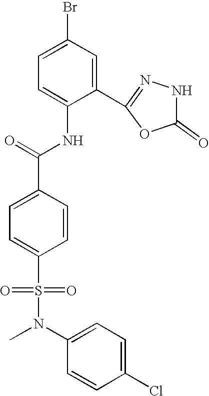 | N-[4-bromo-2-(5- oxo-4,5-dihydro- 1,3,4-oxadiazol-2- yl)phenyl]-4-{[4- chloro(methyl)anilino]sulfonyl}benzamide | 2 |
| | 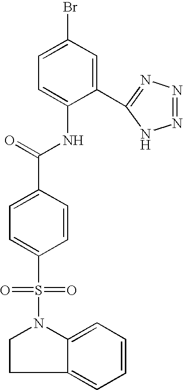 | N-[4-bromo-2-(1H- 1,2,3,4-tetraazol-5- yl)phenyl]-4-(2,3- dihydro-1H-indol-1- ylsulfonyl)benzamide | 2 |
| | 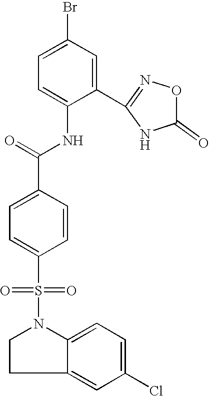 | N-[4-bromo-2-(5- oxo-4,5-dihydro- 1,2,4-oxadiazol-3- yl)phenyl]-4-[(5- chloro-2,3-dihydro- 1H-indol-1-yl) sulfonyl]benzamide | 8 |
| | 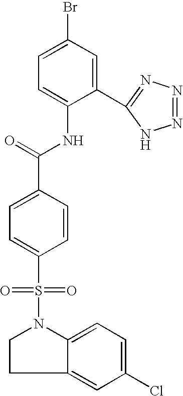 | N-[4-bromo-2-(1H- 1,2,3,4-tetraazol-5- yl)phenyl]-4-[(5- chloro-2,3-dihydro- 1H-indol-1-yl) sulfonyl]benzamide | 4 |
| | 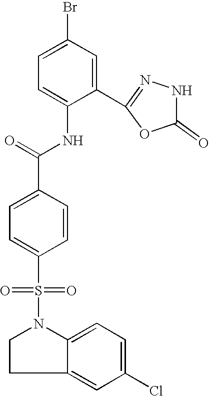 | N-[4-bromo-2-(5- oxo-4,5-dihydro- 1,3,4-oxadiazol-2- yl)phenyl]-4-[(5- chloro-2,3-dihydro- 1H-indol-1-yl) sulfonyl]benzamide | >128 |
| | 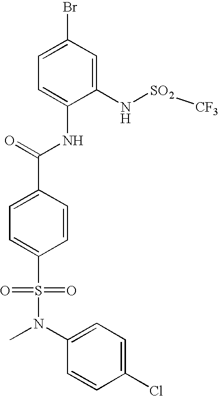 | N-(4-bromo-2- {[(trifluoromethyl)s ulfonyl]amino}phen chloro(methyl)anilino]sulfonyl}benzamide | 32 |
| | 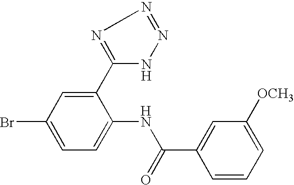 | N-[4-bromo-2-(1H- tetraazol-5- yl)phenyl]-3- methoxybenzamide | 64 |

What is claimed is:

1. A compound of the formula 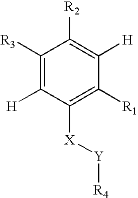
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group; and
R₄ is an optionally substituted aryl provided that the aryl is not simultaneously substituted with a sulfonamide and a urea or thiourea, and further provided that the aryl is not solely substituted at the ortho-position relative to Y, or R₄ is an optionally substituted HET².

2. The compound of claim 1 having the formula II 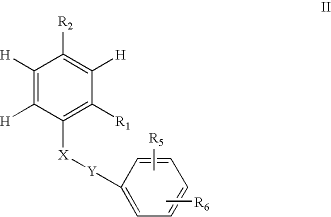
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)-NR₈, substituted aryl, substituted C₁₋₄alkyl, or substituted C₁₋₄alkenyl;
R₆ is selected from H, halo, HET², —CN, NH₂, NO₂, alkyl, substituted alkyl, alkoxy, substituted alkoxy, —NH—CO-HET², and —NH—CO-aryl;
R₇ is selected from alkyl, substituted alkyl, aryl, substituted aryl, —N(Q₁₅)₂, HET², and substituted HET;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ ₆C(S)Q₁₆, —NQ₁₆C(O)NQ₁ 6Q)₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl, the alkyl and cycloalkyl optionally including 1-3 halos;
W is O, S, —(CZ₂)-, or —(CHZ₃)-;
Z₁ is O;
Z₂ is ═O, ═S, N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

3. The compound of claim 1 having the formula III 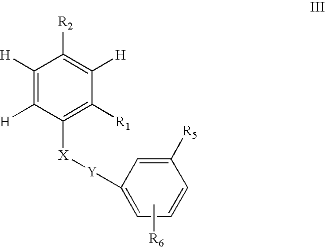
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)—R₈, —NH—(CZ₁)—NR₈, substituted aryl, substituted C₁₋₄alkyl, or substituted C₁₋₄alkenyl;
R₆ is selected from H, halo, HET², —CN, NH₂, NO₂, alkyl, substituted alkyl, alkoxy, substituted alkoxy, —NH—CO-HET², and —NH—CO-aryl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁ 6)₂, —SC(O)Q₁₆, —NQ₁ 6Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)-, or —(CHZ₃)-;
Z₁ is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

4. The compound of claim 1 having the formula IV 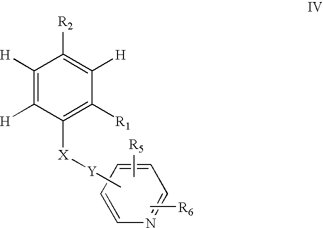
or a pharmaceutically acceptable salt thereof, wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)-NR₈, substituted aryl, substituted C₁₋₄alkyl, or substituted C₁₋₄alkenyl;
R₆ is selected from H, halo, HET², —CN, NH₂, NO₂, alkyl, substituted alkyl, alkoxy, substituted alkoxy, —NH—CO-HET², and —NH—CO-aryl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q 6)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ ₁₆Q 1 ₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁ is O;
Z₂ is ═O, ═S, —N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

5. The compound of claim 1 having the formula V 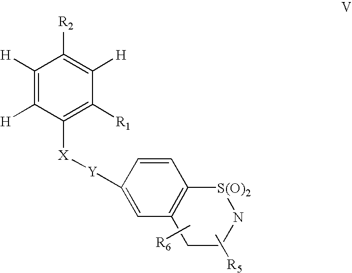
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)-NR₈, substituted aryl, substituted C₁₋₄alkyl, or substituted C₁₋₄alkenyl;
R₆ is selected from H, halo, HET², —CN, NH₂, NO₂, alkyl, substituted alkyl, alkoxy, substituted alkoxy, —NH—CO-HET², and —NH—CO-aryl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q 16)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁ 6S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)-, or —(CHZ₃)-;
Z, is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

6. The compound of claim 1 having the formula VI 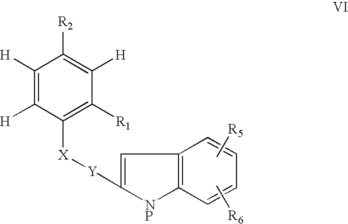
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
P is Q₁₆;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)-NR₈, substituted aryl, substituted C₁₋₄alkyl, or substituted C₁₋₄alkenyl;
R₆ is selected from H, halo, HET², —CN, NH₂, NO₂, alkyl, substituted alkyl, alkoxy, substituted alkoxy, —NH—CO-HET², and —NH—CO-aryl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)-, or —(CHZ₃)-;
Z₁ is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

7. The compound of claim 1 having the formula VII 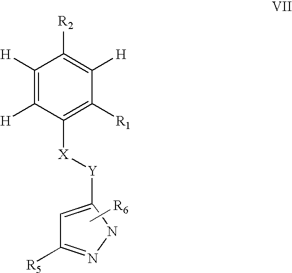
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)-NR₈, substituted aryl, substituted C₁₋₄alkyl, or substituted C₁₋₄alkenyl;
R₆ is selected from H, halo, HET¹, —CN, NH₂, NO₂, alkyl, substituted alkyl, alkoxy, substituted alkoxy, —NH—CO-HET², and —NH—CO-aryl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₆)₂OC(O)Q₁₆, —CN, —NQ ₆C(O)Q₁₆, —NQ ₆C(S)Q 6, —NQ₁₆C(O)NQ 6Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)-;
Z₁ is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

8. The compound of claim 1 having the formula VIII 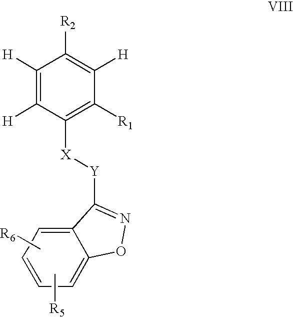
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is H, halo, NO₂, CN, —(CH₂)ₖ—S(O)ᵢ—R₇, —NH—SO₂—R₇, —(CH₂)ₖ—W—R₈—NH—(CZ₁)-R₈, —(CZ₁)-NH—R₈, —NH—(CZ₁)-NR₈R₈, —(CH₂)ₖ—NR₈R₈, substituted aryl, substituted HET, substituted C₁₋₄alkyl, or substituted C₁₋₄alkenyl;
R₆ is selected from H, halo, aryl, substituted aryl, HET, substituted HET, —CN, NH₂, NO₂, alkyl, substituted alkyl, alkoxy, substituted alkoxy, —(CH₂)ₖ—S(O)ᵢ—R₇, —NH—SO₂—R₇, —(CH₂)ₖ—W—R₈, —NH—(CZ₁)-R₈, —(CZ₁)-NH—R₈, —NH—(CZ₁)-NR₈R₈, or substituted C₁₋₄alkenyl;
R₇ is selected from alkyl, substituted alkyl, aryl, substituted aryl, —N(Q₁₅)₂, HET, and substituted HET;
Each R₈ is independently H, alkyl, substituted alkyl, —OQ₁₆, aryl, substituted aryl, HET, substituted HET, cycloalkyl, and substituted cycloalkyl, or two R₈ substituents when attached to the same atom may be taken together to form a 5-8 membered ring, wherein the ring includes the atom to which the two R₉ substituents attach;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁ 6Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —(O)C(Q₁₆)₂OC(O)Q₁₆, —CN, —NQ₁₆C(O)Q₁₆, —NQ₁₆C(S)Q₁₆, —NQ₁₆C(O)NQ₁ 6Q₁₆, —NQ₁₆C(S)NQ₁ 6Q₁₆, —S(O)₂NQ₁₆Q₁₆, —NQ₁ 6S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, cycloalkyl, phenyl, benzyl, —CH₂-substituted phenyl, and Het in which each of alkyl, cycloalkyl, phenyl, and Het optionally include 1-3 halos;
W is O, S, —(CZ₂)-, or —(CHZ₃)-, provided that W is not S or O when R₅ or R₆ are —(CH₂)ₖ—W—OR₁₆;
Z₁ is ═O;
Z₂ is ═O, ═S, ═N—OH, ═N—O-alkyl, or ═N—O-substituted alkyl;
Z₃ is —OH, —N═N-alkyl, —NH-alkyl, or —NH-substituted alkyl;
i is 0, 1, or 2; and
k is 0, 1, or 2.

9. The compound of claim 8, wherein at least one of R₅ and R₆ is a substituted phenyl or substituted HET.

10. The compound of claim 9, wherein at least one of R₅ and R₆ is pyridine, pyrimidine, pyridazine, or pyrazine, each of which is optionally substituted with the substituents described for substituted HET.

11. The compound of claim 9, wherein the substituted phenyl has the formula 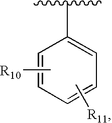
wherein each R₁₀ and R₁₁ is selected from —F, —Cl, —Br, —I, —OQ₁₆, -Q₁₆, —SQ₁₆, —S(O)₂Q₁₆, —S(O)Q₁₆, —OS(O)₂Q₁₆, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁6SQ₁₆, —NO₂, and —SNQ₁₆Q₁₆.

12. The compound of claim 8, wherein the substituted phenyl has the formula

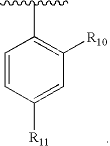

13. The compound of claim 8, wherein one of R₅ or R₆ is —NH—(CZ₁)-NR₈R₈.

14. The compound of claim 13, wherein —NR₈R₈ forms a 5-8 membered ring.

15. The compound of claim 14, wherein the ring is morpholino, pyrrolidinyl, or piperdinyl.

16. The compound of 13, wherein at least one of the R₈ substituents is benzyl or —CH₂-substituted phenyl.

17. The compound of claim 8, wherein one of R₅ or R is —(CH₂)ₖ—S(O)ᵢ—R₇ or —NH—SO₂—R₇.

18. The compound of claim 17, wherein R₇ is het, substituted het, alkyl, or substituted alkyl.

19. The compound of claim 18, wherein het is indolinyl, pyrrolindinyl, or indolyl, pyrrolyl.

20. The compound of claim 18, wherein sustituted het includes a het substituent substituted with 1-3 of halo or CN.

21. The compound of claim 18, wherein substituted alkyl is an alkyl substituted with 1-3 of OH, NH₂, NHQ₁₆, —NR₈R₈.

22. The compound of claim 1 having the formula XXX 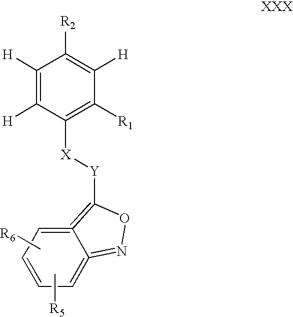
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is H, halo, NO₂, CN, —(CH₂)ₖ—S(O)ᵢ—R₇, —NH—SO₂—R₇, —(CH₂)ₖ—W—R₈—NH—(CZ₁)-R₈, —(CZ₁)-NH—R₈, —NH—(CZ₁)-NR₈R₈, —(CH₂)ₖ—NR₈R₈, substituted aryl, substituted HET, substituted C₁₋₄alkyl, or substituted C₁₋₄alkenyl;
R₆ is selected from H, halo, aryl, substituted aryl, HET, substituted HET, —CN, NH₂, NO₂, alkyl, substituted alkyl, alkoxy, substituted alkoxy, —(CH₂)ₖ—S(O)ᵢ—R₇, —NH—SO₂—R₇, —(CH₂)ₖ—W—R₈, —NH—(CZ₁)-R₈, —(CZ₁)-NH—R₈, —NH—(CZ₁)-NR₈R₈, or substituted C₁₋₄alkenyl;
R₇ is selected from alkyl, substituted alkyl, aryl, substituted aryl, —N(Q₁₅)₂, HET, and substituted HET;
Each R₈ is independently H, alkyl, substituted alkyl, —OQ₁₆, aryl, substituted aryl, HET, substituted HET, cycloalkyl, and substituted cycloalkyl, or two R₈ substituents when attached to the same atom may be taken together to form a 5-8 membered ring, wherein the ring includes the atom to which the two R₉ substituents attach;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ 16)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —(O)C(Q₆)₂OC(O)Q 6, —CN, —NQ ₁₆C(O)Q₁₆, —NQ ₁₆C(S)Q₁₆, —NQ_{1 6}C(O)NQ 16Q₁₆, —NQ ₁₆C(S)NQ₁₆Q 6, —S(O)₂NQ₁ 6Q₁₆, —NQ₁ 6S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, cycloalkyl, phenyl, benzyl, —CH₂-substituted phenyl, and Het in which each of alkyl, cycloalkyl, phenyl, and Het optionally include 1-3 halos;
W is O, S, —(CZ₂)-, or —(CHZ₃)-, provided that W is not S or O when R₅ or R₆ are —(CH₂)ₖ—W—OR₁₆;
Z₁ is ═O;
Z₂ is ═O, ═S, ═N—OH, ═N—O-alkyl, or ═N—O-substituted alkyl;
Z₃ is —OH, —N═N-alkyl, —NH-alkyl, or —NH-substituted alkyl;
i is 0, 1, or 2; and
k is 0, 1, or 2.

23. The compound of claim 1 having the formula IX 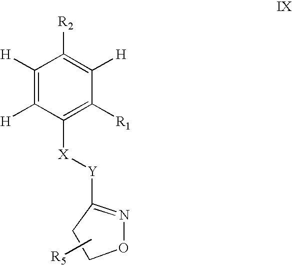
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)-NR₈, substituted aryl, substituted C₁₋₄alkyl, or substituted C₁₋₄alkenyl;
R₆ is selected from H, halo, HET², —CN, NH₂, NO₂, alkyl, substituted alkyl, alkoxy, substituted alkoxy, —NH—CO-HET², and —NH—CO-aryl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)-, or —(CHZ₃)-;
Z₁ is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

24. The compound of claim 1 having the formula X 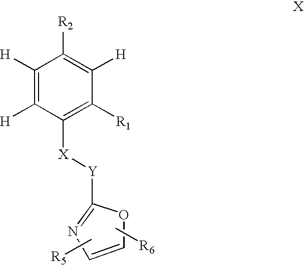
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q₁, the HET¹ being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)-NR₈, substituted aryl, substituted C₁₋₄alkyl, or substituted C₁₋₄alkenyl;
R₆ is selected from H, halo, —CN, NH₂, NO₂, alkyl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q[₆, —C(O)NQ₁₆Q₁₆, —C(S)NQ₁₆Q₁₆, —C(O)C(Q₁₆)₂OC(O)Q₁₆, —CN, —NQ ₁₆C(O)Q₁₆, —NQ₁₆C(S)Q₁₆, —NQ₁₆C(O)NQ₁ 6Q₁₆, —NQ₁₆C(S)NQ₁₆Q₁₆, —S(O)₂NQ₁₆Q₁₆, —NQ₁₆S(O)₂Q₁₆, —NQ₁ 6S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)-, or —(CHZ₃)-;
Z₁ is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

25. The compound of claim 1 having the formula XI 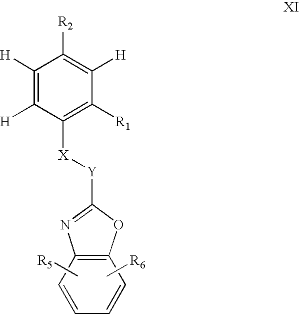
or a pharmaceutically acceptable salt thereof,
wherein
X═NH
Y═CO, CS, —C(═N—CN) or
X and Y together form an alkene, or C₃-C₅ cycloalkyl;
R is -HET¹, —CO-HET¹, or —NH—S(O)₂-Q¹, the HET¹being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)-NR₈, substituted aryl, substituted C₁₋₄alkyl, or substituted C₁₋₄alkenyl;
R₆ is selected from H, halo, HET², —CN, NH₂, NO₂, alkyl, substituted alkyl, alkoxy, substituted alkoxy, —NH—CO-HET², and —NH—CO-aryl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q)₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q)₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)-, or —(CHZ₃)-;
Z₁ is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

26. The compound of claim 1 having the formula XII 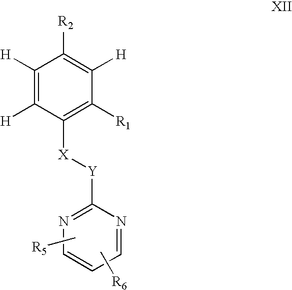
or a pharmaceutically acceptable salt thereof,
wherein
X═NH
Y═CO, CS, —C(═N—CN) or
X and Y together form an alkene, or C₃-C₅ cycloalkyl;
R₁ is -HET¹, —CO-HET¹, or —NH—S(O)₂-Q¹, the HET¹ being an optionally substituted HET¹;
Q₁ is selected from the group consisting of H, optionally substituted alkyl, or optionally substituted aryl;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)-R₈, —NH—(CZ₁)—NR₈, substituted aryl, substituted C₁₋₄alkyl, or substituted C₁₋₄alkenyl;
R₆ is selected from H, halo, HET², —CN, NH₂, NO₂, alkyl, substituted alkyl, alkoxy, substituted alkoxy, —NH—CO-HET¹, and —NH—CO-aryl;
R₇ is selected from alkyl, substituted alkyl, aryl, substituted aryl, —N(Q₁₅)₂, HET², and substituted HET²;
R₈ is H, alkyl, substituted alkyl, aryl, substituted aryl, HET², substituted HET², cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₆, —SQ₁₆, —S(O)₂Q)₆, —S(O)Q)₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)-, or —(CHZ₃)-;
Z₁ is O;
Z₂ is =0, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

27. The compound of claim 1, wherein Y is —CO—.

28. The compound of claim 1, wherein Y is —CS—.

29. The compound of claim 1, wherein X—Y is —C═C—.

30. The compound of claim 1, wherein is cyclopropyl.

31. The compound of claim 1, wherein R₂ is halo, —CN, —NO₂, HET², substituted HET², aryl, substituted aryl, —(CO)-alkyl, —(CO)-substituted alkyl, —(CO)-aryl, —(CO)-substituted aryl, —(CO)—O-alkyl, —(CO)—O-substituted alkyl, —(CO)—O-aryl, —(CO)—O-substituted aryl, —OC(Zₙ)₃, —C(Zₙ)₃, —C(Zₙ)₂—O—C(Zₘ)₃, —SO₂—C(Zₙ)₃, —SO₂-aryl, —CN(Q₁₇)₂, —C(NQ₁₇)Q₁₇.—CH═C(Q₁₇)₂, or —C—C-Q₁₇, in which each Zn and Zm is independently H, halo, —CN, —NO₂—OH, or C₁₋₄alkyl optionally substituted with 1-3 halo, —OH, NO₂, provided that at least one of Zn is halo, —CN, or NO₂.

32. The compound of claim 31, wherein R₂ is Br, Cl, F, I, —CN, formyl, acetyl, methoxyimino, hydroxyimino, —CH₂-halo, CH₂—CN, phenyl, thienyl, pyrazinyl, 1-methyl-1H-pyrrol-2-yl, pyridin-2-yl, chlorophenyl, nitrophenyl, cyanophenyl, chlorothienyl, methylthienyl, fluorophenyl, (trifluoromethy)phenyl, di (trifluoromethy)phenyl, difluorophenyl, dimethylisoxazolyl, dimethoxypyrimidinyl.

33. The compound of claim 1, wherein R₅ is —NH₂, —SO₂—NH-alkyl, —SO₂—NH-substituted alkyl, —SO₂—NH-aryl, —NH—SO₂-aryl, —SO₂—NH-substituted aryl, —NH—SO₂-substituted aryl, —SO₂—NH-HET², —SO₂—NH-substituted HET², —SO₂—N(alkyl)(substituted alkyl), —SO₂—N(alkyl)(aryl), —SO₂—N(alkyl)(substituted aryl), —SO₂—N(alkyl)(HET²), —SO₂—N(alkyl)(substituted HET 2), —S-alkyl, —S-substituted alkyl, —O-alkyl, —O-aryl, —S-substituted alkyl, —CH₂—S-alkyl, —CH₂—S-substituted alkyl, —(CH₂)₂—S-alkyl, —(CH₂)₂—S-substituted alkyl, —C(O)-aryl, —C(O)H, —C(OH)-aryl, —C(N—OCH₃)— aryl, —C(N—OH)-aryl, —C(O)—C₁₋₆cycloalkyl, —NH—C(O)—O—C₁₋₄alkyl, —NH—C(O)-aryl, —NH—C(O)-substituted aryl, —NH—C(O)-HET², —NH—C(O)-substituted HET², —NHC(O)NH-aryl, —NHC(O)NH-substituted aryl, —NHC(O)NH-HET¹, —NHC(O)NH-substituted HET².

34. The compound of claim 33, wherein R₅ is (diethylamino)sulfonyl, (1H-indol-5-yl)aminosulfonyl, (furylmethylamino)sulfonyl, (ethoxycarbonyl)-1-piperazinylsulfonyl, pyridinylethylaminosulfonyl, (benzylamino)sulfonyl, (2-hydroxy-1-methylethyl)aminosulfonyl, (4-carboxyanilino)sulfonyl, (3,4-dihydro-[(2H)-quinolinyl)sulfonyl, [2-(3,5-dimethoxyphenyl)ethyl]aminosulfonyl, [(3S)-3-hydroxypyrrolidinyl]sulfonyl, (ethylanilino)sulfonyl, (3,5-dimethoxyanilino)sulfonyl, (2-hydroxy-2-phenylethyl)(methyl)amino]sulfonyl, (2,3-dihydro-1H-indol-1-yl)sulfonyl, (5-methoxy-2,3-dihydro-1H-indol-1-yl)sulfonyl, (5-fluoro-2,3-dihydro-1H-indol-1-yl)sulfonyl, (1H-benzimidazol-1-yl)sulfonyl, (5-fluoro-1H-indol-1-yl)sulfonyl, (1H-indol-1-yl)sulfonyl, (6-fluoro-1H-indol-1-yl)sulfonyl, (5-chloro-1H-indol-1-yl)sulfonyl, (6-chloro-1H-indol-1-yl)sulfonyl, (6-chloro-5-fluoro-1H-indol-1-yl)sulfonyl, (1H-pyrrol-1-yl)sulfonyl, (5-methoxy-1H-indol-1-yl)sulfonyl, (1H-pyrrolo[2,3-b]pyridin-1-yl)sulfonyl, (5-bromo-2,3-dihydro-1H-indol-1-yl)sulfonyl, (3,3-dimethyl-2,3-dihydro-1H-indol-1-yl)sulfonyl, (4-chlorophenyl)(methyl)amino]sulfonyl, benzylthio, methyl(pyridin-2-yl)amino]sulfonyl, (1H-indol-1-yl)sulfonyl, (pyrrolidin-1-yl)sulfonyl, (2-methylpyrrolidin-1-yl)sulfonyl, (morpholin-4-yl)sulfonyl, (piperidin-1-yl)sulfonyl, (methoxy-1H-indol-1-yl)sulfonyl, {methyl[(1R)-1-phenylethyl]amino}sulfonyl, {methyl[(1S)-1-phenylethyl]amino} sulfonyl, [(2-aminophenyl)(methyl)amino]sulfonyl, (dipropylamino)sulfonyl, benzylsulfanyl, (dipropylamino)sulfanyl, (dipropylamino)sulfinyl, [4-chloro(methyl)anilino]sulfonyl, (phenylthio)methyl, benzyloxy, 3-(ethylthio), (pyridin-4-ylmethyl)thio, phenoxy, phenylthio, (pyridin-4-ylmethyl)thio, benzylthio, (1-phenylethyl)thio, cyclopentylthio, cyclopentylsulfinyl, benzoyl, hydroxy(phenyl)methyl, (methoxyimino)(phenyl)methyl, (hydroxyimino)(phenyl)methyl, cyclopentylcarbonyl, benzoylamino, furoylamino, (thien-2-ylacetyl)amino, (mesitylcarbonyl)amino, (1,3-benzodioxol-5-ylcarbonyl)amino, 3-(2,4-dimethoxybenzoyl)amino, (phenylthio)acetylamino, (anilinocarbonyl)amino, (2,4-difluorophenyl)amino carbonylamino, (3-cyanophenyl)aminocarbonylamino, (3-acetylphenyl)aminocarbonylamino, -(trifluoromethoxy)phenylsulfonylamino, (thien-2-ylacetyl)amino, (5-nitro-2-furoyl)amino, (5-chloro-2-methoxyphenyl)aminocarbonylamino, (4-phenoxyphenyl)aminocarbonylamino, (4-acetylphenyl)aminocarbonylamino, phenylethynyl, 2-phenylethyl, 4-Chlorophenyl, benzyloxy, phenoxy, alkylthio, phenyl, dihalophenyl, amino, acetylamino, benzoylamino, phenylacetylamino, methylsulfonylamino, phenylsulfonylamino, benzylsulfonylamino, benzyloxy, hydroxy, 3-phenoxypropoxy, (2,3-dihydro-1,4-benzodioxin-2-yl)methoxy, cyclobutylmethoxy, (2,2-dimethyl-1,3-dioxolan-4-yl)methoxy, 2,3-dihydroxypropoxy, cyclobutyloxy, 2-methoxy-1-methylethoxy, isopropoxy, cyclopropylmethoxy, cyclohexylmethoxy, 2-methoxyethoxy, tetrahydro-2H-pyran-2-yl-methoxy, (oxiran-2-yl)methoxy, 2-hydroxy-3-isopropoxypropoxy, furylmethoxy, pentyloxy, phenylacetylamino, Benzoylamino, Acetyloxyacetylamino, cyclopentylcarbonylamino, 6-Chloropyridin-3-ylcarbonylamino, isoxazol-5-ylcarbonylamino, 2,4-difluorobenzoylamino, fluoroacetylamino, Acetylamino, 4-Chlorophenylacetylamino, 4-methoxyphenylacetylamino, cyclopentylacetylamino, 3-fluorobenzoylamino, 3-cyanophenylacetylamino, cyclohexylcarbonylamino, propionylamino, 5-methoxy-5-oxopentanoylamino, Butyrylamino, 4-Bromobenzoylamino, 3-phenylpropanoylamino, phenoxyacetylamino, 3-cyclopentylpropanoylamino, 3-methoxy-3-oxopropanoylamino, 2-ethylhexanoylamino, 3,4-dimethoxyphenylacetylamino, 3,5,5-trimethylhexanoylamino, cyclopropylcarbonylamino, methoxyacetylamino, 3-methylbutanoylamino, pentanoylamino, 4,7,7-trimethyl-3-oxo-2-oxabicyclo[2.2.1]hept-1-ylcarbonylamino, Chloro(phenyl)acetylamino, Benzyloxyacetylamino, 3-ethoxy-3-oxopropanoylamino, 1-Adamantylcarbonylamino, hexanoylamino, 2-phenylcyclopranolyamino, 2-phenylbutanoylamino, heptanoylamino, Acetyloxyphenylacetylamino, thien-2-ylcarbonylamino, 2-methylbutanoylamino, 8-methoxy-8-oxooctanoylamino, 2-ethylbutanoylamino, octanoylamino, cyclobutylcarbonylamino, 1,3-dioxo-1,3-dihydro-2H-isoindol-2-yl, Benzylthio, morpholin-4-ylsulfonylbenzoylamino, 1H-indol-2-ylcarbonylamino, 1-methyl-1H-indol-2-ylcarbonylamino, 5-phenylisoxazol-3-ylcarbonylamino, 5-phenylpentanoylamino, 4-phenylbutanoylamino, 4-(4-methoxyphenyl)butanoylamino, 2-Chlorophenylacetylamino, 2,4-dichlorophenylacetylamino, 3,4-dichlorophenylacetylamino, 3-Chlorophenylacetylamino, 3-(trifluoromethyl)phenylacetylamino, 3-methylphenylacetylamino, 4-tert-Butylphenylacetylamino, 3-methoxyphenylacetylamino, 2-methoxyphenylacetylamino, 2-methylphenylacetylamino, 4-(trifluoromethyl)phenylacetylamino, 4-isopropylphenylacetylamino, 4-methylphenylacetylamino, 4-fluorophenylacetylamino, 2-(trifluoromethyl)phenylacetylamino, 3-fluorophenylacetylamino, phenylthioacetylamino, naphthylacetylamino, naphthyloxyacetylamino, 2-propoxybenzoylamino, tetrahydrofuran-3-ylcarbonylamino, 1-methylcyclopropylcarbonylamino, 4-ethoxyphenylacetylamino, 1-Benzothien-3-ylacetylamino, 1,1′-Biphenyl-4-ylcarbonylamino, 4-Butoxybenzoylamino, 2-(2-phenylethyl)benzoylamino, 1,1′-Biphenyl-2-ylcarbonylamino, 4-(ethylthio)benzoylamino, 2-(methylsulfonyl)benzoylamino, 2,6-dichlorophenylacetylamino, 1,1′-Biphenyl-4-ylacetylamino, 1,3-Benzodioxol-5-ylacetylamino, 3,3-dimethylbutanoylamino, thien-2-ylacetylamino, 3-methyl-5-phenylisoxazol-4-ylcarbonylamino, [2-(2-methoxyethoxy)ethoxy]acetylamino, (2-hydroxybenzoyl)amino, prolylamino, (3-methylisoxazol-5-yl)acetylamino, 4-Azido-3-iodobenzoylamino, (diethylamino)sulfonyl, (1H-indol-5-yl)aminosulfonyl, (furylmethylamino)sulfonyl, (ethoxycarbonyl)-1-piperazinylsulfonyl, pyridinylethylaminosulfonyl, (benzylamino)sulfonyl, (2-hydroxy-1-methylethyl)aminosulfonyl, (4-carboxyanilino)sulfonyl, (3,4-dihydro-[(2H)-quinolinyl)sulfonyl, [2-(3,5-dimethoxyphenyl)ethyl]aminosulfonyl, [(3S)-3-hydroxypyrrolidinyl]sulfonyl, (ethylanilino)sulfonyl, (3,5-dimethoxyanilino)sulfonyl, (2-hydroxy-2-phenylethyl)(methyl)amino]sulfonyl, (2,3-dihydro-1H-indol-1-yl)sulfonyl, (5-methoxy-2,3-dihydro-1H-indol-1-yl)sulfonyl, (5-fluoro-2,3-dihydro-1H-indol-1-yl)sulfonyl, (1H-benzimidazol-1-yl)sulfonyl, (5-fluoro-1H-indol-1-yl)sulfonyl, (1H-indol-1-yl)sulfonyl, (6-fluoro-1H-indol-1-yl)sulfonyl, (5-chloro-1H-indol-1-yl)sulfonyl, (6-chloro-1H-indol-1-yl)sulfonyl, (6-chloro-5-fluoro-1H-indol-1-yl)sulfonyl, (1H-pyrrol-1-yl)sulfonyl, (5-methoxy-1H-indol-1-yl)sulfonyl, (1H-pyrrolo[2,3-b]pyridin-1-yl)sulfonyl, (5-bromo-2,3-dihydro-1H-indol-1-yl)sulfonyl, (3,3-dimethyl-2,3-dihydro-1H-indol-1-yl)sulfonyl, (4-chlorophenyl)(methyl)amino]sulfonyl, benzylthio, methyl(pyridin-2-yl)amino]sulfonyl, (1H-indol-1-yl)sulfonyl, (pyrrolidin-1-yl)sulfonyl, (2-methylpyrrolidin-1-yl)sulfonyl, (morpholin-4-yl)sulfonyl, (piperidin-1-yl)sulfonyl, (methoxy-1H-indol-1-yl)sulfonyl, {methyl[(1R)-1-phenylethyl]amino}sulfonyl, {methyl[(1S)-1-phenylethyl]amino}sulfonyl, [(2-aminophenyl)(methyl)amino]sulfonyl, (dipropylamino)sulfonyl, benzylsulfanyl, (dipropylamino)sulfanyl, (dipropylamino)sulfinyl, [4-chloro(methyl)anilino]sulfonyl, (phenylthio)methyl, benzyloxy, 3-(ethylthio), (pyridin-4-ylmethyl)thio, phenoxy, phenylthio, (pyridin-4-ylmethyl)thio, benzylthio, (1-phenylethyl)thio, cyclopentylthio, cyclopentylsulfinyl, benzoyl, hydroxy(phenyl)methyl, (methoxyimino)(phenyl)methyl, (hydroxyimino)(phenyl)methyl, cyclopentylcarbonyl, benzoylamino, furoylamino, (thien-2-ylacetyl)amino, (mesitylcarbonyl)amino, (1,3-benzodioxol-5-ylcarbonyl)amino, 3-(2,4-dimethoxybenzoyl)amino, (phenylthio)acetylamino, (anilinocarbonyl)amino, (2,4-difluorophenyl)amino carbonylamino, (3-cyanophenyl)aminocarbonylamino, (3-acetylphenyl)aminocarbonylamino, -(trifluoromethoxy)phenylsulfonylamino, (thien-2-ylacetyl)amino, (5-nitro-2-furoyl)amino, (5-chloro-2-methoxyphenyl)aminocarbonylamino, (4-phenoxyphenyl)aminocarbonylamino, (4-acetylphenyl)aminocarbonylamino, phenylethynyl, 2-phenylethyl, 4-Chlorophenyl, benzyloxy, phenoxy, alkylthio, phenyl, dihalophenyl, amino, acetylamino, benzoylamino, phenylacetylamino, methylsulfonylamino, phenylsulfonylamino, and benzylsulfonylamino.

35. The compound of claim 1, wherein R₆, is H, halo, —CN, NH₂, NO₂, methyl, methoxy, —(CH₂)₂—OH, morpholinyl, and —(CH₂)₂—O—CO—CH₃.

36. The compound of claim 1, wherein R₁ is 5-oxo-4,5-dihydro-1,2,4-oxadiazol-3-yl, 5-oxo-4,5-dihydro-1,3,4-oxadiazol-2-yl, methylsulfonylaminocarbonyl, 4-methylphenylsulfonylaminocarbonyl, 1H-tetraazol-5-yl, hydrazinocarbonylphenyl, 5-thioxo-4,5-dihydro-1,3,4-oxadiazol-2-yl, 1,1-dioxido-2H-1,2,4-benzothiadiazin-3-yl, 4-oxo-3,4-dihydroquinazolin-2-yl, amino(hydroxyimino)methyl, 2H-tetraazol-2-yl-methyl pivalate.

37. A method for sanitizing or disinfecting comprising administering an effective amount of the antibacterial compound of claim 1.
